SEQUENCE LISTING (915 sequences)

SEQ ID NO: 1 - skipped

SEQ ID NO: 2 - protein, 550 aa, synthetic construct
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 3 - RNA, 1843 nt, synthetic construct
  source 1..1843: mol_type = other RNA
acatttgcttctgacacaactgtgttcactagcaacctcaaacagccaccatggaagatgccaaaaacatcaagaagggtccggccccattctacccactggaagatggtaccgcgggcgaacagctgcataaggccatgaagagatacgccctggtccctggaaccatcgcgttcaccgacgcccacattgaagtggacatcacctacgccgaatacttcgagatgtccgtgcggctggccgaagccatgaagcgctacggcctgaacacgaaccataggatcgtggtctgctccgaaaacagcctgcagtttttcatgccggtgctcggagccctgtttatcggcgtggcggtcgcaccagcaaacgacatctacaatgagcgggaactactgaactcgatgggtatttcccaacccactgtggtgttcgtcagcaaaaagggcctgcaaaagatcctgaacgtgcagaagaagctgcccatcatccagaagatcattattatggacagcaagacggattaccaaggcttccagtcgatgtacaccttcgtgacctcgcatctgccaccgggtttcaacgaatacgacttcgtgcccgaatcgtttgaccgcgacaagactattgccttgatcatgaacagcagcggtagtactggtttgcctaagggggtggccctgccacaccggaccgcctgcgtgagattctcgcacgcgcgcgatcccatattcggaaatcagatcatccctgacacggctatcctgtccgtggtcccattccaccacggcttcggcatgttcaccactctcggatacctgatttgcggattccgggtggtgctgatgtaccgcttcgaagaggagctgtttctccggagccttcaggactacaaaatccagagcgccctcctggtgcccaccctgttctccttcttcgctaagtctaccttgattgacaaatacgacctctcgaacctccacgagatcgcatccggtggtgccccattgtcgaaggaagtcggagaagcagtggcgaagcggttccacctcccgggcattcggcagggttacgggctgactgaaaccacctcagcgatcctcattactcctgagggggacgacaagccgggcgccgtgggaaaggttgtgccgttcttcgaggccaaggtcgtcgacctggataccggaaagaccctgggagtcaaccaaaggggggagctgtgtgtgcgcggacctatgattatgtccggatacgtcaacaaccctgaggccactaacgccctgatcgacaaggacggttggctgcactccggagatatcgcatactgggacgaggacgaacacttcttcattgtggatcggttgaagtccctgatcaagtacaagggctatcaagtggcaccggccgaacttgaatcgatccttctgcaacaccccaacatcttcgatgccggcgtcgccgggctgcccgacgacgacgcgggagagctgccggctgcggtggtggtgcttgagcacggcaaaaccatgactgaaaaagagatcgtggactacgtggcgtcccaagtcaccaccgcgaagaagctcaggggcggcgtggtgtttgtggatgaggtgcccaaggggctcaccggaaagttggacgcccgcaagattcgcgagattctgattaaggccaagaaggggggaaagattgccgtgtgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 4 - RNA, 628 nt, synthetic construct
  source 1..628: mol_type = other RNA
acatttgcttctgacacaactgtgttcactagcaacctcaaacagacaccatggtgcatctgactcctgaggagaagtctgccgttactgccctgtggggcaaggtgaacgtggatgaagttggtggtgaggccctgggcaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcaggctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 5 - protein, 567 aa, unidentified
MENIVLLLAIVSLVKSDQICIGYHANNSTEQVDTIMEKNVTVTHAQDILEKTHNGKLCDLNGVKPLILKDCSVAGWLLGNPMCDEFIRVPEWSYIVERANPANDLCYPGSLNDYEELKHMLSRINHFEKIQIIPKSSWPNHETSLGVSAACPYQGAPSFFRNVVWLIKKNDAYPTIKISYNNTNREDLLILWGIHHSNNAEEQTNLYKNPITYISVGTSTLNQRLAPKIATRSQVNGQRGRMDFFWTILKPDDAIHFESNGNFIAPEYAYKIVKKGDSTIMKSGVEYGHCNTKCQTPVGAINSSMPFHNIHPLTIGECPKYVKSNKLVLATGLRNSPLREKRRKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESVGTYQILSIYSTAASSLALAIMMAGLSLWMCSNGSLQCRICI

SEQ ID NO: 6 - RNA, 1894 nt, synthetic construct
  source 1..1894: mol_type = other RNA
acatttgcttctgacacaactgtgttcactagcaacctcaaacagccaccatggagaacatcgtgctcttgctggcaatcgtgtccctggtcaagtccgatcagatctgcatcggctaccacgcaaacaactccaccgaacaagtggacactatcatggaaaagaacgtgacggtcacacacgcccaggacattctggaaaagacccacaatgggaaactgtgtgacctcaacggcgtgaagccgctgatcctcaaggattgctccgtggctggatggctcctgggcaaccctatgtgcgatgagttcattcgggtgcccgagtggtcgtacattgtggagagggccaacccggccaatgacctgtgctaccccggctcgctgaacgattacgaagaactcaagcacatgctgtcccgcatcaaccatttcgagaagatccagatcattccgaagtccagctggcctaaccacgaaacttccctgggagtgtctgctgcctgcccataccaaggcgccccctcatttttccggaacgtcgtgtggctcatcaagaagaacgatgcgtaccccactatcaagatcagctacaacaacaccaaccgcgaggatctgctgattctctggggaattcaccattccaacaacgctgaagaacagaccaacctgtacaaaaaccctatcacttacatctccgtgggtacctccaccctcaatcagcgcttggcgccgaagatcgcgaccagaagccaagtcaacggacagcggggaagaatggacttcttctggactatactgaagcctgacgatgcaatccacttcgaatccaacggaaacttcatcgcccctgagtatgcctacaagatcgtgaagaagggcgacagcaccatcatgaagtcaggagtggagtatggacactgtaacactaagtgccagacccccgtcggggccattaacagctccatgccgttccacaacattcaccccctgactattggagagtgtccgaaatacgtgaagagtaacaagttggtgctggccactggcctgcgcaactcgccgcttcgggagaagaggaggaagagaggactgtttggggcgattgcaggattcattgagggaggttggcaggggatggtggacggttggtacggttaccaccactcgaacgagcagggatcgggctatgccgccgacaaggagagcacccagaaggccatcgacggggtcaccaacaaggtcaactccatcattgacaagatgaatacccagttcgaagccgtgggccgcgaattcaacaacctggaacgacggatcgagaacctgaacaagaagatggaggacggcttcctggacgtgtggacctacaatgccgaactgctcgtgctgatggagaatgagcggaccctcgacttccatgactcaaacgtgaagaacctgtacgacaaagtccggctgcaactgcgggacaatgccaaggaactggggaacggatgctttgagttctaccataagtgcgataacgagtgcatggaatcagtgcggaacggaacctacgactacccacagtactccgaggaagcccgcctgaagagagaagaaatctccggcgtgaaactcgaaagcgtggggacctaccagatcctttccatctactccacggctgcgagctcgttggcccttgcgatcatgatggccggcctttcgctgtggatgtgctccaacggtagcctgcagtgccgcatttgtatctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 7 - protein, 584 aa, synthetic construct
MENIVLLLAIVSLVKSDQICIGYHANNSTEQVDTIMEKNVTVTHAQDILEKTHNGKLCDLNGVKPLILKDCSVAGWLLGNPMCDEFIRVPEWSYIVERANPANDLCYPGSLNDYEELKHMLSRINHFEKIQIIPKSSWPNHETSLGVSAACPYQGAPSFFRNVVWLIKKNDAYPTIKISYNNTNREDLLILWGIHHSNNAEEQTNLYKNPITYISVGTSTLNQRLAPKIATRSQVNGQRGRMDFFWTILKPDDAIHFESNGNFIAPEYAYKIVKKGDSTIMKSGVEYGHCNTKCQTPVGAINSSMPFHNIHPLTIGECPKYVKSNKLVLATGLRNSPLREKRRKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESVGTYQIGIVAGLAVLAVVVIGAVVAAVMCRRKSSGGKGGSYSQAACSDSAQGSDVSLTA

SEQ ID NO: 8 - RNA, 1945 nt, synthetic construct
  source 1..1945: mol_type = other RNA
acatttgcttctgacacaactgtgttcactagcaacctcaaacagccaccatggagaacatcgtgctcttgctggcaatcgtgtccctggtcaagtccgatcagatctgcatcggctaccacgcaaacaactccaccgaacaagtggacactatcatggaaaagaacgtgacggtcacacacgcccaggacattctggaaaagacccacaatgggaaactgtgtgacctcaacggcgtgaagccgctgatcctcaaggattgctccgtggctggatggctcctgggcaaccctatgtgcgatgagttcattcgggtgcccgagtggtcgtacattgtggagagggccaacccggccaatgacctgtgctaccccggctcgctgaacgattacgaagaactcaagcacatgctgtcccgcatcaaccatttcgagaagatccagatcattccgaagtccagctggcctaaccacgaaacttccctgggagtgtctgctgcctgcccataccaaggcgccccctcatttttccggaacgtcgtgtggctcatcaagaagaacgatgcgtaccccactatcaagatcagctacaacaacaccaaccgcgaggatctgctgattctctggggaattcaccattccaacaacgctgaagaacagaccaacctgtacaaaaaccctatcacttacatctccgtgggtacctccaccctcaatcagcgcttggcgccgaagatcgcgaccagaagccaagtcaacggacagcggggaagaatggacttcttctggactatactgaagcctgacgatgcaatccacttcgaatccaacggaaacttcatcgcccctgagtatgcctacaagatcgtgaagaagggcgacagcaccatcatgaagtcaggagtggagtatggacactgtaacactaagtgccagacccccgtcggggccattaacagctccatgccgttccacaacattcaccccctgactattggagagtgtccgaaatacgtgaagagtaacaagttggtgctggccactggcctgcgcaactcgccgcttcgggagaagaggaggaagagaggactgtttggggcgattgcaggattcattgagggaggttggcaggggatggtggacggttggtacggttaccaccactcgaacgagcagggatcgggctatgccgccgacaaggagagcacccagaaggccatcgacggggtcaccaacaaggtcaactccatcattgacaagatgaatacccagttcgaagccgtgggccgcgaattcaacaacctggaacgacggatcgagaacctgaacaagaagatggaggacggcttcctggacgtgtggacctacaatgccgaactgctcgtgctgatggagaatgagcggaccctcgacttccatgactcaaacgtgaagaacctgtacgacaaagtccggctgcaactgcgggacaatgccaaggaactggggaacggatgctttgagttctaccataagtgcgataacgagtgcatggaatcagtgcggaacggaacctacgactacccacagtactccgaggaagcccgcctgaagagagaagaaatctccggcgtgaaactcgaaagcgtggggacctaccagatcgggattgtggccggactggctgtgttggccgtggtggtcatcggcgccgtcgtggcggccgtgatgtgccggcgcaagtcctccgggggcaaagggggaagctattcccaagccgcctgctctgactccgcccaggggtcggatgtgtccctgaccgcctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 9 - protein, 535 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAICSGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 10 - RNA, 1798 nt, synthetic construct
  source 1..1798: mol_type = other RNA
acatttgcttctgacacaactgtgttcactagcaacctcaaacagccaccatggaactcctcatcctgaaagccaacgccatcaccacaattctcactgctgtgaccttctgttttgcctcgggacagaacatcaccgaggagttctaccagtccacatgctccgctgtgtcgaagggctacctgtcggcgctgaggactggctggtacacctccgtgattaccatcgaactgtcaaacatcaaggagaacaagtgcaacggtactgacgcgaaggtcaagctgattaagcaggaattggacaagtacaagaacgccgtgactgagctccagctgctgatgcagtcaacccctgcgacgggaagcgggtccgcaatctgctccggagtggccgtgtgcaaagtgctgcatctggagggcgaagtcaacaagatcaagtcggccctgctgagcaccaacaaggccgtggtgtccttgagtaacggagtctccgtcctgaccttcaaagttctggacctgaagaattacatcgataagcagctcctgcccatcctcaacaagcagagctgctccatttcgaacatcgaaactgtgatcgagtttcagcaaaagaacaaccggctgctggaaatcaccagagagtttagcgtgaacgcaggagtgactacccccgtgtctacctacatgctaaccaactcagaactgcttagcttgatcaatgacatgccgatcactaacgatcagaagaagcttatgtccaacaacgtgcaaatcgtgcgccagcaatcctactcgattatgtgcattatcaaggaagaggtgctggcctatgtggtgcagctcccgctgtacggcgtgattgacactccttgctggaagctgcacacctcccccctgtgtaccacgaataccaaggaggggtccaatatctgcctgacccggaccgaccgcggatggtactgcgacaacgccggttcggtgtccttcttcccacaagccgagacttgcaaagtccagtccaatcgcgtgttctgcgacactatgaactcccggaccttgccgtccgaagtgaacctgtgtaacgtggacatcttcaaccctaaatacgactgcaagattatgacttccaagaccgacgtcagcagctcagtgatcaccagcctgggcgccatcgtgtcgtgttatggaaagaccaagtgcactgcgtccaacaagaacagaggcatcatcaagaccttcagcaacggctgcgattacgtcagcaacaagggggtggataccgtgtccgtgggaaacactctctactgtgtgaacaagcaggagggaaagtccctctacgtcaagggcgaaccgatcatcaacttctacgatcccctggtgttcccatccgacgagttcgacgcctccatttcacaagtcaacgaaaagatcaaccagtctctggcgttcatccggaagtcggatgaactccttcacaacgtcaatgccggaaagtccactactaacatcatgattaccaccatcatcattgtcattattgtgatacttctgagcctcatcgccgtgggtctgctgttgtactgcaaagctcgctccacgcctgtgaccctgtccaaggatcagctcagcgggattaacaacatcgccttctcaaactgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 11 - skipped

SEQ ID NO: 12 - protein, 566 aa, Alphainfluenzavirus influenzae
MKAILVVMLYTFTTANADTLCIGYHANNSTDTVDTVLEKNVTVTHSVNLLEDKHNGKLCKLRGVAPLHLGQCNIAGWILGNPECESLSTARSWSYIVETSNSDNGTCYPGDFINYEELREQLSSVSSFERFEIFPKTSSWPNHDSDNGVTAACSHAGARSFYKNLIWLVKKGKSYPKINQTYINDKGKEVLVLWGIHHPPTITDQESLYQNADAYVFVGTSRYSKKFKPEIATRPKVRDQAGRMNYYWTLVEPGDKITFEATGNLVAPRYAFTMEKEAGSGIIISDTPVHDCNATCQTPEGAINTSLPFQNVHPITIGKCPKYVRSTKLRLATGLRNVPSIQSRGLFGAIAGFIEGGWTGMVDGWYGYHHQNDQGSGYAADLKSTQNAIDKITNKVNSVIEKMNTQFTAVGKEFNHLEKRIENLNKKVDDGFLDVWTYNAELLVLLENERTLDYHDSNVKNLYEKVRHQLKNNAKEIGNGCFEFYHKCDNTCMESVKNGTYDYPKYSEEAKLNREKIDGVKLDSTRIYQILAIYSTVASSLVLVVSLGAISFWMCSNGSLQCRICI

SEQ ID NO: 13 - RNA, 1891 nt, synthetic construct
  source 1..1891: mol_type = other RNA
acatttgcttctgacacaactgtgttcactagcaacctcaaacagccaccatgaaagccatcctcgtcgtaatgctgtacaccttcactaccgccaatgccgacaccctgtgcatcggataccatgccaacaactccaccgacaccgtggacactgtgctggagaagaacgtgaccgtgacccactcggtgaacttgctggaagataagcacaacggaaagctgtgcaagctcaggggggtcgcacctctgcatctgggacagtgcaacatcgctggctggatcctgggaaacccggagtgcgagagcctgtccacggccagaagctggagctacatcgtggaaacttcgaattcggataacggcacctgttatcccggggatttcattaactacgaggaactgcgcgagcagctctcatcggtgtcctccttcgaacgcttcgaaatcttccctaaaacctcatcctggcctaaccacgacagcgacaacggcgtgactgcagcctgctcccacgccggggcgcggtcgttctacaagaacctcatctggctggtcaagaagggaaagtcctatcctaaaatcaaccagacctacatcaacgacaagggaaaggaagtgttggtgctctgggggatccatcatcccccgaccatcaccgaccaggaaagcctgtaccagaacgccgatgcctacgtgtttgtcggaacctcgcgctactcaaagaagtttaagccggagattgccacccggccgaaagtccgggaccaggctggacggatgaactactactggaccctggtggaaccaggggacaagatcacattcgaagcgaccggaaacctggtggcacctagatacgcgttcaccatggagaaggaggccggttccggtatcatcattagcgacaccccggtgcacgactgcaacgctacttgccaaactcccgaaggcgccatcaataccagccttcccttccaaaacgtgcacccaattaccatcggaaagtgtcccaaatacgtgcggtccactaagttgaggttggcgactgggctgaggaatgtgcccagcattcagtcccggggactgttcggagccatcgccggtttcatcgagggcggctggactggaatggtggacggttggtacggctaccaccaccaaaacgaccagggctcaggctacgcggctgaccttaagtccactcagaatgcgatcgataagatcacgaacaaggtcaactccgtgattgaaaagatgaacactcagtttaccgccgtcggaaaggaattcaaccatctggagaagcgcattgaaaacctgaacaagaaggtcgatgacggtttcctcgacgtgtggacctacaacgcggagctgcttgtgctgctcgagaacgagcggaccctggactatcacgatagtaacgtgaagaacctgtacgagaaggtccgccaccaactgaagaacaacgcaaaggaaatcggcaacggctgtttcgagttctaccacaaatgcgataacacttgcatggaatccgtgaagaatggcacctacgactacccgaagtactccgaagaggccaagctcaaccgagagaagattgatggagtgaagctggactctactagaatctaccagatacttgccatctactccaccgtggcctcaagcctggtgctcgtggtgtccctgggagccatttccttctggatgtgctccaacgggtcgctccagtgtcgcatttgcatttgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 14 - protein, 566 aa, Alphainfluenzavirus influenzae
MKTIIALSNILCLVFAQKIPGNDNSTATLCLGHHAVPNGTIVKTITNDRIEVTNATELVQNSSIGEICGSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRGIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAMSCFLLCIALLGFIMWACQKGNIRCNICI

SEQ ID NO: 15 - protein, 567 aa, synthetic construct
MENIVLLLAIVSLVKSDQICIGYHANNSTEQVDTIMEKNVTVTHAQDILEKTHNGKLCDLNGVKPLILKDCSVAGWLLGNPMCDEFIRVPEWSYIVERANPANDLCFPGSLNDYEELKHMLSRINHFEKIQIIPKSSWPNHETSLGVSAACPYQGAPSFFRNVVWLIKKNDAYPTIKISYNNTNREDLLILWGIHHSNNAEEQTNLYKNPITYISVGTSTLNQRLAPKIATRSQVNGQRGRMDFFWTILKPDDAIHFESNGNFIAPEYAYKIVKKGDSTIMKSGVEYGHCNTKCQTPVGAINSSMPFHNIHPLTIGECPKYVKSNKLVLATGLRNSPLREKRRKRGLFGAIAGFIEGGWQGMVDGWYGYHFSNEQGSGYAADKESTQKAIDGVTNMVNSIIDKMNTQFEAVGMEFNNLERRIENLNKKMEDGFIDVWTYNAELLVLMLNERTLDFHDSNVKNLYDKVRLQLRDNAKELGNGCFEFYHKCDNECMESVRNGTYDYPQYSEEARLKREEISGVKLESVGTYQILSIYSTAASSLALAIMMAGLSLWMCSNGSLQCRICI

SEQ ID NO: 16 - RNA, 1894 nt, synthetic construct
  source 1..1894: mol_type = other RNA
acatttgcttctgacacaactgtgttcactagcaacctcaaacagccaccatggagaacatcgtgctcttgctggcaatcgtgtccctggtcaagtccgatcagatctgcatcggctaccacgcaaacaactccaccgaacaagtggacactatcatggaaaagaacgtgacggtcacacacgcccaggacattctggaaaagacccacaatgggaaactgtgtgacctcaacggcgtgaagccgctgatcctcaaggattgctccgtggctggatggctcctgggcaaccctatgtgcgatgagttcattcgggtgcccgagtggtcgtacattgtggagagggccaacccggccaatgacctgtgcttccccggctcgctgaacgattacgaagaactcaagcacatgctgtcccgcatcaaccatttcgagaagatccagatcattccgaagtccagctggcctaaccacgaaacttccctgggagtgtctgctgcctgcccataccaaggcgccccctcatttttccggaacgtcgtgtggctcatcaagaagaacgatgcgtaccccactatcaagatcagctacaacaacaccaaccgcgaggatctgctgattctctggggaattcaccattccaacaacgctgaagaacagaccaacctgtacaaaaaccctatcacttacatctccgtgggtacctccaccctcaatcagcgcttggcgccgaagatcgcgaccagaagccaagtcaacggacagcggggaagaatggacttcttctggactatactgaagcctgacgatgcaatccacttcgaatccaacggaaacttcatcgcccctgagtatgcctacaagatcgtgaagaagggcgacagcaccatcatgaagtcaggagtggagtatggacactgtaacactaagtgccagacccccgtcggggccattaacagctccatgccgttccacaacattcaccccctgactattggagagtgtccgaaatacgtgaagagtaacaagttggtgctggccactggcctgcgcaactcgccgcttcgggagaagaggaggaagagaggactgtttggggcgattgcaggattcattgagggaggttggcaggggatggtggacggttggtacggttaccacttctcgaacgagcagggatcgggctatgccgccgacaaggagagcacccagaaggccatcgacggggtcaccaacatggtcaactccatcattgacaagatgaatacccagttcgaagccgtgggcatggaattcaacaacctggaacgacggatcgagaacctgaacaagaagatggaggacggcttcatcgacgtgtggacctacaatgccgaactgctcgtgctgatgctgaatgagcggaccctcgacttccatgactcaaacgtgaagaacctgtacgacaaagtccggctgcaactgcgggacaatgccaaggaactggggaacggatgctttgagttctaccataagtgcgataacgagtgcatggaatcagtgcggaacggaacctacgactacccacagtactccgaggaagcccgcctgaagagagaagaaatctccggcgtgaaactcgaaagcgtggggacctaccagatcctttccatctactccacggctgcgagctcgttggcccttgcgatcatgatggccggcctttcgctgtggatgtgctccaacggtagcctgcagtgccgcatttgtatctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 17 - RNA, 1939 nt, synthetic construct
  source 1..1939: mol_type = other RNA
acatttgcttctgacacaactgtgttcactagcaacctcaaacagccaccatgaaggccattattgtgctcctcatggtcgtgacttcaaacgcggacaggatctgcactgggattacctcctcgaactccccgcacgtggtcaaaactgcaactcaaggagaagtcaacgtgaccggggtcatacccctcactacgaccccgaccaaatcccatttcgcgaaccttaagggtaccgagactcggggaaaactctgcccgaagtgtctgaactgtactgacctggatgtggcgctcgggcgcccaaagtgcacgggaaagattccgtccgcacgcgtcagcatcttgcacgaagtcaggcccgtgacctccggatgctttccaattatgcacgaccggacgaagatccgccagctcccgaacctcttgaggggttatgagcacgtccgcctctcgactcataacgtcattaacaccgaggatgcgccgggaggaccctacgaaattggtacctcgggatcatgccttaacatcaccaacgggaagggattctttgccaccatggcctgggcggtgcccaagaacaagaccgccaccaaccctcttaccatcgaagtgccatacatctgcaccgaggaagaggaccagatcactgtgtggggtttccattccgacgacgaaactcagatggcccgcctctacggggactccaagccgcaaaagttcacctcgtccgccaatggagtgactacccactacgtgtcccaaattggagggttcccgaatcaaactgaggatggtggactgccccagagcggccggattgtggtggattacatggtccagaaaagcggcaagaccggcaccatcacctaccagcggggaatcctgctgcctcagaaggtgtggtgtgcttccggcaagagcaaggtgatcaagggcagcctgcctctgatcggcgaagccgactgcctgcacgagaagtacggcggcctgaacaagagcaagccttactacacaggcgaacatgccaaggccatcggcaactgccccatctgggtgaagacccccctgaagctggccaacggaaccaagtacagacctcccgccaagctgctgaaggaacggggcttcttcggagccatcgccggcttcctggaaggaggctgggagggcatgattgccgggtggcacggctacacctctcatggagcacacggggtggccgtggctgctgacctgaagtctacacaggaggccatcaacaagatcaccaagaacctgaactctttgtctgagctggaggtgaaaaacctgcagagactgtcaggcgccatggacgaactgcacaatgagatcctggaactggatgaaaaggtggacgacctgagagccgacaccatcagctcccagatcgaactggctgtgctgctgagcaacgagggaatcatcaattccgaggacgaacacctgctggccctggagcggaagctgaagaagatgctgggcccttctgccgtggaaatcggcaacggctgcttcgaaaccaagcacaagtgcaaccagacctgcctggaccggattgcagccggcacctttgacgccggagagttctccttgccgactttcgactcgctcaacatcactgcggcctcgcttaacgacgatggtctggataaccacactatccttttgtattactccaccgcggcgtcgtccctcgccgtgaccctcatgatcgctattttcgtggtgtacatggtgtcccgcgataacgtgtcctgttcaatctgcctgtgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 18 - protein, 566 aa, unidentified
MKAIIALSNILCLVFAQKIPGNDNSTATLCLGHHAVPNGTIVKTITNDRIEVTNATELVQNSSIGKICNSPHQILDGGNCTLIDALLGDPQCDGFQNKEWDLFVERSRANSSCYPYDVPDYASLRSLVASSGTLEFKDESFNWTGVKQNGTSSACKRGSSNSFFSRLNWLTSLNNIYPAQNVTMPNKEQFDKLYIWGVHHPDTDKNQFSLFAQSSGRITVSTKRSQQAVIPNIGSRPRVRDIPSRISIYWTIVKPGDILLINSTGNLIAPRGYFKIRSGKSSIMRSDAPIGECKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQISGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAMSCFLLCIALLGFIMWACQKGNIRCNICI

SEQ ID NO: 19-31 - skipped (13 entries)

SEQ ID NO: 32 - protein, 17 aa, Alphainfluenzavirus influenzae
MKAILVVMLYTFTTANA

SEQ ID NO: 33 - protein, 36 aa, Alphainfluenzavirus influenzae
LAIYSTVASSLVLVVSLGAISFWMCSNGSLQCRICI

SEQ ID NO: 34 - skipped

SEQ ID NO: 35 - protein, 28 aa, Homo sapiens
MAAPGSARRPLLLLLLLLLLGLMHCASA

SEQ ID NO: 36 - protein, 35 aa, Homo sapiens
LIPIAVGGALAGLVLIVLIAYLVGRKRSHAGYQTI

SEQ ID NO: 37 - protein, 469 aa, Alphainfluenzavirus influenzae
MNPNQKIITIGSVCMTIGTANLILQIGNIISIWVSHSIQIGNQSQIETCNKSVITYENNTWVNQTFVNISNTNSAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVLSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDN

SEQ ID NO: 38 - protein, 469 aa, Alphainfluenzavirus influenzae
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDLDKCYQFALGQGTTLNNVHSNNTVRDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 39 - protein, 466 aa, Betainfluenzavirus influenzae
MLPSTIQTLTLFLTSGGVLLSLYVSASLSYLLYSDILLKFSQTEITAPTMPLDCANASNVQAVNRSATKGATLLLPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDEGSGGIKGGFVHQRMKSKIGRWYSRTMSKTERMGMGLYVKYGGDPWADSDALVFSGVMISMKEPGWYSFGFEIKDKKCDVPCIGIEMVHDGGKETWHSAATAIYCLMGSGQLLWDTITGVDMAL

SEQ ID NO: 40-400 - skipped (361 entries)

SEQ ID NO: 401 - protein, 1123 aa, synthetic construct
MRVTAPRTLILLLSGALALTETWAAEMKTDAATLAQEAGNFERISGDLKTQIDQVESTAGSLQGQWRGAAGTAAQAAVVRFQEAANKQKQELDEISTNIRQAGVQYSRADEEQQQALSSQMGFGPGPGVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMGPGPGTEQQWNFAGIEAAASAIQGNVTSIHSLLDEGKQSLTKLAAAWGGSGSEAYQGVQQKWDATATELNNALQNLARTISEAGQAMASTEGNVTGMFAGPGPGTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSGPGPGSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGGGPGPGTINYQFGDVDAHGAMIRAQAGSLEAEHQAIISDVLTASDFWGGAGSAACQGFITQLGRNFQVIYEQANAHGQKVQAAGNNMAQTDSAVGSSWAGPGPGPVGGQSSFYSDWYSPACGKAGCQTYKWETFLTSELPQWLSANRAVKPTGSAAIGLSMAGSSAMILAAYHPQQFIYAGSLSALLDPSQGMGGPGPGAQIYQAVSAQAAAIHGPGPGPSPSMGRDIKVQFQSGPGPGGINTIPIAINEAEYVGPGPGAAFQGAHARFVAAAAGPGPGAGWLAFFRDLVARGLGPGPGASIIRLVGAVLAEQHGPGPGMSFVTTQPEALAAAAGPGPGMHVSFVMAYPEMLAAGPGPGAYGSFVRTVSLPVGAGPGPGLENDNQLLYNYPGALGIVAGLAVLAVVVIGAVVAAVMCRRKSSGGKGGSYSQAACSDSAQGSDVSLTA

SEQ ID NO: 402 - RNA, 3562 nt, synthetic construct
  source 1..3562: mol_type = other RNA
acatttgcttctgacacaactgtgttcactagcaacctcaaacagccaccatgcgggtcactgcccccagaaccttgattttactactgtcgggagccctcgctctgactgaaacatgggccgctgagatgaaaaccgacgccgcgaccctggcccaggaagccggaaacttcgagcggatttccggagatcttaagactcagatcgaccaggtcgaatccaccgccggatcgctccagggacagtggagaggcgccgcgggaaccgcggcccaggccgccgtcgtgcggttccaagaggccgccaacaagcagaagcaggaactggacgaaatttccactaacattcgccaagctggcgtgcagtactcgagagccgatgaagaacagcagcaagccctctcctcacaaatgggtttcggacctgggcccggcgtggacttcggagccctgccacctgaaatcaactccgcccggatgtacgcaggacctggatccgccagcctggtggcggccgcgcagatgtgggatagcgtggcctccgacctgttcagcgcggcttcagctttccaaagcgtggtctggggactgactgtgggatcctggatcggatcaagcgcaggcctcatggtggccgcggcgtccccttacgtggcctggatgagcgtgaccgccggccaggcagaactgaccgcagcccaagtccgcgtggccgcagccgcctacgagactgcctacggtctgacggtgccgccgccagtgatcgccgagaacagagcagagctcatgatcctcatcgcgaccaacctactgggccagaacactccggcgattgccgtgaacgaagccgaatatggggagatgtgggctcaggacgctgcagccatgttcggatatgcggccgcgactgctaccgccaccgccactctgctgccgttcgaggaggcccccgaaatgacttccgcgggaggcctgttggagcaagctgccgcggtggaggaagctagcgacaccgccgcagccaaccagctcatgaacaacgtgccacaggccctgcagcagctggcccagcccactcaagggaccaccccgagctcaaagctgggcggtctgtggaaaaccgtgtccccccaccgctcgcccatttccaacatggtgtcaatggcgaacaaccacatgggtcctggacctggaacggagcagcagtggaacttcgccgggatcgaagccgccgcctcggctatccaagggaatgtcacctcgatccattcccttctggacgaaggaaagcagtccctgaccaagcttgcggcagcctggggcggaagcggcagcgaagcctaccagggcgtgcagcaaaaatgggacgcaaccgccactgagctgaacaacgccctccaaaacctggctagaactatttccgaggccggacaggctatggccagcaccgagggcaacgtgaccgggatgttcgctggccctgggccgggcacctcgcggttcatgaccgatcctcatgctatgcgggatatggctggacggttcgaggtgcacgcccaaactgtggaggacgaggcccgccggatgtgggccagcgcgcagaacatctcgggggccggctggtccggaatggccgaagccacctccctcgataccatgacccagatgaaccaggccttccggaacattgtgaacatgctgcatggagtgcgggacggcctggtccgggacgccaacaattacgagcagcaggagcaggcctctcagcaaatcctgtccagcgggccggggcctggatcacaaatcatgtacaactacccggcaatgctgggccacgctggagacatggccggctacgccggcacgctgcagtcactcggtgccgagatcgccgtcgagcaggcggcactgcagagcgcttggcagggagacactggcattacctaccaagcgtggcaggctcagtggaatcaagctatggaagatctggtccgcgcgtaccacgccatgtcctccactcacgaagccaacaccatggctatgatggccagggacactgccgaggcagccaaatgggggggaggaccggggccgggaaccataaactaccagtttggcgacgtcgacgcccacggagccatgatcagggcgcaggccgggtcgctggaagcagaacaccaggccatcatctccgatgtgctgaccgcctccgacttttggggaggagccggttcggctgcctgccaagggttcatcacacaattgggaaggaacttccaggtcatctacgagcaggccaatgcacacggtcaaaaggtccaagcggcgggcaacaacatggcccagactgactcggccgtgggcagcagttgggccggtcccggccctggccctgtcggggggcagtcatccttctactccgactggtactcccccgcctgcggaaaggccggctgtcagacctataagtgggaaaccttcctgacctccgaactcccgcagtggctctcggcgaatcgggcagtgaagcccaccgggtcggcggctattggactgtccatggccggttccagcgcgatgatcctggccgcctatcacccccagcaattcatctacgctggctccctgtccgccctgcttgacccatcgcaggggatgggtggaccgggccctggggcccagatctaccaagcagtgtcggcccaagccgcagcgatccatggaccaggaccgggaccgtccccgagcatgggccgggatattaaggttcagttccagtccggacccggcccggggggtattaacaccattccgattgccattaatgaggcagaatacgtcggccctggtcccggcgccgcgttccaaggagcccacgcacgattcgtggcggcagctgcgggacccgggcctggtgccggttggctggcttttttccgcgacctggtggccagaggacttggacccggtccgggcgccagcattatcaggctggtcggggccgtgctggcggaacagcatggacctggcccgggaatgagcttcgtgaccacccaacctgaggcgcttgccgcggcggcaggccctggacccgggatgcatgtgtcttttgtgatggcctacccggaaatgctggccgcgggtccgggccccggagcctacggatcattcgtgcgcactgtgtccctccccgtgggagccggacctggaccggggttggagaatgataaccagctgctctacaactatccgggggccctcggcatcgtggcgggcctcgctgtcctcgccgtggtggtgatcggagccgtcgtcgccgctgtgatgtgccgccgcaagtccagcggcggaaaggggggcagctatagccaggcagcctgttccgactccgcgcaggggtcggatgtgtccttgaccgcctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 403 - protein, 1109 aa, synthetic construct
MAAPGSARRPLLLLLLLLLLGLMHCASAAEMKTDAATLAQEAGNFERISGDLKTQIDQVESTAGSLQGQWRGAAGTAAQAAVVRFQEAANKQKQELDEISTNIRQAGVQYSRADEEQQQALSSQMGFGPGPGVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMGPGPGTEQQWNFAGIEAAASAIQGNVTSIHSLLDEGKQSLTKLAAAWGGSGSEAYQGVQQKWDATATELNNALQNLARTISEAGQAMASTEGNVTGMFAGPGPGTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSGPGPGSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGGGPGPGTINYQFGDVDAHGAMIRAQAGSLEAEHQAIISDVLTASDFWGGAGSAACQGFITQLGRNFQVIYEQANAHGQKVQAAGNNMAQTDSAVGSSWAGPGPGPVGGQSSFYSDWYSPACGKAGCQTYKWETFLTSELPQWLSANRAVKPTGSAAIGLSMAGSSAMILAAYHPQQFIYAGSLSALLDPSQGMGGPGPGAQIYQAVSAQAAAIHGPGPGPSPSMGRDIKVQFQSGPGPGGINTIPIAINEAEYVGPGPGAAFQGAHARFVAAAAGPGPGAGWLAFFRDLVARGLGPGPGASIIRLVGAVLAEQHGPGPGMSFVTTQPEALAAAAGPGPGMHVSFVMAYPEMLAAGPGPGAYGSFVRTVSLPVGAGPGPGLENDNQLLYNYPGALLIPIAVGGALAGLVLIVLIAYLVGRKRSHAGYQTI

SEQ ID NO: 404 - RNA, 3520 nt, synthetic construct
  source 1..3520: mol_type = other RNA
acatttgcttctgacacaactgtgttcactagcaacctcaaacagccaccatggccgctcctggatccgcccgcagaccgttgttgctcctgttactactgctcttacttggactcatgcactgcgcctccgccgctgagatgaaaaccgacgccgcgaccctggcccaggaagccggaaacttcgagcggatttccggagatcttaagactcagatcgaccaggtcgaatccaccgccggatcgctccagggacagtggagaggcgccgcgggaaccgcggcccaggccgccgtcgtgcggttccaagaggccgccaacaagcagaagcaggaactggacgaaatttccactaacattcgccaagctggcgtgcagtactcgagagccgatgaagaacagcagcaagccctctcctcacaaatgggtttcggacctgggcccggcgtggacttcggagccctgccacctgaaatcaactccgcccggatgtacgcaggacctggatccgccagcctggtggcggccgcgcagatgtgggatagcgtggcctccgacctgttcagcgcggcttcagctttccaaagcgtggtctggggactgactgtgggatcctggatcggatcaagcgcaggcctcatggtggccgcggcgtccccttacgtggcctggatgagcgtgaccgccggccaggcagaactgaccgcagcccaagtccgcgtggccgcagccgcctacgagactgcctacggtctgacggtgccgccgccagtgatcgccgagaacagagcagagctcatgatcctcatcgcgaccaacctactgggccagaacactccggcgattgccgtgaacgaagccgaatatggggagatgtgggctcaggacgctgcagccatgttcggatatgcggccgcgactgctaccgccaccgccactctgctgccgttcgaggaggcccccgaaatgacttccgcgggaggcctgttggagcaagctgccgcggtggaggaagctagcgacaccgccgcagccaaccagctcatgaacaacgtgccacaggccctgcagcagctggcccagcccactcaagggaccaccccgagctcaaagctgggcggtctgtggaaaaccgtgtccccccaccgctcgcccatttccaacatggtgtcaatggcgaacaaccacatgggtcctggacctggaacggagcagcagtggaacttcgccgggatcgaagccgccgcctcggctatccaagggaatgtcacctcgatccattcccttctggacgaaggaaagcagtccctgaccaagcttgcggcagcctggggcggaagcggcagcgaagcctaccagggcgtgcagcaaaaatgggacgcaaccgccactgagctgaacaacgccctccaaaacctggctagaactatttccgaggccggacaggctatggccagcaccgagggcaacgtgaccgggatgttcgctggccctgggccgggcacctcgcggttcatgaccgatcctcatgctatgcgggatatggctggacggttcgaggtgcacgcccaaactgtggaggacgaggcccgccggatgtgggccagcgcgcagaacatctcgggggccggctggtccggaatggccgaagccacctccctcgataccatgacccagatgaaccaggccttccggaacattgtgaacatgctgcatggagtgcgggacggcctggtccgggacgccaacaattacgagcagcaggagcaggcctctcagcaaatcctgtccagcgggccggggcctggatcacaaatcatgtacaactacccggcaatgctgggccacgctggagacatggccggctacgccggcacgctgcagtcactcggtgccgagatcgccgtcgagcaggcggcactgcagagcgcttggcagggagacactggcattacctaccaagcgtggcaggctcagtggaatcaagctatggaagatctggtccgcgcgtaccacgccatgtcctccactcacgaagccaacaccatggctatgatggccagggacactgccgaggcagccaaatgggggggaggaccggggccgggaaccataaactaccagtttggcgacgtcgacgcccacggagccatgatcagggcgcaggccgggtcgctggaagcagaacaccaggccatcatctccgatgtgctgaccgcctccgacttttggggaggagccggttcggctgcctgccaagggttcatcacacaattgggaaggaacttccaggtcatctacgagcaggccaatgcacacggtcaaaaggtccaagcggcgggcaacaacatggcccagactgactcggccgtgggcagcagttgggccggtcccggccctggccctgtcggggggcagtcatccttctactccgactggtactcccccgcctgcggaaaggccggctgtcagacctataagtgggaaaccttcctgacctccgaactcccgcagtggctctcggcgaatcgggcagtgaagcccaccgggtcggcggctattggactgtccatggccggttccagcgcgatgatcctggccgcctatcacccccagcaattcatctacgctggctccctgtccgccctgcttgacccatcgcaggggatgggtggaccgggccctggggcccagatctaccaagcagtgtcggcccaagccgcagcgatccatggaccaggaccgggaccgtccccgagcatgggccgggatattaaggttcagttccagtccggacccggcccggggggtattaacaccattccgattgccattaatgaggcagaatacgtcggccctggtcccggcgccgcgttccaaggagcccacgcacgattcgtggcggcagctgcgggacccgggcctggtgccggttggctggcttttttccgcgacctggtggccagaggacttggacccggtccgggcgccagcattatcaggctggtcggggccgtgctggcggaacagcatggacctggcccgggaatgagcttcgtgaccacccaacctgaggcgcttgccgcggcggcaggccctggacccgggatgcatgtgtcttttgtgatggcctacccggaaatgctggccgcgggtccgggccccggagcctacggatcattcgtgcgcactgtgtccctccccgtgggagccggacctggaccggggttggagaatgataaccagctgctctacaactatccgggggccctcctgattcctatcgccgtgggcggtgccttggccggcctggtgctgatcgtcctgattgcctatctcgtggggaggaagagatcgcatgccggctaccagaccatctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 405 - protein, 1109 aa, synthetic construct
MRVTAPRTLILLLSGALALTETWATAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAGPGPGAEMKTDAATLAQEAGNFERISGDLKTQIDQVESTAGSLQGQWRGAAGTAAQAAVVRFQEAANKQKQELDEISTNIRQAGVQYSRADEEQQQALSSQMGFGPGPGVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMGPGPGTEQQWNFAGIEAAASAIQGNVTSIHSLLDEGKQSLTKLAAAWGGSGSEAYQGVQQKWDATATELNNALQNLARTISEAGQAMASTEGNVTGMFAGPGPGTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSGPGPGSFVMAYPEMLAAAADTLQSIGATTVASNAAAAAPTTGVVPPAADEVSALTAAHFAAHAAMYQSVSARAAAIHDQFVATLASSASSYAATEVANAAAASGPGPGTINYQFGDVDAHGAMIRAQAGSLEAEHQAIISDVLTASDFWGGAGSAACQGFITQLGRNFQVIYEQANAHGQKVQAAGNNMAQTDSAVGSSWAGPGPGPVGGQSSFYSDWYSPACGKAGCQTYKWETFLTSELPQWLSANRAVKPTGSAAIGLSMAGSSAMILAAYHPQQFIYAGSLSALLDPSQGMGGPGPGTVPPPVVAANRAELAVLAGPGPGMVDPVVVAANRSAFVQLVGIVAGLAVLAVVVIGAVVAAVMCRRKSSGGKGGSYSQAACSDSAQGSDVSLTA

SEQ ID NO: 406 - RNA, 3520 nt, synthetic construct
  source 1..3520: mol_type = other RNA
acatttgcttctgacacaactgtgttcactagcaacctcaaacagccaccatgcgcgtgactgcgcctaggaccctgattctgctcctgtcgggtgccctcgctctgaccgaaacctgggccaccgccgcgagcgacaattttcagttaagccagggtggccagggatttgccatcccgatcggacaggccatggctatcgcgggccagatcagatccggaggcggatccccgactgtacacatcggtccgaccgccttcttgggactgggagtggtggacaacaacgggaatggcgcacgggtgcagagggtcgtgggctcggcaccagccgcctctctgggaatcagcaccggggacgttatcaccgctgtggatggtgcccccatcaattccgccacggcgatggccgatgccctgaacggccatcaccccggggatgtgatatccgtgacctggcagaccaagtccggcggtacaaggaccggcaacgtcactctggccgagggtcctcctgcgggccctggtcctggtgctgagatgaaaaccgacgccgcgaccctggcccaggaagccggaaacttcgagcggatttccggagatcttaagactcagatcgaccaggtcgaatccaccgccggatcgctccagggacagtggagaggcgccgcgggaaccgcggcccaggccgccgtcgtgcggttccaagaggccgccaacaagcagaagcaggaactggacgaaatttccactaacattcgccaagctggcgtgcagtactcgagagccgatgaagaacagcagcaagccctctcctcacaaatgggtttcggacctgggcccggcgtggacttcggagccctgccacctgaaatcaactccgcccggatgtacgcaggacctggatccgccagcctggtggcggccgcgcagatgtgggatagcgtggcctccgacctgttcagcgcggcttcagctttccaaagcgtggtctggggactgactgtgggatcctggatcggatcaagcgcaggcctcatggtggccgcggcgtccccttacgtggcctggatgagcgtgaccgccggccaggcagaactgaccgcagcccaagtccgcgtggccgcagccgcctacgagactgcctacggtctgacggtgccgccgccagtgatcgccgagaacagagcagagctcatgatcctcatcgcgaccaacctactgggccagaacactccggcgattgccgtgaacgaagccgaatatggggagatgtgggctcaggacgctgcagccatgttcggatatgcggccgcgactgctaccgccaccgccactctgctgccgttcgaggaggcccccgaaatgacttccgcgggaggcctgttggagcaagctgccgcggtggaggaagctagcgacaccgccgcagccaaccagctcatgaacaacgtgccacaggccctgcagcagctggcccagcccactcaagggaccaccccgagctcaaagctgggcggtctgtggaaaaccgtgtccccccaccgctcgcccatttccaacatggtgtcaatggcgaacaaccacatgggtcctggacctggaacggagcagcagtggaacttcgccgggatcgaagccgccgcctcggctatccaagggaatgtcacctcgatccattcccttctggacgaaggaaagcagtccctgaccaagcttgcggcagcctggggcggaagcggcagcgaagcctaccagggcgtgcagcaaaaatgggacgcaaccgccactgagctgaacaacgccctccaaaacctggctagaactatttccgaggccggacaggctatggccagcaccgagggcaacgtgaccgggatgttcgctggccctgggccgggcacctcgcggttcatgaccgatcctcatgctatgcgggatatggctggacggttcgaggtgcacgcccaaactgtggaggacgaggcccgccggatgtgggccagcgcgcagaacatctcgggggccggctggtccggaatggccgaagccacctccctcgataccatgacccagatgaaccaggccttccggaacattgtgaacatgctgcatggagtgcgggacggcctggtccgggacgccaacaattacgagcagcaggagcaggcctctcagcaaatcctgtccagcgggccggggcctggatcattcgtgatggcctaccctgagatgctggcagccgcggccgacaccctgcaatccattggcgccactaccgtggcatcgaacgcggcagccgccgcaccgactaccggcgtggtgccgcccgccgctgatgaagtgtccgcccttactgccgcccacttcgccgcccacgccgcgatgtaccagtccgtgtccgcgcgcgccgccgctatccacgatcagttcgtcgccacactggccagctcagcctcctcatacgccgctactgaagtggcaaacgcagccgctgcatccggcccgggacccggaacgatcaactaccagtttggcgacgtggacgcgcacggggcgatgatcagggctcaagcggggtccctcgaggccgaacaccaagcgattatctcggatgtcctcaccgcgtccgacttctggggtggagccggaagcgccgcctgccaaggtttcatcacccaactgggtcgcaactttcaagtcatctacgaacaggccaacgcccacggacagaaggtccaagccgccggaaacaacatggcacagaccgactccgcggtcggatcgagctgggccggacccgggccaggtcccgtgggcggacagagctcgttctattctgactggtactcacccgcatgtggaaaggccgggtgccagacctacaaatgggaaaccttcttgacttccgagctgcctcagtggttgtccgccaatcgggccgtgaagccgaccggctcggcggccatcggcctctccatggccggctcgtccgcgatgattcttgccgcctaccacccgcagcagttcatctacgccgggtccctctcggcactgctggaccccagtcagggaatgggcggacccggaccgggaactgtcccgcccccggtggtggctgctaaccgcgccgaacttgccgtgctggccggtccagggccgggaatggtggacccagtggtggtggcagcgaacagatccgcattcgtgcaactggtcggcattgtggccggactggccgtgttggccgtcgtcgtgatcggcgctgtggtcgccgccgtgatgtgccgaagaaagtccagcggtggaaaggggggatcctactcacaagcagcctgcagcgactcagcgcaggggtcggacgtgtcactgacagcatgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 407 - protein, 1095 aa, synthetic construct
MAAPGSARRPLLLLLLLLLLGLMHCASATAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAGPGPGAEMKTDAATLAQEAGNFERISGDLKTQIDQVESTAGSLQGQWRGAAGTAAQAAVVRFQEAANKQKQELDEISTNIRQAGVQYSRADEEQQQALSSQMGFGPGPGVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMGPGPGTEQQWNFAGIEAAASAIQGNVTSIHSLLDEGKQSLTKLAAAWGGSGSEAYQGVQQKWDATATELNNALQNLARTISEAGQAMASTEGNVTGMFAGPGPGTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSGPGPGSFVMAYPEMLAAAADTLQSIGATTVASNAAAAAPTTGVVPPAADEVSALTAAHFAAHAAMYQSVSARAAAIHDQFVATLASSASSYAATEVANAAAASGPGPGTINYQFGDVDAHGAMIRAQAGSLEAEHQAIISDVLTASDFWGGAGSAACQGFITQLGRNFQVIYEQANAHGQKVQAAGNNMAQTDSAVGSSWAGPGPGPVGGQSSFYSDWYSPACGKAGCQTYKWETFLTSELPQWLSANRAVKPTGSAAIGLSMAGSSAMILAAYHPQQFIYAGSLSALLDPSQGMGGPGPGTVPPPVVAANRAELAVLAGPGPGMVDPVVVAANRSAFVQLVLIPIAVGGALAGLVLIVLIAYLVGRKRSHAGYQTI

SEQ ID NO: 408 - RNA, 3478 nt, synthetic construct
  source 1..3478: mol_type = other RNA
acatttgcttctgacacaactgtgttcactagcaacctcaaacagccaccatggcggctccgggaagcgcacgacgcccgctgctgctcttgttgctgctcctcctgctgggtctgatgcactgcgcctcggccaccgccgcgagcgacaattttcagttaagccagggtggccagggatttgccatcccgatcggacaggccatggctatcgcgggccagatcagatccggaggcggatccccgactgtacacatcggtccgaccgccttcttgggactgggagtggtggacaacaacgggaatggcgcacgggtgcagagggtcgtgggctcggcaccagccgcctctctgggaatcagcaccggggacgttatcaccgctgtggatggtgcccccatcaattccgccacggcgatggccgatgccctgaacggccatcaccccggggatgtgatatccgtgacctggcagaccaagtccggcggtacaaggaccggcaacgtcactctggccgagggtcctcctgcgggccctggtcctggtgctgagatgaaaaccgacgccgcgaccctggcccaggaagccggaaacttcgagcggatttccggagatcttaagactcagatcgaccaggtcgaatccaccgccggatcgctccagggacagtggagaggcgccgcgggaaccgcggcccaggccgccgtcgtgcggttccaagaggccgccaacaagcagaagcaggaactggacgaaatttccactaacattcgccaagctggcgtgcagtactcgagagccgatgaagaacagcagcaagccctctcctcacaaatgggtttcggacctgggcccggcgtggacttcggagccctgccacctgaaatcaactccgcccggatgtacgcaggacctggatccgccagcctggtggcagccgcgcagatgtgggatagcgtggcctccgacctgttcagcgcggcttcagctttccaaagcgtggtctggggactgactgtgggatcctggatcggatcaagcgcaggcctcatggtggccgcggcgtccccttacgtggcctggatgagcgtgaccgccggccaggcagaactgaccgcagcccaagtccgcgtggccgcagccgcctacgagactgcctacggtctgacggtgccgccgccagtgatcgccgagaacagagcagagctcatgatcctcatcgcgaccaacctactgggccagaacactccggcgattgccgtgaacgaagccgaatatggggagatgtgggctcaggacgctgcagccatgttcggatatgcagccgcgactgctaccgccaccgccactctgctgccgttcgaggaggcccccgaaatgacttccgcgggaggcctgttggagcaagctgccgcggtggaggaagctagcgacaccgccgcagccaaccagctcatgaacaacgtgccacaggccctgcagcagctggcccagcccactcaagggaccaccccgagctcaaagctgggcggtctgtggaaaaccgtgtccccccaccgctcgcccatttccaacatggtgtcaatggcgaacaaccacatgggtcctggacctggaacggagcagcagtggaacttcgccgggatcgaagccgccgcctcggctatccaagggaatgtcacctcgatccattcccttctggacgaaggaaagcagtccctgaccaagcttgcggcagcctggggcggaagcggcagcgaagcctaccagggcgtgcagcaaaaatgggacgcaaccgccactgagctgaacaacgccctccaaaacctggctagaactatttccgaggccggacaggctatggccagcaccgagggcaacgtgaccgggatgttcgctggccctgggccgggcacctcgcggttcatgaccgatcctcatgctatgcgggatatggctggacggttcgaggtgcacgcccaaactgtggaggacgaggcccgccggatgtgggccagcgcgcagaacatctcgggggccggctggtccggaatggccgaagccacctccctcgataccatgacccagatgaaccaggccttccggaacattgtgaacatgctgcatggagtgcgggacggcctggtccgggacgccaacaattacgagcagcaggagcaggcctctcagcaaatcctgtccagcgggccggggcctggatcattcgtgatggcctaccctgagatgctggcagccgcggccgacaccctgcaatccattggcgccactaccgtggcatcgaacgcggcagccgccgcaccgactaccggcgtggtgccgcccgccgctgatgaagtgtccgcccttactgccgcccacttcgccgcccacgccgcgatgtaccagtccgtgtccgcgcgcgccgccgctatccacgatcagttcgtcgccacactggccagctcagcctcctcatacgccgctactgaagtggcaaacgcagccgctgcatccggcccgggacccggaacgatcaactaccagtttggcgacgtggacgcgcacggggcgatgatcagggctcaagcggggtccctcgaggccgaacaccaagcgattatctcggatgtcctcaccgcgtccgacttctggggtggagccggaagcgccgcctgccaaggtttcatcacccaactgggtcgcaactttcaagtcatctacgaacaggccaacgcccacggacagaaggtccaagccgccggaaacaacatggcacagaccgactccgcggtcggatcgagctgggccggacccgggccaggtcccgtgggcggacagagctcgttctattctgactggtactcacccgcatgtggaaaggccgggtgccagacctacaaatgggaaaccttcttgacttccgagctgcctcagtggttgtccgccaatcgggccgtgaagccgaccggctcggcggccatcggcctctccatggccggctcgtccgcgatgattcttgccgcctaccacccgcagcagttcatctacgccgggtccctctcggcactgctggaccccagtcagggaatgggcggacccggaccgggaactgtcccgcccccggtggtggctgctaaccgcgccgaacttgccgtgctggccggtccagggccgggaatggtggacccagtggtggtggcagcgaacagatccgcattcgtgcaactggtcctgattcctattgcggtcggcggggctctcgccgggctcgtgctcattgtcctgattgcctacctggtcggacgcaagaggtcccatgcgggctaccagaccatctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 409 - protein, 1179 aa, synthetic construct
MAVMAPRTLLLLLLGALALTQTWAGSTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAGGSGGGGSGGAEMKTDAATLAQEAGNFERISGDLKTQIDQVESTAGSLQGQWRGAAGTAAQAAVVRFQEAANKQKQELDEISTNIRQAGVQYSRADEEQQQALSSQMGFGGSGGGGSGGVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMGGSGGGGSGGTEQQWNFAGIEAAASAIQGNVTSIHSLLDEGKQSLTKLAAAWGGSGSEAYQGVQQKWDATATELNNALQNLARTISEAGQAMASTEGNVTGMFAGGSGGGGSGGTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSGGSGGGGSGGSFVMAYPEMLAAAADTLQSIGATTVASNAAAAAPTTGVVPPAADEVSALTAAHFAAHAAMYQSVSARAAAIHDQFVATLASSASSYAATEVANAAAASGGSGGGGSGGTINYQFGDVDAHGAMIRAQAGSLEAEHQAIISDVLTASDFWGGAGSAACQGFITQLGRNFQVIYEQANAHGQKVQAAGNNMAQTDSAVGSSWAGGSGGGGSGGPVGGQSSFYSDWYSPACGKAGCQTYKWETFLTSELPQWLSANRAVKPTGSAAIGLSMAGSSAMILAAYHPQQFIYAGSLSALLDPSQGMGGGSGGGGSGGFAATVPPPVVAANRAELAVLAATNGGSGGGGSGGRAAMVDPVVVAANRSAFVQLVLSNEPSSQSTVPIVGIVAGLAVLAVVVIGAVVAAVMCRRKSSGGKGGSYSQAACSDSAQGSDVSLTA

SEQ ID NO: 410 - RNA, 3730 nt, synthetic construct
  source 1..3730: mol_type = other RNA
acatttgcttctgacacaactgtgttcactagcaacctcaaacagccaccatggccgtgatggcccccagaactctcttgctgcttctgttgggagctctggcgctgacccagacctgggctggatcgaccgctgcctccgacaacttccaactgtcacaaggaggacaagggtttgccatcccgatcggccaagccatggccatcgccggacaaattcggagcgggggaggaagccccactgtgcacattggtccgaccgccttcctcgggctgggcgtggtggacaacaacggcaacggcgccagggtgcagagggtcgtgggatccgcaccggccgcctctctcggaatttcgaccggcgacgtgattaccgccgtggacggggcgccgattaactccgccacggcaatggccgacgcgctcaacggtcaccacccgggagatgtcatctccgtgacctggcagaccaagagcggaggcactcgcaccggaaacgtcactctcgccgagggcccacccgccggaggttctggaggaggcggctccggtggcgcggagatgaaaaccgatgcggccaccctggcacaggaggctggaaacttcgaaagaatcagcggcgacctcaagactcagatcgaccaggtcgaatcgactgccggaagcctccagggccagtggaggggagccgctggaactgcggcgcaggcggccgtggtcagattccaggaggccgccaacaagcagaagcaggaactggatgaaatttccactaacatcagacaggctggggtgcaatactcccgcgcggacgaagaacagcagcaggccctgtccagccaaatgggattcggtggcagcgggggtggcggctcagggggagtggatttcggggctctgccacctgagatcaactctgcccggatgtacgcggggccgggcagcgcatcccttgtggccgccgcccagatgtgggatagcgtggcttccgacctcttctccgccgcatccgcctttcaatccgtggtctgggggctgactgtgggctcgtggatcggatcatctgctggcctcatggtggccgcggcatccccatacgtggcctggatgagtgtgaccgctggccaggctgaattgaccgccgcacaagtccgcgtcgctgccgccgcatacgaaaccgcctacgggctgaccgttccgccccccgtgattgccgaaaatagggccgaactcatgatcctcatcgcgactaatctgctgggtcagaacaccccggctatcgccgtgaatgaggccgaatacggcgaaatgtgggcccaggacgccgctgccatgttcggctacgccgccgccactgcgaccgcgaccgccactctgctgcctttcgaagaggcccctgagatgacctccgcgggtggcctgctagaacaagcggcagctgtggaagaagcctcggataccgccgccgctaaccagctgatgaacaacgtgccccaggcgctgcagcagctcgcccagccgactcagggaaccaccccgagctcaaagcttggtggcctgtggaaaaccgtatcgcctcatcgctcaccaatcagcaacatggtcagcatggcgaacaaccacatgggtggctcaggcggcgggggctccggaggcaccgagcagcagtggaacttcgccggaatcgaggccgcagcgagcgcaatccagggcaatgtcacctccattcactccctgctggatgagggcaaacagtccctgactaagcttgccgcagcgtggggaggttccggctccgaagcctaccagggcgtgcagcagaaatgggacgctactgctaccgaactgaacaacgccctgcagaatcttgctcggaccattagcgaagccggacaagcgatggcctccaccgagggaaacgtgaccgggatgtttgccggggggtccggaggaggaggctcaggaggaaccagccgcttcatgactgaccctcatgccatgcgggacatggccggacgcttcgaagtgcatgcgcagactgtggaggacgaagcccggcggatgtgggcatcggcccagaacatctccggagcgggatggtccggaatggccgaggccacctccctcgacaccatgacgcagatgaaccaggcctttcgcaatatcgtgaacatgctgcacggcgtgcgcgacggcctcgtgcgggacgccaacaactacgagcaacaggagcaggcgtcacagcagattctgtcctcgggcggttcgggaggcggtggatcgggggggtcattcgtgatggcttaccccgagatgttggctgctgcggctgacacccttcagagcatcggagccactaccgtcgcctcgaacgccgcggcagccgcccctaccaccggggtggtgcctccagccgccgacgaagtctccgccctgactgccgcgcacttcgccgcccacgcagccatgtaccagtcggtgtcggcccgggcggcagctatccacgatcagttcgtggccacgttagcatccagcgcgtcaagctatgccgccacggaagtggccaacgcagctgcagcctctggaggaagcgggggcggaggaagtggcggcactatcaactaccaattcggagatgtggacgcgcacggcgccatgatccgcgcccaagcgggatcccttgaagcggagcatcaggccattatctccgacgtcttgaccgcctccgacttttggggtggagccgggagcgccgcgtgccagggattcatcacccaactggggcggaatttccaggtcatctacgaacaggctaacgcccacggtcaaaaggttcaggctgccggcaacaacatggcccagaccgactccgccgtggggtcctcctgggccggaggctccggcggtggcggatcaggaggccctgtgggaggacagtcctccttctattccgattggtactcccccgcctgcggaaaggccggatgtcagacgtataagtgggagacattcctgacctccgagctgccccagtggctctcggccaaccgggctgtgaagccgactggatccgccgcaattgggctgtccatggcgggctcgtcggcgatgatcctggccgcttaccacccacaacagttcatctacgccggatccttgtcggcactgctggacccgagccaggggatgggcggtggttccgggggcgggggatccggaggtttcgccgcgaccgtgccacctccggtcgtggcagccaaccgcgccgaactggccgtcctggcggctaccaacgggggatcgggtggaggggggtcgggaggcagagccgccatggtcgaccctgtggtggtcgccgccaatagatcagccttcgtgcaactcgtgctgtcgaacgagccctccagccaatccaccgtgccgatcgtcggaattgtggccggcctggctgtgctggcagtggtggtcattggagccgtggtggctgccgtgatgtgcagaagaaagagcagcggaggaaagggaggatcctactcacaagccgcgtgctccgatagcgcccagggttcggatgtgtcactcaccgcctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 411 - protein, 1189 aa, synthetic construct
MAVMAPRTLLLLLLGALALTQTWAGSTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAGGSGGGGSGGVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMGGSGGGGSGGAEMKTDAATLAQEAGNFERISGDLKTQIDQVESTAGSLQGQWRGAAGTAAQAAVVRFQEAANKQKQELDEISTNIRQAGVQYSRADEEQQQALSSQMGFGGGGSGGGGSTEQQWNFAGIEAAASAIQGNVTSIHSLLDEGKQSLTKLAAAWGGSGSEAYQGVQQKWDATATELNNALQNLARTISEAGQAMASTEGNVTGMFAGGSGGGGSGGSFVMAYPEMLAAAADTLQSIGATTVASNAAAAAPTTGVVPPAADEVSALTAAHFAAHAAMYQSVSARAAAIHDQFVATLASSASSYAATEVANAAAASGGSGGGGSGGTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSGGGGSGGGGSTINYQFGDVDAHGAMIRAQAGSLEAEHQAIISDVLTASDFWGGAGSAACQGFITQLGRNFQVIYEQANAHGQKVQAAGNNMAQTDSAVGSSWAGGSGGGGSGGPVGGQSSFYSDWYSPACGKAGCQTYKWETFLTSELPQWLSANRAVKPTGSAAIGLSMAGSSAMILAAYHPQQFIYAGSLSALLDPSQGMGGGSGGGGSGGFAATVPPPVVAANRAELAVLAATNGGSGGGGSGGRAAMVDPVVVAANRSAFVQLVLSNGGSGGGGSGGEPSSQSTVPIVGIVAGLAVLAVVVIGAVVAAVMCRRKSSGGKGGSYSQAACSDSAQGSDVSLTA

SEQ ID NO: 412 - RNA, 3760 nt, synthetic construct
  source 1..3760: mol_type = other RNA
acatttgcttctgacacaactgtgttcactagcaacctcaaacagccaccatggccgtgatggcccccagaactctcttgctgcttctgttgggagctctggcgctgacccagacctgggctggatcgaccgctgcctccgacaacttccaactgtcacaaggaggacaagggtttgccatcccgatcggccaagccatggccatcgccggacaaattcggagcgggggaggaagccccactgtgcacattggtccgaccgccttcctcgggctgggcgtggtggacaacaacggcaacggcgccagggtgcagagggtcgtgggatccgcaccggccgcctctctcggaatttcgaccggcgacgtgattaccgccgtggacggggcgccgattaactccgccacggcaatggccgacgcgctcaacggtcaccacccgggagatgtcatctccgtgacctggcagaccaagagcggaggcactcgcaccggaaacgtcactctcgccgagggcccacccgccggtggcagcgggggtggcggctcagggggagtggatttcggggctctgccacctgagatcaactctgcccggatgtacgcggggccgggcagcgcatcccttgtggccgccgcccagatgtgggatagcgtggcttccgacctcttctccgccgcatccgcctttcaatccgtggtctgggggctgactgtgggctcgtggatcggatcatctgctggcctcatggtggccgcggcatccccatacgtggcctggatgagtgtgaccgctggccaggctgaattgaccgccgcacaagtccgcgtcgctgccgccgcatacgaaaccgcctacgggctgaccgttccgccccccgtgattgccgaaaatagggccgaactcatgatcctcatcgcgactaatctgctgggtcagaacaccccggctatcgccgtgaatgaggccgaatacggcgaaatgtgggcccaggacgccgctgccatgttcggctacgccgccgccactgcgaccgcgaccgccactctgctgcctttcgaagaggcccctgagatgacctccgcgggtggcctgctagaacaagcggcagctgtggaagaagcctcggataccgccgccgctaaccagctgatgaacaacgtgccccaggcgctgcagcagctcgcccagccgactcagggaaccaccccgagctcaaagcttggtggcctgtggaaaaccgtatcgcctcatcgctcaccaatcagcaacatggtcagcatggcgaacaaccacatgggtggctcaggcggcgggggctccggaggcgccgaaatgaagactgacgcagccactctggcccaggaggctgggaacttcgaacgcatctcaggagatctcaagacccagatcgatcaggtggaatccactgccggcagcctccaaggccagtggcgaggagccgccggtactgccgctcaggccgcagtggtgcgcttccaggaggccgccaacaagcagaagcaggaactggacgagattagcaccaatatccgccaagccggagtgcaatatagccgggccgacgaggaacagcaacaagccctctcctcccaaatggggttcggcggaggaggaagcggaggcggcggcagcaccgagcagcagtggaacttcgccggaatcgaggccgcagcgagcgcaatccagggcaatgtcacctccattcactccctgctggatgagggcaaacagtccctgactaagcttgccgcagcgtggggaggttccggctccgaagcctaccagggcgtgcagcagaaatgggacgctactgctaccgaactgaacaacgccctgcagaatcttgctcggaccattagcgaagccggacaagcgatggcctccaccgagggaaacgtgaccgggatgtttgccggaggatcgggcgggggcggctccggagggtcctttgtcatggcatacccagagatgctggccgccgccgcggataccctgcagtcgattggagccactaccgtggcctcaaacgccgcagcggctgcccccaccactggagtggtgcccccagcagccgacgaagtctccgccttaactgccgcccactttgccgcccatgcggcgatgtaccagtccgtgtcagcccgcgccgccgcaatccacgatcagttcgtcgcaacactggcgtccagcgcgtcgtcgtacgccgccaccgaggtggccaacgcggcggcggcctcaggggggtccggaggaggaggctcaggaggaaccagccgcttcatgactgaccctcatgccatgcgggacatggccggacgcttcgaagtgcatgcgcagactgtggaggacgaagcccggcggatgtgggcatcggcccagaacatctccggagcgggatggtccggaatggccgaggccacctccctcgacaccatgacgcagatgaaccaggcctttcgcaatatcgtgaacatgctgcacggcgtgcgcgacggcctcgtgcgggacgccaacaactacgagcaacaggagcaggcgtcacagcagattctgtcctcgggcggtggaggaagcgggggcggaggaagtactatcaactaccaattcggagatgtggacgcgcacggcgccatgatccgcgcccaagcgggatcccttgaagcggagcatcaggccattatctccgacgtcttgaccgcctccgacttttggggtggagccgggagcgccgcgtgccagggattcatcacccaactggggcggaatttccaggtcatctacgaacaggctaacgcccacggtcaaaaggttcaggctgccggcaacaacatggcccagaccgactccgccgtggggtcctcctgggccggaggctccggcggtggcggatcaggaggccctgtgggaggacagtcctccttctattccgattggtactcccccgcctgcggaaaggccggatgtcagacgtataagtgggagacattcctgacctccgagctgccccagtggctctcggccaaccgggctgtgaagccgactggatccgccgcaattgggctgtccatggcgggctcgtcggcgatgatcctggccgcttaccacccacaacagttcatctacgccggatccttgtcggcactgctggacccgagccaggggatgggcggtggttccgggggcgggggatccggaggtttcgccgcgaccgtgccacctccggtcgtggcagccaaccgcgccgaactggccgtcctggcggctaccaacgggggatcgggtggaggggggtcgggaggcagagccgccatggtcgaccctgtggtggtcgccgccaatagatcagccttcgtgcaactcgtgctgtcgaacgggggatccggaggggggggttccggcggcgagccctccagccaatccaccgtgccgatcgtcggaattgtggccggcctggctgtgctggcagtggtggtcattggagccgtggtggctgccgtgatgtgcagaagaaagagcagcggaggaaagggaggatcctactcacaagccgcgtgctccgatagcgcccagggttcggatgtgtcactcaccgcctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 413 - protein, 1252 aa, synthetic construct
MAVMAPRTLLLLLLGALALTQTWAGSTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAGGSGGGGSGGAEMKTDAATLAQEAGNFERISGDLKTQIDQVESTAGSLQGQWRGAGGSGGGGSGGTSRFMTDPHAMRDMAGRFEVHAQTVEDEARRMWASAQNISGAGWSGGGSGGGGSGGTEQQWNFAGIEAAASAIQGNVTSIHSLLDEGKQSLTKLAAAWGGSGSGGSGGGGSGGTINYQFGDVDAHGAMIRAQAGSLEAEHQAIISDVLTASDFWGGAGSGGSGGGGSGGVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMGGSGGGGSGGSFVMAYPEMLAAAADTLQSIGATTVASNAAAAAPTTGVVPPAADEVSALTAAHFAAHAAMYQSVSARAAAIHDQFVATLASSASSYAATEVANAAAASGGSGGGGSGGGQWRGAAGTAAQAAVVRFQEAANKQKQELDEISTNIRQAGVQYSRADEEQQQALSSQMGFGGSGGGGSGGSGAGWSGMAEATSLDTMTQMNQAFRNIVNMLHGVRDGLVRDANNYEQQEQASQQILSSGGSGGGGSGGGGSGSEAYQGVQQKWDATATELNNALQNLARTISEAGQAMASTEGNVTGMFAGGSGGGGSGGGGAGSAACQGFITQLGRNFQVIYEQANAHGQKVQAAGNNMAQTDSAVGSSWAGGSGGGGSGGPVGGQSSFYSDWYSPACGKAGCQTYKWETFLTSELPQWLSANRAVKPTGSAAIGLSMAGSSAMILAAYHPQQFIYAGSLSALLDPSQGMGGGSGGGGSGGFAATVPPPVVAANRAELAVLAATNGGSGGGGSGGRAAMVDPVVVAANRSAFVQLVLSNGGSGGGGSGGEPSSQSTVPIVGIVAGLAVLAVVVIGAVVAAVMCRRKSSGGKGGSYSQAACSDSAQGSDVSLTA

SEQ ID NO: 414 - RNA, 3949 nt, synthetic construct
  source 1..3949: mol_type = other RNA
acatttgcttctgacacaactgtgttcactagcaacctcaaacagccaccatggccgtgatggcccccagaactctcttgctgcttctgttgggagctctggcgctgacccagacctgggctggatcgaccgctgcctccgacaacttccaactgtcacaaggaggacaagggtttgccatcccgatcggccaagccatggccatcgccggacaaattcggagcgggggaggaagccccactgtgcacattggtccgaccgccttcctcgggctgggcgtggtggacaacaacggcaacggcgccagggtgcagagggtcgtgggatccgcaccggccgcctctctcggaatttcgaccggcgacgtgattaccgccgtggacggggcgccgattaactccgccacggcaatggccgacgcgctcaacggtcaccacccgggagatgtcatctccgtgacctggcagaccaagagcggaggcactcgcaccggaaacgtcactctcgccgagggcccacccgccggaggttctggaggaggcggctccggtggcgcggagatgaaaaccgatgcggccaccctggcacaggaggctggaaacttcgaaagaatcagcggcgacctcaagactcagatcgaccaggtcgaatcgactgccggaagcctccagggccagtggaggggagccggtggatccggaggagggggatccggcggaaccagccgcttcatgaccgatcctcacgctatgcgcgacatggcgggtcgcttcgaagtgcatgcccagactgtcgaggacgaggcccggcgaatgtgggccagcgcgcagaacatcagcggcgccggttggtcaggaggcgggtccggggggggaggttctggcggtaccgagcagcaatggaacttcgcgggtattgaagccgcggcatccgcgatccagggcaacgtgacctccattcacagcctgttagacgaaggaaagcagagcctgaccaagttggccgccgcctggggggggtcagggtccggaggttccggaggcggtggctcgggcggcaccatcaattaccagttcggggacgtggacgcacacggagccatgatccgggcccaggcgggctccctggaagcggagcatcaggccatcattagcgatgtgctcacggctagcgatttctgggggggagccggaagcggtggcagcgggggtggcggctcagggggagtggatttcggggctctgccacctgagatcaactctgcccggatgtacgcggggccgggcagcgcatcccttgtggccgccgcccagatgtgggatagcgtggcttccgacctcttctccgccgcatccgcctttcaatccgtggtctgggggctgactgtgggctcgtggatcggatcatctgctggcctcatggtggccgcggcatccccatacgtggcctggatgagtgtgaccgctggccaggctgaattgaccgccgcacaagtccgcgtcgctgccgccgcatacgaaaccgcctacgggctgaccgttccgccccccgtgattgccgaaaatagggccgaactcatgatcctcatcgcgactaatctgctgggtcagaacaccccggctatcgccgtgaatgaggccgaatacggcgaaatgtgggcccaggacgccgctgccatgttcggctacgccgccgccactgcgaccgcgaccgccactctgctgcctttcgaagaggcccctgagatgacctccgcgggtggcctgctagaacaagcggcagctgtggaagaagcctcggataccgccgccgctaaccagctgatgaacaacgtgccccaggcgctgcagcagctcgcccagccgactcagggaaccaccccgagctcaaagcttggtggcctgtggaaaaccgtatcgcctcatcgctcaccaatcagcaacatggtcagcatggcgaacaaccacatgggtggctcaggcggcgggggctccggaggcagctttgtgatggcctaccccgaaatgctcgcagccgcagcggataccttgcagtcaattggcgccaccaccgtggcctccaacgccgctgccgccgccccgactacgggggtggtcccgcccgccgcagatgaagtgtccgctcttaccgcagcgcactttgccgcccatgctgccatgtaccagtccgtgtcggctagagctgccgcgatccacgaccagttcgtggctaccctggccagctcggcatcgagctacgccgccaccgaggtggctaacgccgccgccgccagcggggggtccggaggaggaggctcaggaggaggccaatggcggggtgccgccggaaccgccgcccaagcggctgtcgtgcgcttccaggaggccgccaacaagcagaagcaggaactcgacgaaatatcaaccaacatccggcaagctggggtgcagtacagcagagccgatgaggaacagcaacaggcgctctcctcgcaaatgggcttcggaggctcaggagggggcggatcgggtggctccggagcgggatggtccggaatggccgaggccacctccctcgacaccatgacgcagatgaaccaggcctttcgcaatatcgtgaacatgctgcacggcgtgcgcgacggcctcgtgcgggacgccaacaactacgagcaacaggagcaggcgtcacagcagattctgtcctcgggcggttcgggaggcggtggatcgggggggggaggatccggatcagaagcctatcaaggtgtgcagcagaagtgggacgccaccgcgaccgaacttaataacgccttgcagaacttggcccggacgatatccgaagccggccaagccatggcctccacggaaggcaatgtgactggcatgttcgcgggaggaagcgggggcggaggaagtggcggcggtggagccgggagcgccgcgtgccagggattcatcacccaactggggcggaatttccaggtcatctacgaacaggctaacgcccacggtcaaaaggttcaggctgccggcaacaacatggcccagaccgactccgccgtggggtcctcctgggccggaggctccggcggtggcggatcaggaggccctgtgggaggacagtcctccttctattccgattggtactcccccgcctgcggaaaggccggatgtcagacgtataagtgggagacattcctgacctccgagctgccccagtggctctcggccaaccgggctgtgaagccgactggatccgccgcaattgggctgtccatggcgggctcgtcggcgatgatcctggccgcttaccacccacaacagttcatctacgccggatccttgtcggcactgctggacccgagccaggggatgggcggtggttccgggggcgggggatccggaggtttcgccgcgaccgtgccacctccggtcgtggcagccaaccgcgccgaactggccgtcctggcggctaccaacgggggatcgggtggaggggggtcgggaggcagagccgccatggtcgaccctgtggtggtcgccgccaatagatcagccttcgtgcaactcgtgctgtcgaacgggggctccggcggtggtggctcgggcggggagccctccagccaatccaccgtgccgatcgtcggaattgtggccggcctggctgtgctggcagtggtggtcattggagccgtggtggctgccgtgatgtgcagaagaaagagcagcggaggaaagggaggatcctactcacaagccgcgtgctccgatagcgcccagggttcggatgtgtcactcaccgcctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 415 - protein, 1162 aa, synthetic construct
MAAPGSARRPLLLLLLLLLLGLMHCASATAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAGGSGGGGSGGAEMKTDAATLAQEAGNFERISGDLKTQIDQVESTAGSLQGQWRGAAGTAAQAAVVRFQEAANKQKQELDEISTNIRQAGVQYSRADEEQQQALSSQMGFGGSGGGGSGGVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMGGSGGGGSGGTEQQWNFAGIEAAASAIQGNVTSIHSLLDEGKQSLTKLAAAWGGSGSEAYQGVQQKWDATATELNNALQNLARTISEAGQAMASTEGNVTGMFAGGSGGGGSGGTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSGGSGGGGSGGSFVMAYPEMLAAAADTLQSIGATTVASNAAAAAPTTGVVPPAADEVSALTAAHFAAHAAMYQSVSARAAAIHDQFVATLASSASSYAATEVANAAAASGGSGGGGSGGTINYQFGDVDAHGAMIRAQAGSLEAEHQAIISDVLTASDFWGGAGSAACQGFITQLGRNFQVIYEQANAHGQKVQAAGNNMAQTDSAVGSSWAGGSGGGGSGGPVGGQSSFYSDWYSPACGKAGCQTYKWETFLTSELPQWLSANRAVKPTGSAAIGLSMAGSSAMILAAYHPQQFIYAGSLSALLDPSQGMGGGSGGGGSGGFAATVPPPVVAANRAELAVLAATNGGSGGGGSGGRAAMVDPVVVAANRSAFVQLVLSNGGSGGGGSGGLIPIAVGGALAGLVLIVLIAYLVGRKRSHAGYQTI

SEQ ID NO: 416 - RNA, 3679 nt, synthetic construct
  source 1..3679: mol_type = other RNA
acatttgcttctgacacaactgtgttcactagcaacctcaaacagccaccatggccgcccctggcagcgccagaagacctctgctgctgctgctgctgctgctgctgctgggcctgatgcactgcgcctcagccaccgccgcttctgacaacttccagctgagccagggcggccagggattcgccatccctattggtcaggccatggccatcgccggccagatcagaagtggaggtggctctccaaccgtgcacatcggccccactgccttcctgggcctaggagtggtggataacaacggcaatggcgccagagtgcaaagagtggtgggttcagctcctgccgccagcctggggatcagcaccggcgacgtgatcaccgcagtggacggcgctcctatcaactccgccacagccatggccgatgccctgaacggccaccaccctggagatgtcatctctgtgacctggcagacaaagagcggcggcaccagaacaggaaacgtgaccctggccgaggggcctcctgccggcggctccggaggcggtggatctggaggcgccgagatgaagaccgacgccgctacactggcgcaggaggccggcaacttcgagagaatctcaggggatctgaagacccagatcgaccaggtggaaagcaccgcaggatctcttcagggccagtggcggggcgccgctggcacagccgcccaggctgctgtggtgcggttccaggaggccgccaacaagcagaaacaggagctggatgagatcagcaccaacattcgccaggccggcgtgcaatacagcagagccgacgaagaacagcagcaggctctgtctagccagatgggatttggcggttctggtggtggcggatcaggtggcgtcgacttcggcgccctcccccccgaaatcaacagcgccagaatgtacgccggacctggcagcgccagcctggtggcagcagcccaaatgtgggatagcgtggcttctgacctgttctccgccgctagcgcttttcagtccgtggtgtggggcctgaccgtgggcagctggatcggcagcagcgctggactgatggtggcagctgccagcccctacgtggcctggatgagcgtgacagctggacaggctgaactgacagccgcccaagtgcgggtggctgccgccgcctatgagacagcttacggcctgacagtgccccccccagtgattgccgaaaaccgggccgagctgatgatcctgatcgccaccaatctgctgggccagaacacccctgccatcgccgtgaacgaggcggaatacggcgagatgtgggcccaggacgccgccgccatgttcggctacgcagccgctaccgccaccgccacagccaccctgctgcctttcgaggaagctccagagatgaccagcgctggcggcctattggagcaggccgccgcagttgaggaagcttctgatacagccgccgcgaaccagctgatgaacaacgtgccacaggccctgcagcagctggctcaacctacccagggcaccacacctagctccaagctgggcggtctgtggaagaccgtgagccctcatagatctcccatcagcaatatggtctctatggccaacaaccacatgggcggctccggtggaggcgggagcggcggcacagagcagcaatggaacttcgccggcatcgaggcagccgcttctgccatccagggcaatgtgaccagcatccattctctgttggacgagggcaaacagagcctgaccaagctggccgccgcctggggaggcagcggctctgaggcctaccagggcgttcaacaaaaatgggacgccactgccaccgaactgaacaacgccctgcagaatttagcccggacaatcagcgaggccggccaggctatggcctctactgaaggcaacgtgaccggcatgttcgccggcggcagcggaggcggcggtagtggaggcaccagcagattcatgacggatccccacgccatgcgggatatggccggcagattcgaggtgcatgctcagaccgtcgaggacgaggccaggcggatgtgggctagcgcccagaacatcagcggcgccggctggagcggaatggccgaagctacaagcctggacaccatgacgcaaatgaaccaggcctttagaaacatcgtgaatatgctgcacggcgtgcgcgacgggctggtgcgggacgctaacaactacgagcagcaggagcaggccagccagcagatcctgtcttctggcggcagcggtggaggaggctctggcggctcatttgtaatggcgtatcctgaaatgctggcagccgccgcggataccctccagagcatcggcgccaccaccgtggccagcaacgccgccgctgccgcccccaccacaggtgtggtgccacctgctgccgacgaggtgagcgccctgaccgccgcccacttcgccgctcacgccgcgatgtaccagagcgtgtccgctagagccgctgcaatccacgaccagttcgtggctaccctggccagcagcgcctcgagctacgccgcaactgaggtcgccaacgccgctgctgccagcggcggaagcggcggtggtggaagcggcggaacaatcaactaccagtttggcgacgtggacgcccacggagctatgatcagagcccaggccggcagcctggaagccgaacaccaggccatcatttcagacgtgctgacagcttctgatttctggggtggggccggctctgctgcctgccagggctttatcacacagctgggcagaaatttccaagtcatctacgagcaggcgaatgcccacggccagaaggtgcaggccgcggggaacaacatggcccagacagactcggctgtcggatcttcttgggccgggggcagcggtggcggcggatctggcgggcctgtgggcggacagtctagcttctacagtgactggtactcccctgcatgcggcaaggctggctgtcagacctacaagtgggaaaccttcctgaccagcgagctccctcagtggctgagcgccaatagagcagttaagcctaccggctctgccgccatcggcctgagcatggccgggagtagcgccatgatcctggccgcctaccacccccagcagttcatctatgccggaagcctgagcgccctgctggaccctagccaggggatgggaggaggttcaggtggaggtggatctggcggcttcgccgcgaccgtgcctccacctgtggtcgccgccaatagagctgaactggccgtgctggccgcgacaaacgggggttctggaggtggcgggtctggcggcagagccgctatggtggaccccgtggtggtggccgccaaccggagcgccttcgtgcagctggtgctgagcaacggcggcagcggcggtggcggatcaggcggcctgatccccatcgccgttggcggtgccctggctggcctggtcctgatcgtgctgatcgcctacctggtggggagaaagcggagccacgccggttaccagaccatctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 417 - protein, 1162 aa, synthetic construct
MAAPGSARRPLLLLLLLLLLGLMHCASATAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAGGSGGGGSGGVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMGGSGGGGSGGAEMKTDAATLAQEAGNFERISGDLKTQIDQVESTAGSLQGQWRGAAGTAAQAAVVRFQEAANKQKQELDEISTNIRQAGVQYSRADEEQQQALSSQMGFGGGGSGGGGSTEQQWNFAGIEAAASAIQGNVTSIHSLLDEGKQSLTKLAAAWGGSGSEAYQGVQQKWDATATELNNALQNLARTISEAGQAMASTEGNVTGMFAGGSGGGGSGGSFVMAYPEMLAAAADTLQSIGATTVASNAAAAAPTTGVVPPAADEVSALTAAHFAAHAAMYQSVSARAAAIHDQFVATLASSASSYAATEVANAAAASGGSGGGGSGGTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSGGGGSGGGGSTINYQFGDVDAHGAMIRAQAGSLEAEHQAIISDVLTASDFWGGAGSAACQGFITQLGRNFQVIYEQANAHGQKVQAAGNNMAQTDSAVGSSWAGGSGGGGSGGPVGGQSSFYSDWYSPACGKAGCQTYKWETFLTSELPQWLSANRAVKPTGSAAIGLSMAGSSAMILAAYHPQQFIYAGSLSALLDPSQGMGGGSGGGGSGGFAATVPPPVVAANRAELAVLAATNGGSGGGGSGGRAAMVDPVVVAANRSAFVQLVLSNGGSGGGGSGGLIPIAVGGALAGLVLIVLIAYLVGRKRSHAGYQTI

SEQ ID NO: 418 - RNA, 3679 nt, synthetic construct
  source 1..3679: mol_type = other RNA
acatttgcttctgacacaactgtgttcactagcaacctcaaacagccaccatggccgcccctggcagcgccagaagacccctgttactgttgctgctgttattgctgctgggactgatgcactgcgccagcgctaccgctgcgtctgataacttccagctgagccagggtgggcagggctttgctattcccatcgggcaggccatggccatcgccggccagatccgcagcggaggtggctctccaaccgtgcacatcggccctacagctttcctgggcctgggagtggtggacaacaacggcaacggcgcaagggtgcaaagagtggtgggctcagcccctgctgcctccctgggcatcagcaccggcgacgtgatcaccgccgtggacggcgcccctatcaacagcgccacagccatggccgacgccctcaacggccaccatcctggagatgtgatcagcgtgacctggcagacaaagagcggcggcaccagaaccggcaatgtgaccctggccgaaggccctcctgccggaggctctggaggcggtggatctggcggagttgatttcggcgccctgccccccgaaattaacagcgccagaatgtacgccggccccggctccgctagcctggttgcagccgctcagatgtgggatagcgtggccagcgacctgttcagcgcggcctctgccttccaaagcgtggtgtggggcctgacagtggggagctggatcggctccagcgccggactgatggtggctgccgcaagcccttacgtggcctggatgagcgtcacagccgggcaggccgagctgaccgccgctcaggttcgggtggccgctgccgcctatgaaaccgcctacggattaacagtgccacctcctgtcatcgccgagaaccgggctgagctgatgatcctgatcgccaccaatctgctgggccagaatacccctgctatcgccgtgaacgaggccgaatacggcgagatgtgggcccaggacgcagccgctatgttcggctacgccgccgccaccgccaccgctaccgctaccctgctccccttcgaagaagcccctgagatgacatctgccggcggcctgcttgagcaggccgccgccgtggaggaagcctccgacaccgccgcagctaaccagctgatgaacaacgtgccccaggctctgcagcagctggcccaacctacccagggcaccacccctagcagcaagctgggaggactgtggaagaccgtctcccctcacagaagccctatctctaacatggtctccatggctaacaaccacatgggcggcagcggtggtggcggatcaggcggagccgaaatgaaaaccgacgccgccaccctggcccaagaggcgggcaacttcgagcggatcagcggggacctgaagacccagattgaccaggtggagagcaccgccggctccctgcaaggccaatggagaggcgccgccggtacggctgcccaggccgctgtggttagatttcaggaggctgctaacaagcagaagcaggagctggacgagatcagcaccaacatccggcaggcaggcgtgcagtacagtagagccgacgaggaacagcagcaagctctgagcagccagatgggcttcggtggaggcgggagcggaggcggcggtagtacagagcagcagtggaactttgccggaatcgaggcggcagccagtgccatccagggcaacgtgacatcaattcacagcctgctggacgagggcaaacagagcctgaccaagctggccgcagcctggggaggctctggctctgaagcttaccagggcgttcagcaaaagtgggacgcaacagccaccgaactgaacaacgccctgcagaacctggcccggaccataagcgaggccggacaggcaatggccagcaccgagggcaatgtgaccggaatgttcgccggcggcagcggtggaggaggctctggaggtagttttgtgatggcttacccagaaatgctggccgctgccgccgacaccctgcagtctatcggcgcaacaaccgtggcctctaatgccgccgccgccgcccctaccacaggcgtggtgccgcctgccgctgacgaggtgtcagctctgaccgcggcgcatttcgcggcccacgccgccatgtaccagagcgtgtctgccagagccgccgccatccacgaccagttcgtggccaccctggccagcagcgccagtagctacgctgccacagaagtggctaacgcagccgccgccagcggtggttccggcggtggtggaagcggcggaacctccagattcatgaccgatccccacgccatgagagacatggccggtagattcgaggtgcacgcccagaccgttgaagatgaagcccggagaatgtgggctagcgcccaaaatatctccggcgccggatggtctggcatggctgaggccacaagcttggatacgatgacccagatgaaccaggcctttagaaacatcgtgaacatgctccacggcgttagagacggcctggtgcgggacgccaacaattacgagcagcaggaacaggcctctcagcagatcctgagctctggtggcggcggatctggcggaggaggttcaacaatcaactaccagtttggcgatgtggatgcccacggagccatgatcagagcccaggctggctctcttgaagccgagcaccaggccatcatcagcgatgtgctgactgcaagcgacttctggggcggtgccggctccgctgcttgtcagggcttcatcacacagctgggaagaaacttccaggtgatctacgagcaggccaatgcgcatggccaaaaagtgcaggcagccggaaacaacatggcccagaccgacagcgctgtgggaagcagctgggccggcggcagcggtggaggtggatctggcggtccagtgggcggccagagcagcttctacagcgactggtacagccctgcgtgcggcaaggccggctgccagacctacaaatgggagacattcctgacatctgagctgcctcagtggctgagcgccaatcgggccgtgaagcccaccggcagtgccgccatcggactgagcatggctggctcctccgccatgattctggctgcctaccacccccagcagttcatctatgccgggagcctgagcgccctgctggaccccagccagggaatgggtggcgggtctggcggtggcggatcaggcggcttcgccgccaccgtgcctccacctgtggtcgccgccaacagagccgagctggctgtgctggccgctacaaacgggggcagcggaggcggaggtagtggcggcagagccgccatggtggaccctgtggtggtggccgccaaccggagcgcttttgtgcagctggtgctgagcaatggcggctctggtggtggagggtcaggcgggctgatccctatcgccgtgggcggtgccctggccggcctagtgctgatcgtgctgatcgcctacctggtgggcagaaagcggagccacgccggctaccagacaatctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 419 - protein, 1225 aa, synthetic construct
MAAPGSARRPLLLLLLLLLLGLMHCASATAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAGGSGGGGSGGAEMKTDAATLAQEAGNFERISGDLKTQIDQVESTAGSLQGQWRGAGGSGGGGSGGTSRFMTDPHAMRDMAGRFEVHAQTVEDEARRMWASAQNISGAGWSGGGSGGGGSGGTEQQWNFAGIEAAASAIQGNVTSIHSLLDEGKQSLTKLAAAWGGSGSGGSGGGGSGGTINYQFGDVDAHGAMIRAQAGSLEAEHQAIISDVLTASDFWGGAGSGGSGGGGSGGVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMGGSGGGGSGGSFVMAYPEMLAAAADTLQSIGATTVASNAAAAAPTTGVVPPAADEVSALTAAHFAAHAAMYQSVSARAAAIHDQFVATLASSASSYAATEVANAAAASGGSGGGGSGGGQWRGAAGTAAQAAVVRFQEAANKQKQELDEISTNIRQAGVQYSRADEEQQQALSSQMGFGGSGGGGSGGSGAGWSGMAEATSLDTMTQMNQAFRNIVNMLHGVRDGLVRDANNYEQQEQASQQILSSGGSGGGGSGGGGSGSEAYQGVQQKWDATATELNNALQNLARTISEAGQAMASTEGNVTGMFAGGSGGGGSGGGGAGSAACQGFITQLGRNFQVIYEQANAHGQKVQAAGNNMAQTDSAVGSSWAGGSGGGGSGGPVGGQSSFYSDWYSPACGKAGCQTYKWETFLTSELPQWLSANRAVKPTGSAAIGLSMAGSSAMILAAYHPQQFIYAGSLSALLDPSQGMGGGSGGGGSGGFAATVPPPVVAANRAELAVLAATNGGSGGGGSGGRAAMVDPVVVAANRSAFVQLVLSNGGSGGGGSGGLIPIAVGGALAGLVLIVLIAYLVGRKRSHAGYQTI

SEQ ID NO: 420 - RNA, 3868 nt, synthetic construct
  source 1..3868: mol_type = other RNA
acatttgcttctgacacaactgtgttcactagcaacctcaaacagccaccatggccgcacctggctcagcccgcagacctctgctgctgctgctattgctgctgctcctgggactgatgcactgcgcctctgccaccgccgcttccgacaacttccagctgagccagggcggccaaggcttcgccatccccatcggccaggctatggcaatcgcgggacaaatcagaagcggaggtggctctcccactgtgcacatcggccctaccgcattcctgggcctgggcgtggtggacaacaacggcaacggcgcccgggtgcagagagtggtgggctcagctcccgccgccagcctgggcataagcaccggggatgtgataacagccgtggatggcgcccctatcaacagcgctactgccatggccgatgccctgaacggccaccaccccggcgacgtgatcagcgtgacctggcagactaagagcggcggcacaagaacaggcaatgtgaccctggccgaaggccctcctgccggcggctctggaggcggtggatctggcggcgccgaaatgaagacagacgccgccaccctggcccaggaggccggaaatttcgaaagaatcagcggcgatctgaagacccagatcgaccaggtcgagagcacagcgggaagcctgcagggccagtggagaggcgccggcggctccggtggtggcggatcaggcggcacaagccgattcatgaccgatccgcacgccatgcgggacatggccgggaggttcgaggtgcacgcccagaccgtggaagatgaagcccggcggatgtgggctagcgctcagaacatcagcggggccggatggagcggaggcgggagcggaggcggcggtagtggaggcaccgagcagcaatggaacttcgctggaatcgaggccgccgccagcgctattcagggcaacgtgaccagcatccactctctgctggacgagggcaaacagtccctgaccaaactggccgccgcctggggcggcagcggctctggcggaagcggtggaggaggctctggcggaaccataaactaccagttcggcgacgtggatgctcacggcgccatgatccgggcccaggccggaagcctggaagccgaacaccaggccatcatcagcgacgtgctgaccgcatctgacttctggggcggcgcgggctccggaggttccggcggtggtggaagcggaggcgtggacttcggcgctctcccccctgagatcaacagcgccagaatgtacgccggaccaggctcagcctccctggtagcggcggctcagatgtgggacagcgtggctagcgacctgttcagcgccgccagcgccttccagagcgtggtctggggactgaccgtgggcagctggatcggaagcagcgccggcctgatggtggccgccgccagcccctacgtggcctggatgagcgttaccgccggtcaagccgagctgacagccgctcaggtgcgcgtggccgccgctgcctacgagacagcctatggcctgaccgtgcccccccccgtgattgctgaaaaccgggccgagctgatgatcctcatcgccaccaacctgcttggccagaacacccctgccatcgccgtgaacgaggccgaatacggcgagatgtgggctcaggacgccgccgccatgttcggctacgccgccgccacagctaccgccaccgctacactattaccattcgaagaggcccctgaaatgacctccgccggcggcctgctggaacaagccgcggccgtggaagaggccagcgataccgctgccgccaaccagctgatgaacaatgtgccacaggccctgcagcagctggctcaacctacccagggcaccacacctagcagcaagctgggaggcctgtggaagaccgtgtccccacacagaagccccatcagcaacatggtctccatggctaacaaccacatgggcggaagcggtggcggcggatctggcggcagttttgtgatggcttatcctgagatgctggccgctgctgccgacaccctgcagagtatcggtgccaccaccgtggccagcaacgcagccgcggctgcccctacaaccggcgtggtgcctcccgctgccgacgaggtgtctgccctgacggccgcccatttcgccgctcatgccgctatgtaccagagcgtgtccgccagagccgccgccattcatgaccagttcgtagcgacactggcctcttctgcctcctcatatgccgccacagaggtggccaacgccgctgccgcatccgggggcagcggcggaggaggttcaggcggcggacagtggcggggcgctgctggcaccgccgcccaggcggcagtggtgagatttcaggaggctgccaacaagcagaaacaggagctggacgagatctccaccaatatcagacaggccggcgttcagtacagccgggctgacgaggaacagcagcaggccctgtcaagccagatgggatttggcggtagcggtggaggtggatctggcggcagcggagccggatggtccggcatggccgaggccacctctctggatacaatgacccaaatgaaccaagcattccggaacatcgtgaacatgctgcacggcgttagagacggccttgtgcgggacgccaacaactacgagcagcaggagcaggcaagccagcagatcctatctagcggtggcagcggaggtggcgggtctggcggtggcggatcaggctctgaagcctaccagggagtccagcagaaatgggacgccacagctacggaactgaacaatgccctgcaaaatctggctagaaccatcagcgaggcgggacaggctatggccagcaccgaaggcaatgtcacaggcatgttcgccggcggctctggaggcggaggtagtgggggcggcggcgctggctcggccgcttgccaggggttcatcacccagctgggcagaaacttccaggtgatctacgagcaggccaacgctcacggccagaaggtgcaggcggccggaaacaacatggcccagaccgacagcgccgtggggtcaagctgggccggcggaagcggtggtggagggtcaggcggccctgtgggcggacagtctagcttctacagcgactggtacagccctgcctgcgggaaggccggttgtcagacatacaagtgggagacattcctgaccagcgagctgcctcagtggctgtctgcgaatagagccgtgaagcctaccgggagcgcagccatcggcctgagtatggccggctcgtctgccatgatcctggccgcttaccaccctcagcaatttatctacgcaggatctctgagcgccctgctggacccttctcaaggcatgggcggtggaagcggaggaggcggttctggaggctttgccgccaccgtgccacctcctgtggttgccgctaaccgggccgaactggctgtgctggctgcgacgaacggcggatctggcggcggcggtagcggaggcagagccgcaatggtggaccccgtggtcgtggccgcaaatagaagcgcctttgtgcagctggtgctgtctaatggcgggagcggcgggggcggtagcggcggcctgatccctattgccgtcggcggcgcactggccggcctggtgctgatcgtgctgatcgcctacctggttggcagaaagagaagtcacgccggttatcaaacaatctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 421 - protein, 1185 aa, synthetic construct
MAVMAPRTLLLLLLGALALTQTWAGSSRAFIIDPTISAIDGLYDLLGIGIPNQGGILYSSLEYFEKALEELAAAFPGDGWLGSAADKYAGKNRNHVNFFQELADLDRQLISLIHDQANAVQTTRDILEGAKKGLEFVRPVAVDLTYIPVVGHALSAAFQAPFCAGAMAVVGGALAYLVVKTLINATQLLKLLAKLAELVAAAIADIISDVADIIKGTLGEVWEFITNALNGLKELWDKLTGWVTGLFSRGWSNLESFFAGVPGLTGATSGLSQVTGLFGAAGLSASSGLAHADSLASSASLPALAGIGGGSGFGGLPSLAQVHAASTRQALRPRADGPVGAAAEQVGGQSQLVSAQGSQGMGGPVGMGGMHPSSGASKGTTTKKYSEGAAAGTEDAERAPVEADAGGGQKVLVRNVVGGSGGGGSGGITDRLPAKAPLLELDGGSGGGGSGGAITINMVNPTGFIRYEDVEQEAMTSDVTVGPAPGQYQLSHLRLLEAEAIHVIREVAAEFERPVLLFSGGKDSIVMLHLALKAFRPGRLPFPVMHVDTGHNFDEVIATRDELVAAAGVRLVVASVQDDIDAGRVVETIPSRNPIQTVTLLRAIRENQFDAAFGGARRDEEKARAKERVFSFRDEFGQWDPKAQRPELWNLYNGRHHKGEHIRVFPLSNWTEFDIWSYIGAEQVRLPSIYFAHRRKVFQRDGMLLAVHRHMQPRADEPVFEATVRFRTVGDVTCTGCVESSASTVAEVIAETAVARLTERGATRADDRISEAGMEDRKRQGYFGGSGGGGSGGKRVEIEVDDDLIQKVIRGGSGGGGSGGAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDSGGPVVNGLGQVVGMNGGSGGGGSGGALAWMCDRAVDLLLLGGSGGGGSGGAIHVMTTVLATLPADGGSGGGGSGGTTRVMATRRNVLDRQGGSGGGGSGGVARVLRPGGHFLYTDGGSGGGGSGGEPSSQSTVPIVGIVAGLAVLAVVVIGAVVAAVMCRRKSSGGKGGSYSQAACSDSAQGSDVSLTA

SEQ ID NO: 422 - RNA, 3748 nt, synthetic construct
  source 1..3748: mol_type = other RNA
acatttgcttctgacacaactgtgttcactagcaacctcaaacagccaccatggctgtgatggccccgcgcaccctcctgctgctcctgctgggagcgctcgcattgacccagacttgggccgggtcatcccgcgccttcattattgaccctaccatttccgccattgacggtctgtacgatctgctgggaatcggcatcccaaaccaaggagggatcctctactcctcgttggaatacttcgagaaggcactcgaggaacttgccgccgcattcccgggcgatggatggctgggaagcgctgctgacaaatacgcggggaagaaccggaaccatgtcaacttcttccaagagctcgccgacctggaccggcagctcatatccctgattcacgaccaggccaatgctgtgcagaccactcgggatatcctggagggagctaagaagggattggaattcgtgcggcctgtggctgtggacctgacctacattccggtggtcggccacgcgctgtccgcggcgttccaggcgcctttctgtgccggagctatggccgtggtcggtggagctctcgcctacctcgtggtcaaaaccctgatcaatgccacccagctgctgaagctgctggctaagctcgcggagttggtcgccgccgcgattgccgatatcattagcgacgtggcagacatcatcaagggcaccctcggagaagtctgggaattcatcaccaacgccctgaacggtctgaaggaactgtgggataagctgactggatgggtcaccggactgttcagccgcggatggtccaacctggagagcttcttcgccggagtgcccggactcaccggtgccacctccgggctttcccaagttaccggactcttcggggccgccggcctgtcggcctcatccgggctggcacacgccgatagcctggcatcctcggcatcgctccccgcgctcgcgggcatcgggggtggcagcggatttggcggactgccctcgctggcacaagtgcacgccgccagcactcggcaggcgctgcgcccgagagccgacggccccgtgggagccgctgcggaacaagtcggtggccagtctcaacttgtgtccgcgcagggaagccaggggatgggtggccctgtgggaatgggcggaatgcatccaagctccggggcctcgaagggcactaccaccaagaagtactccgagggcgctgcggccggcactgaagatgctgaacgggccccggtggaggccgatgccggtggcgggcaaaaggtgctcgtacggaatgtcgtgggaggttctggtggcgggggctccggtggaatcaccgaccggctcccggccaaggctcccctgctggagcttgatggtggatccgggggaggcggttccggcggtgccatcaccatcaacatggtcaaccccactggctttatccgctatgaggatgtggaacaggaggccatgacctcagacgtgaccgtgggacccgcccccggccagtaccagctgtcccacctccgcctgctcgaggccgaagcgattcacgtgatcagggaagtggcggccgaatttgagcgcccggtgctgctgttcagcggtggaaaggatagcattgtgatgttgcatttggccctcaaggccttccgacctggtcggctcccgttccctgtgatgcatgtggataccggacacaacttcgacgaggtcatcgcaacgcgcgacgagttggtggcagccgccggcgtgcgcctggtagtggccagcgtgcaagacgacatcgacgccggcagagtggtggaaaccatcccctcccgcaaccctattcagaccgtgaccctgctgagagccattagggaaaaccagttcgacgctgccttcggaggcgcccggcgggatgaggagaaagcgcgcgcgaaggaaagagtgttctcctttcgggacgagtttggacagtgggaccctaaggcgcaacggcccgagttgtggaacctgtacaacggacggcaccataagggggagcacatccgggtctttcctctgtccaactggacagagttcgatatttggagctacattggagccgaacaagtccggctgccgtccatctacttcgcacaccggagaaaagtgttccagcgcgacggcatgctcctcgcggtgcacaggcatatgcagcccagggcagacgagccggtgtttgaagccaccgtcagattccgcacagtgggcgacgtgacttgcaccggctgcgtggaatcctccgcgtccaccgtggccgaagtgatcgctgagactgcggtcgccagattgaccgagcggggtgccacaagagccgatgaccgcattagcgaagcggggatggaggatcggaagagacaaggatactttggaggctcagggggtggaggctcgggcggaaagcgcgtcgagatcgaagtcgacgatgatctgatccaaaaggtcatccggggtggaagcggcggagggggaagcgggggcgcgccgcccgcgttaagtcaggaccggttcgccgacttcccggccctgcccctggacccttcggcgatggtggcccaagttggcccccaagtggtgaacattaacactaagctgggatacaacaacgctgtgggcgcgggcactggaatcgtcatcgaccctaacggcgtggtgctaaccaacaaccatgtgatagccggggccaccgatatcaacgccttcagcgtggggtccggacagacttacggagtggacgtcgtgggttacgacaggacccaggacgtggccgtgctgcaactgaggggagccggaggactgcccagcgccgcgatcggaggaggggtggccgtgggtgaaccggtggtggccatgggaaattccggcggacagggaggcaccccgagggcggtgcccggccgcgtggtcgctctcggtcaaaccgtgcaggcctccgacagcctgactggcgccgaggaaactctgaacggcctgatccagttcgatgcggccatccagccgggagactcggggggacctgtggtcaacggactgggacaggtcgtgggcatgaacggcggctcgggagggggtggctccgggggcgccctggcctggatgtgcgacagagctgtggatttgctccttctggggggatcgggtggtggcggatctggaggggccatccacgtgatgacgactgtcctggccaccctgccggccgatggcggctccggcggaggaggttcgggcggaactactagagtgatggccacacgacggaacgtgctggaccgccaggggggatccggcggcgggggcagcggaggagtggcccgcgtccttagacctggaggacacttcctgtataccgacggaggatccggaggaggaggatcaggcggagaaccgagttcccagtcaaccgtgcccatcgtcgggatcgtggcaggactcgccgtgttggcagtcgtcgtcattggcgccgtggtggccgccgtgatgtgccgccggaagtcctcaggggggaagggaggctcgtacagccaggccgcctgttccgactcggcccaggggtccgacgtgtcccttactgcatgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 423 - protein, 1158 aa, synthetic construct
MAAPGSARRPLLLLLLLLLLGLMHCASASRAFIIDPTISAIDGLYDLLGIGIPNQGGILYSSLEYFEKALEELAAAFPGDGWLGSAADKYAGKNRNHVNFFQELADLDRQLISLIHDQANAVQTTRDILEGAKKGLEFVRPVAVDLTYIPVVGHALSAAFQAPFCAGAMAVVGGALAYLVVKTLINATQLLKLLAKLAELVAAAIADIISDVADIIKGTLGEVWEFITNALNGLKELWDKLTGWVTGLFSRGWSNLESFFAGVPGLTGATSGLSQVTGLFGAAGLSASSGLAHADSLASSASLPALAGIGGGSGFGGLPSLAQVHAASTRQALRPRADGPVGAAAEQVGGQSQLVSAQGSQGMGGPVGMGGMHPSSGASKGTTTKKYSEGAAAGTEDAERAPVEADAGGGQKVLVRNVVGGSGGGGSGGITDRLPAKAPLLELDGGSGGGGSGGAITINMVNPTGFIRYEDVEQEAMTSDVTVGPAPGQYQLSHLRLLEAEAIHVIREVAAEFERPVLLFSGGKDSIVMLHLALKAFRPGRLPFPVMHVDTGHNFDEVIATRDELVAAAGVRLVVASVQDDIDAGRVVETIPSRNPIQTVTLLRAIRENQFDAAFGGARRDEEKARAKERVFSFRDEFGQWDPKAQRPELWNLYNGRHHKGEHIRVFPLSNWTEFDIWSYIGAEQVRLPSIYFAHRRKVFQRDGMLLAVHRHMQPRADEPVFEATVRFRTVGDVTCTGCVESSASTVAEVIAETAVARLTERGATRADDRISEAGMEDRKRQGYFGGSGGGGSGGKRVEIEVDDDLIQKVIRGGSGGGGSGGAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDSGGPVVNGLGQVVGMNGGSGGGGSGGALAWMCDRAVDLLLLGGSGGGGSGGAIHVMTTVLATLPADGGSGGGGSGGTTRVMATRRNVLDRQGGSGGGGSGGVARVLRPGGHFLYTDGGSGGGGSGGLIPIAVGGALAGLVLIVLIAYLVGRKRSHAGYQTI

SEQ ID NO: 424 - RNA, 3667 nt, synthetic construct
  source 1..3667: mol_type = other RNA
acatttgcttctgacacaactgtgttcactagcaacctcaaacagccaccatggccgctcctggcagcgctcggcggccactgctgctgctactcctcctgctgctgctgggactgatgcactgcgcgagcgcctccagagcatttatcatcgatcctaccatttctgccatcgacgggctgtacgatctgctgggcatcggcatccctaaccagggcggcatcctgtatagctctctggagtactttgagaaggctctggaggaactggctgccgcgtttcctggcgatggatggctgggcagcgccgccgacaaatacgccggaaaaaacagaaaccacgttaacttcttccaggagctggccgacctcgacagacagctcatctccctgatacatgatcaggccaacgccgtccagaccacccgggacatcctggaaggcgccaaaaagggcctggagttcgtgcggcccgtggccgtggatctgacttacatccccgtggtggggcacgctctgagcgccgccttccaagcccctttctgcgccggtgctatggccgtcgtaggcggcgccctggcttatctggtggttaagaccctgatcaacgccacacagctgctgaagttacttgccaagctggccgagctggtcgctgctgccatcgctgatataatcagcgatgtggcggacatcatcaagggcaccctgggcgaggtgtgggagttcatcaccaacgccctgaacggcctgaaggagctgtgggataagctgacagggtgggtcacaggacttttcagcagaggctggtccaatctggaaagcttcttcgctggggtgcctggcctaaccggcgccacaagcggcctgagtcaggtgacaggactgttcggagccgctggcctgtctgcttctagcggcctggctcacgccgacagtctggcctctagcgccagcctgcctgccctggccggcatcggaggtggctctggcttcggcggcctgccaagcctggcacaggtgcacgccgccagcacacggcaagccctcagacctagagccgatggccctgtgggcgccgccgccgaacaggtcggaggccagagccagctggtgtccgcccaaggtagccagggcatgggaggacctgtaggcatgggcggcatgcatcctagcagcggcgcctcaaagggcaccaccacaaagaagtacagcgagggcgcagccgccggaactgaggacgcagaacgggcccctgtcgaagccgacgccggcggcggccaaaaagtgctggttcggaacgtggtggggggatctggaggcggtggatctggcggcatcaccgacagactgcctgccaaggcccctctgctggaactggacggcggcagcggtggtggcggatcaggaggcgctatcacaatcaacatggtcaatcccaccggttttatcagatacgaggacgtggaacaggaggccatgacaagcgacgtgaccgtgggccccgctccaggacagtaccagctgagccacctgcggctgcttgaggccgaagccatccacgtgattagagaagtggccgccgaatttgagagacccgtgcttctgttctccggaggcaaggacagcatcgtgatgctgcacctggcattgaaagcctttcggcctggcagactgccttttcctgtgatgcacgtggacaccggccacaacttcgacgaggtgatcgccaccagagacgaactggtggcggcagccggagtgaggctggtggtggccagcgtgcaggatgatatcgatgccggcagagtggtggaaaccattcccagcagaaaccccatccaaacagtgaccttactgcgggccatcagagagaaccagttcgacgcggccttcggcggcgcccggagagatgaggaaaaggcccgggccaaagagagagtgttttctttcagagacgagttcggccagtgggatcccaaggcccagcggcctgagctgtggaacctgtacaacggaagacaccacaagggagaacacatccgggtgttccctctgagcaactggaccgagttcgatatttggagctacataggcgccgagcaggtgcgtctgcccagcatctacttcgcccatagaagaaaggtgttccaaagagatggcatgctgctggccgtgcaccggcacatgcagcctagagccgatgaacctgtgttcgaggccaccgtgcgcttccggacagtgggcgacgtcacctgcaccggctgtgtggagtcttctgcctctaccgtcgcagaagtgatcgccgagacagccgtggccagactgaccgagagaggcgctacccgggccgacgatcggatctctgaggccggcatggaagatagaaagagacagggctacttcggaggatccggtggaggcgggagcggcggcaagcgggtcgagatcgaggtggacgatgacctgatccagaaggtgattagaggaggcagcggaggcggcggtagtggcggggctccacctgccctgtcccaggaccgcttcgccgacttcccagccctccctctggaccctagcgccatggtggcccaggtgggccctcaggtggtgaacatcaataccaagctgggctacaacaacgctgtgggggccggcacaggcatcgtgatcgaccccaatggagtggtgctgacaaacaatcacgtgatcgccggcgccaccgatatcaacgccttcagcgtgggcagcggccagacatacggcgtggacgtggttggatacgacagaacacaggacgtggccgtgctgcagctgagaggcgcgggcggactaccaagcgccgccatcggcggaggtgtggccgtgggcgagcctgtggtggccatgggcaacagcggcggtcagggcggcacacctcgggctgtgcccggcagagtggtggctttgggccagaccgtgcaggctagcgacagcctgaccggggccgaggaaaccctgaacggcctgatccagttcgacgctgctatccagcccggcgacagcggggggcccgtggtgaacggccttgggcaggttgtgggcatgaacggcggaagcggtggaggaggctctggcggcgccctggcctggatgtgcgacagagctgttgatctgctgctgctgggcggcagcggcggtggtggaagcggtggtgcgatccacgtgatgaccacagtgttggccacactgccggcagacggcggttccggtggcggcggatctggcggcaccaccagggtgatggccacgcgcagaaatgtgctggaccgtcaaggcggctctggcggaggaggttcaggcggcgtggcccgggtcctgagacctggcggacacttcctgtacaccgatggcggatctggtggaggtggatctggcggcctgattcctatcgccgtggggggcgccctagcaggcctggtcctgatcgtgctgatcgcctacctggtgggcagaaagcgcagccacgccggctatcagaccatctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 425 - protein, 1185 aa, synthetic construct
MAVMAPRTLLLLLSGALALTQTWAGSSRAFIIDPTISAIDGLYDLLGIGIPNQGGILYSSLEYFEKALEELAAAFPGDGWLGSAADKYAGKNRNHVNFFQELADLDRQLISLIHDQANAVQTTRDILEGAKKGLEFVRPVAVDLTYIPVVGHALSAAFQAPFCAGAMAVVGGALAYLVVKTLINATQLLKLLAKLAELVAAAIADIISDVADIIKGTLGEVWEFITNALNGLKELWDKLTGWVTGLFSRGWSNLESFFAGVPGLTGATSGLSQVTGLFGAAGLSASSGLAHADSLASSASLPALAGIGGGSGFGGLPSLAQVHAASTRQALRPRADGPVGAAAEQVGGQSQLVSAQGSQGMGGPVGMGGMHPSSGASKGTTTKKYSEGAAAGTEDAERAPVEADAGGGQKVLVRNVVGGSGGGGSGGITDRLPAKAPLLELDGGSGGGGSGGAITINMVNPTGFIRYEDVEQEAMTSDVTVGPAPGQYQLSHLRLLEAEAIHVIREVAAEFERPVLLFSGGKDSIVMLHLALKAFRPGRLPFPVMHVDTGHNFDEVIATRDELVAAAGVRLVVASVQDDIDAGRVVETIPSRNPIQTVTLLRAIRENQFDAAFGGARRDEEKARAKERVFSFRDEFGQWDPKAQRPELWNLYNGRHHKGEHIRVFPLSNWTEFDIWSYIGAEQVRLPSIYFAHRRKVFQRDGMLLAVHRHMQPRADEPVFEATVRFRTVGDVTCTGCVESSASTVAEVIAETAVARLTERGATRADDRISEAGMEDRKRQGYFGGSGGGGSGGKRVEIEVDDDLIQKVIRGGSGGGGSGGAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDSGGPVVNGLGQVVGMNGGSGGGGSGGALAWMCDRAVDLLLLGGSGGGGSGGAIHVMTTVLATLPADGGSGGGGSGGTTRVMATRRNVLDRQGGSGGGGSGGVARVLRPGGHFLYTDGGSGGGGSGGEPSSQSTVPIVGIVAGLAVLAVVVIGAVVAAVMCRRKSSGGKGGSYSQAACSDSAQGSDVSLTA

SEQ ID NO: 426 - RNA, 3748 nt, synthetic construct
  source 1..3748: mol_type = other RNA
acatttgcttctgacacaactgtgttcactagcaacctcaaacagccaccatggctgtgatggccccgcgcaccctcctgctgctcctgtcgggagcgctcgcattgacccagacttgggccgggtcatcccgcgccttcattattgaccctaccatttccgccattgacggtctgtacgatctgctgggaatcggcatcccaaaccaaggagggatcctctactcctcgttggaatacttcgagaaggcactcgaggaacttgccgccgcattcccgggcgatggatggctgggaagcgctgctgacaaatacgcggggaagaaccggaaccatgtcaacttcttccaagagctcgccgacctggaccggcagctcatatccctgattcacgaccaggccaatgctgtgcagaccactcgggatatcctggagggagctaagaagggattggaattcgtgcggcctgtggctgtggacctgacctacattccggtggtcggccacgcgctgtccgcggcgttccaggcgcctttctgtgccggagctatggccgtggtcggtggagctctcgcctacctcgtggtcaaaaccctgatcaatgccacccagctgctgaagctgctggctaagctcgcggagttggtcgccgccgcgattgccgatatcattagcgacgtggcagacatcatcaagggcaccctcggagaagtctgggaattcatcaccaacgccctgaacggtctgaaggaactgtgggataagctgactggatgggtcaccggactgttcagccgcggatggtccaacctggagagcttcttcgccggagtgcccggactcaccggtgccacctccgggctttcccaagttaccggactcttcggggccgccggcctgtcggcctcatccgggctggcacacgccgatagcctggcatcctcggcatcgctccccgcgctcgcgggcatcgggggtggcagcggatttggcggactgccctcgctggcacaagtgcacgccgccagcactcggcaggcgctgcgcccgagagccgacggccccgtgggagccgctgcggaacaagtcggtggccagtctcaacttgtgtccgcgcagggaagccaggggatgggtggccctgtgggaatgggcggaatgcatccaagctccggggcctcgaagggcactaccaccaagaagtactccgagggcgctgcggccggcactgaagatgctgaacgggccccggtggaggccgatgccggtggcgggcaaaaggtgctcgtacggaatgtcgtgggaggttctggtggcgggggctccggtggaatcaccgaccggctcccggccaaggctcccctgctggagcttgatggtggatccgggggaggcggttccggcggtgccatcaccatcaacatggtcaaccccactggctttatccgctatgaggatgtggaacaggaggccatgacctcagacgtgaccgtgggacccgcccccggccagtaccagctgtcccacctccgcctgctcgaggccgaagcgattcacgtgatcagggaagtggcggccgaatttgagcgcccggtgctgctgttcagcggtggaaaggatagcattgtgatgttgcatttggccctcaaggccttccgacctggtcggctcccgttccctgtgatgcatgtggataccggacacaacttcgacgaggtcatcgcaacgcgcgacgagttggtggcagccgccggcgtgcgcctggtagtggccagcgtgcaagacgacatcgacgccggcagagtggtggaaaccatcccctcccgcaaccctattcagaccgtgaccctgctgagagccattagggaaaaccagttcgacgctgccttcggaggcgcccggcgggatgaggagaaagcgcgcgcgaaggaaagagtgttctcctttcgggacgagtttggacagtgggaccctaaggcgcaacggcccgagttgtggaacctgtacaacggacggcaccataagggggagcacatccgggtctttcctctgtccaactggacagagttcgatatttggagctacattggagccgaacaagtccggctgccgtccatctacttcgcacaccggagaaaagtgttccagcgcgacggcatgctcctcgcggtgcacaggcatatgcagcccagggcagacgagccggtgtttgaagccaccgtcagattccgcacagtgggcgacgtgacttgcaccggctgcgtggaatcctccgcgtccaccgtggccgaagtgatcgctgagactgcggtcgccagattgaccgagcggggtgccacaagagccgatgaccgcattagcgaagcggggatggaggatcggaagagacaaggatactttggaggctcagggggtggaggctcgggcggaaagcgcgtcgagatcgaagtcgacgatgatctgatccaaaaggtcatccggggtggaagcggcggagggggaagcgggggcgcgccgcccgcgttaagtcaggaccggttcgccgacttcccggccctgcccctggacccttcggcgatggtggcccaagttggcccccaagtggtgaacattaacactaagctgggatacaacaacgctgtgggcgcgggcactggaatcgtcatcgaccctaacggcgtggtgctaaccaacaaccatgtgatagccggggccaccgatatcaacgccttcagcgtggggtccggacagacttacggagtggacgtcgtgggttacgacaggacccaggacgtggccgtgctgcaactgaggggagccggaggactgcccagcgccgcgatcggaggaggggtggccgtgggtgaaccggtggtggccatgggaaattccggcggacagggaggcaccccgagggcggtgcccggccgcgtggtcgctctcggtcaaaccgtgcaggcctccgacagcctgactggcgccgaggaaactctgaacggcctgatccagttcgatgcggccatccagccgggagactcggggggacctgtggtcaacggactgggacaggtcgtgggcatgaacggcggctcgggagggggtggctccgggggcgccctggcctggatgtgcgacagagctgtggatttgctccttctggggggatcgggtggtggcggatctggaggggccatccacgtgatgacgactgtcctggccaccctgccggccgatggcggctccggcggaggaggttcgggcggaactactagagtgatggccacacgacggaacgtgctggaccgccaggggggatccggcggcgggggcagcggaggagtggcccgcgtccttagacctggaggacacttcctgtataccgacggaggatccggaggaggaggatcaggcggagaaccgagttcccagtcaaccgtgcccatcgtcgggatcgtggcaggactcgccgtgttggcagtcgtcgtcattggcgccgtggtggccgccgtgatgtgccgccggaagtcctcaggggggaagggaggctcgtacagccaggccgcctgttccgactcggcccaggggtccgacgtgtcccttactgcatgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 427 - protein, 1195 aa, synthetic construct
MAVMAPRTLLLLLLGALALTQTWAGSSRAFIIDPTISAIDGLYDLLGIGIPNQGGILYSSLEYFEKALEELAAAFPGDGWLGSAADKYAGKNRNHVNFFQELADLDRQLISLIHDQANAVQTTRDILEGAKKGLEFVRPVAVDLTYIPVVGHALSAAFQAPFCAGAMAVVGGALAYLVVKTLINATQLLKLLAKLAELVAAAIADIISDVADIIKGTLGEVWEFITNALNGLKELWDKLTGWVTGLFSRGWSNLESFFAGVPGLTGATSGLSQVTGLFGAAGLSASSGLAHADSLASSASLPALAGIGGGSGFGGLPSLAQVHAASTRQALRPRADGPVGAAAEQVGGQSQLVSAQGSQGMGGPVGMGGMHPSSGASKGTTTKKYSEGAAAGTEDAERAPVEADAGGGQKVLVRNVVGGSGGGGSGGITDRLPAKAPLLELDGGSGGGGSGGAITINMVNPTGFIRYEDVEQEAMTSDVTVGPAPGQYQLSHLRLLEAEAIHVIREVAAEFERPVLLFSGGKDSIVMLHLALKAFRPGRLPFPVMHVDTGHNFDEVIATRDELVAAAGVRLVVASVQDDIDAGRVVETIPSRNPIQTVTLLRAIRENQFDAAFGGARRDEEKARAKERVFSFRDEFGQWDPKAQRPELWNLYNGRHHKGEHIRVFPLSNWTEFDIWSYIGAEQVRLPSIYFAHRRKVFQRDGMLLAVHRHMQPRADEPVFEATVRFRTVGDVTCTGCVESSASTVAEVIAETAVARLTERGATRADDRISEAGMEDRKRQGYFGGSGGGGSGGKRVEIEVDDDLIQKVIRGGSGGGGSGGAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDSGGPVVNGLGQVVGMNGGSGGGGSGGALAWMCDRAVDLLLLGGSGGGGSGGAIHVMTTVLATLPADGGSGGGGSGGTTRVMATRRNVLDRQGGSGGGGSGGVARVLRPGGHFLYTDGGSGGGGSGGENLRMKLPKPEPSSQSTVPIVGIVAGLAVLAVVVIGAVVAAVMCRRKSSGGKGGSYSQAACSDSAQGSDVSLTA

SEQ ID NO: 428 - RNA, 3778 nt, synthetic construct
  source 1..3778: mol_type = other RNA
acatttgcttctgacacaactgtgttcactagcaacctcaaacagccaccatggctgtgatggccccgcgcaccctcctgctgctcctgctgggagcgctcgcattgacccagacttgggccgggtcatcccgcgccttcattattgaccctaccatttccgccattgacggtctgtacgatctgctgggaatcggcatcccaaaccaaggagggatcctctactcctcgttggaatacttcgagaaggcactcgaggaacttgccgccgcattcccgggcgatggatggctgggaagcgctgctgacaaatacgcggggaagaaccggaaccatgtcaacttcttccaagagctcgccgacctggaccggcagctcatatccctgattcacgaccaggccaatgctgtgcagaccactcgggatatcctggagggagctaagaagggattggaattcgtgcggcctgtggctgtggacctgacctacattccggtggtcggccacgcgctgtccgcggcgttccaggcgcctttctgtgccggagctatggccgtggtcggtggagctctcgcctacctcgtggtcaaaaccctgatcaatgccacccagctgctgaagctgctggctaagctcgcggagttggtcgccgccgcgattgccgatatcattagcgacgtggcagacatcatcaagggcaccctcggagaagtctgggaattcatcaccaacgccctgaacggtctgaaggaactgtgggataagctgactggatgggtcaccggactgttcagccgcggatggtccaacctggagagcttcttcgccggagtgcccggactcaccggtgccacctccgggctttcccaagttaccggactcttcggggccgccggcctgtcggcctcatccgggctggcacacgccgatagcctggcatcctcggcatcgctccccgcgctcgcgggcatcgggggtggcagcggatttggcggactgccctcgctggcacaagtgcacgccgccagcactcggcaggcgctgcgcccgagagccgacggccccgtgggagccgctgcggaacaagtcggtggccagtctcaacttgtgtccgcgcagggaagccaggggatgggtggccctgtgggaatgggcggaatgcatccaagctccggggcctcgaagggcactaccaccaagaagtactccgagggcgctgcggccggcactgaagatgctgaacgggccccggtggaggccgatgccggtggcgggcaaaaggtgctcgtacggaatgtcgtgggaggttctggtggcgggggctccggtggaatcaccgaccggctcccggccaaggctcccctgctggagcttgatggtggatccgggggaggcggttccggcggtgccatcaccatcaacatggtcaaccccactggctttatccgctatgaggatgtggaacaggaggccatgacctcagacgtgaccgtgggacccgcccccggccagtaccagctgtcccacctccgcctgctcgaggccgaagcgattcacgtgatcagggaagtggcggccgaatttgagcgcccggtgctgctgttcagcggtggaaaggatagcattgtgatgttgcatttggccctcaaggccttccgacctggtcggctcccgttccctgtgatgcatgtggataccggacacaacttcgacgaggtcatcgcaacgcgcgacgagttggtggcagccgccggcgtgcgcctggtagtggccagcgtgcaagacgacatcgacgccggcagagtggtggaaaccatcccctcccgcaaccctattcagaccgtgaccctgctgagagccattagggaaaaccagttcgacgctgccttcggaggcgcccggcgggatgaggagaaagcgcgcgcgaaggaaagagtgttctcctttcgggacgagtttggacagtgggaccctaaggcgcaacggcccgagttgtggaacctgtacaacggacggcaccataagggggagcacatccgggtctttcctctgtccaactggacagagttcgatatttggagctacattggagccgaacaagtccggctgccgtccatctacttcgcacaccggagaaaagtgttccagcgcgacggcatgctcctcgcggtgcacaggcatatgcagcccagggcagacgagccggtgtttgaagccaccgtcagattccgcacagtgggcgacgtgacttgcaccggctgcgtggaatcctccgcgtccaccgtggccgaagtgatcgctgagactgcggtcgccagattgaccgagcggggtgccacaagagccgatgaccgcattagcgaagcggggatggaggatcggaagagacaaggatactttggaggctcagggggtggaggctcgggcggaaagcgcgtcgagatcgaagtcgacgatgatctgatccaaaaggtcatccggggtggaagcggcggagggggaagcgggggcgcgccgcccgcgttaagtcaggaccggttcgccgacttcccggccctgcccctggacccttcggcgatggtggcccaagttggcccccaagtggtgaacattaacactaagctgggatacaacaacgctgtgggcgcgggcactggaatcgtcatcgaccctaacggcgtggtgctaaccaacaaccatgtgatagccggggccaccgatatcaacgccttcagcgtggggtccggacagacttacggagtggacgtcgtgggttacgacaggacccaggacgtggccgtgctgcaactgaggggagccggaggactgcccagcgccgcgatcggaggaggggtggccgtgggtgaaccggtggtggccatgggaaattccggcggacagggaggcaccccgagggcggtgcccggccgcgtggtcgctctcggtcaaaccgtgcaggcctccgacagcctgactggcgccgaggaaactctgaacggcctgatccagttcgatgcggccatccagccgggagactcggggggacctgtggtcaacggactgggacaggtcgtgggcatgaacggcggctcgggagggggtggctccgggggcgccctggcctggatgtgcgacagagctgtggatttgctccttctggggggatcgggtggtggcggatctggaggggccatccacgtgatgacgactgtcctggccaccctgccggccgatggcggctccggcggaggaggttcgggcggaactactagagtgatggccacacgacggaacgtgctggaccgccaggggggatccggcggcgggggcagcggaggagtggcccgcgtccttagacctggaggacacttcctgtataccgacggaggatccggaggaggaggatcaggcggagagaacttgagaatgaagctgcccaagcccgaaccgagttcccagtcaaccgtgcccatcgtcgggatcgtggcaggactcgccgtgttggcagtcgtcgtcattggcgccgtggtggccgccgtgatgtgccgccggaagtcctcaggggggaagggaggctcgtacagccaggccgcctgttccgactcggcccaggggtccgacgtgtcccttactgcatgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 429 - protein, 1006 aa, synthetic construct
MAAPGSARRPLLLLLLLLLLGLMHCASASRAFIIDPTISAIDGLYDLLGIGIPNQGGILYSSLEYFEKALEELAAAFPGDGWLGSAADKYAGKNRNHVNFFQELADLDRQLISLIHDQANAVQTTRDILEGAKKGLEFVRPVAVDLTYIPVVGHALSAAFQAPFCAGAMAVVGGALAYLVVKTLINATQLLKLLAKLAELVAAAIADIISDVADIIKGTLGEVWEFITNALNGLKELWDKLTGWVTGLFSRGWSNLESFFAGVPGLTGATSGLSQVTGLFGAAGLSASSGLAHADSLASSASLPALAGIGGGSGFGGLPSLAQVHAASTRQALRPRADGPVGAAAEQVGGQSQLVSAQGSQGMGGPVGMGGMHPSSGASKGTTTKKYSEGAAAGTEDAERAPVEADAGGGQKVLVRNVVGGSGGGGSGGAITINMVNPTGFIRYEDVEQEAMTSDVTVGPAPGQYQLSHLRLLEAEAIHVIREVAAEFERPVLLFSGGKDSIVMLHLALKAFRPGRLPFPVMHVDTGHNFDEVIATRDELVAAAGVRLVVASVQDDIDAGRVVETIPSRNPIQTVTLLRAIRENQFDAAFGGARRDEEKARAKERVFSFRDEFGQWDPKAQRPELWNLYNGRHHKGEHIRVFPLSNWTEFDIWSYIGAEQVRLPSIYFAHRRKVFQRDGMLLAVHRHMQPRADEPVFEATVRFRTVGDVTCTGCVESSASTVAEVIAETAVARLTERGATRADDRISEAGMEDRKRQGYFGGSGGGGSGGAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDSGGPVVNGLGQVVGMNGGSGGGGSGGLIPIAVGGALAGLVLIVLIAYLVGRKRSHAGYQTI

SEQ ID NO: 430 - RNA, 3211 nt, synthetic construct
  source 1..3211: mol_type = other RNA
acatttgcttctgacacaactgtgttcactagcaacctcaaacagccaccatggccgcccctggatcagccaggcgcccccttctgctgctgctcctcctgctgcttctgggtctgatgcattgcgccagcgccagccgcgccttcattattgaccctaccatttccgccattgacggtctgtacgatctgctgggaatcggcatcccaaaccaaggagggatcctctactcctcgttggaatacttcgagaaggcactcgaggaacttgccgccgcattcccgggcgatggatggctgggaagcgctgctgacaaatacgcggggaagaaccggaaccatgtcaacttcttccaagagctcgccgacctggaccggcagctcatatccctgattcacgaccaggccaatgctgtgcagaccactcgggatatcctggagggagctaagaagggattggaattcgtgcggcctgtggctgtggacctgacctacattccggtggtcggccacgcgctgtccgcggcgttccaggcgcctttctgtgccggagctatggccgtggtcggtggagctctcgcctacctcgtggtcaaaaccctgatcaatgccacccagctgctgaagctgctggctaagctcgcggagttggtcgccgccgcgattgccgatatcattagcgacgtggcagacatcatcaagggcaccctcggagaagtctgggaattcatcaccaacgccctgaacggtctgaaggaactgtgggataagctgactggatgggtcaccggactgttcagccgcggatggtccaacctggagagcttcttcgccggagtgcccggactcaccggtgccacctccgggctttcccaagttaccggactcttcggggccgccggcctgtcggcctcatccgggctggcacacgccgatagcctggcatcctcggcatcgctccccgcgctcgcgggcatcgggggtggcagcggatttggcggactgccctcgctggcacaagtgcacgccgccagcactcggcaggcgctgcgcccgagagccgacggccccgtgggagccgctgcggaacaagtcggtggccagtctcaacttgtgtccgcgcagggaagccaggggatgggtggccctgtgggaatgggcggaatgcatccaagctccggggcctcgaagggcactaccaccaagaagtactccgagggcgctgcggccggcactgaagatgctgaacgggccccggtggaggccgatgccggtggcgggcaaaaggtgctcgtacggaatgtcgtgggtggatccgggggaggcggttccggcggtgccatcaccatcaacatggtcaaccccactggctttatccgctatgaggatgtggaacaggaggccatgacctcagacgtgaccgtgggacccgcccccggccagtaccagctgtcccacctccgcctgctcgaggccgaagcgattcacgtgatcagggaagtggcggccgaatttgagcgcccggtgctgctgttcagcggtggaaaggatagcattgtgatgttgcatttggccctcaaggccttccgacctggtcggctcccgttccctgtgatgcatgtggataccggacacaacttcgacgaggtcatcgcaacgcgcgacgagttggtggcagccgccggcgtgcgcctggtagtggccagcgtgcaagacgacatcgacgccggcagagtggtggaaaccatcccctcccgcaaccctattcagaccgtgaccctgctgagagccattagggaaaaccagttcgacgctgccttcggaggcgcccggcgggatgaggagaaagcgcgcgcgaaggaaagagtgttctcctttcgggacgagtttggacagtgggaccctaaggcgcaacggcccgagttgtggaacctgtacaacggacggcaccataagggggagcacatccgggtctttcctctgtccaactggacagagttcgatatttggagctacattggagccgaacaagtccggctgccgtccatctacttcgcacaccggagaaaagtgttccagcgcgacggcatgctcctcgcggtgcacaggcatatgcagcccagggcagacgagccggtgtttgaagccaccgtcagattccgcacagtgggcgacgtgacttgcaccggctgcgtggaatcctccgcgtccaccgtggccgaagtgatcgctgagactgcggtcgccagattgaccgagcggggtgccacaagagccgatgaccgcattagcgaagcggggatggaggatcggaagagacaaggatactttggtggaagcggcggagggggaagcgggggcgcgccgcccgcgttaagtcaggaccggttcgccgacttcccggccctgcccctggacccttcggcgatggtggcccaagttggcccccaagtggtgaacattaacactaagctgggatacaacaacgctgtgggcgcgggcactggaatcgtcatcgaccctaacggcgtggtgctaaccaacaaccatgtgatagccggggccaccgatatcaacgccttcagcgtggggtccggacagacttacggagtggacgtcgtgggttacgacaggacccaggacgtggccgtgctgcaactgaggggagccggaggactgcccagcgccgcgatcggaggaggggtggccgtgggtgaaccggtggtggccatgggaaattccggcggacagggaggcaccccgagggcggtgcccggccgcgtggtcgctctcggtcaaaccgtgcaggcctccgacagcctgactggcgccgaggaaactctgaacggcctgatccagttcgatgcggccatccagccgggagactcggggggacctgtggtcaacggactgggacaggtcgtgggcatgaacggcggctcgggagggggtggctccggtggcctgattccaatcgccgtcgggggagcactggccggactggtgctcattgtgctgatcgcttacctcgtgggaagaaagcgctcccacgccggataccagaccatctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 431 - protein, 844 aa, synthetic construct
MAAPGSARRPLLLLLLLLLLGLMHCASADFGALPPEINSARMYAGAGAGPMMAAGAAWNGLAAELGTTAASYESVITRLTTESWMGPASMAMVAAAQPYLAWLTYTAEAAAHAGSQAMASAAAYEAAYAMTVPPEVVAANRALLAALVATNVLGINTPAIMATEALYAEMWAQDALAMYGYAAASGAAGMLQPLSPPSQTTNPGGLAAQSAAVGSAAATAAVNQVSVADLISSLPNAVSGLASPVTSVLDSTGLSGIIADIDALLATPFVANIINSAVNTAAWYVNAAIPTAIFLANALNSGAPVAIAEGAIEAAEGAASAAAAGLADSVTPAGLGASLGEATLVGRLSVPAAWSTAAPATTAGATALEGSGWTVAAEEAGPVTGMMPGMASAAKGTGAYAGPRYGFKPTVMPKQVVVGGSGGGGSGGDFALLPPEVNSARMYTGPGAGSLLAAAGGWDSLAAELATTAEAYGSVLSGLAALHWRGPAAESMAVTAAPYIGWLYTTAEKTQQTAIQARAAALAFEQAYAMTLPPPVVAANRIQLLALIATNFFGQNTAAIAATEAQYAEMWAQDAAAMYGYATASAAAALLTPFSPPRQTTNPAGLTAQAAAVSQATDPLSLLIETVTQALQALTIPSFIPEDFTFLDAIFAGYATVGVTQDVESFVAGTIGAESNLGLLNVGDENPAEVTPGDFGIGELVSATSPGGGVSASGAGGAASVGNTVLASVGRANSIGQLSVPPSWAAPSTRPVSALSPAGLTTLPGTDVAEHGMPGVPGVPVAAGRASGVLPRYGVRLTVMAHPPAAGGSLIPIAVGGALAGLVLIVLIAYLVGRKRSHAGYQTI

SEQ ID NO: 432 - RNA, 2725 nt, synthetic construct
  source 1..2725: mol_type = other RNA
acatttgcttctgacacaactgtgttcactagcaacctcaaacagccaccatggccgcacctggatccgcaagacgccctttactccttctcctgcttctgctgctgctgggcctcatgcattgcgcctccgccgatttcggagccctgccacctgaaatcaactcggccaggatgtacgccggagctggagctggcccgatgatggccgcaggagctgcttggaacggtctggcagcggaactcggcactactgccgcctcctacgagagcgtgatcactcgccttaccaccgaaagctggatgggaccggcgtccatggccatggtcgcggctgctcaaccctacctggcgtggcttacctacaccgctgaagcggcagcccatgctggatcccaagcaatggcgtccgcagctgcatacgaggcagcctacgccatgactgtgccaccggaggtcgtggcagccaatcgagccctccttgccgctttggtggccacgaacgtgctgggcatcaacactcccgcaattatggctaccgaggcgctctacgcggagatgtgggcccaggacgctctggctatgtacggctacgctgccgcatcgggagcagccggcatgctacaacctctgtcacctccaagccaaaccaccaatcccggaggccttgctgcccaatcagctgccgtgggatctgcggcagccactgctgcggtcaatcaagtctccgtggccgatctcatttcctccctgcccaacgccgtgtcgggcttggcatcgcccgtgacttcggtcctggacagcaccggtctgtccggcattatcgccgacatcgatgccctgcttgcgacccccttcgtcgccaatatcatcaactccgccgtgaacaccgccgcttggtacgtgaacgcggccatcccaaccgccattttcctggcgaacgctctcaactcgggagctcctgtcgccatcgccgaaggcgccattgaagcagcggaaggagccgcgtcagcagctgcagccggattggcggatagcgtgactccagccggacttggggcttccttgggggaagccactctggtcggtcggcttagcgtgccagccgcatggtccactgccgcaccagcaaccaccgcgggagctactgccttggaagggtccggttggacggtcgccgcagaggaagccggacccgtgactgggatgatgcccggaatggcttccgcggccaagggaactggagcctatgccgggcctagatatggcttcaagcctaccgtcatgcctaaacaggtcgtcgtgggagggagtgggggtggaggatcgggaggagacttcgcgctgcttccgcctgaagtgaacagcgcccggatgtacaccggacctggagcaggatccctgttggccgctgcaggaggttgggactcgttggctgcggaactggccaccaccgctgaggcctacgggtccgtgctctccggcttggcggcactgcactggaggggaccagctgccgaatccatggccgtgactgccgctccgtacattggctggctgtacaccaccgccgaaaagacccagcagaccgccattcaagcacgggcagcagctcttgcctttgagcaggcgtacgcgatgactctccctccgcctgtggtcgctgccaaccgcatccagctgctggccctgatcgcgaccaacttcttcggccagaacacagccgccatcgctgccaccgaagcacagtacgccgaaatgtgggcgcaggatgctgccgcgatgtacggatacgccaccgcatcagccgctgcggcattgctgacaccgttctcccctccgcgccaaacgaccaacccagcgggtctgactgcacaggcagccgctgtgtcccaagccactgatccgctctccctgctgatcgaaactgtgactcaagccctgcaagccctgaccatcccttccttcatccctgaggacttcaccttcctcgacgccatcttcgctggctacgcgaccgtgggagtgacccaggacgtggaaagctttgtggccggaaccatcggagcagagtccaacctgggactgctgaacgtcggggatgagaaccccgcggaagtgacccccggcgatttcggcattggggagctggtgtccgctacctcacccggtggaggggtgtcggcatccggtgctggaggagctgccagcgtgggcaatactgtgctcgcctcagtcggacgcgccaacagcattggccagctgagcgtgcctccgtcatgggccgcaccctcaactcgccctgtgtcagccctgagccctgctgggctgaccaccctcccgggaacagacgtggctgagcacggcatgccgggagtcccaggagtgccagttgcagccgggagagctagcggggtgctccctagatacggagtccggctcaccgtgatggcccatccaccggcagcaggaggctcgctgatcccgatagcagtgggaggagcgcttgcgggcttggtgttgattgtcctcatcgcctacctcgtgggaagaaagcggtcacacgccggataccagaccatttgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 433 - protein, 19 aa, Homo sapiens
MGFLPKLLLLASFFPAGQA

SEQ ID NO: 434 - protein, 31 aa, Homo sapiens
LLWVLGLLVGLLLLLLGLGACYTWRRRRVCK

SEQ ID NO: 435-436 - skipped (2 entries)

SEQ ID NO: 437 - protein, 725 aa, Mycobacterium tuberculosis
MHHTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAEFMVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMSMTNSGVSMTNTLSSMLKGFAPAAAAQAVQTAAQNGVRAMSSLGSSLGSSGLGGGVAANLGRAASVGSLSVPQAWAAANQAVTPAARALPLTSLTSAAERGPGQMLGGLPVGQMGARAGGGLSGVLRVPPRPYVMPHSPAAGDIAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDAGGPVVNGLGQVVGMNTAAS

SEQ ID NO: 438 - protein, 775 aa, synthetic construct
MGFLPKLLLLASFFPAGQAMTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAEFMVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMSMTNSGVSMTNTLSSMLKGFAPAAAAQAVQTAAQNGVRAMSSLGSSLGSSGLGGGVAANLGRAASVGSLSVPQAWAAANQAVTPAARALPLTSLTSAAERGPGQMLGGLPVGQMGARAGGGLSGVLRVPPRPYVMPHSPAAGDIAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDAGGPVVNGLGQVVGMNTAASGSLLWVLGLLVGLLLLLLGLGACYTWRRRRVCK

SEQ ID NO: 439 - RNA, 2518 nt, synthetic construct
  source 1..2518: mol_type = other RNA
acatttgcttctgacacaactgtgttcactagcaacctcaaacagccaccatgggctttttgccgaagctgttactgctggcctcattctttccggccggtcaagcgatgacggcggccagcgacaactttcagttgtctcagggcggtcaaggctttgccatacccatcggtcaagctatggccattgcgggccagattcgttcgggcggcggcagtccgactgtgcatatcggtccgactgcgtttttaggtctgggtgtggtcgacaacaacggaaatggtgcacgcgtccaacgcgttgtgggctcagcaccagcggcgtcactgggaatcagcacgggtgacgtaattactgcagtggacggtgcgccgattaactctgccacggccatggcggatgctctgaacggtcaccacccgggggatgttatttccgtcacttggcaaaccaaatctggtgggacccgtacgggcaacgttaccctggcggaagggccgccggcagagttcatggttgatttcggtgcccttccgcccgagatcaactcggcccgtatgtatgctggccccggctctgctagtctggtggctgcggcgcaaatgtgggatagtgtagcctctgaccttttcagtgcggctagcgcgttccagtcggtcgtgtggggtcttactgttggctcgtggattggctcatcagcgggtctgatggtggcggctgctagcccatatgtcgcgtggatgtccgtgacggccgggcaggctgaacttaccgctgcccaggtaagagttgctgcagccgcgtacgaaacagcgtatggcctgacggttcccccgcccgtgatcgcggaaaaccgcgccgaactgatgattctcatcgcgactaacctcctcggccagaacaccccggcaattgcggtgaacgaggcggaatatggtgaaatgtgggcgcaggacgcagccgccatgttcggctacgcagcggcaacagcgacagcaacagcaaccctgctgcctttcgaagaagcgcctgaaatgaccagtgccggcggcctgttggaacaggcagcagccgtggaggaagcgtccgatactgctgccgccaatcagctgatgaacaatgtgcctcaggcgctgcagcaactggcccagcctacccagggcaccaccccgagcagcaaactcggcgggctgtggaaaaccgtctccccgcaccgcagcccgatttctaatatggtgtccatggccaataatcatatgagcatgaccaatagcggcgtcagcatgaccaacactctgagcagcatgctgaaaggctttgccccggcggctgccgcgcaggccgtccagaccgccgcgcaaaatggtgtgcgcgctatgtcgtccctgggaagttcactcggttcttccggtctggggggtggtgtggcagccaacttaggtcgtgccgcgagcgttggcagcttatcggtaccacaggcatgggcagcagcgaaccaggctgtaacgccagcagcacgcgcgttgccgctaacctcgctaacctccgcggcagagcgtgggccgggccagatgcttggaggacttccagttgggcaaatgggggcacgtgcaggtggtgggctttcaggagtattgcgggtacctcctcgtccctatgtgatgccacattccccggctgctggtgatatcgcaccaccagcgctatctcaagatcggtttgcagattttccggcgctgccgctggaccctagcgccatggtagcgcaggtgggcccgcaggttgtcaatataaataccaagttaggttataataacgcggtcggcgctggcacgggcattgtcatcgatccgaatggtgtggttctgaccaacaatcatgtgatcgcgggcgcgaccgatattaacgcctttagtgtggggagtggacagacgtacggcgtggatgtcgttggctacgaccgaacacaggatgttgccgtgctgcagctccgtggtgccggtgggttaccgtcggcggcgattggtggaggcgtagccgtcggtgaaccagttgtagcaatgggcaattcgggcggacaaggggggacacctcgagcagtaccgggaagggtggtggccctgggccaaaccgttcaagccagcgatagtttgacgggagcagaggaaacgttaaatggtctgattcagtttgatgcggccatccaaccgggcgatgctggcggtccggttgttaatggcctgggccaggttgtgggtatgaacacggcggcttctggtagcctcctgtgggtgctgggcctgttggtcggtctgttgttgttactgttaggcctgggggcgtgttacacctggcgtcgccgccgcgtctgcaaatgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 440 - protein, 778 aa, synthetic construct
MKAILVVMLYTFTTANAMTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAEFMVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMSMTNSGVSMTNTLSSMLKGFAPAAAAQAVQTAAQNGVRAMSSLGSSLGSSGLGGGVAANLGRAASVGSLSVPQAWAAANQAVTPAARALPLTSLTSAAERGPGQMLGGLPVGQMGARAGGGLSGVLRVPPRPYVMPHSPAAGDIAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDAGGPVVNGLGQVVGMNTAASGSLAIYSTVASSLVLVVSLGAISFWMCSNGSLQCRICI

SEQ ID NO: 441 - RNA, 2527 nt, synthetic construct
  source 1..2527: mol_type = other RNA
acatttgcttctgacacaactgtgttcactagcaacctcaaacagccaccatgaaagccattttggtcgtgatgctgtacaccttcaccactgccaacgcgatgaccgctgcctcggataacttccagttatcacaggggggccaaggcttcgcaatcccgataggtcaggcgatggcaatcgccggacaaattcgttctgggggtggttcgcctaccgtgcacatcggccccaccgcctttctgggtttaggtgtggtggataacaatggcaacggcgcgagggtgcagcgcgtggtcggaagcgcccctgccgccagtctcggtatttccacgggcgacgtcatcacggctgtcgatggtgcaccgatcaattcagccacggccatggccgacgcattaaacgggcatcacccgggtgacgtgatttcggtcacttggcagacgaaatccggtggcacccgtacgggcaacgtcaccctggctgaaggccctcccgccgaatttatggttgacttcggcgcactgccgccagaaatcaatagtgctcggatgtatgctggcccggggagcgcgtctcttgtggcggcagcccaaatgtgggatagtgtcgcctcggatctcttttctgcggcgagcgcgtttcaatcggttgtgtgggggcttacggtagggtcgtggattggctcctccgctggcctgatggttgccgcagcgagtccgtatgtggcgtggatgtcagtaactgcaggacaggcggaacttacggcagcacaggtacgcgtagcagctgcagcgtacgaaacggcgtacggcctgaccgtcccaccgccagtgatcgcggaaaatcgcgccgagttaatgatcctgattgccactaacctcttgggccagaacacccccgctatcgcggtaaacgaggccgaatacggtgaaatgtgggcgcaggatgcggcagcgatgttcggttatgcggcggctacggcgactgctacagcaaccctgttaccgtttgaggaggcacctgagatgaccagcgcaggtggcctgctggaacaagccgctgcggttgaagaagcctcagacaccgccgctgcgaatcagctgatgaataatgttccgcaagcgctgcagcagctggcgcagccgacccagggtacaacgccgagctccaaactgggcggtctgtggaagaccgtgtcacctcatcgtagcccgatttctaatatggtgagcatggccaataatcatatgagcatgaccaattcgggcgtctctatgacgaacacactcagctccatgctcaaaggttttgctccggccgctgcagctcaggcggttcagaccgcggcgcagaatggcgttcgtgcgatgagctccctagggtcaagcttgggaagctctggtttgggtggcggtgtagctgcgaacttgggtcgtgccgcgtcagtgggctcattgtcggtaccgcaggcttgggcagctgcgaatcaagcggtaactcccgccgcacgtgccctgcctcttacatctctgacaagtgctgccgaacgcggtccgggtcaaatgcttggaggcttacctgtgggtcagatgggcgcaagagcgggtggtgggctgagtggcgtcttgcgtgtgccgccacgcccatatgttatgccccatagtccagcggccggagatattgccccgccagcgttgtcccaagatcgctttgcggattttccggctctgcctctggatccgtccgccatggttgcgcaggttggcccgcaggtggtgaacattaacaccaagctgggctataacaatgcggtcggcgctggcaccggcatcgttattgatccgaatggcgtcgtcctgaccaacaaccacgtgattgcgggcgccaccgatatcaatgcattctcggtcggtagcggtcagacttatggcgtggacgtggttggttatgaccgcacacaggatgtggccgtactgcagctgcgcggggcgggcggtctgccatccgcagcaattggaggtggcgttgcagtaggagaacccgttgtggcaatggggaacagcggcggacaaggtgggactccacgggcggttccggggcgagttgtagcgctaggacagacagtgcaggcaagcgacagtcttacgggggcagaagagaccctgaatggcctgatccaatttgatgcggcgatacaaccgggcgatgccggcggaccggtggtgaacggtctgggccaagttgtcggtatgaacaccgcggcctctggtagcctcgcaatttacagtacggttgccagcagtttagtgctggtcgtttctttaggtgcgatttctttctggatgtgctcgaacggcagcctgcaatgtcgtatttgcatttgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 442 - protein, 776 aa, synthetic construct
MKAILVVMLYTFTTANAMTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAEFMVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMSMTNSGVSMTNTLSSMLKGFAPAAAAQAVQTAAQNGVRAMSSLGSSLGSSGLGGGVAANLGRAASVGSLSVPQAWAAANQAVTPAARALPLTSLTSAAERGPGQMLGGLPVGQMGARAGGGLSGVLRVPPRPYVMPHSPAAGDIAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDAGGPVVNGLGQVVGMNTAASGSLIPIAVGGALAGLVLIVLIAYLVGRKRSHGYQTI

SEQ ID NO: 443 - RNA, 1388 nt, Mycobacterium tuberculosis
  source 1..1388: mol_type = genomic RNA
acatttgcttctgacacaactgtgttcactagcaacctcaaacagccaccatgaaggccattttagttgtcatgttatatacgttcaccacggcgaacgcgatgacggccgccagcgataatttccaactaagccagggcggccaaggctttgcaattcccatcggacaggcgatggccatcgctggccaaattcgtagtggcggcggaagcccgacggtgcatatcggcccgactgcttttcttggcctgggcgtcgtagataataacggtaacggggcccgagtacagcgcgtggtaggttcggcgccagcggcatcactgggtatatcgacgggggacgttatcacagctgtggatggggcgccaattaacagcgcaaccgcgatggccgatgccctgaatggccatcatccgggggatgtgattagtgtcacgtggcaaaccaagtcaggtggcacccgcaccggcaacgttaccctggccgagggtccgcctgcggagtttatggtggatttcggcgcactgccgccggaaatcaactctgcgcgtatgtatgctggtccgggcagcgcctcactggtcgcggcggctcaaatgtgggactcagtggctagtgatctcttctcagcagcgtcggccttccaatcggtggtttggggcttgactgtgggtagttggatcggcagttccgctggtctcatggtggccgccgcgtcgccgtatgtggcttggatgagtgtgacagcaggtcaggccgaacttactgccgctcaggtaagagtcgccgcggctgcgtatgaaactgcctatggcctgaccgttccgccgccggtcattgccgaaaatcgcgctgaactcatgattctcattgccacgaatctgctgggtcaaaatacgcctgccatcgcagtgaatgaagccgagtacggcgagatgtgggcccaggatgcggcggcaatgttcggttacgctgcagcaacggcgactgcaaccgccacccttttgccgtttgaagaagcgcctgaaatgacgtcggcaggcggcctgttggaacaggcggctgccgtcgaagaggccagcgataccgcggcagccaaccagttaatgaataacgtccctcaagccctgcagcagctggcccagcctactcagggtaccactccgagctctaaattaggtggcctctggaaaacggtgagcccccaccgctccccgattagtaacatggtctccatggcgaacaaccatatgagcatgacgaattcgggtgtcagcatgacaaacaccctgtcgtccatgctgaaaggctttgcgccggcggctgcggcgcaggcggttcaaaccgcagctcagaacggagtgcgtgcaatgtcttctctgggctcc

SEQ ID NO: 444 - protein, 338 aa, Mycobacterium tuberculosis
MQLVDRVRGAVTGMSRRLVVGAVGAALVSGLVGAVGGTATAGAFSRPGLPVEYLQVPSPSMGRDIKVQFQSGGANSPALYLLDGLRAQDDFSGWDINTPAFEWYDQSGLSVVMPVGGQSSFYSDWYQPACGKAGCQTYKWETFLTSELPGWLQANRHVKPTGSAVVGLSMAASSALTLAIYHPQQFVYAGAMSGLLDPSQAMGPTLIGLAMGDAGGYKASDMWGPKEDPAWQRNDPLLNVGKLIANNTRVWVYCGNGKPSDLGGNNLPAKFLEGFVRTSNIKFQDAYNAGGGHNGVFDFPDSGTHSWEYWGAQLNAMKPDLQRALGATPNTGPAPQGA

SEQ ID NO: 445 - protein, 347 aa, synthetic construct
MGFLPKLLLLASFFPAGQAFSRPGLPVEYLQVPSPSMGRDIKVQFQSGGANSPALYLLDGLRAQDDFSGWDINTPAFEWYDQSGLSVVMPVGGQSSFYSDWYQPACGKAGCQTYKWETFLTSELPGWLQANRHVKPTGSAVVGLSMAASSALTLAIYHPQQFVYAGAMSGLLDPSQAMGPTLIGLAMGDAGGYKASDMWGPKEDPAWQRNDPLLNVGKLIANNTRVWVYCGNGKPSDLGGNNLPAKFLEGFVRTSNIKFQDAYNAGGGHNGVFDFPDSGTHSWEYWGAQLNAMKPDLQRALGATPNTGPAPQGAGSLLWVLGLLVGLLLLLLGLGACYTWRRRRVCK

SEQ ID NO: 446 - RNA, 1234 nt, synthetic construct
  source 1..1234: mol_type = other RNA
acatttgcttctgacacaactgtgttcactagcaacctcaaacagccaccatgggctttctcccgaaactgctgttactggcaagctttttcccagccggccaggcgttctcccgcccgggattgcctgtagaatatctgcaagtaccgagcccgtctatgggtcgggacatcaaagtgcagtttcaaagtggtggcgccaatagccccgcgctgtacctgcttgatgggctgcgtgcgcaagacgattttagcggctgggacattaatacgccggcgtttgaatggtatgatcagtcggggctgtcggttgtgatgcctgtgggcgggcagtctagcttttatagcgattggtatcaaccagcctgtggaaaagccggctgccagacctacaaatgggaaacctttctgactagtgagctgccgggatggctgcaggctaaccgtcatgtcaaaccaaccggcagtgcggtggtcggtttatcgatggctgcatctagcgcgctgacgctggccatttatcacccgcagcagttcgtatacgcaggcgcgatgtccggtttgttagatccctcacaggctatgggtccaaccctcattggtctggccatgggtgatgctggcggctataaagcctcagatatgtggggtccgaaagaggacccggcttggcaacgtaacgaccctttgttgaacgttgggaagttaatcgcgaacaacacgcgagtttgggtttactgtggcaatggcaaaccatcggatctggggggtaataacctgccggccaagtttttggaaggttttgtgcgtacatccaacatcaaattccaggatgcctacaacgcgggcggcggccataatggtgttttcgacttcccggatagtggtactcactcatgggaatactggggcgcacaacttaatgcaatgaagccggatcttcagcgcgcactgggtgcaacacctaataccggaccggcgccccagggcgcgggctctctgctatgggtcttaggtttactggtcggtctacttctcctgctgctcgggttgggagcgtgctatacctggagacgccgccgcgtgtgcaaatgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 447 - protein, 350 aa, synthetic construct
MKAILVVMLYTFTTANAFSRPGLPVEYLQVPSPSMGRDIKVQFQSGGANSPALYLLDGLRAQDDFSGWDINTPAFEWYDQSGLSVVMPVGGQSSFYSDWYQPACGKAGCQTYKWETFLTSELPGWLQANRHVKPTGSAVVGLSMAASSALTLAIYHPQQFVYAGAMSGLLDPSQAMGPTLIGLAMGDAGGYKASDMWGPKEDPAWQRNDPLLNVGKLIANNTRVWVYCGNGKPSDLGGNNLPAKFLEGFVRTSNIKFQDAYNAGGGHNGVFDFPDSGTHSWEYWGAQLNAMKPDLQRALGATPNTGPAPQGAGSLAIYSTVASSLVLVVSLGAISFWMCSNGSLQCRICI

SEQ ID NO: 448 - RNA, 1243 nt, synthetic construct
  source 1..1243: mol_type = other RNA
acatttgcttctgacacaactgtgttcactagcaacctcaaacagccaccatgaaagccattctggtggtgatgttatatacctttaccaccgctaacgctttctcacgccctgggttgccggtagaatacctgcaagtgcccagtccgtcgatgggacgggacattaaggttcaatttcaatctggaggtgcgaattcgcccgcgctgtacttactggatggcctgcgtgcccaggatgattttagcggttgggatattaataccccggcctttgagtggtatgaccagagtggcctgtcggttgtgatgcctgtcggcggccagtccagcttttattccgactggtaccagccggcatgtggcaaagctggctgccaaacttataaatgggaaacttttctcacgagtgaactgccaggctggctacaagcgaaccgccacgttaaaccgacggggagtgccgtagtcggcttgagtatggctgcgagctctgcgctgactctcgcgatctatcatccacagcagtttgtttatgcgggtgctatgagcggacttctggatcccagccaggccatgggaccgacgttaatcgggcttgccatgggcgatgccgggggttacaaagcaagcgatatgtggggtcctaaagaggatccggcgtggcagcgaaacgatccgctgctcaatgttgggaagctgatcgcgaataatacccgtgtctgggtctattgcggtaacggcaaaccatcggacctaggtggtaataacctgccggccaagttcctggaaggttttgtacgcaccagcaatataaaattccaggacgcgtacaacgcaggcggtggccataatggcgtgttcgacttcccggattcaggcacgcactcttgggaatactggggcgcccagttgaacgctatgaaaccggatctgcaacgtgcgttaggtgcaacacctaacacagggccggcgccacagggtgcaggtagcctggcaatttattcaaccgtggcgtcttcactggtgcttgtggtctccctgggtgcaatctccttctggatgtgttccaacggcagcttgcagtgccgcatttgtatctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 449 - protein, 374 aa, Mycobacterium tuberculosis
VKIRLHTLLAVLTAAPLLLAAAGCGSKPPSGSPETGAGAGTVATTPASSPVTLAETGSTLLYPLFNLWGPAFHERYPNVTITAQGTGSGAGIAQAAAGTVNIGASDAYLSEGDMAAHKGLMNIALAISAQQVNYNLPGVSEHLKLNGKVLAAMYQGTIKTWDDPQIAALNPGVNLPGTAVVPLHRSDGSGDTFLFTQYLSKQDPEGWGKSPGFGTTVDFPAVPGALGENGNGGMVTGCAETPGCVAYIGISFLDQASQRGLGEAQLGNSSGNFLLPDAQSIQAAAAGFASKTPANQAISMIDGPAPDGYPIINYEYAIVNNRQKDAATAQTLQAFLHWAITDGNKASFLDQVHFQPLPPAVVKLSDALIATISS

SEQ ID NO: 450 - protein, 159 aa, Mycobacterium tuberculosis
VKRGLTVAVAGAAILVAGLSGCSSNKSTTGSGETTTAAGTTASPGAASGPKVVIDGKDQNVTGSVVCTTAAGNVNIAIGGAATGIAAVLTDGNPPEVKSVGLGNVNGVTLGYTSGTGQGNASATKDGSHYKITGTATGVDMANPMSPVNKSFEIEVTCS

SEQ ID NO: 451 - protein, 554 aa, synthetic construct
MKAILVVMLYTFTTANACGSKPPSGSPETGAGAGTVATTPASSPVTLAETGSTLLYPLFNLWGPAFHERYPNVTITAQGTGSGAGIAQAAAGTVNIGASDAYLSEGDMAAHKGLMNIALAISAQQVNYNLPGVSEHLKLNGKVLAAMYQGTIKTWDDPQIAALNPGVNLPGTAVVPLHRSDGSGDTFLFTQYLSKQDPEGWGKSPGFGTTVDFPAVPGALGENGNGGMVTGCAETPGCVAYIGISFLDQASQRGLGEAQLGNSSGNFLLPDAQSIQAAAAGFASKTPANQAISMIDGPAPDGYPIINYEYAIVNNRQKDAATAQTLQAFLHWAITDGNKASFLDQVHFQPLPPAVVKLSDALIATISSGGSGGGGSGGCSSNKSTTGSGETTTAAGTTASPGAASGPKVVIDGKDQNVTGSVVCTTAAGNVNIAIGGAATGIAAVLTDGNPPEVKSVGLGNVNGVTLGYTSGTGQGNASATKDGSHYKITGTATGVDMANPMSPVNKSFEIEVTCSGSLAIYSTVASSLVLVVSLGAISFWMCSNGSLQCRICI

SEQ ID NO: 452 - RNA, 1855 nt, synthetic construct
  source 1..1855: mol_type = other RNA
acatttgcttctgacacaactgtgttcactagcaacctcaaacagccaccatgaaggcgattttggtcgttatgctttataccttcaccaccgcgaacgcttgcgggtcaaaaccgccaagcggcagcccggaaactggagctggggcaggaactgtagcaacaacaccagcatcatcccccgtgacgcttgcggagacgggatcaaccctgttgtatccgctctttaacctgtggggcccagcctttcacgagcgctatcccaatgttaccattacggctcaggggactggatctggtgcaggcatcgcacaggcggcagcaggcacagttaatataggcgcgagcgatgcatatctctcggaaggcgatatggctgcgcataagggcctgatgaacattgctttagcgattagcgcgcagcaggtcaattacaacctgccgggtgtcagcgaacatctgaagctgaatggcaaagtcctcgccgccatgtaccaaggtaccatcaaaacctgggatgatccccaaatcgcggccctgaaccctggcgttaacctgccaggtaccgcggtggtgcctttgcatcgttcggacggctcgggtgacacttttctgttcacgcagtacttatctaaacaagatccggaaggttgggggaaatcaccgggcttcggtactacggtggactttcctgcagtaccgggtgctctgggggaaaacggaaatggtgggatggtgaccgggtgtgcggaaacaccgggttgtgtggcctatattggtatttcgtttctggatcaggctagccagcgtggcttaggcgaagcgcaattaggtaattccagcggcaactttttactaccggacgcacagagcatccaagcagccgccgcggggtttgcgtcaaaaaccccggccaatcaggcaattagcatgattgatggcccggcgccagacggctaccccatcatcaactatgaatatgcgattgttaataatcggcagaaagacgcggcgaccgcgcagacccttcaggcgtttctccactgggcgatcaccgatggcaacaaggcctcgttcctggatcaagtgcattttcaaccgctaccgccggccgtagtaaaactgtcggatgcgctgatcgccaccatcagtagtggtggcagtggtggtggaggtagtggcggatgctcaagcaacaaaagtacaacgggatctggcgagacgactaccgcagcgggcacaacggcttcccctggtgcggcaagtggccctaaagttgtgatcgacggcaaggatcaaaatgtgacggggtctgtcgtttgtaccacggccgccggtaacgtcaacatcgccattgggggtgcagccactggcatagcggccgtcctgaccgatggcaatccgccagaagtgaaatcggttggcttgggcaatgttaacggcgtaacgcttggctacacgtccggtaccggtcagggcaacgcttccgctactaaagacgggtctcactataaaatcaccggcaccgctaccggtgtcgatatggccaatccgatgtctccggttaacaaatccttcgagattgaagtgacgtgcagcggttctctggctatttacagtaccgtagccagctctctggtgttggtggtgtcgctgggtgccattagcttctggatgtgcagcaatggttccctgcagtgtcgcatttgcatttgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 453-500 - skipped (48 entries)

SEQ ID NO: 501 - RNA, 50 nt, synthetic construct
  source 1..50: mol_type = other RNA
acatttgcttctgacacaactgtgttcactagcaacctcaaacagccacc

SEQ ID NO: 502 - RNA, 134 nt, synthetic construct
  source 1..134: mol_type = other RNA
gctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 503-504 - skipped (2 entries)

SEQ ID NO: 505 - RNA, 1796 nt, synthetic construct
  source 1..1796: mol_type = other RNA
acatttgcttctgacacaactgtgttcactagcaacctcaaacagccaccatggaactgctgatcctgaaggccaacgccatcaccactattctgaccgccgtgaccttttgcttcgcctccggccagaacatcacagaggaattctaccagagcacctgttctgccgtcagcaagggctacctgtctgccctgagaacaggctggtacacctctgtgatcaccatagagctgagtaacatcaaagagaacaagtgtaatggaaccgatgcaaaggtgaaactgattaaacaggagctggacaagtacaagaacgccgtcaccgagctgcagctactgatgcagagcacccccgccaccggctcaggcagcgccatctgcagcggcgtcgccgtgtgcaaggtgctgcacctggaaggcgaagtgaacaaaatcaagtctgctctgctgtccacaaacaaggctgttgtgagcctgagcaatggcgtgagcgtgctgacattcaaggtgctagatctgaaaaactacatcgataagcaactgctgcctatcctgaacaagcagagctgcagcatcagcaacatcgagacagtgatcgaatttcagcagaaaaacaaccggctgctggaaatcacaagagagttcagcgtgaacgccggagtgaccacccctgtgtctacctacatgctgacaaatagcgagctgctgagcctgatcaacgacatgcctattaccaacgaccagaagaagctgatgagcaacaacgtgcagatcgtgcggcagcaatcatactctatcatgtgcatcatcaaggaagaagtgctggcttacgtggtgcaactgcctctgtacggcgtaatcgacacaccatgctggaagctgcacaccagccctctgtgcacaaccaacaccaaggaaggctccaacatctgtctgaccaggacagatagaggctggtattgtgacaacgccggcagcgtgagcttcttcccccaggccgagacatgcaaagtgcagagcaatagagtgttttgcgacaccatgaacagcagaaccctgccatctgaggtgaacctgtgtaacgtggacatcttcaaccccaagtacgactgcaagatcatgaccagcaaaacagatgtgagttctagcgtcatcaccagcctgggtgccatcgtatcttgctacggcaaaaccaagtgcaccgccagcaataagaatcggggaatcatcaagacattcagcaacggctgcgactacgtttctaataagggagtggataccgtgtctgtgggaaataccctgtactgtgtgaacaagcaggagggcaagagcctgtatgtgaagggcgagcctatcatcaacttctacgatcctctggtgttccctagcgacgagttcgacgctagcatcagccaggtgaatgagaagatcaaccagtccctcgccttcatcagaaagtccgacgagctgctccacaacgtgaacgctggcaagagcacaacaaacatcatgatcaccacaattatcatcgtgatcatcgtgatcctgctgagcctgatcgccgtgggcctgctgctgtactgcaaggccagaagcacccccgtgaccctgtccaaggaccagctgagcggcatcaacaacatcgcctttagcaactgataataggctcgctttcttgctgtccaatttctattaaaggttcctttgttccctaagtccaactactaaactgggggatattatgaagggccttgagcatctggattctgcctaataaaaaacatttattttcattgc

SEQ ID NO: 506 - RNA, 1798 nt, synthetic construct
  source 1..1798: mol_type = other RNA
acatttgcttctgacacaactgtgttcactagcaacctcaaacagccaccatggagctgctgatcctgaaggccaacgccatcaccaccattctgacagccgtcaccttctgctttgcctctggtcagaacatcaccgaggagttctaccagagcacctgcagcgccgtgagcaaggggtacttgtccgccctgcggaccggctggtacaccagcgtcattaccattgagctctccaacatcaaggagaacaagtgcaatggtactgacgccaaggtgaagctgatcaagcaggagctggacaagtacaagaatgccgtcacggagctgcagctgctcatgcagagcacacccgccaccgggtccggcagcgccatctgcagcggcgtggccgtctgcaaggtgctgcacctggagggcgaggtgaacaagatcaagtctgctctgctgagcaccaacaaggcagtggtgagcctgtcaaatggggtgagcgtgttgaccttcaaggtgctcgacctgaagaattacattgacaagcagctgctgccaattctgaacaagcagagttgcagcatctcgaacatcgagaccgtgatcgagttccagcagaagaacaatcggctgctggagatcaccagggagttctccgtgaacgccggggtgaccacccccgtgagcacatacatgctgaccaatagcgagctgctgtctctgatcaacgacatgcccatcaccaacgaccagaagaagctgatgagcaacaacgtgcagatcgtgagacagcagagctacagcattatgtgcatcatcaaggaggaggtcctggcttacgtggtgcagctgccgctgtacggcgtgatcgataccccttgttggaagctgcacactagtccactgtgcacaaccaacaccaaggagggcagcaacatctgcctgacgaggacggaccggggctggtattgcgacaatgccggctccgtgtccttcttccctcaggcagagacctgcaaggtgcagtccaaccgggtgttctgtgacaccatgaacagccggacgctgccctccgaggtgaacctgtgcaatgtggacatcttcaatcccaagtacgattgcaagatcatgaccagcaagaccgatgtgtcctccagcgtgatcacctcgctgggggccatcgtgtcttgctacggcaagaccaagtgtacagcttccaacaagaacaggggtatcatcaagacgttcagcaacggctgcgactacgtgagcaacaagggggtggacaccgtgagcgtggggaacaccctgtactgcgtgaacaagcaggaggggaagagcctgtacgtgaagggggagcccatcatcaacttttacgaccccctggtcttcccctccgacgagttcgacgccagtatcagccaggtgaatgagaagatcaatcagagcctggctttcatcagaaagtctgatgagctgctgcacaatgtgaacgccgggaagagcaccaccaacatcatgatcacgaccatcatcatcgtgatcatcgtgatcctgctcagcctgatcgctgtgggtctgctgctgtactgcaaggcccgcagtacaccagtgaccctcagcaaggatcagctgagcgggatcaacaacatcgccttctccaactgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 507 - RNA, 1798 nt, synthetic construct
  source 1..1798: mol_type = other RNA
acatttgcttctgacacaactgtgttcactagcaacctcaaacagccaccatggagttgctgatcctgaaggctaatgccatcaccacgattctgacggccgtgacattttgcttcgcctccgggcagaacatcacggaggagttctatcagtctacttgttctgcagtcagcaaggggtacttgtccgccctgcggaccggctggtacactagcgtcattaccattgagctctccaacatcaaggagaacaagtgcaatggtactgacgctaaggtgaagctgatcaagcaggagctggacaagtacaagaacgccgtgaccgagctgcagctgctgatgcagagcactcccgccaccgggagtggctctgccatctgcagcggggtcgcggtgtgcaaggtcctgcaccttgagggtgaggtcaacaagatcaagtctgctctgctgagcaccaacaaggcggtggtcagcctgagcaacggggtctctgtcctcaccttcaaggtgctggacctgaagaactacatcgataagcagctgctgccaattctgaacaagcagagttgcagcatcagcaatatcgagacggtgatcgagttccagcagaagaacaatcggctgctggagatcacccgggagttcagcgtgaacgccggggtgacaaccccggtgtccacctacatgctgaccaactccgagcttctgtctctgatcaatgatatgcctatcaccaatgatcagaagaagctgatgtccaacaatgtgcagatcgtgcgccagcagtcctactccataatgtgcatcattaaggaggaggtgctggcgtacgtggtgcagcttccactgtacggggtgatcgacaccccgtgctggaagctgcacacctctcctttgtgtaccaccaacacaaaggaggggtccaacatctgcctgacgaggacggaccggggctggtattgcgacaatgccggctccgtgtccttcttccctcaggcagagacctgcaaggtgcagagtaaccgggtgttctgtgacaccatgaacagccggactctgccctcggaggtcaatctgtgcaatgtggacatcttcaaccccaagtatgactgcaagatcatgacaagtaagactgatgtgagctcctccgtgatcaccagcctgggggcgatcgtgtcttgctacggcaagacaaagtgcacggcctccaacaagaatcgtgggatcattaagaccttcagtaatggttgtgattatgtgtccaacaagggggtggacactgtcagcgttggcaacacgctgtactgcgtgaacaagcaggagggcaagtcgctctacgtgaagggcgagccgattattaacttctacgatcccctggtgttccctagcgacgagttcgacgctagcatcagccaggtgaacgagaagattaatcagtcgctcgccttcatcagtaagagcgacgagctcctgcacaatgtcaacgctggcaagtccaccaccaacatcatgatcacaaccattatcatcgtgatcattgtgatcctgctcagcctgatcgctgtgggtctgctgctgtactgcaaggcccgcagtacaccagtgaccctcagcaaggatcagctgagcgggatcaacaatatcgcgttcagcaactgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 508 - RNA, 1798 nt, synthetic construct
  source 1..1798: mol_type = other RNA
acatttgcttctgacacaactgtgttcactagcaacctcaaacagccaccatggagttgctgatcctgaaggcgaatgctattaccaccatcctgacagctgtgaccttctgttttgcctccgggcagaacatcacggaggagttctatcagtctacttgttctgcagtcagcaaggggtacctgtccgcgttgcgcacggggtggtacacctccgtgatcacgattgagctctctaacatcaaggagaataagtgtaacggcactgacgccaaggtcaagctgatcaagcaggagctggacaagtataagaacgctgtgaccgagttacagcttcttatgcagtccactcctgctacaggcagcggctccgctatctgtagcggagtcgctgtctgtaaggtcttgcacctggagggcgaggtgaacaagatcaagtccgccttgctctccaccaacaaggccgtggtcagcttgtccaatggcgtcagtgtccttacatttaaggtccttgatctcaagaattacattgataagcagctgctgcctattctgaataagcagagctgctctatcagtaatattgagacggtgatcgagttccagcagaagaacaatcggctgctggagatcaccagagagttcagcgtgaacgcgggggtgaccaccccggtcagcacctacatgctgaccaactctgagctgttgtcgctcatcaacgacatgccgatcaccaatgatcagaagaagttgatgagcaacaacgttcagatcgtgcgccagcagtcctactccataatgtgcatcattaaggaggaggtgctggcgtacgtggtgcagcttccactgtacggggtgatcgacaccccgtgctggaagctgcacacctctcctttgtgtaccaccaacacaaaggaggggtccaacatctgcctgacgaggacggaccggggctggtattgcgacaatgccggctccgtgtccttcttccctcaggcagagacctgcaaggtgcagagtaaccgggtgttctgtgacaccatgaacagccggactctgccctcggaggtcaacctgtgcaacgtggacatcttcaaccccaagtatgactgcaagatcatgacaagtaagactgatgtgagctcctccgtgatcacgtcactgggggcgatcgtgtcttgctacggcaagaccaagtgcaccgccagcaataagaacagggggatcattaagacatttagtaatggttgtgattatgtgtccaacaagggggtggacactgtcagcgttggcaacacgctgtactgcgtgaacaagcaggagggcaagtcgctctacgtgaagggcgagccgattattaacttctatgacccgctggtgttcccgtcggacgagttcgacgcgagcatcagccaggtcaacgagaagattaatcagtcgctcgcctttatacgtaagagcgacgaactcctgcacaatgtcaacgctggcaagtccaccaccaacatcatgatcacaaccattattatcgtgatcatagtgatcctcctgtctcttattgcggtgggcctcttgctgtactgcaaggcacggtcgaccccagtgacgctgagcaaggaccagctgtcaggaattaataacatcgccttcagcaactgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 509 - protein, 321 aa, Human respiratory syncytial virus A
MSKTKDQRTAKTLERTWDTLNHLLFISSCLYKLNLKSIAQITLSILAMIISTSLIIAAIIFIASANHKVTPTTAIIQDATNQIKNTTPTHLTQNPQLGISLSNLSGTTSQSTTILASTTPSAESTPQSTTVKIINTTTTQILPSKPTTKQRQNKPQNKPNNDFHFEVFNFVPCSICSNNPTCWAICKRIPNKKPGKKTTTKPTKKPTLKTTKKDPKPQTTKPKGVLTTKPTGKPTINTTKTNSRTTLLTSNTKGNPEHTSQKETIHSTTSEGYPSPSQVYTTSDQEETLHSTTSEGYPSPSQVYTTSEYLSQSLSSSNTTK

SEQ ID NO: 510 - protein, 48 aa, Human respiratory syncytial virus A
RQNKPQNKPNNDFHFEVFNFVPCSICSNNPTCWAICKRIPNKKPGKKT

SEQ ID NO: 511 - protein, 310 aa, Human respiratory syncytial virus B
MSKNKNQRTARTLEKTWDTLNHLIVISSCLYKLNLKSIAQIALSVLAMIISTSLIIAAIIFIISANHKVTLTTATVQTIKNHTEKNITTYLTQVSPERVSPSKQPTATPPIHTNSATISPNTKSETHHTTTQTKGTISTPTQNNKPSTKPRPKNPPKKDDYHFEVFNFVPCSICGNNQLCKSICKTIPSNKPKKKPTTKPTNKPPTKTTNKRDPKTLAKTPKKETTINPTKKPTPKTTERDTSTPQSTVLDITTSKHTERDTSTSQSIALDTTTSKHTTQQQSLYSTIPENTPNSTQTPTASEPSTSNSI

SEQ ID NO: 512 - protein, 43 aa, Human respiratory syncytial virus B
KNPPKKDDYHFEVFNFVPCSICGNNQLCKSICKTIPSNKPKKK

SEQ ID NO: 513 - protein, 612 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAICSGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGKSLYVKGEPIINFYDPLVFPSDEFDASISQVNEKINQSLAFIRKSDELLSAIGGYIPEAPRDGQAYVRKDGEWVLLSTFLGSGGGSGGRQNKPQNKPNNDFHFEVFNFVPCSICSNNPTCWAICKRIPNKKPGKKTGSGGGSGGKNPPKKDDYHFEVFNFVPCSICGNNQLCKSICKTIPSNKPKKK

SEQ ID NO: 514 - RNA, 2029 nt, synthetic construct
  source 1..2029: mol_type = other RNA
acatttgcttctgacacaactgtgttcactagcaacctcaaacagccaccatggaactcctcatcctgaaagccaacgccatcaccacaattctcactgctgtgaccttctgttttgcctcgggacagaacatcaccgaggagttctaccagtccacatgctccgctgtgtcgaagggctacctgtcggcgctgaggactggctggtacacctccgtgattaccatcgaactgtcaaacatcaaggagaacaagtgcaacggtactgacgcgaaggtcaagctgattaagcaggaattggacaagtacaagaacgccgtgactgagctccagctgctgatgcagtcaacccctgcgacgggaagcgggtccgcaatctgctccggagtggccgtgtgcaaagtgctgcatctggagggcgaagtcaacaagatcaagtcggccctgctgagcaccaacaaggccgtggtgtccttgagtaacggagtctccgtcctgaccttcaaagttctggacctgaagaattacatcgataagcagctcctgcccatcctcaacaagcagagctgctccatttcgaacatcgaaactgtgatcgagtttcagcaaaagaacaaccggctgctggaaatcaccagagagtttagcgtgaacgcaggagtgactacccccgtgtctacctacatgctaaccaactcagaactgcttagcttgatcaatgacatgccgatcactaacgatcagaagaagcttatgtccaacaacgtgcaaatcgtgcgccagcaatcctactcgattatgtgcattatcaaggaagaggtgctggcctatgtggtgcagctcccgctgtacggcgtgattgacactccttgctggaagctgcacacctcccccctgtgtaccacgaataccaaggaggggtccaatatctgcctgacccggaccgaccgcggatggtactgcgacaacgccggttcggtgtccttcttcccacaagccgagacttgcaaagtccagtccaatcgcgtgttctgcgacactatgaactcccggaccttgccgtccgaagtgaacctgtgtaacgtggacatcttcaaccctaaatacgactgcaagattatgacttccaagaccgacgtcagcagctcagtgatcaccagcctgggcgccatcgtgtcgtgttatggaaagaccaagtgcactgcgtccaacaagaacagaggcatcatcaagaccttcagcaacggctgcgattacgtcagcaacaagggggtggataccgtgtccgtgggaaacactctctactgtgtgaacaagcaggagggaaagtccctctacgtcaagggcgaaccgatcatcaacttctacgatcccctggtgttcccatccgacgagttcgacgcctccatttcacaagtcaacgaaaagatcaaccagtctctggcgttcatccggaagtcggatgaactcctttccgccattggtggatacattcctgaggctcctcgcgatggccaggcctacgtcaggaaagacggagaatgggtgctgctttcaaccttcctgggatccgggggtggctcaggcggcagacaaaacaagccccagaacaagccgaacaacgacttccactttgaagtgttcaatttcgtaccgtgctcgatttgttccaacaaccccacttgctgggcaatctgcaagcggatcccaaacaagaagcctggaaagaaaaccgggagcggcggaggttccggagggaagaacccgcctaagaaggacgactaccacttcgaggtgttcaacttcgtcccgtgttcgatctgcggcaacaatcagctgtgcaagtcaatttgcaagaccatcccctccaacaagcccaagaagaagtgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 515 - protein, 165 aa, synthetic construct
MSKTKDQRTAKTLERTWDTLNHLLFISSCLYKLNLKSIAQITLSILALIISTSLIIAAIIFIAGGSRQNKPQNKPNNDFHFEVFNFVPCSICSNNPTCWAICKRIPNKKPGKKTGSGGGSGGKNPPKKDDYHFEVFNFVPCSICGNNQLCKSICKTIPSNKPKKK

SEQ ID NO: 516 - RNA, 688 nt, synthetic construct
  source 1..688: mol_type = other RNA
acatttgcttctgacacaactgtgttcactagcaacctcaaacagccaccatgagcaagaccaaggaccagcggaccgccaaaaccctcgaaaggacttgggatacccttaatcatctgctgttcatttcctcgtgcttatacaagctgaacctgaagtccattgcacaaattaccctgtcaattctggccctcatcatctccacctccctgatcattgccgctatcatcttcatcgcgggcggtagcagacaaaacaagccccagaacaagccgaacaacgacttccactttgaagtgttcaatttcgtaccgtgctcgatttgttccaacaaccccacttgctgggcaatctgcaagcggatcccaaacaagaagcctggaaagaaaaccgggagcggcggaggttccggagggaagaacccgcctaagaaggacgactaccacttcgaggtgttcaacttcgtcccgtgttcgatctgcggcaacaatcagctgtgcaagtcaatttgcaagaccatcccctccaacaagcccaagaagaagtgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 517-800 - skipped (284 entries)

SEQ ID NO: 801 - protein, 1010 aa, synthetic construct
MAAPGSARRPLLLLLLLLLLGLMHCASASASKEIKSFLWTQSLRRELSGYCSNIKLQVVKDAQALLHGLDFSEVSNVQRLMRKERRDDNDLKRLRDLNQAVNNLVELKSTQQKSILRVGTLTSDDLLILAADLEKLKSKVIRTERPLSAGVYMGNLSSQQLDQRRALLNMIGMSGGNQGARAGRDGVVRVWDVKNAELLNNQFGTMPSLTLACLTKQGQVDLNDAVQALTDLGLIYTAKYPNTSDLDRLTQSHPILNMIDTKKSSLNISGYNFSLGAAVKAGACMLDGGNMLETIKVSPQTMDGILKSILKVKKALGMFISDTPGERNPYENILYKICLSGDGWPYIASRTSITGRAWENTVVDLESDGKPQKADSNNSSKSLQSAGFTAGLTYSQLMTLKDAMLQLDPNAKTWMDIEGRPEDPVEIALYQPSSGCYIHFFREPTDLKQFKQDAKYSHGIDVTDLFATQPGLTSAVIDALPRNMVITCQGSDDIRKLLESQGRKDIKLIDIALSKTDSRKYENAVWDQYKDLCHMHTGVVVEKKKRGGKEEITPHCALMDCIMFDAAVSGGLNTSVLRAVLPRDMVFRTSTPRVVLGPGPGTSLYKGVYELQTLELNMETLNMTMPLSCTKNNSHHYIMVGNETGLELTLTNTSIINHKFCNLSDAHKKNLYDHALMSIISTFHLSIPNFNQYEAMSCDFNGGKISVQYNLSHSYAGDAANHCGTVANGVLQTFMRMAWGGSYIALDSGRGNWDCIMTSYQYLIIQNTTWEDHCQFSRPSPIGYLGLLSQRTRDIYISRRLLGTFTWTLSDSEGKDTPGGYCLTRWMLIEAELKCFGNTAVAKCNEKHDEEFCDMLRLFDFNKQAIQRLKAEAQMSIQLINKAVNALINDQLIMKNHLRDIMGIPYCNYSKYWYLNHTTTGRTSLPKCWLVSNGSYLNETHFSDDIEQQADNMITEMLQKEYMERQGKTPLGLVDLIPIAVGGALAGLVLIVLIAYLVGRKRSHAGYQTI

SEQ ID NO: 802 - RNA, 3223 nt, synthetic construct
  source 1..3223: mol_type = other RNA
acatttgcttctgacacaactgtgttcactagcaacctcaaacagccaccatggccgcccccgggagcgctaggcgccctcttctgctcttgctgttgctgctgcttctggggctgatgcattgcgcatcagcaagcgcctccaaggagatcaagtcgttcctgtggacccagtccctccggcgggagctgtcgggctactgctcgaacatcaagctccaagtcgtgaaagacgcgcaggccctgctgcacggactggatttctcggaagtctccaacgtccaaagattgatgcggaaagaaaggagggacgacaatgatctgaagaggctccgcgacctcaaccaggcggttaacaacctggtggaactgaagtcgacccagcaaaagagcattctgcgcgtgggcaccctcacctccgacgaccttctgatcctggccgccgatctggagaagttgaagtccaaggtcatccgcaccgaacggccgctgtccgccggggtgtacatgggaaacctgtcaagccagcagctggatcagcgcagagcacttcttaacatgatcggaatgtccggtggaaatcagggtgccagagcaggccgggacggagtcgtgcgggtctgggacgtgaagaacgcggagctgctgaacaaccagttcggcaccatgccgtcactgaccctggcctgtctcacaaagcagggccaagtggacctgaatgacgcagtgcaggcccttacggacctgggcctgatctacaccgctaagtacccgaataccagcgacctggatagactgactcagtcgcatccaattctgaatatgatcgacaccaagaagtcctcgctgaacatttccggttataacttctccttgggagcggccgtgaaggccggagcgtgcatgctggacggcgggaacatgctggaaaccatcaaagtgtccccgcaaaccatggacggaatcctcaagtccatcctgaaagtcaagaaggccctgggaatgttcatttctgacaccccgggagagaggaacccatacgagaatatcctgtacaagatttgtctgagcggggacggctggccgtacattgcgagccggactagcatcaccggtagagcctgggagaacaccgtggtcgaccttgaatccgatggaaagccacagaaggcagactccaacaactcttccaagtccctgcaaagcgccggattcaccgcgggactcacctactcccaactcatgaccctgaaggatgcaatgctccagctcgacccaaacgctaagacttggatggacatcgaaggccggcccgaagatcccgtggaaattgccctctaccaaccctcaagcggttgctacatccacttcttccgcgaaccgactgacctgaaacagtttaagcaggacgcaaagtactcccacggtatcgatgtgaccgacctctttgctacccaacctggcctgacctccgccgtgatcgatgccctgccccgcaacatggtcatcacttgtcaaggcagtgatgatatcaggaagctgctcgagagccagggtagaaaggatatcaagctgatcgacatcgcgctgagcaagactgacagccgcaaatacgaaaacgctgtgtgggaccagtacaaggacttgtgccacatgcacacaggggtggtggtcgaaaagaagaagcggggaggaaaggaggagatcaccccacattgcgccctgatggactgcattatgttcgacgcggccgtgtcaggaggactcaacacttccgtgctccgggccgtgctgccccgggacatggtgttcagaacttccactcctcgcgtggtgctgggcccgggacctggaacctcactgtacaagggggtgtacgaactgcagaccctggaattgaacatggaaactctgaacatgaccatgcctctctcctgtaccaagaacaattcccaccactacattatggtcggaaacgaaactgggctcgagctgaccctcaccaacacctccataatcaaccacaagttctgcaacctttctgatgcccacaagaagaacctatacgaccatgccctgatgtccattatctccaccttccacctctcaatccccaacttcaatcaatacgaagcgatgtcctgtgatttcaacggcggaaagatctcggtgcagtacaacctctcgcattcatacgccggagatgccgccaaccactgcggtactgtggccaacggggtgctccagacctttatgaggatggcgtggggaggatcctacatcgccttggactccggacgcggcaactgggactgcatcatgaccagctaccagtacctgattattcagaacaccacctgggaggaccactgccagttctcgcgcccttcgccgattgggtaccttggcctcctgtcgcaaagaaccagagacatctacatcagccgccggttactgggaacttttacttggaccctttcggattccgagggaaaggacacgcccggcggttactgcctgacgcggtggatgctgattgaagctgaactcaagtgtttcggcaacaccgccgtggccaagtgcaacgagaagcacgacgaagagttctgcgatatgctgcgactgttcgatttcaacaagcaggctattcagcggctgaaggccgaggcccagatgagcattcagctgatcaacaaggccgtgaacgccctgattaacgatcagctcattatgaagaaccatctgcgggacatcatgggcatcccttactgcaactactccaagtattggtacctgaaccacactaccactggccgcaccagcctcccgaagtgctggctcgtgtccaacggatcctatctgaacgaaacccacttttccgacgacattgagcagcaggccgacaacatgatcaccgagatgttgcagaaggagtacatggagcgccaggggaaaactcccctgggactggtggaccttatccctattgctgtgggcggtgccttggctggcctggtactgatcgtgctgatcgcgtatctcgtgggccggaaacggtcacacgccgggtaccagactatctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 803 - protein, 1024 aa, synthetic construct
MRVTAPRTLILLLSGALALTETWASASKEIKSFLWTQSLRRELSGYCSNIKLQVVKDAQALLHGLDFSEVSNVQRLMRKERRDDNDLKRLRDLNQAVNNLVELKSTQQKSILRVGTLTSDDLLILAADLEKLKSKVIRTERPLSAGVYMGNLSSQQLDQRRALLNMIGMSGGNQGARAGRDGVVRVWDVKNAELLNNQFGTMPSLTLACLTKQGQVDLNDAVQALTDLGLIYTAKYPNTSDLDRLTQSHPILNMIDTKKSSLNISGYNFSLGAAVKAGACMLDGGNMLETIKVSPQTMDGILKSILKVKKALGMFISDTPGERNPYENILYKICLSGDGWPYIASRTSITGRAWENTVVDLESDGKPQKADSNNSSKSLQSAGFTAGLTYSQLMTLKDAMLQLDPNAKTWMDIEGRPEDPVEIALYQPSSGCYIHFFREPTDLKQFKQDAKYSHGIDVTDLFATQPGLTSAVIDALPRNMVITCQGSDDIRKLLESQGRKDIKLIDIALSKTDSRKYENAVWDQYKDLCHMHTGVVVEKKKRGGKEEITPHCALMDCIMFDAAVSGGLNTSVLRAVLPRDMVFRTSTPRVVLGPGPGTSLYKGVYELQTLELNMETLNMTMPLSCTKNNSHHYIMVGNETGLELTLTNTSIINHKFCNLSDAHKKNLYDHALMSIISTFHLSIPNFNQYEAMSCDFNGGKISVQYNLSHSYAGDAANHCGTVANGVLQTFMRMAWGGSYIALDSGRGNWDCIMTSYQYLIIQNTTWEDHCQFSRPSPIGYLGLLSQRTRDIYISRRLLGTFTWTLSDSEGKDTPGGYCLTRWMLIEAELKCFGNTAVAKCNEKHDEEFCDMLRLFDFNKQAIQRLKAEAQMSIQLINKAVNALINDQLIMKNHLRDIMGIPYCNYSKYWYLNHTTTGRTSLPKCWLVSNGSYLNETHFSDDIEQQADNMITEMLQKEYMERQGKTPLGLVDGIVAGLAVLAVVVIGAVVAAVMCRRKSSGGKGGSYSQAACSDSAQGSDVSLTA

SEQ ID NO: 804 - RNA, 3265 nt, synthetic construct
  source 1..3265: mol_type = other RNA
acatttgcttctgacacaactgtgttcactagcaacctcaaacagccaccatgcgggtcactgcgccgagaaccctcattcttctcctatccggggcgctggctctcaccgaaacctgggcaagcgcctccaaggagatcaagtcgttcctgtggacccagtccctccggcgggagctgtcgggctactgctcgaacatcaagctccaagtcgtgaaagacgcgcaggccctgctgcacggactggatttctcggaagtctccaacgtccaaagattgatgcggaaagaaaggagggacgacaatgatctgaagaggctccgcgacctcaaccaggcggttaacaacctggtggaactgaagtcgacccagcaaaagagcattctgcgcgtgggcaccctcacctccgacgaccttctgatcctggccgccgatctggagaagttgaagtccaaggtcatccgcaccgaacggccgctgtccgccggggtgtacatgggaaacctgtcaagccagcagctggatcagcgcagagcacttcttaacatgatcggaatgtccggtggaaatcagggtgccagagcaggccgggacggagtcgtgcgggtctgggacgtgaagaacgcggagctgctgaacaaccagttcggcaccatgccgtcactgaccctggcctgtctcacaaagcagggccaagtggacctgaatgacgcagtgcaggcccttacggacctgggcctgatctacaccgctaagtacccgaataccagcgacctggatagactgactcagtcgcatccaattctgaatatgatcgacaccaagaagtcctcgctgaacatttccggttataacttctccttgggagcggccgtgaaggccggagcgtgcatgctggacggcgggaacatgctggaaaccatcaaagtgtccccgcaaaccatggacggaatcctcaagtccatcctgaaagtcaagaaggccctgggaatgttcatttctgacaccccgggagagaggaacccatacgagaatatcctgtacaagatttgtctgagcggggacggctggccgtacattgcgagccggactagcatcaccggtagagcctgggagaacaccgtggtcgaccttgaatccgatggaaagccacagaaggcagactccaacaactcttccaagtccctgcaaagcgccggattcaccgcgggactcacctactcccaactcatgaccctgaaggatgcaatgctccagctcgacccaaacgctaagacttggatggacatcgaaggccggcccgaagatcccgtggaaattgccctctaccaaccctcaagcggttgctacatccacttcttccgcgaaccgactgacctgaaacagtttaagcaggacgcaaagtactcccacggtatcgatgtgaccgacctctttgctacccaacctggcctgacctccgccgtgatcgatgccctgccccgcaacatggtcatcacttgtcaaggcagtgatgatatcaggaagctgctcgagagccagggtagaaaggatatcaagctgatcgacatcgcgctgagcaagactgacagccgcaaatacgaaaacgctgtgtgggaccagtacaaggacttgtgccacatgcacacaggggtggtggtcgaaaagaagaagcggggaggaaaggaggagatcaccccacattgcgccctgatggactgcattatgttcgacgcggccgtgtcaggaggactcaacacttccgtgctccgggccgtgctgccccgggacatggtgttcagaacttccactcctcgcgtggtgctgggcccgggacctggaacctcactgtacaagggggtgtacgaactgcagaccctggaattgaacatggaaactctgaacatgaccatgcctctctcctgtaccaagaacaattcccaccactacattatggtcggaaacgaaactgggctcgagctgaccctcaccaacacctccataatcaaccacaagttctgcaacctttctgatgcccacaagaagaacctatacgaccatgccctgatgtccattatctccaccttccacctctcaatccccaacttcaatcaatacgaagcgatgtcctgtgatttcaacggcggaaagatctcggtgcagtacaacctctcgcattcatacgccggagatgccgccaaccactgcggtactgtggccaacggggtgctccagacctttatgaggatggcgtggggaggatcctacatcgccttggactccggacgcggcaactgggactgcatcatgaccagctaccagtacctgattattcagaacaccacctgggaggaccactgccagttctcgcgcccttcgccgattgggtaccttggcctcctgtcgcaaagaaccagagacatctacatcagccgccggttactgggaacttttacttggaccctttcggattccgagggaaaggacacgcccggcggttactgcctgacgcggtggatgctgattgaagctgaactcaagtgtttcggcaacaccgccgtggccaagtgcaacgagaagcacgacgaagagttctgcgatatgctgcgactgttcgatttcaacaagcaggctattcagcggctgaaggccgaggcccagatgagcattcagctgatcaacaaggccgtgaacgccctgattaacgatcagctcattatgaagaaccatctgcgggacatcatgggcatcccttactgcaactactccaagtattggtacctgaaccacactaccactggccgcaccagcctcccgaagtgctggctcgtgtccaacggatcctatctgaacgaaacccacttttccgacgacattgagcagcaggccgacaacatgatcaccgagatgttgcagaaggagtacatggagcgccaggggaaaactcccctgggactggtggacgggattgtggctgggttggcggtcctggcggtggtggtgatcggtgccgtggtggcagccgtgatgtgcaggagaaagtcgtccggcgggaagggagggtcctacagccaagcggcctgttctgactccgcccagggctccgacgtgtcactgacagcctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 805-900 - skipped (96 entries)

SEQ ID NO: 901 - RNA, 1798 nt, synthetic construct
  source 1..1798: mol_type = other RNA
acatttgcttctgacacaactgtgttcactagcaacctcaaacagccaccatggaactgctgatcctgaaggccaacgccatcaccactattctgaccgccgtgaccttttgcttcgcctccggccagaacatcacagaggaattctaccagagcacctgttctgccgtcagcaagggctacctgtctgccctgagaacaggctggtacacctctgtgatcaccatagagctgagtaacatcaaagagaacaagtgtaatggaaccgatgcaaaggtgaaactgattaaacaggagctggacaagtacaagaacgccgtcaccgagctgcagctactgatgcagagcacccccgccaccggctcaggcagcgccatctgcagcggcgtcgccgtgtgcaaggtgctgcacctggaaggcgaagtgaacaaaatcaagtctgctctgctgtccacaaacaaggctgttgtgagcctgagcaatggcgtgagcgtgctgacattcaaggtgctagatctgaaaaactacatcgataagcaactgctgcctatcctgaacaagcagagctgcagcatcagcaacatcgagacagtgatcgaatttcagcagaaaaacaaccggctgctggaaatcacaagagagttcagcgtgaacgccggagtgaccacccctgtgtctacctacatgctgacaaatagcgagctgctgagcctgatcaacgacatgcctattaccaacgaccagaagaagctgatgagcaacaacgtgcagatcgtgcggcagcaatcatactctatcatgtgcatcatcaaggaagaagtgctggcttacgtggtgcaactgcctctgtacggcgtaatcgacacaccatgctggaagctgcacaccagccctctgtgcacaaccaacaccaaggaaggctccaacatctgtctgaccaggacagatagaggctggtattgtgacaacgccggcagcgtgagcttcttcccccaggccgagacatgcaaagtgcagagcaatagagtgttttgcgacaccatgaacagcagaaccctgccatctgaggtgaacctgtgtaacgtggacatcttcaaccccaagtacgactgcaagatcatgaccagcaaaacagatgtgagttctagcgtcatcaccagcctgggtgccatcgtatcttgctacggcaaaaccaagtgcaccgccagcaataagaatcggggaatcatcaagacattcagcaacggctgcgactacgtttctaataagggagtggataccgtgtctgtgggaaataccctgtactgtgtgaacaagcaggagggcaagagcctgtatgtgaagggcgagcctatcatcaacttctacgatcctctggtgttccctagcgacgagttcgacgctagcatcagccaggtgaatgagaagatcaaccagtccctcgccttcatcagaaagtccgacgagctgctccacaacgtgaacgctggcaagagcacaacaaacatcatgatcaccacaattatcatcgtgatcatcgtgatcctgctgagcctgatcgccgtgggcctgctgctgtactgcaaggccagaagcacccccgtgaccctgtccaaggaccagctgagcggcatcaacaacatcgcctttagcaactgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 902 - RNA, 1798 nt, synthetic construct; identical to SEQ ID NO: 506
  source 1..1798: mol_type = other RNA

SEQ ID NO: 903 - RNA, 1798 nt, synthetic construct; identical to SEQ ID NO: 507
  source 1..1798: mol_type = other RNA

SEQ ID NO: 904 - RNA, 1798 nt, synthetic construct; identical to SEQ ID NO: 508
  source 1..1798: mol_type = other RNA

SEQ ID NO: 905 - protein, 566 aa, Alphainfluenzavirus influenzae
MKAILVVMLYTFTTANADTLCIGYHANNSTDTVDTVLEKNVTVTHSVNLLEDKHNGKLCKLRGVAPLHLGQCNIAGWILGNPECESLSTARSWSYIVETSNSDNGTCYPGDFINYEELREQLSSVSSFERFEIFPKTSSWPNHDSDNGVTAACSHAGARSFYKNLIWLVKKGKSYPKINQTYINDKGKEVLVLWGIHHPPTITDQESLYQNADAYVFVGTSRYSKKFKPEIAARPKVRDRAGRMNYYWTLVEPGDKITFEATGNLVAPRYAFTMEKEAGSGIIISDTPVHDCNATCQTPEGAINTSLPFQNVHPITIGKCPKYVRSTKLRLATGLRNVPSIQSRGLFGAIAGFIEGGWTGMVDGWYGYHHQNDQGSGYAADLKSTQNAIDKITNKVNSVIEKMNTQFTAVGKEFNHLEKRIENLNKKVDDGFLDVWTYNAELLVLLENERTLDYHDSNVKNLYEKVRHQLKNNAKEIGNGCFEFYHKCDNTCMESVKNGTYDYPKYSEEAKLNREKIDGVKLDSTRIYQILAIYSTVASSLVLVVSLGAISFWMCSNGSLQCRICI

SEQ ID NO: 906 - protein, 566 aa, Alphainfluenzavirus influenzae; identical to SEQ ID NO: 18

SEQ ID NO: 907 - protein, 566 aa, Alphainfluenzavirus influenzae
MKAIIALSNILCLVFAQKIPGNDNSTATLCLGHHAVPNGTIVKTITNDRIEVTNATELVQNSSIGKICNSPHQILDGGNCTLIDALLGDPQCDGFQNKEWDLFVERSRANSSCYPYDVPDYASLRSLVASSGTLEFKDESFNWTGVKQNGTSSACKRGSSNSFFSRLNWLTSLNNIYPAQNVTMPNKEQFDKLYIWGVHHPATDKNQFSLFAQSSGRITVSTKRSQQAVIPNIGSRPRVRDIPSRISIYWTIVKPGDILLINSTGNLIAPRGYFKIRSGKSSIMRSDAPIGECKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQISGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAMSCFLLCIALLGFIMWACQKGNIRCNICI

SEQ ID NO: 908-910 - skipped (3 entries)

SEQ ID NO: 911 - protein, 188 aa, unidentified
MFFKCNQVFTLVFLLLLYFNNIVYTHVDDIKNTSQKKITYDKYNKNKENMNNEKNDNKDNKDNIYNDNINNDNINNDNINNEDEYKFLSMKHYKDSLSNKLNNENDHMNYLIRKRKDNTQGSQHFNENIENNENVENNENIENNENNENIENIENNENNENNENIENNENNENNENSSIMNSESYNNI

SEQ ID NO: 912 - protein, 320 aa, synthetic construct
KNVNFLQYHFKELSNYNIANSIDILQEKEGHLDFVIIPHYTFLDYYKHLSYNSIYHKSSTYGKCIAVDAFIKKINETYDKVKSKCNDIKNDLIATIKKLEHPYDINNKNMNRTFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFN

SEQ ID NO: 913 - skipped

SEQ ID NO: 914 - RNA, 1891 nt, synthetic construct
  source 1..1891: mol_type = other RNA
acatttgcttctgacacaactgtgttcactagcaacctcaaacagccaccatgaaaactatcatcgccctctccaacatactctgcttggtgttcgcacaaaagatccctggaaacgacaatagcaccgcaaccctgtgtctcggacatcacgcggtgccgaacgggaccattgtcaagaccattaccaatgatcgcatcgaagtgacaaacgccaccgagctggtgcagaactcgtccatcggagagatctgcggcagcccgcatcagatcctggacggcggcaactgcactctgatcgatgccctcctgggtgatcctcagtgtgacggcttccaaaacaaggagtgggatctgttcgtggagcggagccgggccaacagcaactgttacccctacgatgtgcctgactacgcctcacttcggtccctggtcgcatcctccgggacgctggaattcaaaaacgagtcgttcaactggacgggagtgaaacagaacggcacctccagtgcttgcatccggggctcctcctcatcctttttctcgcgactgaactggcttacctcgctgaacaatatctacccggcgcagaacgtcaccatgcccaacaaggagcaatttgacaagctctacatttggggggtgcaccaccccgacactgacaagaaccagatctccctgtttgcccaaagctcaggtcgcattactgtgtccaccaagaggtcccagcaggccgtgatcccaaacattggaagcagaccgagaatccgcggcatcccatcccggatctcaatctactggactattgtgaagccgggggacatcctgctgatcaactccaccggaaacctgattgccccgcgcggctatttcaagattcgcagcggaaagtcgtccattatgagatcggacgcgcctatcgggaagtgcaagagcgaatgtatcacccccaacggatccatccccaatgacaagccgttccagaacgtgaaccgcatcacttacggagcttgccctcgctacgtgaagcagtcaaccctcaaactggccaccggcatgcggaacgtgcccgaaaagcagaccagaggaattttcggggccatcgcgggtttcattgaaaatggatgggagggaatggtggacggttggtacggattcaggcaccaaaactccgagggtcggggccaggccgccgacctgaagtccacccaggccgcgattgaccagatcaacgggaagctgaacaggctgattggaaagaccaacgaaaagttccaccaaatcgagaaggaattctcggaggtcgaagggagagtccaggacctcgagaaatacgtggaggacactaagattgatctgtggtcttacaacgccgaactgttggtggccctggaaaaccagcacaccatcgatctgactgatagcgagatgaacaagctgttcgaaaagactaagaagcagttgcgcgagaacgccgaggacatgggaaatggttgcttcaagatctatcataagtgcgacaacgcctgcatcggcagcattcggaacgaaacctacgaccacaacgtctaccgggatgaagctctgaacaaccggttccagatcaagggagtggaactcaagtccggctacaaggactggatcctttggatctcgtttgctatgtcctgcttccttctctgtatcgccctgctgggcttcattatgtgggcgtgccagaagggaaacattaggtgcaatatctgcatctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 915 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

(19) United States
(12) Patent Application Publication
(10) Pub. No.: US 2026/0283973 A1
(43) Pub. Date: Sep. 24, 2026

(54) IONIZABLE LIPIDS AND LIPOSOMAL NANOPARTICLE COMPOSITIONS

(71) Applicant: Akagera Medicines, Inc., Waltham, MA (US)

(72) Inventors: Daryl C. Drummond, Lincoln, MA (US); Dmitri Kirpotin, San Francisco, CA (US); Mark E. Hayes, Mill Valley, CA (US); Ross Fulton, Southborough, MA (US); Alexander Koshkaryev, Norwood, MA (US)

(73) Assignee: Akagera Medicines, Inc., Waltham, MA (US)

(21) Appl. No.: 19/573,533

(22) Filed: Mar. 20, 2026

Related U.S. Application Data
(60) Provisional application No. 63/775,688, filed on Mar. 21, 2025.
(60) Provisional application No. 63/869,090, filed on Aug. 22, 2025.
(60) Provisional application No. 63/944,616, filed on Dec. 19, 2025.
(60) Provisional application No. 64/007,071, filed on Mar. 16, 2026.

(51) Int. Cl. A61K 9/51; A61K 31/7105

(52) U.S. Cl. CPC A61K 9/5123; A61K 31/7105

(57) ABSTRACT

Aspects of the present disclosure provide for improved compositions of bioreducible and/or hydrolysable ionizable lipid compounds, useful in the preparation of lipid nanoparticles (LNP) for the delivery of therapeutic nucleic acids and other compounds to cells. Anionic phospholipids, including a phosphatidyl-L-serine (PS) or a phosphatidylglycerol (PG) can be included in the lipid nanoparticles to increase the transfection efficiency in human dendritic cells. In some examples, the LNP compositions are vaccine compositions comprising mRNA encapsulated in lipid nanoparticles formed from an ionizable cationic lipid compound, cholesterol, an anionic phospholipid and a PEG conjugated lipid.

### RELATED APPLICATIONS

This patent application claims the benefit of and priority to U.S. Provisional Patent Application No. 63/775,688, filed on Mar. 21, 2025, U.S. Provisional Patent Application No. 63/869,090, filed Aug. 22, 2025, U.S. Provisional U.S. Provisional Patent Application No. 63/944,616, filed Dec. 19, 2025, and U.S. Provisional Patent Application No. 64/007,071, filed Mar. 16, 2026, the entire content of each of which is hereby incorporated herein by reference in their entirety.

### REFERENCE TO SEQUENCE LISTING

The application contains a Sequence Listing which has been submitted electronically in XML file format and is hereby incorporated by reference in its entirety. Said XML copy, created on Mar. 20, 2026, is titled 191016-011610_US_SL.xml and is 394,185 bytes in size.

### FIELD

Aspects of the present disclosure relate to ionizable lipids useful for the preparation of lipid nanoparticles (LNP) encapsulating a nucleic acid.

### BACKGROUND

Lipid nanoparticles (LNP) are used for the delivery of therapeutic nucleic acids to cells, including vaccines to deliver mRNA therapeutics. LNP formulations typically include an ionizable lipid, a sterol, and a phospholipid. Ionizable lipids in LNP compositions facilitate delivery of encapsulated nucleic acid by remaining mostly neutral at physiological pH, but becoming protonated in the acidic environment of endosomes after cellular uptake. Ionizable lipids that are hydrolysable or bioreducible offer desirable in vivo clearance properties for vaccine-focused lipid nanoparticle (LNP) delivery systems by enhancing both efficacy and safety. Hydrolysable lipids, which break down through controlled chemical or enzymatic cleavage, and bioreducible lipids, which degrade in response to intracellular reducing conditions, promote timely LNP disassembly after delivering the vaccine payload. There remains a need for ionizable lipids that are hydrolysable and/or bioreducible for use in the preparation of LNP formulations for therapeutic delivery of nucleic acids.

### SUMMARY

In some embodiments, this disclosure provides ionizable cationic lipid compounds with significantly improved hydrolysis (Example 4) with significantly improved mRNA transfection efficiency in dendritic cells from LNPs encapsulating the mRNA (Example 5). In some embodiments, an ionizable lipid compound consists of a dioxolane ring covalently bound to both (x) an alkylamine head group having a pKa of 6-7.5, and (y) a pair of identical geminal functionalized aliphatic chains at the 2-position of the dioxolane ring, each having 18-25 total carbon atoms and each consisting of (i) a 7-10 carbon linear aliphatic group covalently bound to the dioxolane ring and to (ii) an ester functional group, and (iii) a 10-20 carbon aliphatic chain covalently bound to the ester functional group. In some embodiments, the dioxolane ring is a 1,3-dioxolane ring and each of the functionalized aliphatic chains comprises two or more branched C₅ hydrocarbon monomer units such as isopentane (IPN) or 2-methylbut-2-ene (2 MB). For example, in some embodiments, the ionizable lipid is selected from the group consisting of:

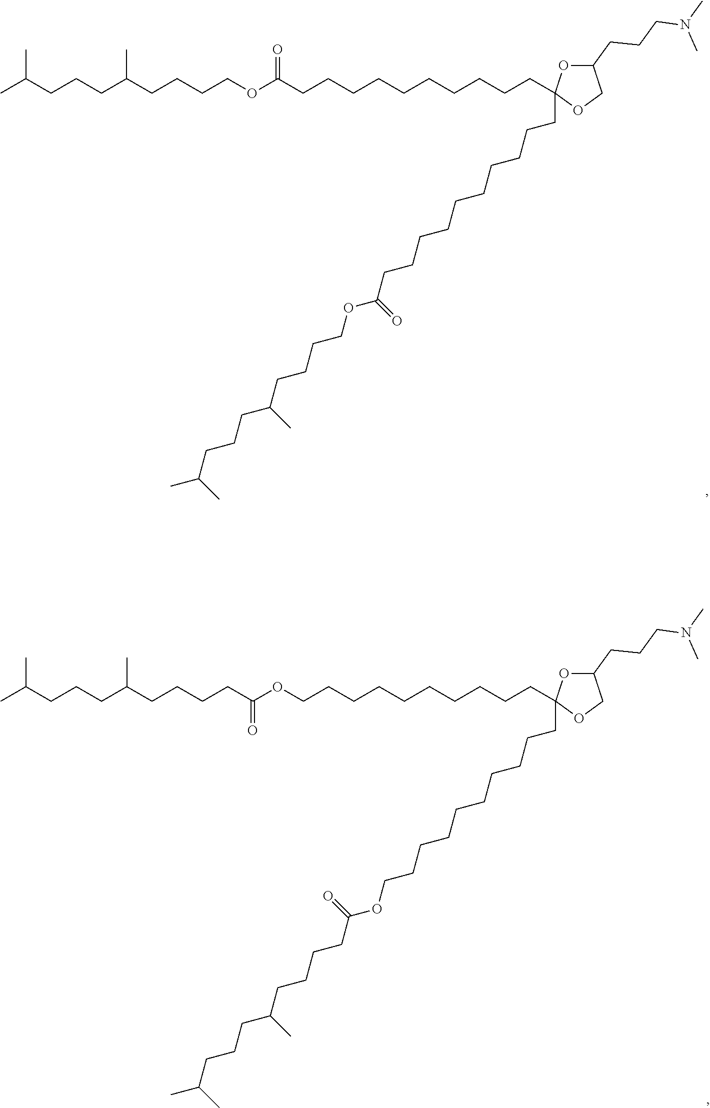

or a salt thereof.

In some embodiments, a lipid nanoparticle (LNP) composition comprises a mRNA nucleic acid encapsulated in lipidic nanoparticles formed from the ionizable lipid disclosed herein at a N/P ratio of 3.5 to 8 relative to the mRNA nucleic acid in a total amount of 40-65 mol % of the total lipid content of the LNP composition; a sterol in a total amount of 25-45 mol % of the total lipid content of the LNP composition; one or more phospholipids in a total amount of phospholipids of 5-25 mol % of the total lipid content of the LNP composition; and a PEG-containing conjugated lipid in a total amount of 0.5-5.0 mol % of the total lipid content of the LNP composition. In some embodiments, the two or more phospholipids in the LNP composition comprise a total of 5 mol % of a phosphatidyl-L-serine (PS). In some embodiments, the LNP composition comprises the conjugated lipid in a total amount of 0.5-2.5 mol % of the total lipid content of the LNP composition. In some embodiments, the phospholipids consist of: 1,2-distearoyl-sn-glycero-3-phosphocholine (DSPC) in a total amount of 5 mol % of the total lipid content of the LNP composition, and a phosphatidyl-L-serine (DPPS), such as dipalmitoylphosphatidyl-L-serine (DPPS) or distearoylphosphatidyl-L-serine (DSPS), in a total amount of 5 mol % of the total lipid content of the LNP composition. In some embodiments, the PEG-containing conjugated lipid in the LNP is PEG(2000)-dimyristoylglycerol (PEG-DMG) or PEG(2000)-dilauroylglycerol (PEG-DLG).

In some embodiments, a lipid nanoparticle (LNP) composition comprises a mRNA nucleic acid encoding an antigenic protein encapsulated at a N/P ratio of 3.25-8 in lipidic nanoparticles consisting of: a KCn-X1 or KCn-X2 ionizable cationic lipid disclosed herein and cholesterol in a molar ratio of the ionizable cationic lipid to the cholesterol of from 65:35 to 45:55, X mol % of an anionic phospholipid such as DPPS (wherein X is 2.5-7.5 mol %), (10-X) mol % phosphocholine phospholipid such as DSPC, and 1.5-4 mol % of a PEG conjugated lipid such as PEG-DMG. In some embodiments, a lipid nanoparticle (LNP) composition comprises a mRNA nucleic acid encapsulated at a N/P ratio of 5.25 in lipidic nanoparticles consisting of: 56 mol % of the KC3-X1-PH12 ionizable cationic lipid, 5 mol % DPPS, 5 mol % DSPC, 1.5 mol % PEG-DMG and 32.5 mol % cholesterol. In some embodiments, a lipid nanoparticle (LNP) composition comprises a mRNA nucleic acid encapsulated at a N/P ratio of 5.25 in lipidic nanoparticles consisting of: 52 mol % of the KC3-X1-PH12 ionizable cationic lipid, 5 mol % DPPS, 5 mol % DSPC, 1.5 mol % PEG-DMG and 36.5 mol % cholesterol.

### BRIEF DESCRIPTION OF THE DRAWINGS

FIG. 1A is a synthetic scheme for preparing certain ionizable cationic lipids.

FIG. 1B is a synthetic scheme for preparing an exemplary ionizable cationic lipid designated KC3-X1-PH12.

FIG. 1C is a table of ionizable cationic lipids.

FIG. 2A is a synthetic scheme for preparing certain ionizable cationic lipids.

FIG. 2B is a synthetic scheme for preparing an exemplary ionizable cationic lipid designated KC3-X2-PH12.

FIG. 2C is a table of ionizable cationic lipids.

FIG. 3A is a table of various ionizable cationic lipids of Formula (I).

FIG. 3B is a table of various ionizable cationic lipids of Formula (I).

FIG. 4A is a graph of the biodegradability of ionizable cationic lipids (ICLs) measured after 15 minutes in the presence of pancreatin enzymes (including trypsin, amylase and lipase) measured according to the assay of Example 4.

FIG. 4B is a graph of the biodegradability of ionizable cationic lipids (ICLs) measured after 3 hours in the presence of pancreatin enzymes (including trypsin, amylase and lipase) measured according to the assay of Example 4.

FIG. 4C is a graph showing the enzymatic degradation of various ionizable lipids.

FIG. 5A-5B are each schematics of a mRNA construct comprising a 5′-cap, a 5′-UTR, a cassette comprising a signal peptide, an open reading frame and a terminal domain encoding a synthetic polypeptide, a 3′-UTR and a poly-A region.

FIGS. 6A-6E are figures describing an experiment comparing B cell responses in BALB/c after prime/boost vaccination with mRNA encoding an influenza A subtypes H1, H3, and B/Victoria and RSV preF mRNA (Example 10). The mRNA was formulated with LNPs comprised with different ionizable lipids. FIG. 6A is a table summarizing the experimental design. FIG. 6B is a graph showing the anti-H1 IgG titers 21 days after vaccination with a high (0.03 mcg) dose. FIG. 6C is a graph showing the anti-H1 IgG titers 21 days after vaccination with a low (0.01 mcg) dose. FIG. 6D is a graph showing the anti-H3 IgG titers 21 days after vaccination with a high (0.045 mcg) dose; FIG. 6E is a graph showing the anti-H3 IgG titers 21 days after vaccination with a low (0.015 mcg) dose.

### DETAILED DESCRIPTION

In some embodiments, the present disclosure provides ionizable cationic lipid compounds of Formula (I):

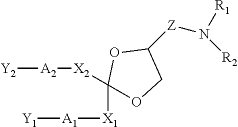

or a pharmaceutically acceptable salt thereof, wherein
R₁ and R₂ are each independently C₁₋₄ alkyl optionally substituted with hydroxyl;
Z is *—(CH₂)ₙ—**, wherein * is a covalent bond to the 1,3-dioxolane ring and ** is a covalent bond to —NR₁R₂, and n is 2, 3 or 4;
—X₁— and —X₂— are the same of different and are each independently —(CH₂)₍ₓ₎—, wherein (x) is 7-10;
-A₁- and -A₂- are the same or different and are each independently ester groups;
—Y₁— and —Y₂— are the same or different and are each an aliphatic chain having (y) total carbon atoms, optionally comprising two or more branched C₅ hydrocarbon monomer units, wherein (y) is 10-17; and
the sum of (x)+(y) is 18-25.

In some embodiments, ionizable lipid is a compound of Formula (I-A):

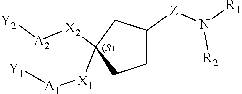

or a pharmaceutically acceptable salt thereof, wherein R₁, R₂, Z, X₁, X₂, A₁, A₂, Y₁ and Y₂ are as defined with respect to Formula (I).

In some embodiments, R₁ and R₂ are each methyl and n is 2, 3 or 4. In some embodiments, n is 2 and R₁ and R₂ are each methyl. In some embodiments, n is 3 and R₁ and R₂ are each methyl.

In some embodiments, n is 4 and R₁ and R₂ are each methyl. In some embodiments, *—Z—N(R₁)(R₂) is selected from —(CH₂)₂₋₄—N(CH₃)₂, and —(CH₂)₂₋₄—N(CH₃)—(CH₂)₂₋₄—OH, wherein * is a covalent bond to the 1,3-dioxolane ring in Formula (I).

In some embodiments, —X₁— and —X₂— are both —(CH₂)₍ₓ₎—, wherein (x) is 7, 8, 9 or 10; A₁ and A₂ are the same and are both independently *—C(O)—O—** or *—O—C(O)—**, wherein * is a covalent bond to Y₁ and Y₂, respectively, and ** is a covalent bond to X₁ and X₂, respectively; and —Y₁— and —Y₂— are each an aliphatic chain having (y) total carbon atoms, comprising two or more branched C₅ hydrocarbon monomer units, wherein (y) is 12, 13, 14, or 15; and wherein the sum of (x)+(y) is 22. In some embodiments, —Y₁ and —Y₂ are each independently *—(CH₂)₀₋₂—(R_{y})₂₋₄—H wherein each R_{y} is independently an isopentane (IPN) or a 2-methylbut-2-ene (2 MB) hydrocarbon monomer unit, and * is a covalent bond to A₁ or A₂. In some embodiments, Ry is —CH₂—CH(—CH₃)—CH₂—CH₂— or —CH₂—C(—CH₃)═CH—CH₂—. In some embodiments, the ionizable lipid is a compound of Formula (I), wherein X₁-A₁-Y₁ and —X₂-A₂-Y₂ are identical.

In some embodiments, the compound is a compound of Formula (I), wherein R₁ and R₂ are each methyl and n is 2, 3 or 4. In some embodiments, the compound is a compound of Formula (I), wherein n is 2 and R₁ and R₂ are each methyl. In some embodiments, the compound is a compound of Formula (I), wherein n is 3 and R₁ and R₂ are each methyl. In some embodiments, the compound is a compound of Formula (I), wherein n is 4 and R₁ and R₂ are each methyl. In some embodiments, the compound is a compound of Formula (I), wherein n is 2, 3 or 4, R₁ is —(CH₂)₂—OH and R₂ is methyl. In some embodiments, the compound is a compound of Formula (I), wherein n is 2, R₁ is —(CH₂)₂—OH and R₂ is methyl. In some embodiments, the compound is a compound of Formula (I), wherein n is 3, R₁ is —(CH₂)₂—OH and R₂ is methyl. In some embodiments, the compound is a compound of Formula (I), wherein n is 4, R₁ is —(CH₂)₂—OH and R₂ is methyl. In some embodiments, the compound is a compound of Formula (I), wherein R₁ and R₂ are each C₁₋₄ alkyl optionally substituted with hydroxyl. In some embodiments, the compound is a compound of Formula (I), wherein R₁ is C₁₋₄ alkyl and R₂ is C₁₋₄ alkyl optionally substituted with hydroxyl. In some embodiments, the compound is a compound of Formula (I), wherein R₁ is methyl and R₂ is —(CH₂)₂₋₄—OH. In some embodiments, the compound is a compound of Formula (I), wherein R₁ is methyl and R₂ is —(CH₂)₂—OH. In some embodiments, the compound is a compound of Formula (I), wherein R₁ is methyl and R₂ is —(CH₂)₃—OH. In some embodiments, the compound is a compound of Formula (I), wherein R₁ is methyl and R₂ is —(CH₂)₄—OH.

In some embodiments, the compound is a compound of Formula (I), wherein —X₁— and —X₂— are each independently —(CH₂)₍ₓ₎—, wherein (x) is 7, 8, 9 or 10; -A₁- and -A₂- are the same or different and are each independently hydrolysable or bioreducible groups; —Y₁— and —Y₂— are the same or different and are each an aliphatic chain comprising two or more branched C₅ hydrocarbon monomer units and having (y) total carbon atoms, wherein (y) is 10, 11, 12, 13, 14, 15, 16 or 17; and the sum of (x)+(y) is 18-25.

In some embodiments, the compound is a compound of Formula (I), wherein each of A₁ and A₂ is a hydrolysable group such as an ester, carbonate, carbamate, acetal, ketal, orthoester, acid-labile dioxolane, phosphoester or phosphoramidate. In some embodiments, the compound is a compound of Formula (I), wherein —Y₁ and —Y₂ comprise one or more branched C₅ hydrocarbon monomer units, such as n-pentane, isopentane, neopentane, 1-pentene, cis-2-pentene, trans-2-pentene, 3-methyl-1-butene, 2-methylbut-2-ene, 2-methylbut-1-ene, 3-methylbut-1-ene (isopentane), isoprene, 1,4-pentadiene, 1,5-pentadiene, 1,3-pentadiene (cis, trans or cis/trans), 2-methyl-1,3-pentadiene, 3-methyl-1,3-pentadiene, 1-pentyne, 2-pentyne, 3-methyl-1-butyne, or 3-methyl-2-butyne (isopentyne). In some embodiments, the compound is a compound of Formula (I), wherein —Y₁ and —Y₂ comprise two, three or four C₅ hydrocarbon monomer units in a configuration forming a covalent bond from the C₁ carbon of a C₅ hydrocarbon monomer to the C₄ carbon of an adjacent C₅ hydrocarbon monomer, such as IPN (—CH₂—CH(—CH₃)—CH₂—CH₂—) and/or 2 MB (—CH₂—C(—CH₃)═CH—CH₂—). In some embodiments, the compound is a compound of Formula (I), wherein —X₁-A₁-Y₁ and —X₂-A₂-Y₂ are identical; —X₁— and —X₂— are each independently —(CH₂)₍ₓ₎—, wherein (x) is 7, 8, 9 or 10; each of A₁ and A₂ is independently *—C(O)—O—** or *—O—C(O)—**, wherein * is a covalent bond to Y₁ and Y₂, respectively, and ** is a covalent bond to X₁ and X₂, respectively; —Y₁ and —Y₂ are each *—(CH₂)₀₋₂—(R_{y})₂₋₄—(CH₂)₀₋₂—H wherein each R_{y} is independently a C₅ hydrocarbon monomer unit such as an IPN or a 2 MB hydrocarbon monomer unit, and * is a covalent bond to A₁ or A₂; (y) is 12, 13, 14 or 15; and the sum of (x)+(y) is 22.

Some aspects of the disclosure relate to bioreducible ionizable cationic lipids of Formula (I) or (I-A), wherein each R₁ is independently linear hydrocarbon alkyl chains each comprising a hydrolysable or bioreducible group, such as an ester group (e.g, —O—CO— or —CO—O—); and R₂ and R₃ are each independently (C₁-C₄) alkyl optionally substituted with hydroxyl; and n is an integer equal to 2, 3 or 4.

In some embodiments, a bioreducible or hydrolysable ionizable cationic lipid comprises a compound of Formula (I) or Formula (I-A) wherein each of R₂ and R₃ is methyl, —Z— is —(CH₂)ₙ— and n is 3 or 4 and each R₁ is the same. In some embodiments, a bioreducible or hydrolysable ionizable cationic lipid comprises a compound of Formula (I) or Formula (I-A) wherein each of R₂ and R₃ is methyl and each R₁ is a hydrocarbon chain comprising a single ester group with a total 15-22 atoms, and optionally comprising one or two olefin groups. In some embodiments, a bioreducible or hydrolysable ionizable cationic lipid comprises a compound of Formula (I) or Formula (I-A) wherein each of R₂ and R₃ is methyl and each R₁ is a hydrocarbon chain comprising a single ester group with a total 15-22 atoms, and optionally comprising one olefin group. In some embodiments, a bioreducible or hydrolysable ionizable cationic lipid comprises a compound of Formula (I) or Formula (I-A) wherein each of R₂ and R₃ is methyl and each R₁ is a hydrocarbon chain comprising a single ester group with a total 15-22 atoms, and optionally comprising two olefin groups separated by at least two methylene groups.

In some embodiments, a bioreducible or hydrolysable ionizable cationic lipid comprises a compound of Formula (I) or Formula (I-A) wherein each of R₁ and R₂ is methyl, —Z— is —(CH₂)ₙ—, n is 3 or 4 and each of —X₁-A₁-Y₁ and —X₂-A₂-Y₂ is the same or different and each comprises an ester joining a proximal saturated C₇₋₈ hydrocarbon at —X₁— and —X₂— and a distal C₁₂₋₁₅ hydrocarbon at —Y₁ and —Y₂ each optionally comprising one or more olefins and one or more methyl substituents.

In some embodiments, a bioreducible or hydrolysable ionizable cationic lipid comprises a compound of Formula (I) or Formula (I-A) wherein each of R₁ and R₂ is methyl, Z is —(CH₂)ₙ— and n is 3 or 4 and each of —X₁-A₁-Y₁ and —X₂-A₂-Y₂ is the same or different and each comprises an ester at -A₁- and -A₂- joining a proximal saturated C₄₋₁₀ hydrocarbon at —X₁— and —X₂— and a distal C₁₀₋₂₀ hydrocarbon at —Y₁ and —Y₂ optionally comprising one or more olefins and one or more methyl substituents.

In some embodiments, a bioreducible or hydrolysable ionizable cationic lipid comprises a compound of Formula (I) or Formula (I-A) wherein each of R₁ and R₂ is methyl, —Z— is —(CH₂)ₙ—, n is 3 or 4 and each of —X₁-A₁-Y₁ and —X₂-A₂-Y₂ is the same and each comprises a proximal C₇₋₁₀ hydrocarbon at —X₁— and —X₂— and a distal saturated isoprenoid alkane group at —Y₁ and -Y₂ joined by an ester at -A₁- and -A₂-. In some embodiments, a bioreducible or hydrolysable ionizable cationic lipid comprises a compound of Formula (I) or Formula (I-A) wherein each of R₁ and R₂ is methyl, —Z— is —(CH₂)ₙ—, n is 3 or 4 and each of —X₁-A₁-Y₁ and —X₂-A₂-Y₂ is the same and each comprises a proximal C₇₋₁₀ hydrocarbon at —X₁— and —X₂— and a distal saturated phytane group at —Y₁ and —Y₂ joined by an ester at -A₁- and -A₂-. In some embodiments, a bioreducible or hydrolysable ionizable cationic lipid comprises a compound of Formula (I) or Formula (I-A) wherein each of R₁ and R₂ is methyl, —Z— is —(CH₂)ₙ—, n is 3 or 4 and each of —X₁-A₁-Y₁ and -X₂-A₂-Y₂ is the same and each comprises a proximal C₇₋₁₀ hydrocarbon at —X₁— and —X₂— and a distal unsaturated phytane group at —Y₁ and —Y₂ comprising one or more olefins joined by an ester at -A₁- and -A₂-.

In some embodiments, a bioreducible or hydrolysable ionizable cationic lipid comprises a compound of Formula (I) or Formula (I-A) wherein each of R₁ and R₂ is methyl, —Z— is —(CH₂)ₙ—, n is 3 and each of —X₁-A₁-Y₁ and —X₂-A₂-Y₂ is the same and each comprises a proximal C₇₋₁₀ hydrocarbon at —X₁— and —X₂— joined by an ester at -A₁- and -A₂- to a distal branched hydrocarbon chain comprising two or three isoprene or isopentane monomer groups at —Y₁ and —Y₂. In some embodiments, a bioreducible or hydrolysable ionizable cationic lipid comprises a compound of Formula (I) or Formula (I-A) wherein each of R₁ and R₂ is methyl, Z— is —(CH₂)ₙ—, n is 3 or 4 and each —X₁-A₁-Y₁ and —X₂-A₂-Y₂ are each —(CH₂)₇₋₁₀-A-Y, wherein A is —C(═O)—O— or —O—C(═O)—, and Y is —(CH₂—CH₂—CH(—CH₃)—CH₂)₃—H or —(CH₂—CH₂—CH(—CH₃)—CH₂)₃—H or —CH₂—CH₂—(CH₂—CH₂—CH(—CH₃)—CH₂)₂—H. In some embodiments, a bioreducible or hydrolysable ionizable cationic lipid comprises a compound of Formula (I) or Formula (I-A) wherein each of R₁ and R₂ is methyl, Z— is —(CH₂)ₙ—, n is 3 or 4 and each —X₁-A₁-Y₁ and —X₂-A₂-Y₂ is —(CH₂)₇-A-Y, wherein A is —C(═O)—O— or —O—C(═O)—, and Y is —(CH₂—CH₂—CH(—CH₃)—CH₂)₃—H. In some embodiments, a bioreducible or hydrolysable ionizable cationic lipid comprises a compound of Formula (I) or Formula (I-A) wherein each of R₂ and R₃ is methyl, Z— is —(CH₂)ₙ—, n is 3 or 4 and each —X₁-A₁-Y₁ and —X₂-A₂-Y₂ is —(CH₂)₈-A-Y, wherein A is —C(═O)—O— or —O—C(═O)—, and Y is —(CH₂—CH₂—CH(—CH₃)—CH₂)₃—H or —(CH₂)₂—CH═CH—(CH₂)₈—H or —(CH₂)₁₂—H. In some embodiments, a bioreducible or hydrolysable ionizable cationic lipid comprises a compound of Formula (I) or Formula (I-A) wherein each of R₂ and R₃ is methyl, Z— is —(CH₂)ₙ—, n is 3 or 4 and each —X₁-A₁-Y₁ and —X₂-A₂-Y₂ is —(CH₂)₁₀-A-Y, wherein A is —C(═O)—O— or —O—C(═O)—, and Y is —CH₂—CH₂—(CH₂—CH₂—CH(—CH₃)—CH₂)₂—H.

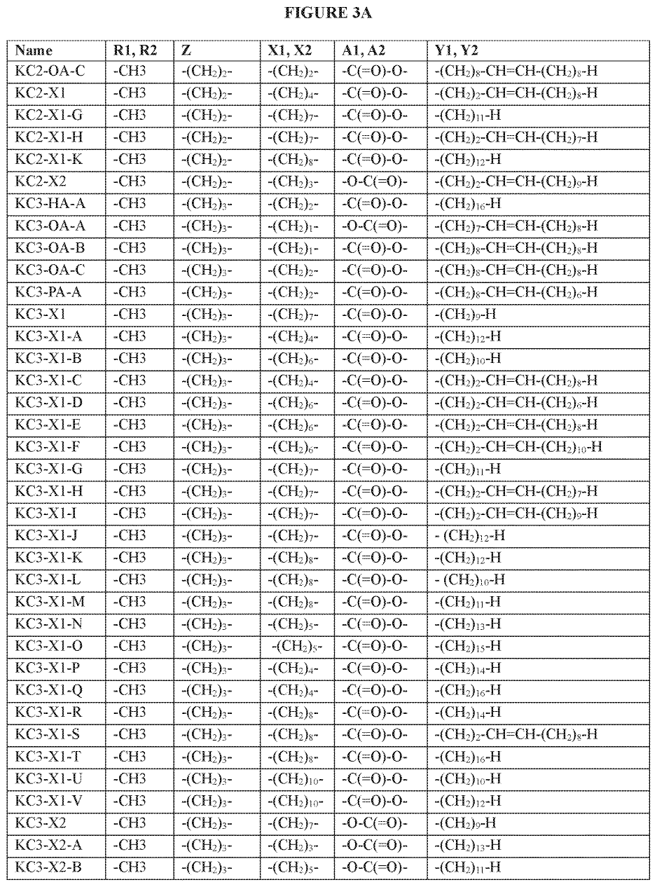

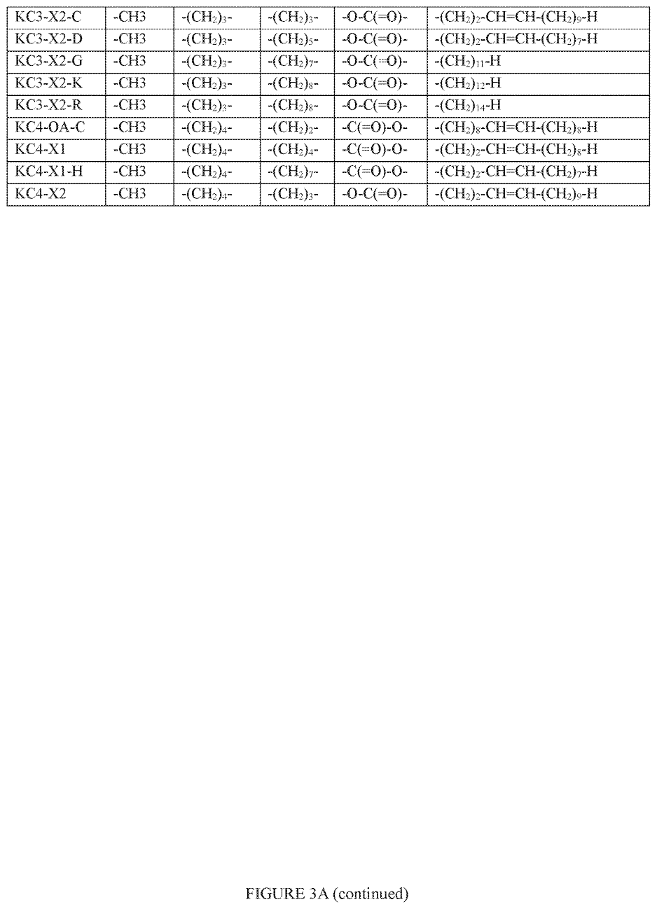

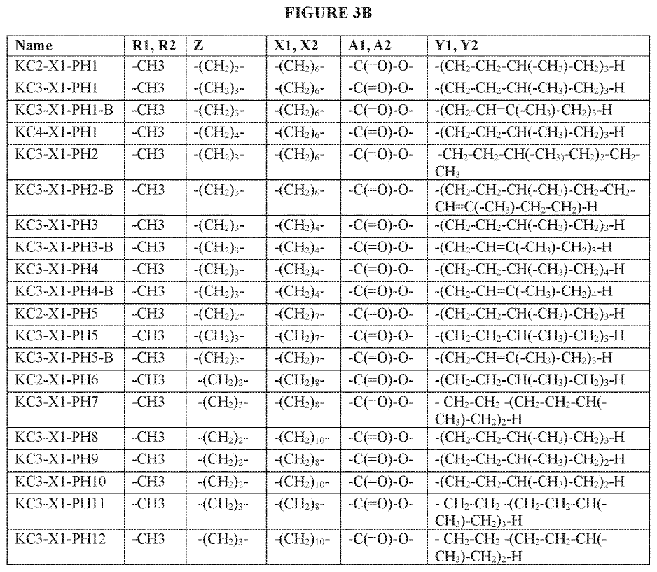

In some embodiments, a bioreducible or hydrolysable ionizable cationic lipid comprises a compound of Formula (I) or Formula (I-A) wherein each of R₁ and R₂ is methyl, Z— is —(CH₂)ₙ—, n is 3 and each —X₁-A₁-Y₁ and —X₂-A₂-Y₂ are each —(CH₂)₇₋₁₀-A-Y, wherein A is —C(═O)—O— or —O—C(═O)—, and Y is —(CH₂—CH₂—CH(—CH₃)—CH₂)₃—H or —(CH₂—CH₂—CH(—CH₃)—CH₂)₃—H or —CH₂—CH₂—(CH₂—CH₂—CH(—CH₃)—CH₂)₂—H. In some embodiments, a bioreducible or hydrolysable ionizable cationic lipid comprises a compound of Formula (I) or Formula (I-A) wherein each of R₁ and R₂ is methyl, Z— is —(CH₂)ₙ—, n is 3 and each —X₁-A₁-Y₁ and —X₂-A₂-Y₂ is —(CH₂)₇-A-Y, wherein A is —C(═O)—O— or —O—C(═O)—, and Y is —(CH₂—CH₂—CH(—CH₃)—CH₂)₃—H. In some embodiments, a bioreducible or hydrolysable ionizable cationic lipid comprises a compound of Formula (I) or Formula (I-A) wherein each of R₂ and R₃ is methyl, Z— is —(CH₂)ₙ—, n is 3 and each —X₁-A₁-Y₁ and —X₂-A₂-Y₂ is —(CH₂)₈-A-Y, wherein A is —C(═O)—O— or —O—C(═O)—, and Y is —(CH₂—CH₂—CH(—CH₃)—CH₂)₃—H or —(CH₂)₂—CH═CH—(CH₂)₈—H or —(CH₂)₁₂—H. In some embodiments, a bioreducible or hydrolysable ionizable cationic lipid comprises a compound of Formula (I) or Formula (I-A) wherein each of R₂ and R₃ is methyl, Z— is —(CH₂)ₙ—, n is 3 and each —X₁-A₁-Y₁ and —X₂-A₂-Y₂ is —(CH₂)₁₀-A-Y, wherein A is —C(═O)—O— or —O—C(═O)—, and Y is —CH₂—CH₂—(CH₂—CH₂—CH(—CH₃)—CH₂)₂—H. In some embodiments, a bioreducible or hydrolysable ionizable cationic lipid comprises a compound of Formula (I) or Formula (I-A) as described in the table in FIG. 3A or the table in FIG. 3B.

In some embodiments, a lipid nanoparticle (LNP) vaccine composition, comprises a mRNA nucleic acid encoding one or more immunogenic proteins, encapsulated in lipidic nanoparticles consisting of:
a. an ionizable cationic lipid of Formula (I) at a N/P ratio of 3.25-8 relative to the mRNA nucleic acid, or a pharmaceutically acceptable salt thereof, wherein R₁ and R₂ are each independently C₁₋₄ alkyl optionally substituted with hydroxyl; Z is *—(CH₂)ₙ—**, wherein * is a covalent bond to the 1,3-dioxolane ring and ** is a covalent bond to —NR₁R₂, and n is 2, 3 or 4; —X₁— and —X₂— are the same of different and are each independently —(CH₂)₍ₓ₎—, wherein (x) is 7-10; -A₁- and -A₂- are the same or different and are each independently ester groups; —Y₁ and —Y₂ comprise one or more branched C₅ hydrocarbon monomer units having a total of (y) carbon atoms each, wherein (y) is 10-17; wherein —Y₁— and —Y₂— each comprise a total of one, two or three isopentane (IPN) or 2-methylbut-2-ene (2 MB) branched C₅ hydrocarbon monomer units; and the sum of (x)+(y) is 20-25;
b. a phosphocholine phospholipid in a total amount of phospholipids of (10-X) mol % of the total lipid content of the LNP composition;
c. an anionic phospholipid in a total amount of phospholipids of X mol % of the total lipid content of the LNP composition, wherein X is 2.5-7.5;
d. a PEG-containing conjugated lipid in a total amount of 1.5-4.0 mol % of the total lipid content of the LNP composition; and
e. cholesterol, in a molar ratio of the ionizable cationic lipid to the cholesterol of from 65:35 to 45:55.

In some embodiments, the disclosure provides certain compounds useful as ionizable lipids in the preparation of lipid nanoparticles encapsulating a nucleic acid such as mRNA. Aspects of the disclosure are based in part on the discovery of ionizable lipids containing one or more functionalized aliphatic tails interrupted by ester linkages with certain defined ratios of aliphatic chain length on either side of the ester emerged from systematic structure-activity relationship studies aimed at improving nucleic acid delivery while enhancing biodegradability. Applicants discovered that positioning an ester within the lipid tail, rather than only at the headgroup-tail junction, allowed control over certain properties of the ionizable lipids. The ionizable lipids with ester-containing, asymmetrically segmented lipid tails are useful in preparing lipid nanoparticle formulations for the delivery of nucleic acids, such as in mRNA vaccine and therapeutic applications.

An August 2025 study shows the biodistribution patterns of ionizable cationic lipid components of FDA-approved vaccines, including ALC-0315 and SM-102 (Yuxiang R, et al., “Impact of ionizable lipid type on the pharmacokinetics and biodistribution of mRNA-lipid nanoparticles after intravenous and subcutaneous injection,” Journal of Controlled Release, v. 384, 2025, 113945, ISSN 0168-3659). The publication WO2022/115645 (PCT/US2021/060877) discloses ionizable cationic lipids including KC3-OA.

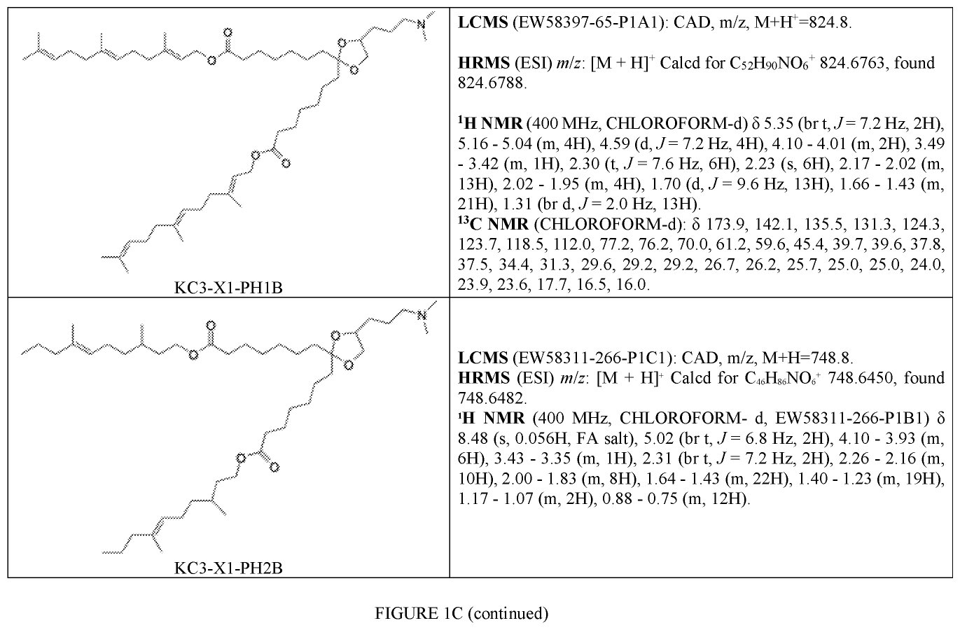

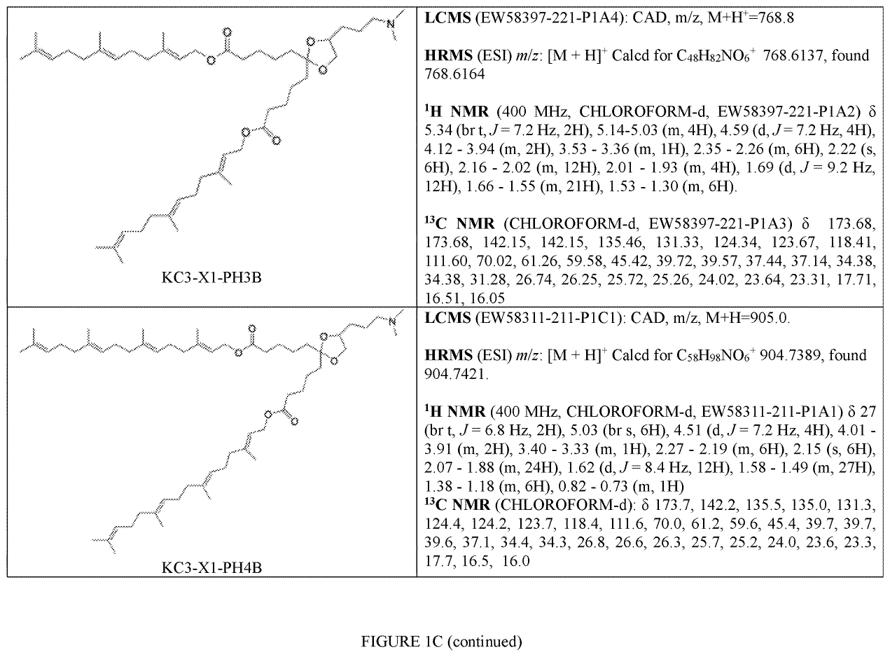

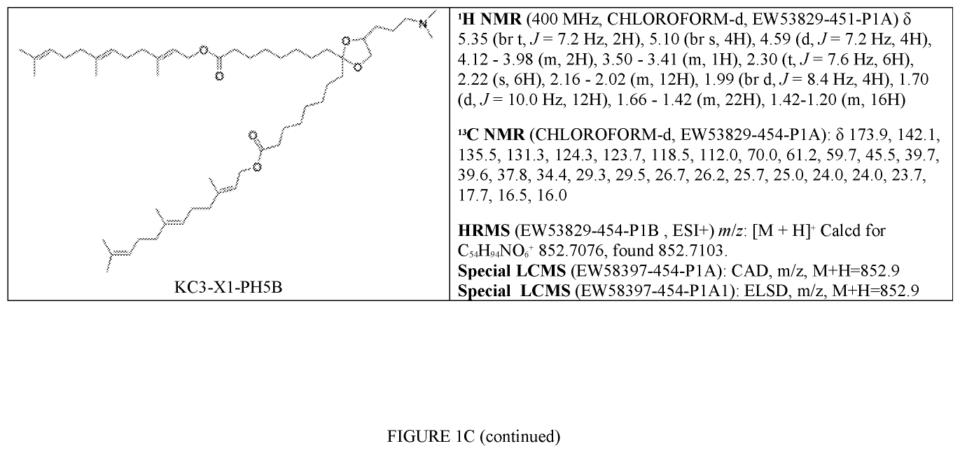

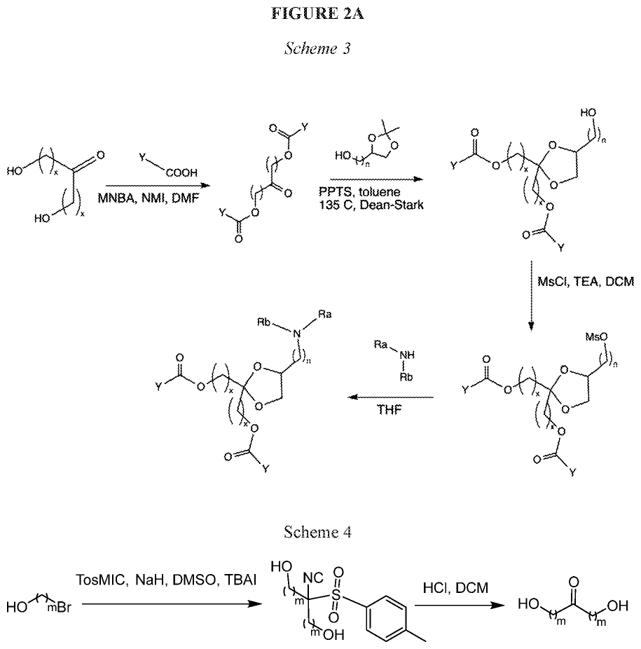

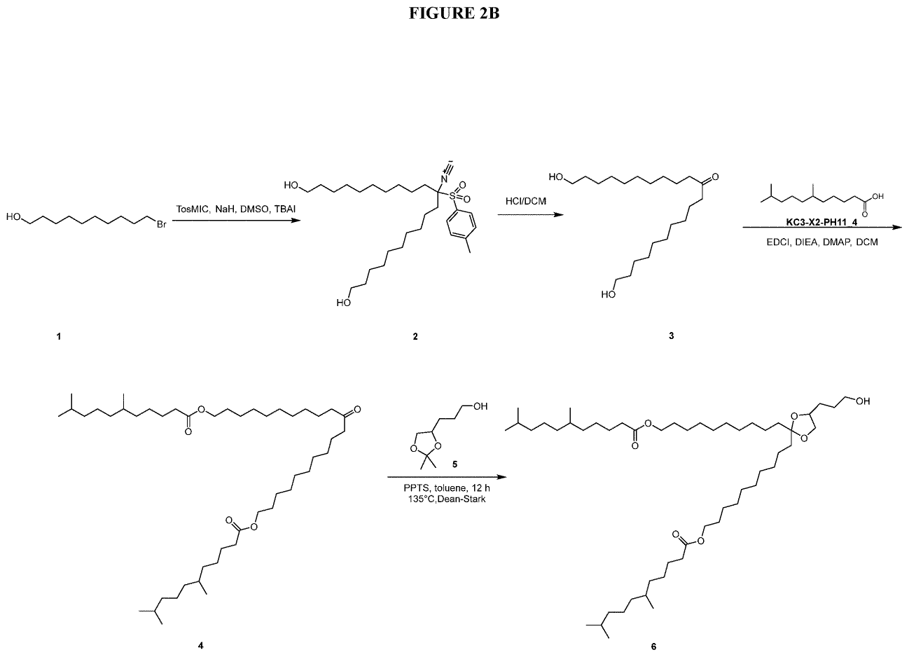

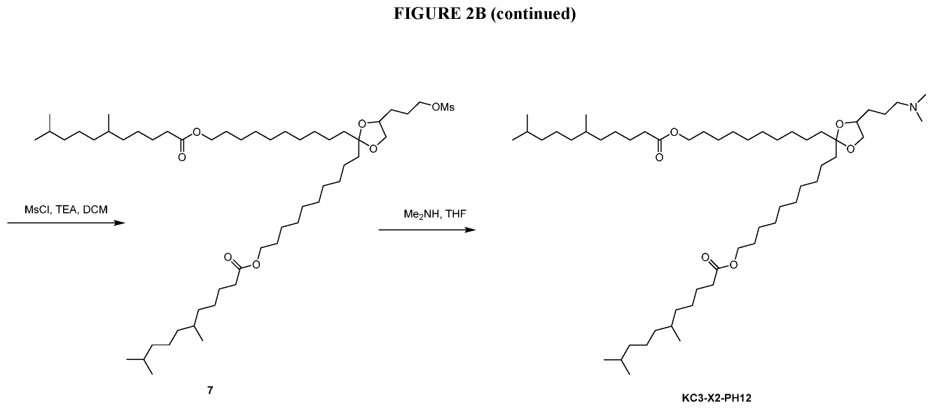

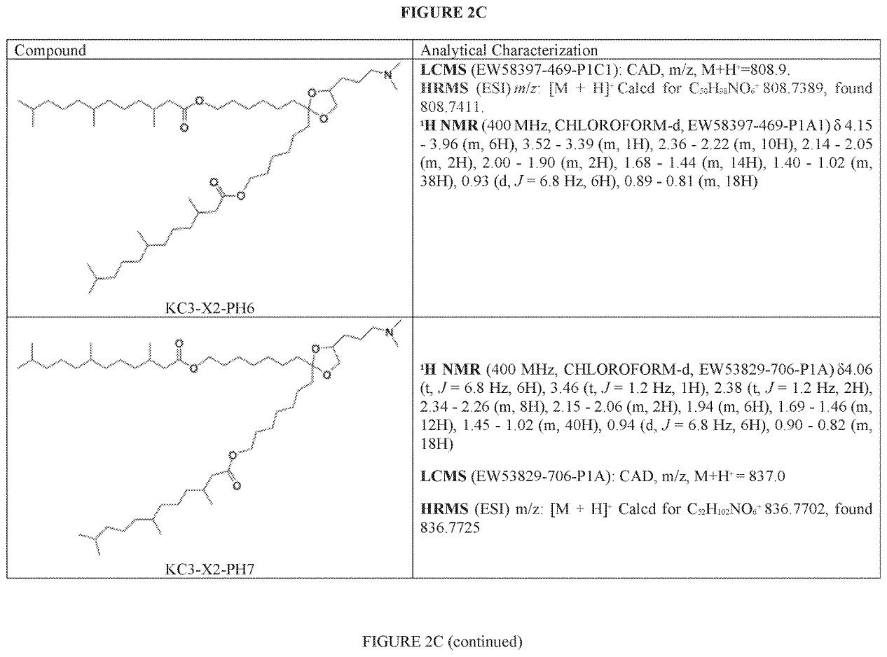

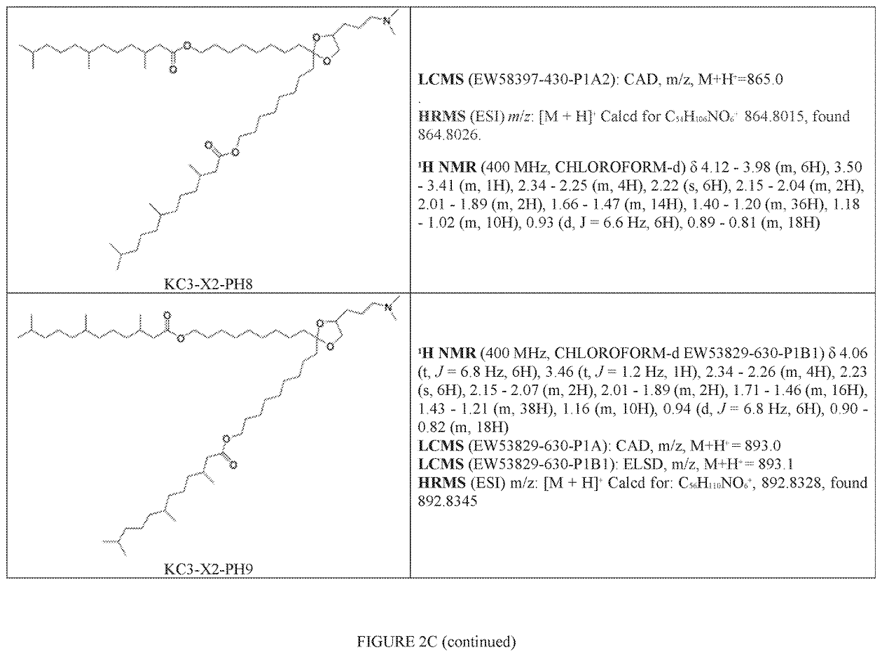

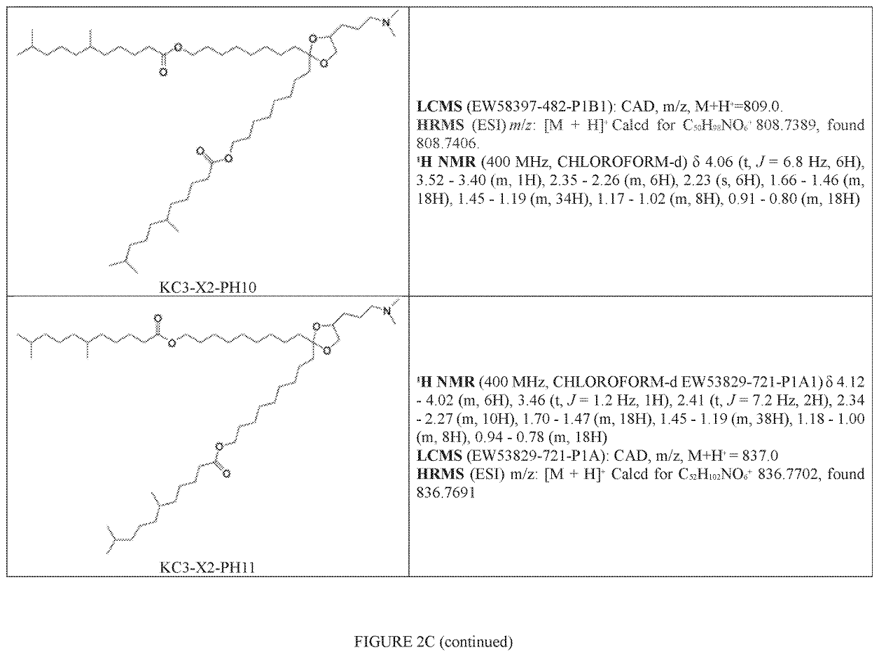

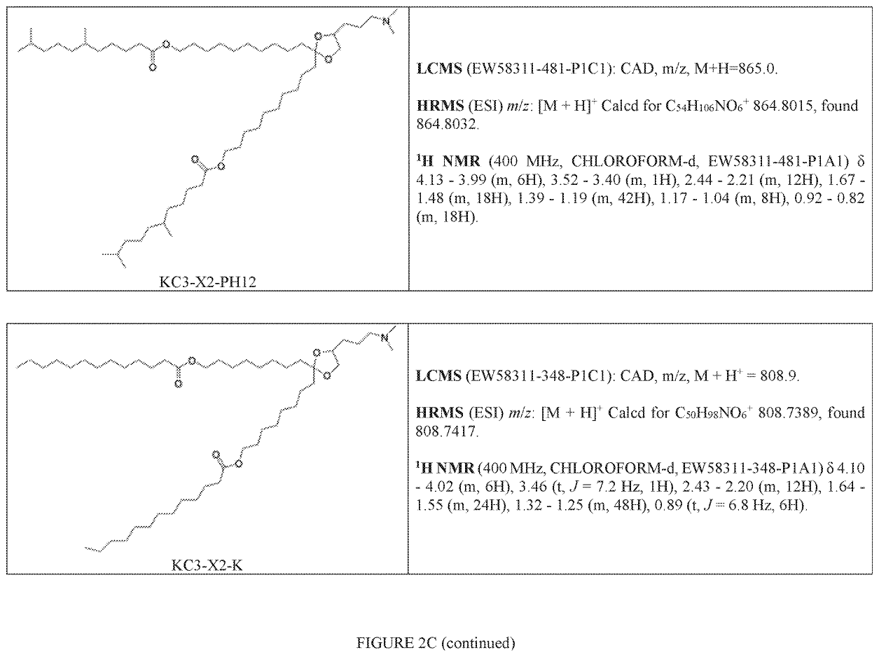

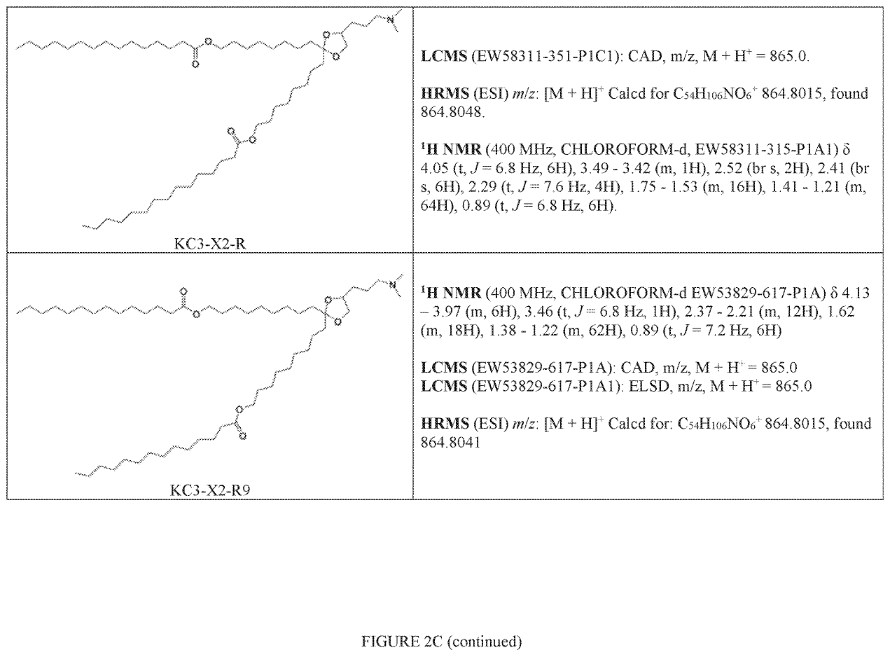

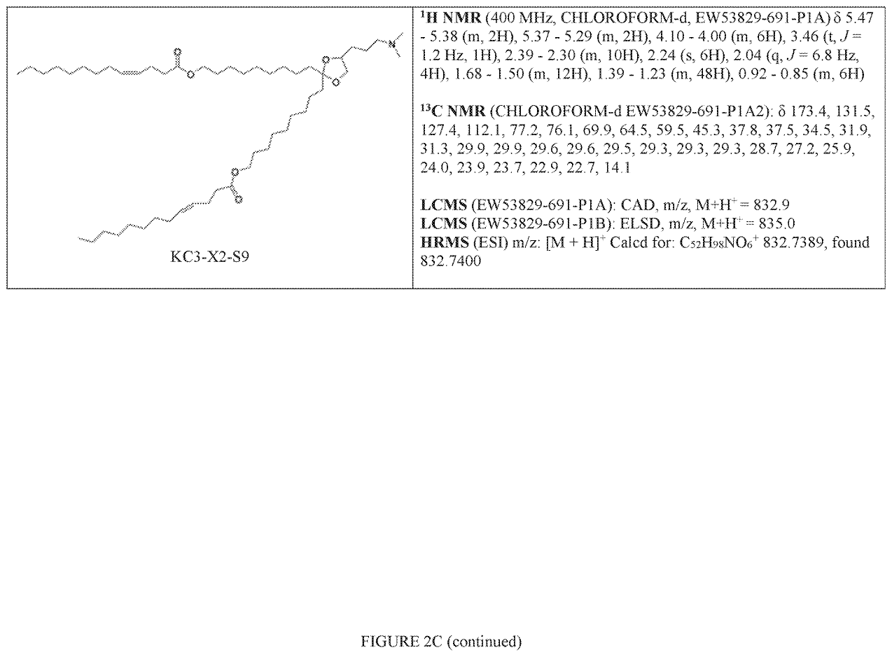

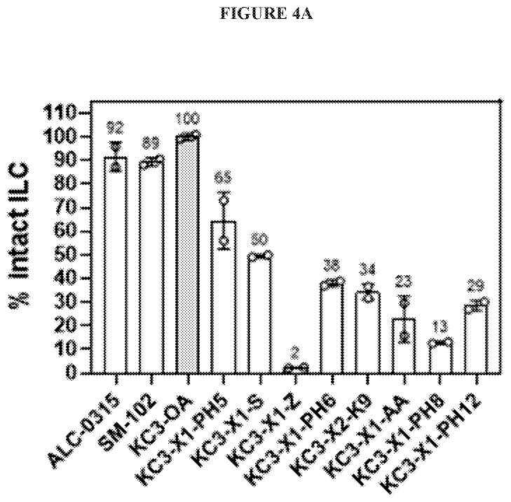

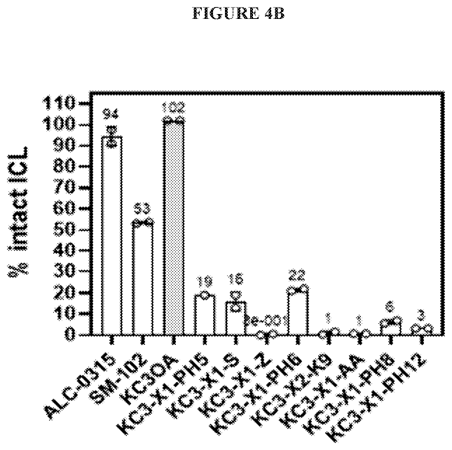

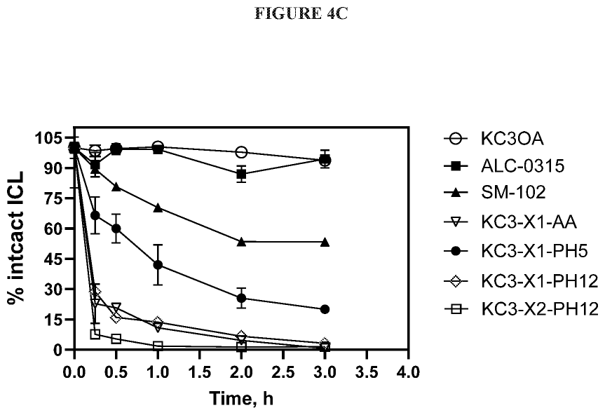

The hydrolysis of various ionizable lipids were evaluated in the pancreatin enzyme assay of Example 4. Pancreatin contains a mixture of digestive enzymes, including lipases and esterases, that mimic the enzymatic environment encountered by LNPs after administration and cellular uptake. FIG. 4A is a graph of the biodegradability of ionizable cationic lipids (ICLs) measured after 15 minutes in the presence of pancreatin enzymes (including trypsin, amylase and lipase) measured according to the assay of Example 4. In FIG. 4A, the compounds of Formula (I) were hydrolyzed about 29%-98% more than ALC-0315, about 27%-98% more than SM-102 and about 35%-98% faster than KC3-OA. FIG. 4B is a graph of the biodegradability of ionizable cationic lipids (ICLs) measured after 3 hours in the presence of pancreatin enzymes (including trypsin, amylase and lipase) measured according to the assay of Example 4. In FIG. 2B, the compounds of Formula (I) were hydrolyzed about 77-99% more than ALC-0315, about 58-99% more than SM-102 and about 81-99% faster than KC3-OA. FIG. 4C is a graph showing the enzymatic degradation of various ionizable cationic lipids. The ionizable lipids of Formula (I) were hydrolyzed significantly faster than ALC-0315 and SM-102 in the assay of Example 4. The hydrolysis of the ionizable lipids of Formula (I) was significantly greater and more rapid than comparator ionizable lipids ALC-0315, SM102 and KC3-OA, when evaluated in an assay incorporating pancreatin enzymes in Example 4.

In some embodiments, the disclosure provides certain LNP compositions comprising one or more ionizable lipids disclosed herein, such as the ionizable lipids of Formula (I). In some aspects, the LNP compositions comprise a nucleic acid encapsulated in lipid nanoparticles formed from: (x) an ionizable lipid of Formula (I) at a N/P ratio of 3 to 8 relative to the nucleic acid in a total amount of 40-65 mol % of the total lipid content of the LNP composition; (y) a sterol in a total amount of 0.5-50 mol % of the total lipid content of the LNP composition; (c) one or more phospholipids in a total amount of phospholipids of 5-50 mol % of the total lipid content of the LNP composition; and (d) a conjugated lipid in a total amount of 0.5-2.5 mol % of the total lipid content of the LNP composition. Unless explicitly indicated otherwise, the isomer form of the phosphatidylserine (PS) lipids used in the Examples is phosphatidyl-L-serine. In some aspects, the LNP composition is further characterized in that: the nucleic acid is mRNA; the ionizable cationic lipid is present in the LNP composition at a N/P ratio of 4 to 7 relative to the nucleic acid; the one or more phospholipids in the LNP comprise a phosphatidylserine (PS) lipid in a total amount of 2.5-10 mol % of the total lipid content of the LNP composition; and the conjugated lipid is a PEG-containing conjugated lipid. In some embodiments, the LNP is a vaccine composition comprising lipid nanoparticles encapsulating mRNA encoding a vaccine antigen (e.g., Tables 5A and 5B), and formed from (x) the ionizable lipid of Formula (I) (e.g., 45-55 mol % KC3-X1-PH5, KC3-X1-PH12, or KC3-X2-PH12), (y) the sterol (e.g., 25-45 mol % cholesterol), (c) a phosphocholine phospholipid (e.g., 1,2-distearoyl-sn-glycero-3-phosphocholine (DSPC), hydrogenated soy phosphatidylcholine (HSPC), or egg sphingomyelin (SM)), (d) a phosphatidyl-L-serine or phosphatidylglycerol phospholipid selected to target dendritic cells (e.g., 2.5-10 mol %, 2.5-7.5 mol %, or 5 mol % dipalmitoylphosphatidyl-L-serine (DPPS), distearoylphosphatidyl-L-serine (DSPS) or (1,2-Distearoyl-sn-glycero-3-phosphoglycerol (DSPG)), and (e) a PEG-conjugated lipid (e.g., 0.5-4.0 mol %, 1.0-4.0 mol %, 1.5-4.0 mol %, 1.5-2.5 mol %, 0.5 mol %, 1.0 mol %, 1.5 mol %, 2.0 mol %, or 2.5 mol % PEG(2000)-dimyristoylglycerol (PEG-DMG) or 1,2-dimyristoyl-rac-glycero-3-methoxypolyethylene glycol-2000 (PEG-DLG)).

In some embodiments, the LNP comprises a phospholipid. A phospholipid is a lipid that comprises a phosphate group and at least one alkyl, alkenyl, or heteroalkyl chain. A phospholipid may be naturally occurring or non-naturally occurring (e.g., a synthetic phospholipid). A phospholipid may comprise an amine, amide, ester, carboxyl, choline, hydroxyl, acetal, ether, carbohydrate, sterol, or a glycerol. In some embodiments, a phospholipid may comprise a phosphocholine, phosphosphingolipid, or a plasmalogen. Exemplary phospholipids include 1,2-distearoyl-sn-glycero-3-phosphocholine (DSPC), 1,2-dipalmitoyl-sn-glycero-3-phosphocholine (DPPC), dipalmitoylphosphatidyl-L-serine (DPPS), distearoylphosphatidyl-L-serine (DSPS), hydrogenated soy phosphatidylcholine (HSPC), and/or sphingomyelin (SM). Additional phospholipids that may be included in an LNP described herein are disclosed in Li, J. et al. (Asian J. Pharm. Sci. 10:81-98 (2015)), which is incorporated herein by reference in its entirety.

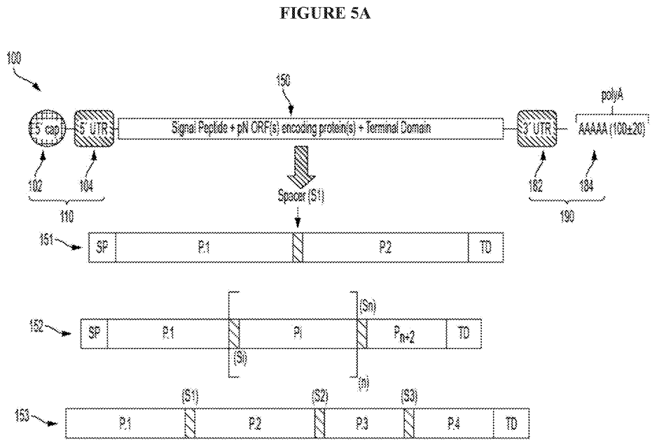

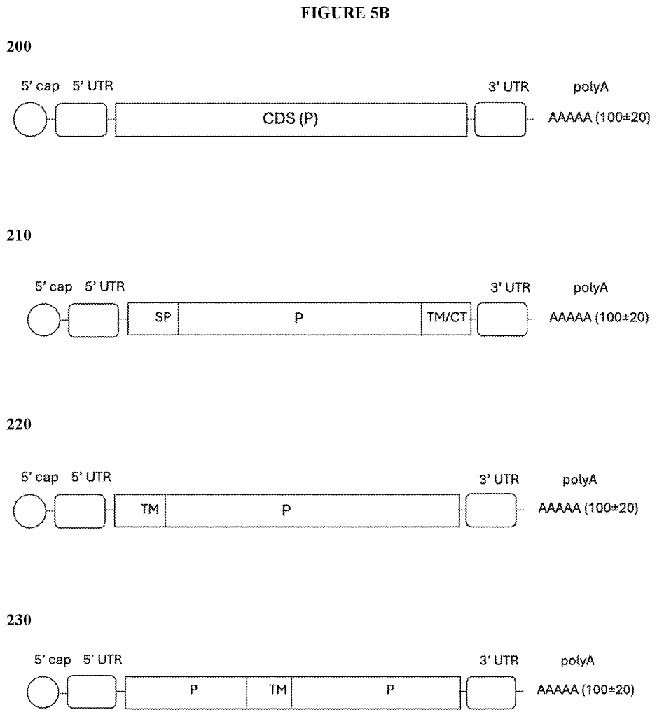

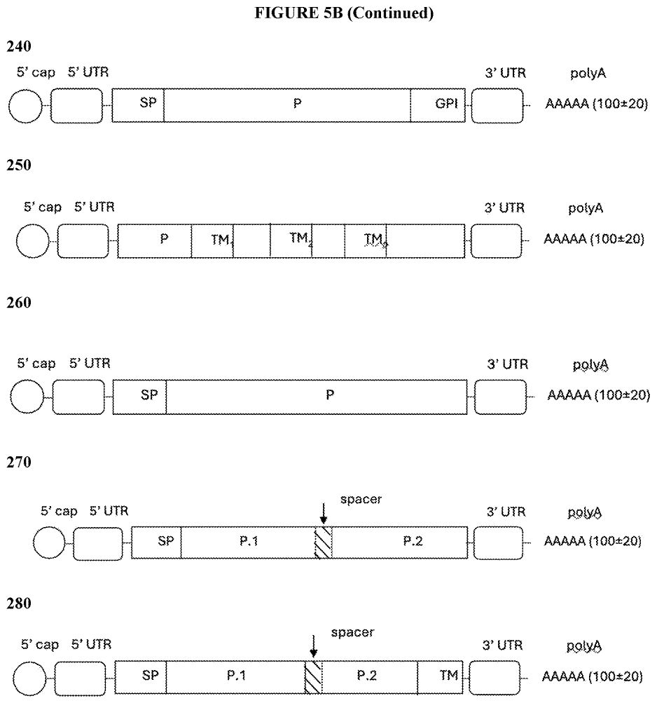

In some embodiments, LNP compositions comprising lipid nanoparticles comprising the ionizable lipid(s) disclosed herein and encapsulating mRNA. FIG. 5A is a schematic of a mRNA construct comprising a 5′-cap, a 5′-UTR, a coding sequence (CDS) that encodes a polypeptide antigen, a 3′-UTR and a poly-A region. The CDS can encode a natural or synthetic polypeptide antigen. In some embodiments, the mRNA (100) is a single mRNA comprising a 5′ region (110) and a 3′ region (190) flanking a CDS (150). The 5′ region of the mRNA (110) can comprise a 5′ cap and a 5′ UTR region. The 3′ region of the mRNA (190) can comprise a 3′UTR (182) and a polyA sequence (184). In one embodiment, the mRNA CDS (150) comprises (from 5′ to 3′) a secreted protein consisting of a signal peptide (SP), and one or more unique polypeptides optionally connected by a spacer. In another embodiment, the mRNA CDS (150) comprises (from 5′ to 3′) a type I single-pass membrane protein consisting of a signal peptide (SP), one or more unique polypeptide optionally connected by a spacer, and a terminal domain comprising a transmembrane and optionally a cytoplasmic domain. In another embodiment, the mRNA CDS (150) comprises (from 5′ to 3′) a type II single-pass membrane protein consisting of a transmembrane domain and one or more unique polypeptides optionally connected by a spacer. In another embodiment, the mRNA CDS (150) comprises (from 5′ to 3′) a type III single-pass membrane protein consisting of an extracellular domain, one or more unique polypeptides optionally connected by a spacer, a transmembrane domain and a intracellular domain comprised of one or more unique polypeptides optionally connected by a spacer. In another embodiment, the mRNA CDS (150) comprises (from 5′ to 3′) a glycosylphosphatidylinositol (GPI)-anchored membrane protein consisting of a SP, one or more unique polypeptides optionally connected by a spacer, and GPI anchor domain. In another embodiment, the mRNA CDS (150) comprises (from 5′ to 3′) a multi-pass membrane protein comprised of one or more unique polypeptides optionally connected by a spacer, and two or more transmembrane domains.

The mRNA CDS (150) can be configured to encode one or more covalently-linked unique polypeptides and comprises one or more spacers between each unique antigen protein. For example, a concatenated mRNA CDS (151) can be constructed that encodes for a polypeptide comprising a signal peptide (SP), a first antigen protein (P.1) and a second antigen protein (P.2) separated by a spacer (S1). A concatenated mRNA CDS of various lengths (152) can be constructed that encodes for a polypeptide with a signal peptide (SP), a first antigen protein (P.1), a spacer (Si) between the first antigen protein (P.1) and each subsequent antigen protein (Pi), and a third or subsequent antigen protein (Pn+2), where each consecutive antigen protein is separated by a spacer (Sn). Another CDS (153) can be constructed that encodes for a polypeptide that comprises a signal peptide (SP), a first antigen protein (P.1), a second antigen protein (P.2), a third antigen protein (P.3), and a fourth antigen protein (P.4), wherein each antigen protein is separated by a spacer (S1), (S2) and (S3) respectively. The mRNA spacer CDSs can encode polypeptides comprised of G/P (gly-pro), G/S (gly-ser) or AAY that are positioned between concatenated antigen proteins. In some embodiments, the mRNA polynucleotide sequence of the CDS (150) further encodes (x) a LAMP-1 signal peptide and a LAMP-1 transmembrane and cytoplasmic domain; or (y) a human HLA Class I signal peptide and a LAMP-1 transmembrane and cytoplasmic domain. SEQ ID NO: 35 is the N-terminal signal peptide of human LAMP-1 (Uniprot ID P11279, amino acids 1-28); SEQ ID NO: 36 is the C-terminal domain of human LAMP-1, including the transmembrane and cytoplasmic domains.

The RNA according to aspects of the disclosure may have modified ribonucleotides in order to increase its stability and/or decrease cytotoxicity. For example, in some embodiments, 5-methylcytidine in the RNA is substituted partially or completely, for cytidine. In some embodiments, 5-methylcytidine in the RNA is substituted completely for cytidine. Alternatively or additionally, in some embodiments, pseudouridine in the RNA used is substituted partially or completely, for uridine. In some embodiments, pseudouridine in the RNA used is substituted completely for uridine. In some aspects, the LNP comprises nucleic acid comprising a 5′ untranslated region (UTR) and 3′ UTR, polyA tail of about 80 to about 140 nucleotides in length, and (i) a 5′ enzymatic or (ii) a 5′ clean cap. In some aspects, the LNP comprises a chemically modified mRNA, wherein the chemically modified mRNA comprises N1-methylpseudouridine. In other embodiments, the mRNA does not include N1-methyl-pseudouridine.

FIG. 5B is a schematic of a mRNA construct (200) comprising a 5′ cap, 5′ UTR, a polypeptide coding sequence (CDS), 3′ UTR, polyA tail. The CDS encodes apolypeptide antigen (P). A Type I single-pass membrane protein mRNA construct (210) encodes a signal peptide encoded at the 5′ end (N-terminus of the protein). The transmembrane domain (TM) with or without a cytoplasmic tail (CT) is encoded at the 3′ end (C-terminus of the protein). Most Type I membrane proteins contain a cleavable SP, but it is not an absolute requirement. A Type II single-pass membrane protein mRNA construct (220) encodes a TM encoded at the 5′ end (N-terminus of the protein). A Type III single-pass membrane protein mRNA construct (230) encodes the N-terminus of the encoded polypeptide is on the extracellular side of the membrane, followed by a TM domain, followed by an intracellular portion at the C-terminus. There is nosignal peptide. A Glycosylphosphatidylinositol (GPI)-anchored protein mRNA construct (240) encodes a SP at the 5′ end (N-terminus), and a GPI-anchor sequence that attaches the polypeptide to a lipid bilayer membrane is located at the 3′ end of the CDS (C-terminus of the polypeptide). A multi-pass membrane protein mRNA construct (250) encodes a CDS containing multiple transmembrane domains. A secreted protein mRNA construct (260) encodes signal peptide is encoded at the 5′ end (N-terminus of the protein) and there is no transmembrane domain. An example of a secreted antigen mRNA construct (270) consists of two concatenated polypeptides. A CDS encoding a polypeptide comprising an N-terminal SP and two unique polypeptides connected with a short polypeptide spacer. An example of a type I membrane protein mRNA construct (280) contains two concatenated polypeptides. A CDS encoding a polypeptide comprising an N-terminal SP, two unique polypeptides connected with a short polypeptide spacer, and a C-terminal TM domain.

The RNA according to aspects of the disclosure may have modified ribonucleotides in order to increase its stability and/or decrease cytotoxicity. For example, in some embodiments, 5-methylcytidine in the RNA is substituted partially or completely, for cytidine. In some embodiments, 5-methylcytidine in the RNA is substituted completely for cytidine. Alternatively or additionally, in some embodiments, pseudouridine in the RNA used is substituted partially or completely, for uridine. In some embodiments, pseudouridine in the RNA used is substituted completely for uridine. In some aspects, the LNP comprises nucleic acid comprising a 5′ untranslated region (UTR) and 3′ UTR, polyA tail of about 80 to about 140 nucleotides in length, and (i) a 5′ enzymatic or (ii) a 5′ clean cap. In some aspects, the LNP comprises a chemically modified mRNA, wherein the chemically modified mRNA comprises N1-methylpseudouridine. In other embodiments, the mRNA does not include N1-methyl-pseudouridine.

Exemplary LNP formulations can deliver mRNA encoding any protein antigen derived from an infectious pathogen or disease that can be targeted with the immune system. Listed in Tables 5A and 5B is a non-exhaustive list of vaccine antigens that could be encoded by mRNA and encapsulated in LNPs to comprise a vaccine using the lipids, methods, and formulations described in the Examples.

TABLE 5A
Examples of antigens that could be encoded by mRNA and
encapsulated in LNPs to comprise a novel vaccine.

| Infectious | | |
|---|---|---|
| pathogen/disease | vaccine antigen description | Reference (PMID) |
| respiratory syncytial | Prefusion stabilized F (DS-Cav1) and | 24179220, 27478931, |
| virus (RSV) | subsequent iterative structural | 30849373, 37689061 |
| | improvements. E.g. sc9-10 DS-Cav1 | |
| | A149C Y458C, DS-Cav1-153-50A | |
| | SEQ ID NO: 9 | |
| | RSV-G antigen | |
| | SEQ ID NO: 509 | |
| | SEQ ID NO: 510 | |
| | SEQ ID NO: 511 | |
| | SEQ ID NO: 512 | |
| | SEQ ID NO: 513 | |
| | SEQ ID NO: 515 | |
| Influenza virus (IAV, | HA (e.g., SEQ ID NO: 5, SEQ ID NO: 12, | 26301691, 26303961, |
| IBV) | SEQ ID NO: 14, SEQ ID NO: 915 , SEQ | 30808695, 36356572, |
| | ID NO: 905-907, and/or SEQ ID NO: 18); | 35869085, 35383176, A |
| | HA stabilized stem (Group 1 and 2), | Dosey et al. bioRxiv |
| | neuraminidase (NA) | 2025.07.30.667762 |
| | (e.g. SEQ ID NO: 37, SEQ ID NO: 38, | |
| | SEQ ID NO: 39); M2; stabilized H5 (e.g., | |
| | SEQ ID NO: 15), | |
| Lassa virus (LASV) | stabilized LASV GPC; nucleoprotein; | 38177144, 32269122, |
| | NP-GPC chimeric fusion protein (e.g., | 32142546 |
| | SEQ ID NO: 801 or SEQ ID NO: 803) | |
| human | gp120 eOD-GT-based immunogens (e.g. | 23539181, 31515132, |
| immunodeficiency | eOD-GT8), stabilized Env trimers (e.g. | 27174988, 37098956, |
| virus (HIV) | BG505 DS-SOSPI), conserved protease | 38997302, 33766848, |
| | cleavage sites, gp41 fusion peptide, HLA- | 26687358 |
| | E-restricted epitopes | |
| Mycobacterium | individual Mtb proteins (e.g. ESAT-6, | 31114037, 36604540, |
| tuberculosis | CFP10, Ag85B, Ag85A Esx family | 15187142, 24349004, |
| (tuberculosis) | proteins), | 22133873, 34795205, |
| | SEQ ID NO: 444 | 29263854, 29996082; N |
| | SEQ ID NO: 445 | Agrawal et al. 2025, |
| | SEQ ID NO: 447 | bioRxiv |
| | SEQ ID NO: 449 | 2025.09.30.679428 |
| | SEQ ID NO: 450 | |
| | synthetic fusion proteins (e.g. M72, H4, | |
| | H56, H65, H107, CysVac2, BNT164B1/2) | |
| | SEQ ID NO: 401 | |
| | SEQ ID NO: 403 | |
| | SEQ ID NO: 405 | |
| | SEQ ID NO: 407 | |
| | SEQ ID NO: 409 | |
| | SEQ ID NO: 411 | |
| | SEQ ID NO: 413 | |
| | SEQ ID NO: 415 | |
| | SEQ ID NO: 417 | |
| | SEQ ID NO: 419 | |
| | SEQ ID NO: 421 | |
| | SEQ ID NO: 423 | |
| | SEQ ID NO: 425 | |
| | SEQ ID NO: 427 | |
| | SEQ ID NO: 429 | |
| | SEQ ID NO: 431 | |
| | SEQ ID NO: 437 | |
| | SEQ ID NO: 438 | |
| | SEQ ID NO: 440 | |
| | SEQ ID NO: 442 | |
| | SEQ ID NO: 451 | |
| Plasmodium | circumsporozoite protein (CPS), RTS, S, | 27355532, 33964223, |
| falciparim | R21, CSP-HBsAg fusion (hepatitis B | 37168860, 35822551, |
| (malaria) | surface Ag); Pfs25; PfGARP; CelTOS, | 27457156, 39019011, |
| | RH5.1, RH5.2 | 39025972, |

TABLE 5B
Examples of antigens that could be encoded by mRNA and
encapsulated in LNPs to comprise a novel vaccine.

| Infectious pathogen/disease | vaccine antigen description | Reference (PMID) |
|---|---|---|
| respiratory syncytial virus | RSVpreF (DS-Cav1) and subsequent | 24179220, 27478931, |
| (RSV) | iterative structural improvements. | 30849373, 37689061 |
| | E.g. sc9-10 DS-Cav1 A149C | |
| | Y458C, DS-Cav1-153-50A | |
| Influenza virus (IAV, IBV) | HA, HA stabilized stem, NA, M2 | 26301691, 26303961, |
| | | 30808695, 36356572, |
| | | 35869085, 35383176, |
| | | 3480945 |
| Lassa virus (LASV) | stabilized LASV GPC, nucleoprotein | 38177144, 32269122, |
| | | 32142546 |
| SARS-CoV-2, | spike (S) protein, nucleocapsid, | 33128877 |
| betacoronaviruses | membrane, ORF3a, ORF1ab | |
| Middle East respiratory | spike (S) glycoprotein | 28807998 |
| syndrome coronavirus | | |
| (MERS-CoV) | | |
| Ebola virus (EBOV) | glycoprotein (GP), nucleoprotein | 32418798, 31589088, |
| | (NP) | 32234486 |
| Marburg virus (MARV) | glycoprotein (GP), nucleoprotein | 31589088, 32234486 |
| | (NP) | |
| human metapneumovirus | F glycoprotein | 34551978 |
| (HMPV) | | |
| parainfluenza virus | Type 1 and type 4. Prefusion | 30420505 |
| | stabilized fusion proteins (e.g. | |
| | prefusion PIV3 F) | |
| human immunodeficiency | gp120 eOD-GT-based immunogens | 23539181, 31515132, |
| virus (HIV) | (e.g. eOD-GT8), stabilized Env | 27174988 |
| | trimers (e.g. BG505 DS-SOSPI), | |
| | conserved protease cleavage sites, | |
| | gp41 fusion peptide | |
| human papilloma viruses | L1 capsid protein, E7, glycoprotein | 38462683, 36867683 |
| (HPV) | D, gDE7 fusion | |
| Zika virus (ZIKV) | prM-E | 28222903 |
| Powassan virus (POWV) | prM, prE | 30566864 |
| MPox virus | A35, B6, M1, H3 | 38366591 |
| norovirus | capsid protein VP1 | 38811605 |
| human cytomegalovirus | glycoprotein B (gB), gH, gL, | 38324766, 29456015 |
| (HCMV) | UL128, UL130, UL131 | |
| Ebstein Barr virus (EBV) | glycoprotein gp350, gp42, gH/gL | 38479361, 38926438 |
| | (gp42/gp350) | |
| human varicella-zoster | envelope glycoprotein E (gE) | 25916341 |
| virus (VZV) | | |
| Rabies virus (RABV) | glycoprotein (G) | 37349310 |
| West Nile virus (WNV) | premembrane (prM), envelope (E) | 37125778 |
| Dengue virus (DENV) | envelope protein (E), premembrane | 35608749 |
| | (prM), NS1, NS3, NS5, NS4B | |
| Chikungunya virus | E1, E2, E3 glycoproteins | 23670192, 38005854, |
| (CHIKV). other | | 36474012 |
| Alphaviruses of | | |
| the Togaviridae family | | |
| (RRV, ONNV, BFV, | | |
| MAYV, SINV, EEEV, | | |
| WEEV, VEEV) | | |
| Nipah virus (NiV) | glycoprotein (G) | 38726013 |
| Mycobacterium | individual Mtb proteins (e.g. ESAT- | 31114037, 36604540 |
| tuberculosis | 6, CFP10, Ag85B, Esx family | |
| (tuberculosis) | proteins), synthetic fusion proteins | |
| | (e.g. M72) | |
| Plasmodium | circumsporozoite protein (CPS); | 27355532, 33964223, |
| falciparim | RTS,S; CSP-HBsAg fusion | 37168860 |
| (malaria) | (hepatitus B surface Ag); Pfs25; | |
| | PfGARP; CelTOS | |
| Borrelia burgdorferi | outer surface protein A (OspA) | 37533256 |
| (Lyme disease) | | |
| cancer | cancer neoantigens | 37165196, 28678784, |
| | | 35970920, 38538867 |
| A. americanum (alpha-gal | Salivary proteins (e.g. Salp- | 34788080, 37407406 |
| hypersensitivity), | family proteins, TSLPI) | |
| I. scapularis | | |
| Mumps (MuV) | Fusion glycoprotein F, prefusion F | 39527740, |
| | variants, HN | PCT/US2020/064619, |
| | | WO2021119497A1 |
| Measles (MeV) | Fusion glycoprotein F, prefusion F | PCT/US2020/064619, |
| | | WO2021119497A1 |

In some embodiments, the mRNA is a nucleic acid comprising or consisting of SEQ ID NOS: 3, 4, 6, 8, 10, 13, 16, 17, 402, 404, 406, 408, 410, 412, 414, 416, 418, 420, 422, 424, 426, 428, 430, 432, 439, 441, 443, 446, 448, 452, 501, 502, 505, 506, 507, 508, 514, 516, 802, 804, 901, 902, 903, 904, 909, or 914.

In some embodiments, the mRNA encodes a peptide or a protein comprising or consisting of SEQ ID NOS: 2, 5, 7, 9, 12, 14, 15, 18, 36, 37, 38, 39, 401, 403, 405, 407, 409, 411, 413, 415, 417, 419, 421, 423, 425, 427, 429, 431, 433, 434, 437, 438, 440, 442, 444, 445, 447, 449, 450, 451, 509, 510, 511, 512, 513, 515, 801, 803,905, 906, 907, 908, 910, 911, 912 or 915.

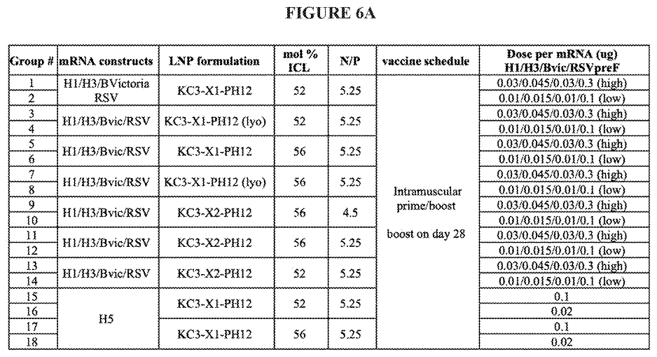

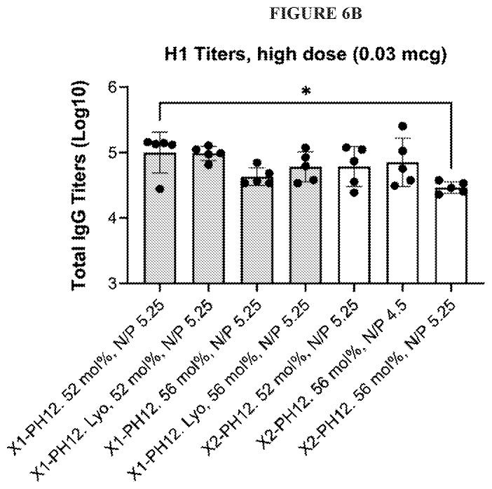

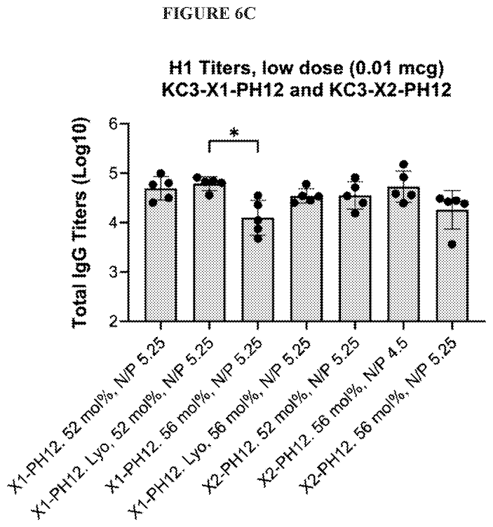

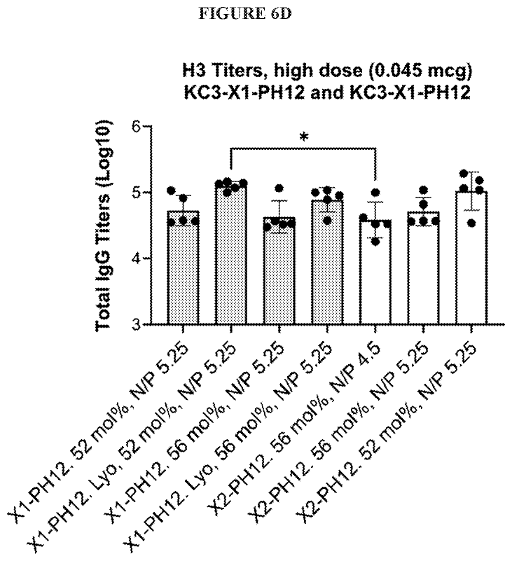

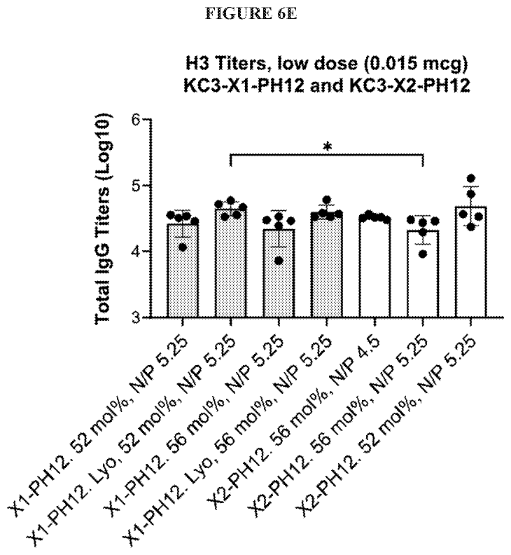

The mRNAs encoding antigens for influenza A subtypes H1 (SEQ ID NO:13 encoding SEQ ID NO:12), H3 (SEQ ID NO:914 encoding SEQ ID NO:14), influenza B/Victoria (SEQ ID NO:17 encoding SEQ ID NO:915), and RSVpreF (SEQ ID NO:10 encoding SEQ ID NO:9) were combined at equal weight ratios and encapsulated into LNPs comprising ionizable lipids of Formula (I) as described in Example 2. To measure the ability of these mRNA-LNPs to induce antibodies, BALB/c mice were immunized at a high and 3-fold lower dose in the caudal thigh in a volume of 50 μL. The experimental design and dosing schema is shown in FIG. 6A. Twenty-one days post vaccination, serum was collected and stored at −80° C. until analysis. Mouse IgG endpoint antibody titers against the matching H1, H3, B-Victora, RSVpreF or H5 antigens were determined by standard ELISA. For H1-specific Ab titers, we found for KC3-X1-PH12 that immunogenicity of freeze-dried material was not deleterious for biological activity (FIG. 6B and FIG. 6C). This was true for 52 and 56 mol % ICL with N/P=5.25. Second, Ab titers were comparable between LNPs comprised of KC3-X1-PH12 and KC3-X2-PH12, showing that either orientation of the biodegradable ester bond can be used for PH12 with a KC3 headgroup. Third, we found for KC3-X1-PH12 that either 52 or 56 mol % with N/P=5.25 elicited similar immune responses; the same was true for X2-PH12. For H3-specific Ab titers (FIG. 6D and FIG. 6E), results were similar to H1 titers: lyophilization of the mRNA-LPN was not deleterious to biological activity (H3 titers elicited by lyophilized material trended higher than for non-lyophilized, X1-PH12 and X2-PH12 were comparably immunogenic, and these ICLs can be used with either 52 or 56 mol % without impacting antibody titers.

The method of making a LNP can comprise mixing a first solution with a second solution. Mixing can be achieved using standard liquid mixing techniques, such as propellor mixing, vortexing solutions or preferably through microfluidic mixing or high efficiency T-mixing. In some embodiments, the first solution comprises a lipid or a plurality of lipids and a nucleic acid, where all components are solubilized, in water/solvent system. The solvent may be any water miscible solvent (e.g., ethanol, methanol, isopropanol, acetonitrile, dimethylformamide, dimethylsulfoxide, dioxane or tetrahydrofuran). In some embodiments, the first solution comprises a small percentage of water or pH buffered water. In some embodiments, the first solution comprises a single type of lipid, for example, an ionizable lipid, a phospholipid, a sterol, or a PEG-containing lipid. In some embodiments, the first solution comprises a plurality of lipids. In some embodiments, the plurality comprises an ionizable lipid, a phospholipid, a sterol, or a PEG-containing lipid. In some embodiments, the second solution is water. In some embodiments, the second solution is an aqueous buffer with a pH between 3-6 (e.g., a pH of about 3, about 4, about 5, or about 6). The second solution may comprise a load component, e.g., a nucleic acid (e.g., mRNA). The second solution may comprise a small percentage of water-miscible organic solvent. In some embodiments, the solution comprising a mixture of the first and second solutions comprising the LNP suspension can be diluted. In some embodiments, the pH of the solution comprising a mixture of the first and second solutions comprising the LNP suspension can be adjusted. Dilution or adjustment of the pH of the LNP suspension can be achieved with the addition of water, acid, base or aqueous buffer. In some embodiments, no dilution or adjustment of the pH of the LNP suspension is carried out. In some embodiments, both dilution and adjustment of the pH of the LNP suspension is carried out. In some embodiments, excess reagents, solvents, unencapsulated nucleic acid maybe removed from the LNP suspension by tangential flow filtration (TFF) (e.g., diafiltration). The organic solvent (e.g., ethanol) and buffer may also be removed from the LNP suspension with TFF. In some embodiments, the LNP suspension is subjected to dialysis and not TFF. In some embodiments, the LNP suspension is subjected to TFF and not dialysis. In some embodiments, the LNP suspension is subjected to both dialysis and TFF. In one aspect, the present disclosure features a method comprising treating a sample of LNPs comprising nucleic acid, with a fluid comprising a detergent (e.g., Triton X-100, or anionic detergents (such as, but not limited to, sodium dodecyl sulfate (SDS), or non-ionic detergent, such as but not limited to β-octylglucoside, or Zwittergent 3-14) for a period of time suitable to degrade the lipid layer and thereby release the encapsulated and/or entrapped nucleic acid(s). In an embodiment, the method further comprises analyzing the sample for the presence, absence, and/or amount of the released nucleic acid(s).

Methods of making lipid nanoparticle (LNP) compositions encapsulating mRNA are provided in Example 7 (Preparing ligand-targeted LNPs) and Example 9 (Preparation of lipidic nanoparticles) of Patent Cooperation Treaty Patent Application PCT/US2021/060877 (WO2022/115645), are incorporated herein by reference in their entirety. Methods of making lipid nanoparticle (LNP) compositions encapsulating mRNA are provided in Example 7 (Preparing ligand-targeted LNPs) and Example 9 (Preparation of lipidic nanoparticles) of Patent Cooperation Treaty Patent Application PCT/US2023/067517 (WO2023/230587), are incorporated herein by reference in their entirety. Methods of characterizing lipid nanoparticle (LNP) compositions encapsulating mRNA are provided in Example 10 (LNP Characterization) and Example 11 (Determination of transfection efficiency in murine dendritic cells of LNPs using mCherry mRNA) of Patent Cooperation Treaty Patent Application PCT/US2021/060877 (WO2022/115645), are incorporated herein by reference in their entirety. Methods of making lipid nanoparticle (LNP) compositions encapsulating mRNA are provided in Example 10 (LNP Characterization) and Example 11 (Determination of transfection efficiency in murine dendritic cells of LNPs using mCherry mRNA) of Patent Cooperation Treaty Patent Application PCT/US2023/067517 (WO2023/230587), are incorporated herein by reference in their entirety. These methods can be modified by using a microfluidics chip from ChipShop (187) to make LNPs at flow rates of 18 mL/min instead of the Nanoprecision Systems microfluidics cassette at 6 ml/min whilst keeping downstream processing and analytical procedures the same as described in the published patents referenced above. Additional disclosure of methods for the manufacture of mRNA lipid nanoparticle compositions are provided in: S. J. Shepherd, X. Han, A. J. Mukalel, R. El-Mayta, A. S. Thatte, J. Wu, M. S. Padilla, M. Alameh, N. Srikumar, D. Lee, D. Weissman, D. Issadore, & M. J. Mitchell, Throughput-scalable manufacturing of SARS-CoV-2 mRNA lipid nanoparticle vaccines, Proc. Natl. Acad. Sci. U.S.A. 120 (33) e2303567120, doi.org/10.1073/pnas.2303567120 (2023), incorporated by reference herein in its entirety.

### EXAMPLES

Certain examples are provided below to illustrate various embodiments of the embodiments disclosed herein. One of ordinary skill in the art will recognize that the various embodiments disclosed herein are not limited to these specific illustrative examples.

### Example 1: Synthesis of Ionizable Cationic Lipids

### Synthesis of KC3-X1Ionizable Cationic Lipids

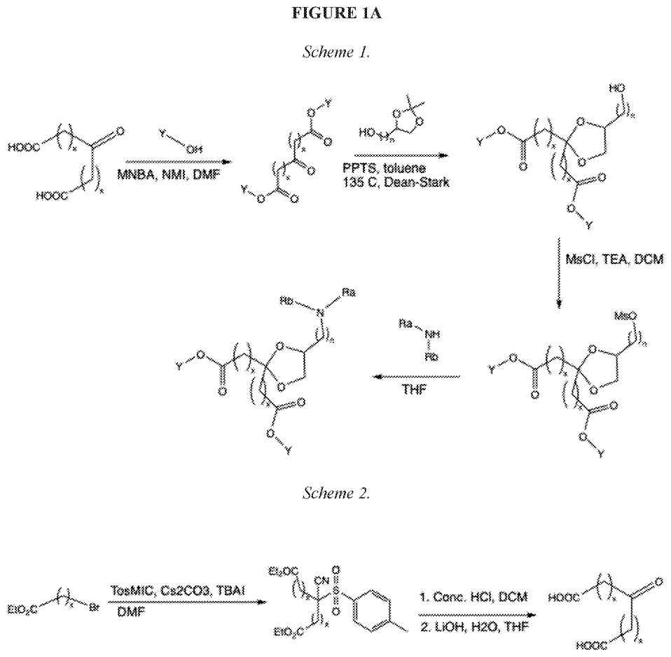

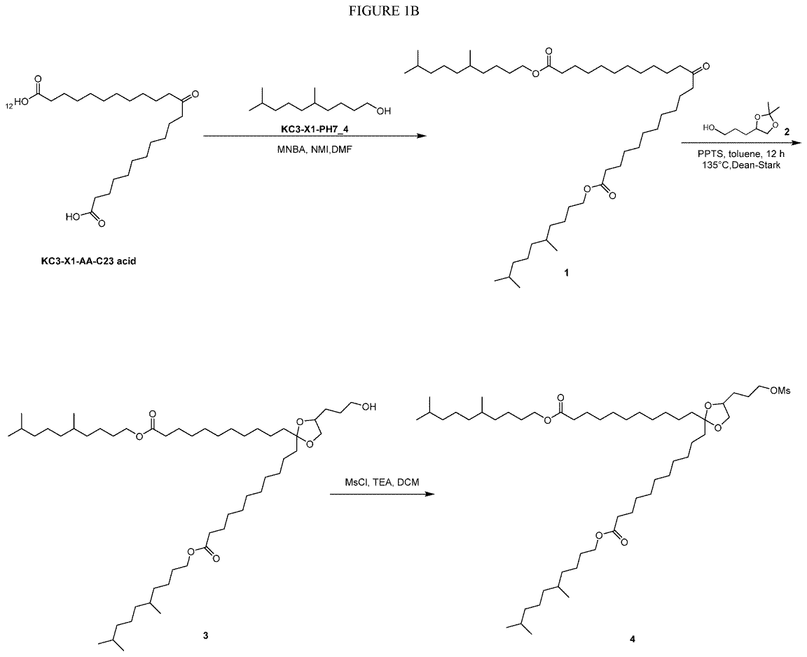

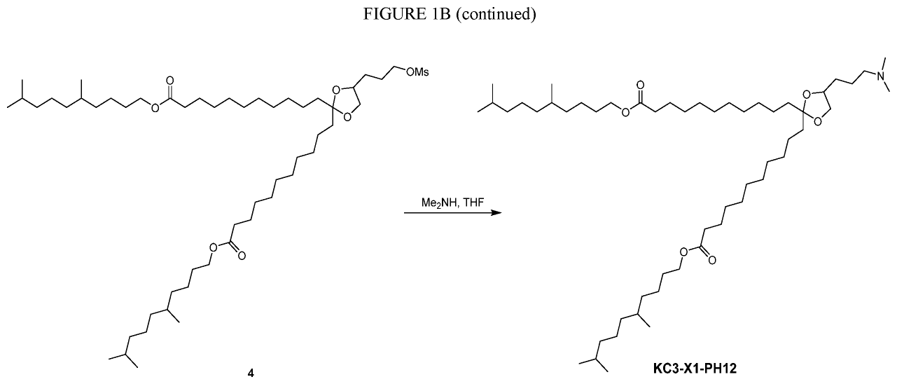

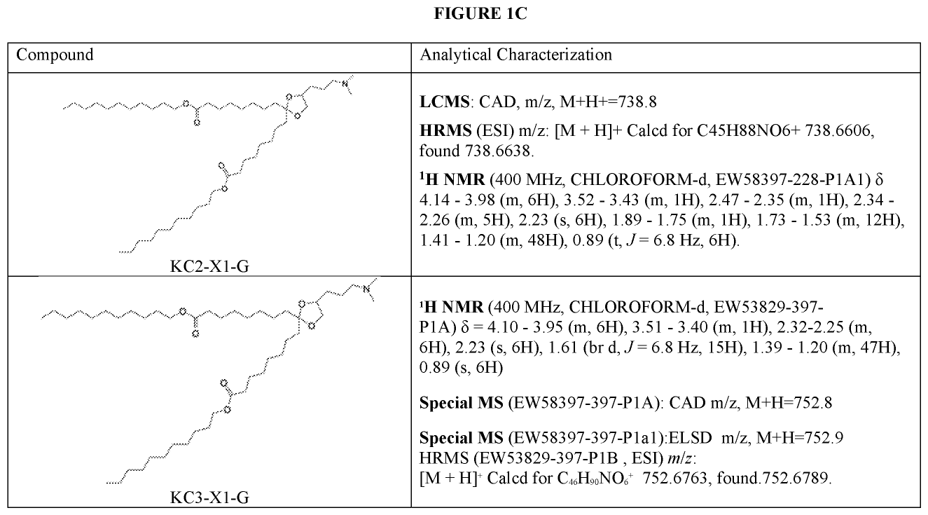

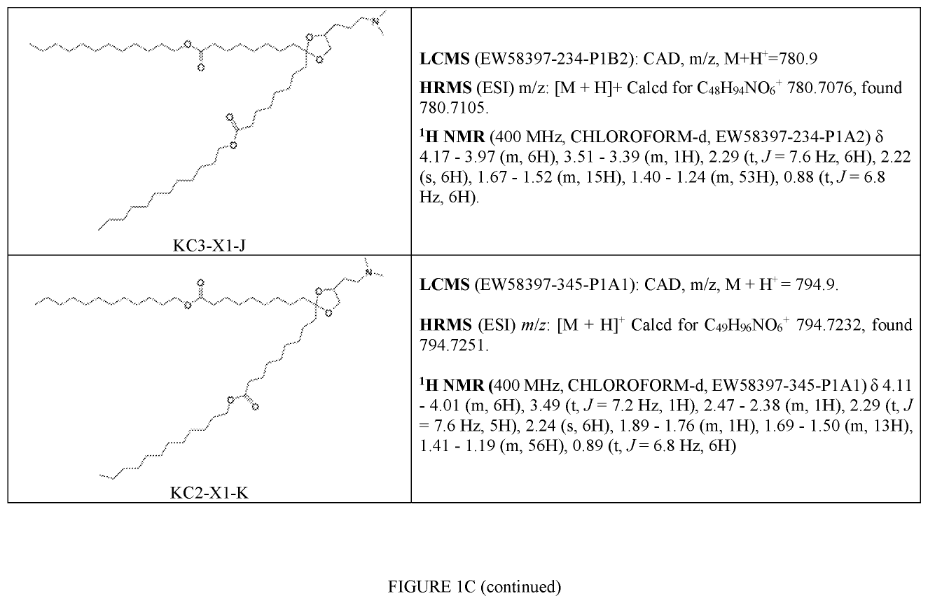

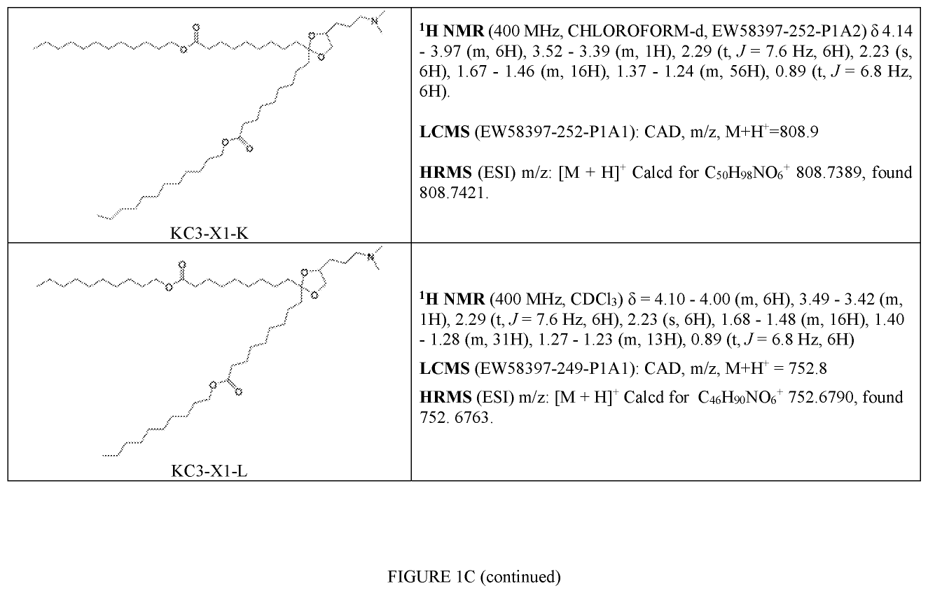

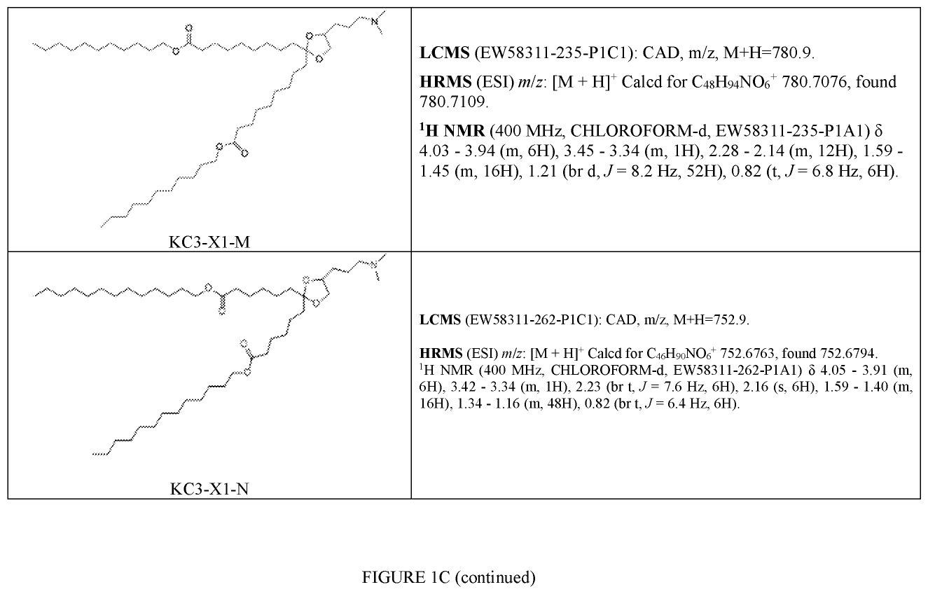

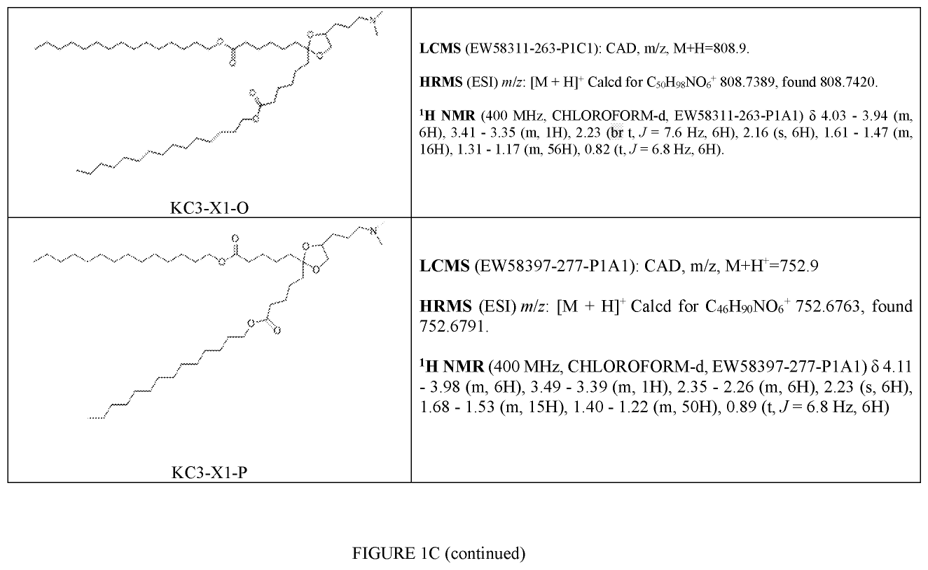

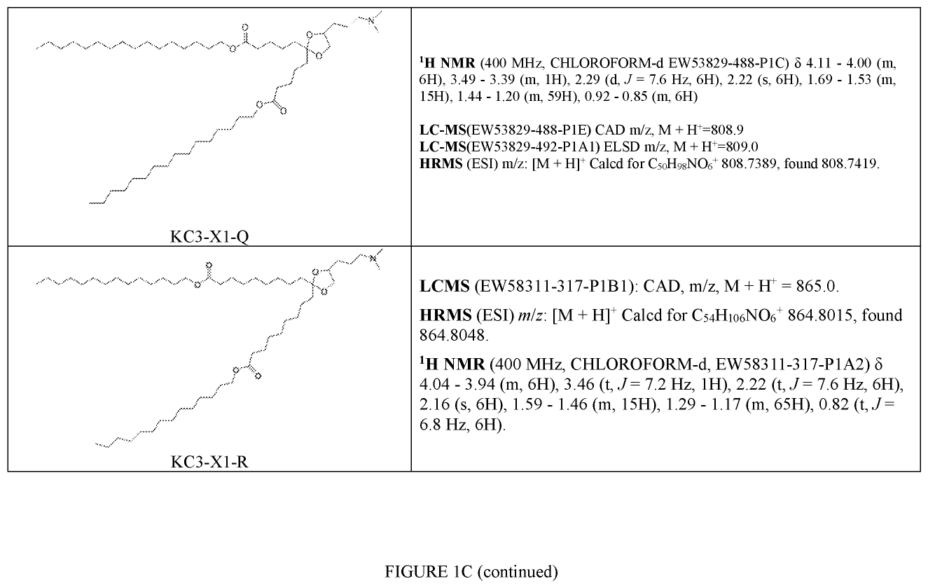

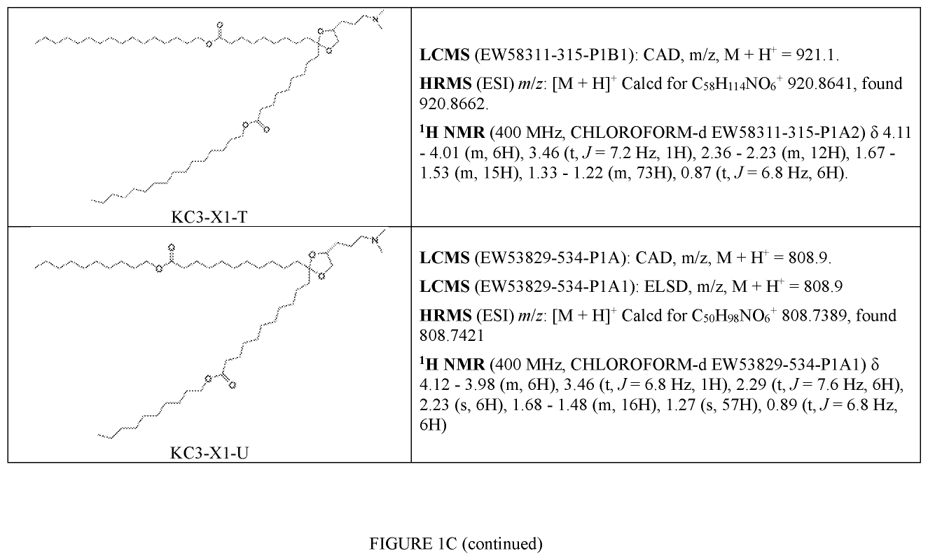

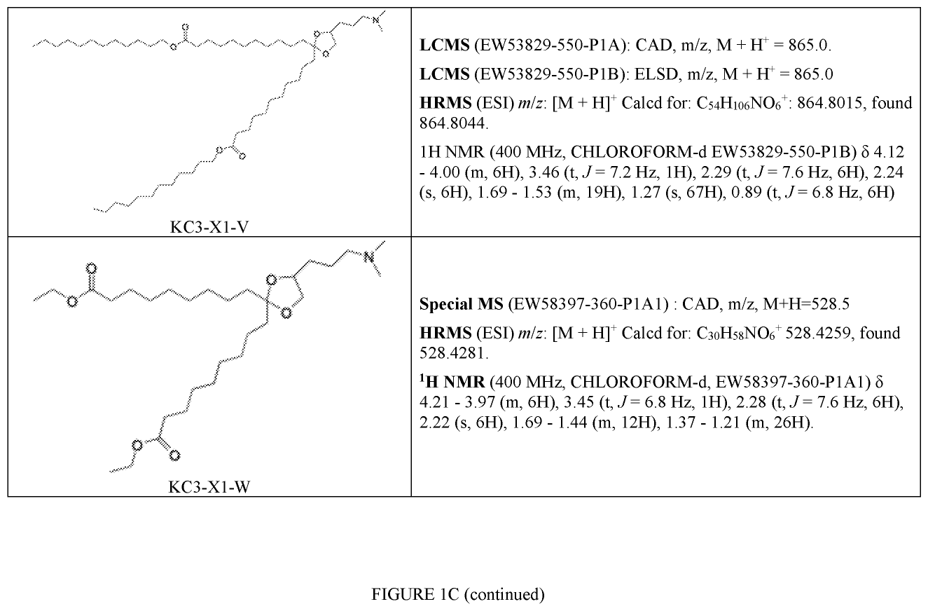

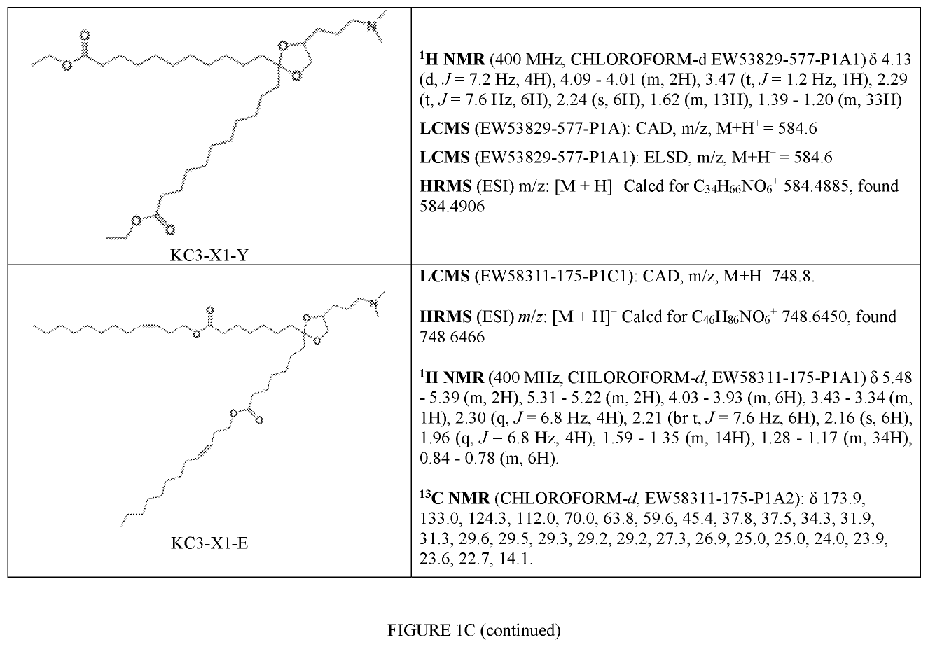

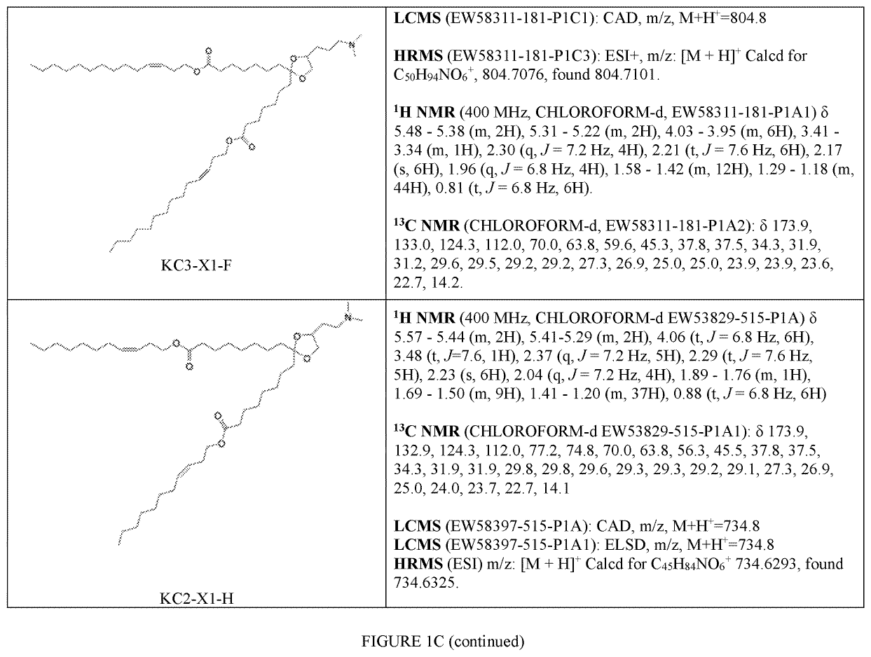

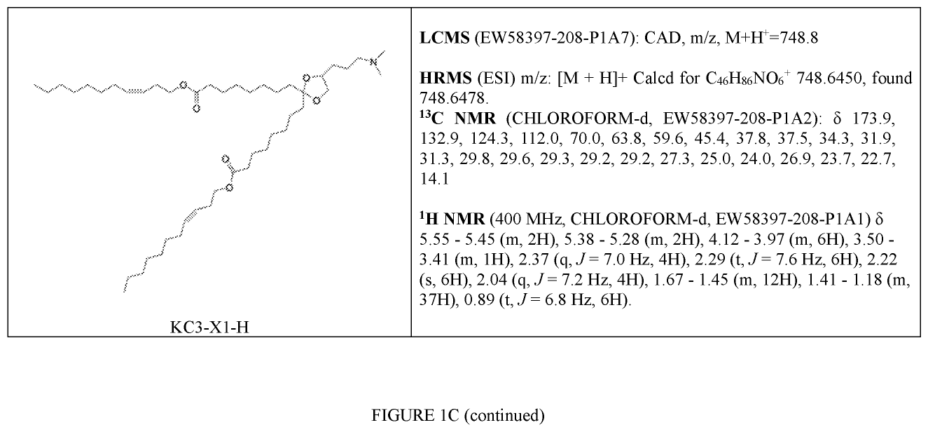

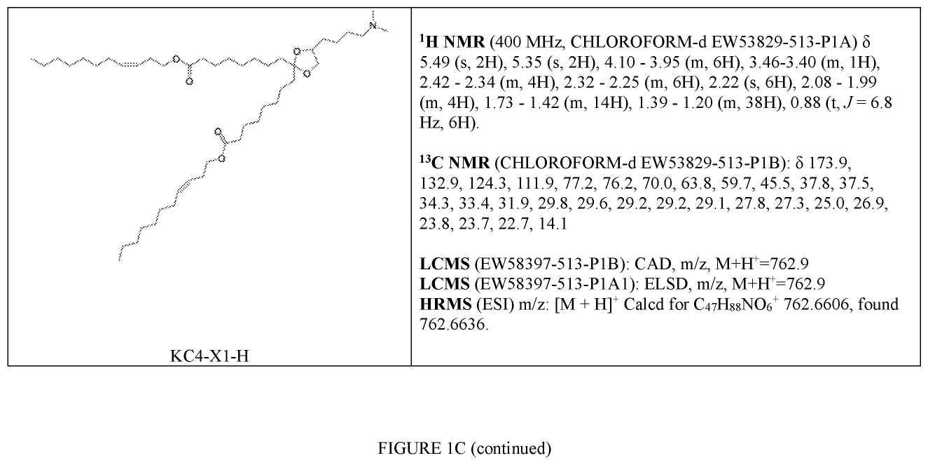

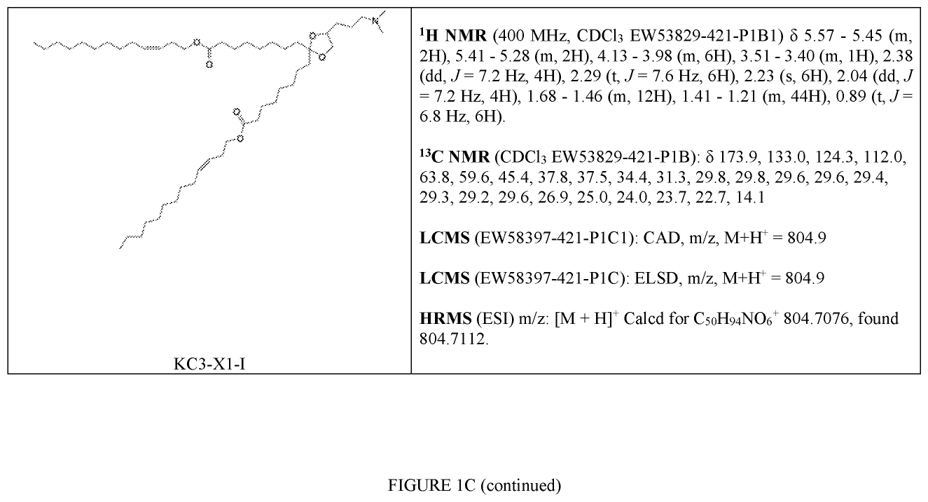

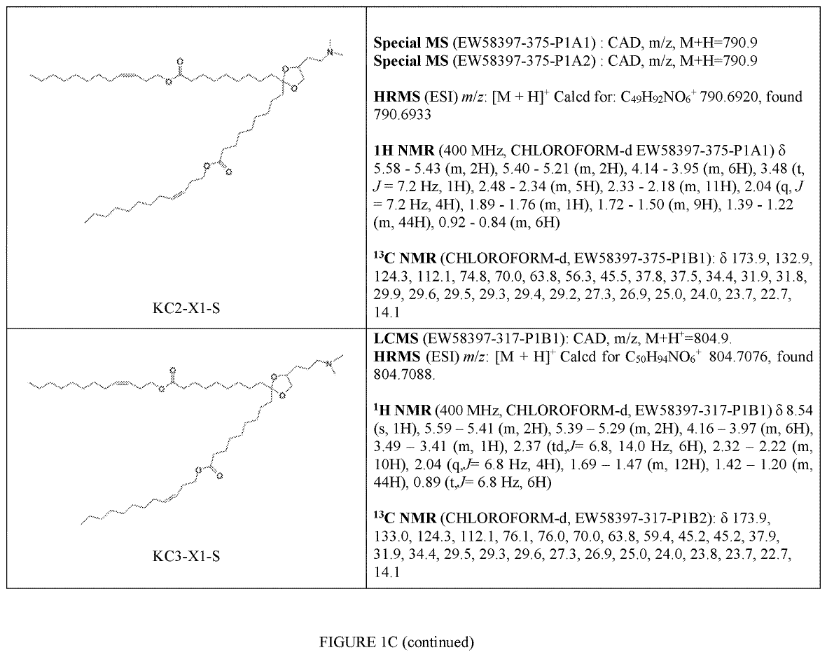

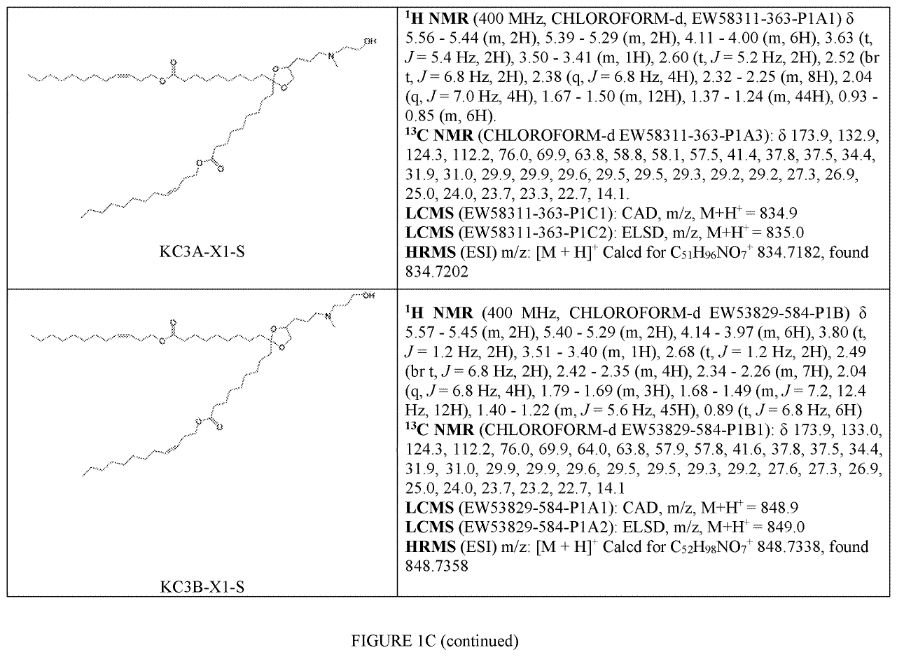

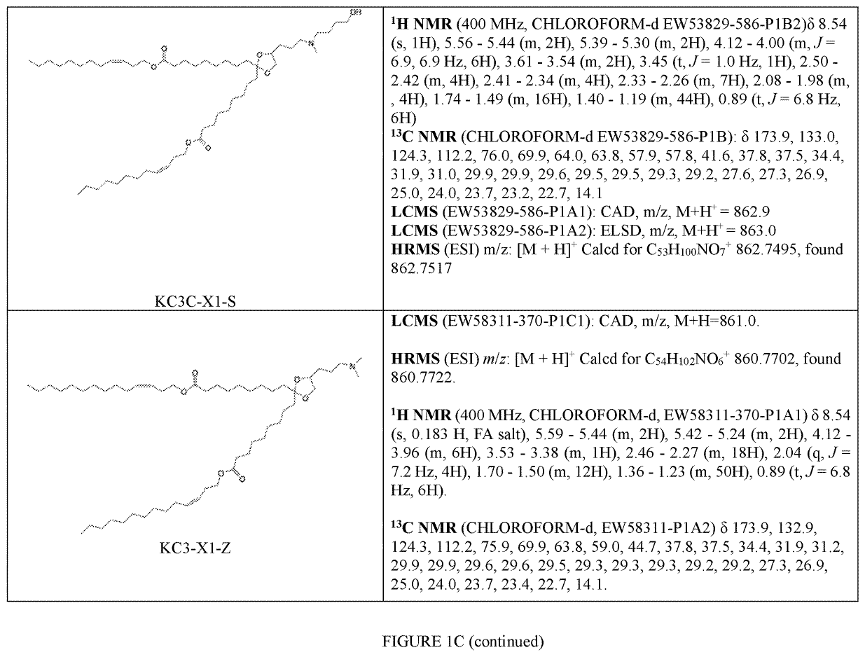

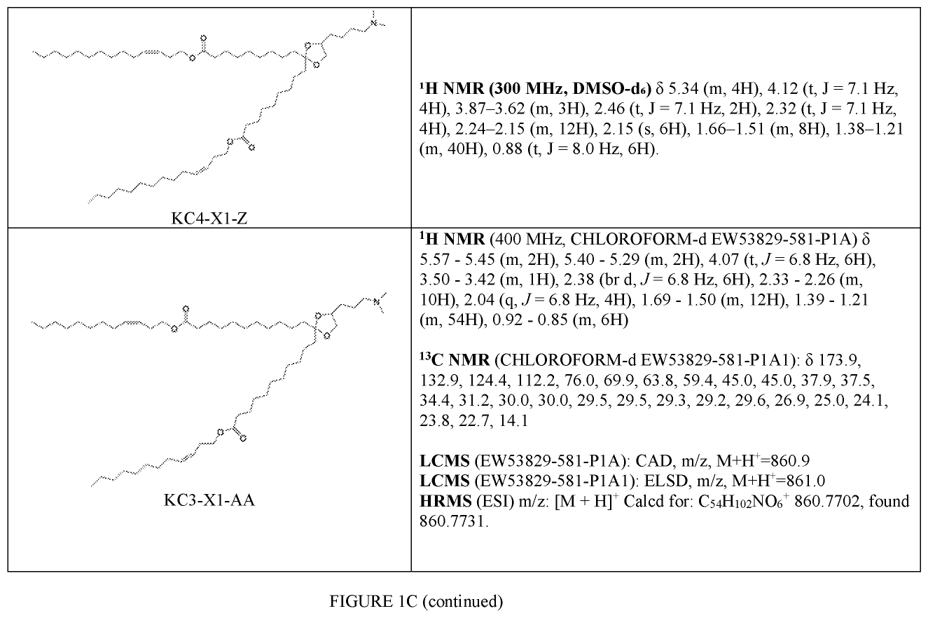

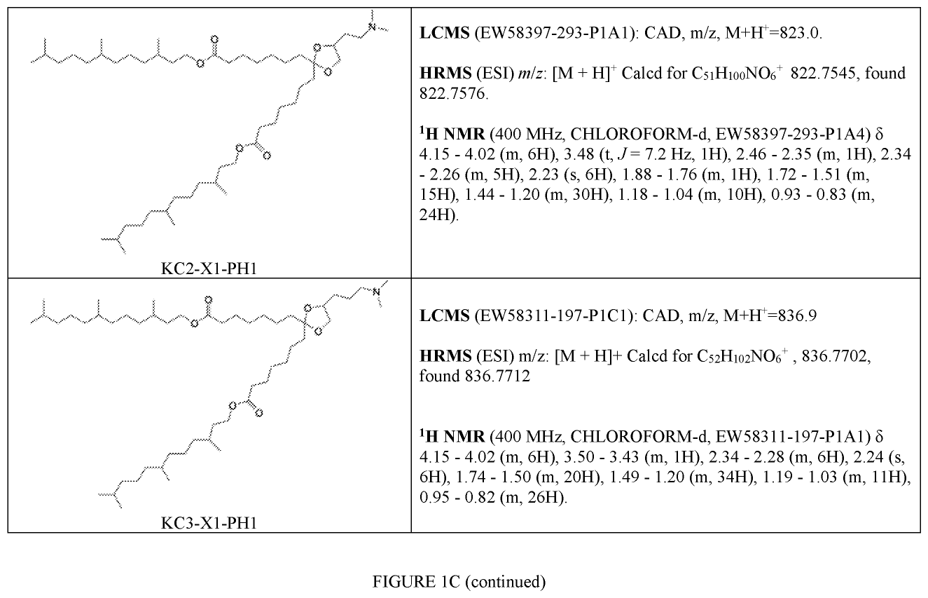

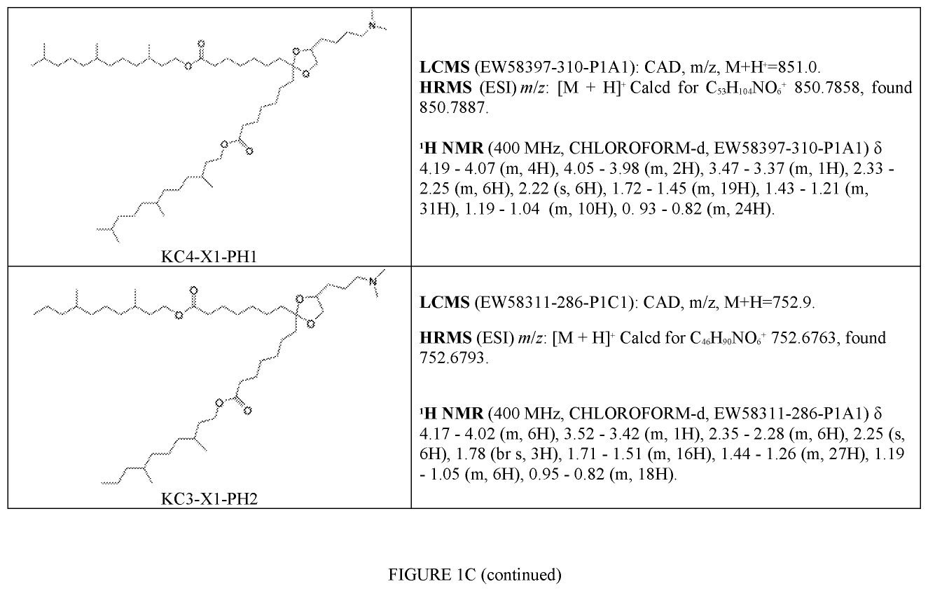

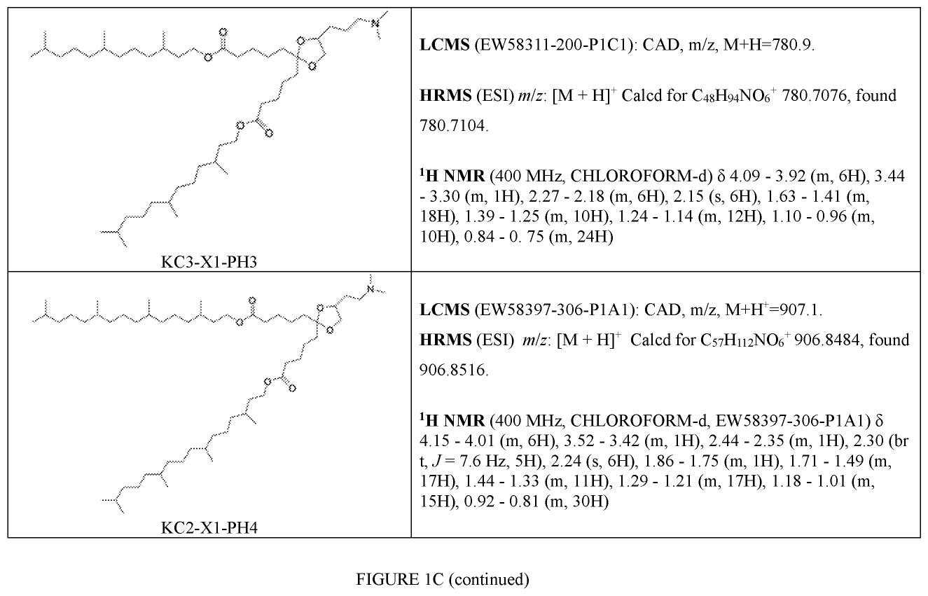

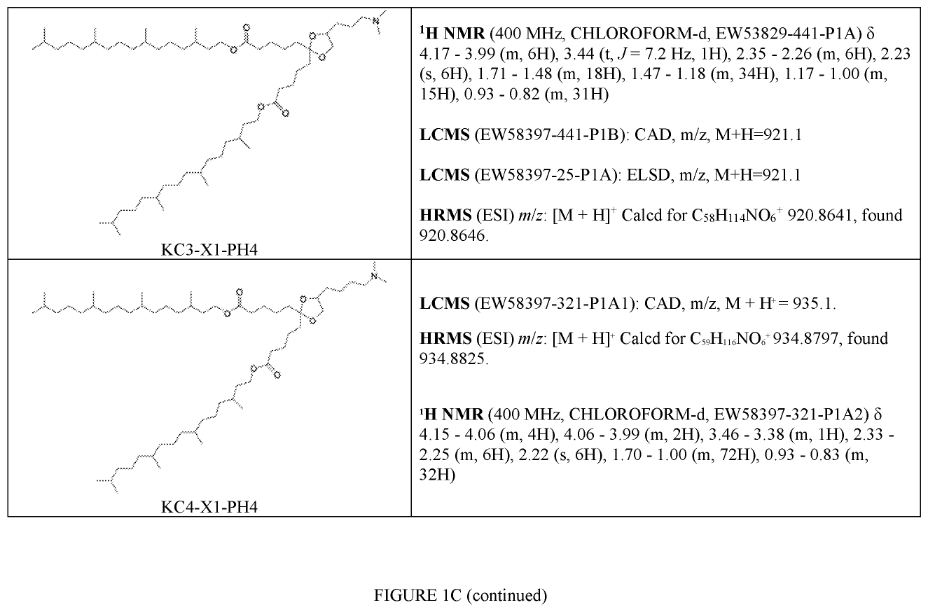

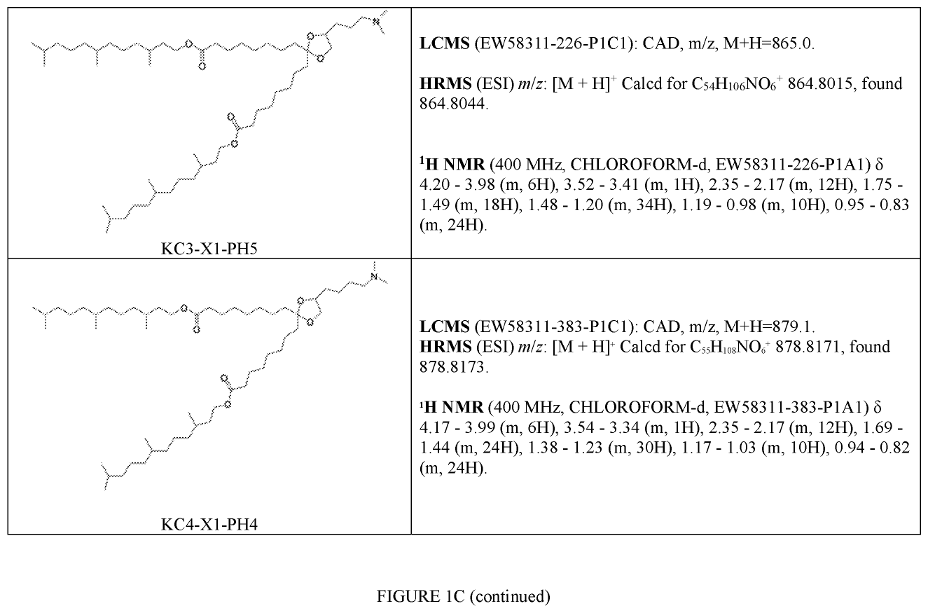

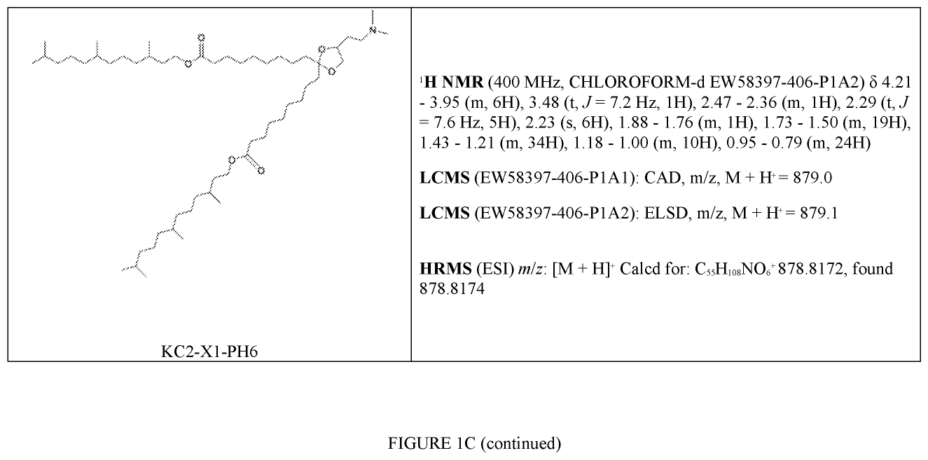

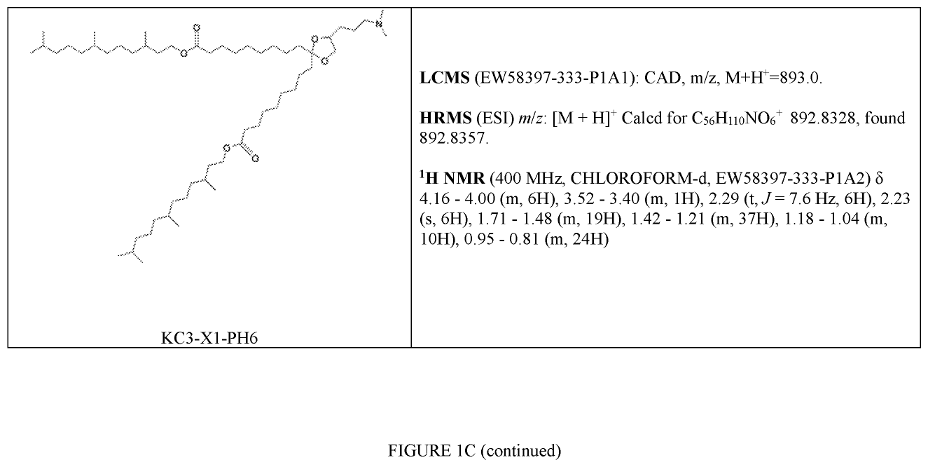

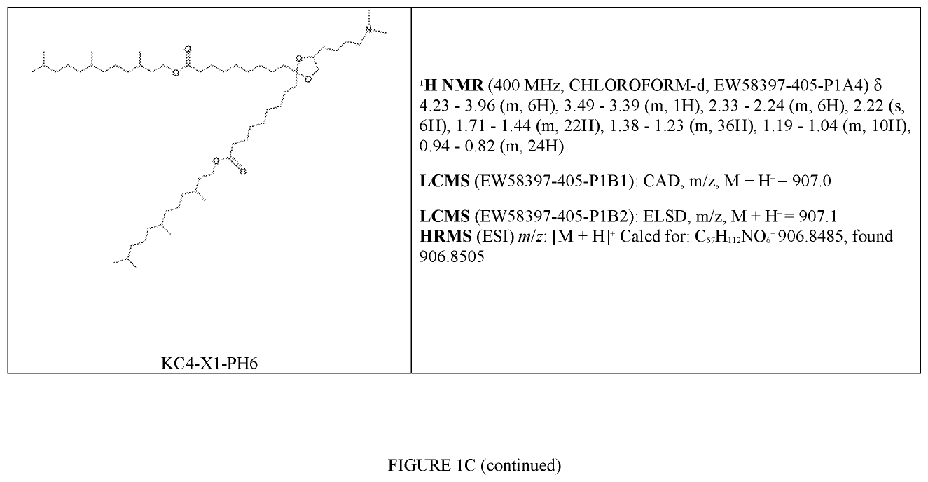

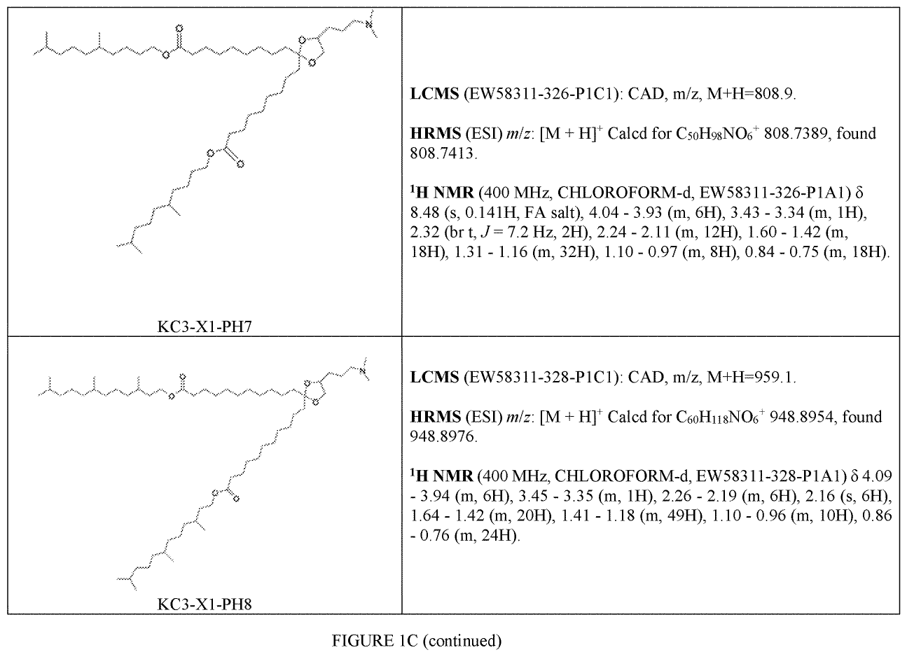

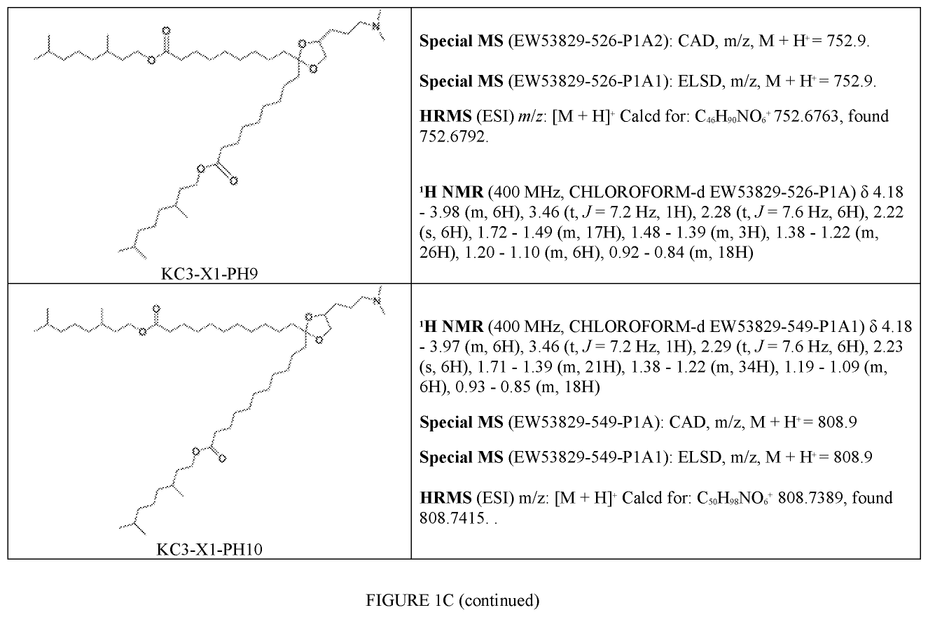

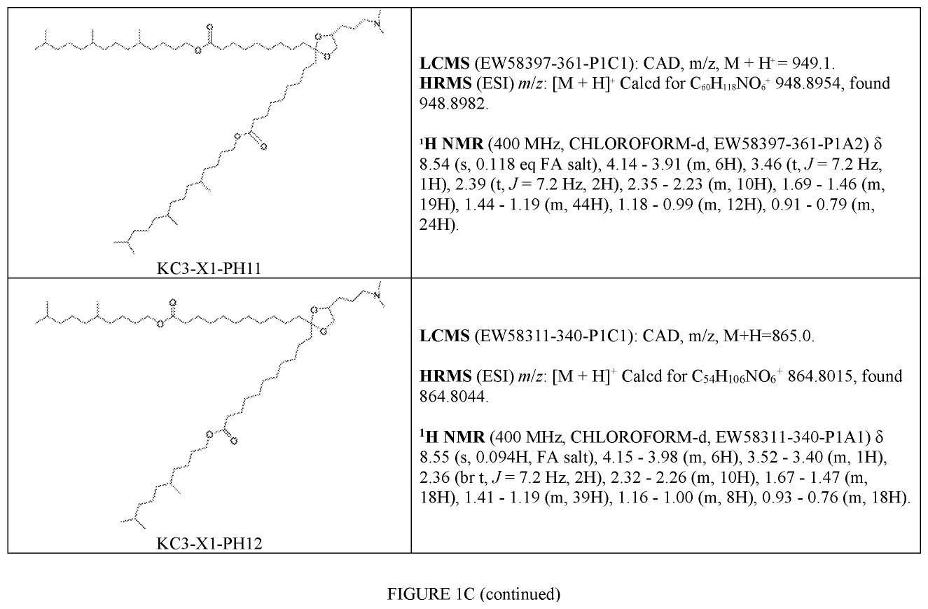

FIG. 1A is a general synthesis for compounds of Formula (I) wherein W₁ and W₂ are each *—O—C(O)—**, * is a covalent bond to Y₁ or Y₂ and ** is a covalent bond to X₁ or X₂ (respectively), X₁ or X₂ are each —(CH₂)ₓ—, and Y₁ or Y₂ are each identical linear or branched hydrocarbon chains with (y) total carbons (i.e., “KCn-X1” ionizable cationic lipids). A general synthesis for compounds of formula (I) shown in Scheme 1 involves i) esterification of the ketone dicarboxylic acid, ii) acid catalyzed trans ketal formation, iii) Mesylation of the primary alcohol, and finally iv) displacement of the mesyl group using dialkylamine resulting in a tertiary amine. A general synthesis of ketone dicarboxylic acid starting material in Scheme 1 is presented below in Scheme 2. A general synthesis of ketone dicarboxylic acid starting material shown in Scheme 2 involves i) treatment of suitable bromo alkyl carboxylate with toluenesulfonylmethyl isocyanide in the presence of a base, ii) conversion of the TosMIC adduct to ketone in the presence of HCl, and finally iv) hydrolysis of the ethyl carboxylate to ketone dicarboxylic acid.

FIG. 1B is a chemical synthesis for preparing an ionizable cationic lipid of Formula (I) designated KC3-X1-PH12, as described below.

To a solution of KC3-X1-AA-C23 acid (800 mg, 2.01 mmol, 1 eq) and KC3-X1-PH7_4 (1.12 g, 6.02 mmol, 3 eq) in DMF (10 mL) was added NMI (1.65 g, 20.07 mmol, 1.60 mL, 10 eq) and MNBA (2.07 g, 6.02 mmol, 3 eq). The mixture was stirred at 70° C. for 12 hr. The reaction mixture was quenched by addition aq. NaHCO₃ (sat. 30 mL) at 25° C., and extracted with EtOAc (10 mL*3). The combined organic layers were washed with brine (10 mL*3), dried over Na₂SO₄, filtered and concentrated under reduced pressure to give a residue. The residue was purified by flash silica gel chromatography (ISCO®; 40 g SepaFlash® Silica Flash Column, Eluent of 0~3% Ethylacetate/Petroleum ethergradient @60 mL/min) to give compound 1 (1.5 g, 1.43 mmol, 71.2% yield, 70.1% purity) as a white solid. ¹H NMR (400 MHz, CHLOROFORM-d, EW58311-330-P1A1) δ 4.00 (t, J=6.8 Hz, 4H), 2.31 (t, J=7.4 Hz, 4H), 2.22 (t, J=7.6 Hz, 4H), 1.58-1.42 (m, 16H), 1.32-1.19 (m, 36H), 1.09-1.00 (m, 8H), 0.81-0.78 (m, 18H).

A mixture of compound 1 (1.5 g, 2.04 mmol, 1 eq), PPTS (256.36 mg, 1.02 mmol, 0.5 eq) and compound 2 (588.36 mg, 3.67 mmol, 1.8 eq) in Tol. (20 mL) was heated to reflux and remove water by Dean-Stark trap. The mixture was stirred at 135° C. for 12 hr. The reaction mixture was quenched by addition aq.NaHCO₃ (sat. 60 mL) at 25° C., and extracted with EtOAc (30 mL*3). The combined organic layers were washed with brine (20 mL*3), dried overNa₂SO₄, filtered and concentrated under reduced pressure to give a residue. The residue was purified by flash silica gel chromatography (ISCO®; 40 g SepaFlash® Silica Flash Column, Eluent of 0~10% Ethyl acetate/Petroleum ether gradient @60 mL/min) to give compound 3 (1 g, 1.19 mmol, 58.5% yield) as a yellow oil. ¹H NMR (400 MHz, CHLOROFORM-d, EW58311-334-P1A1) δ 4.13-4.05 (m, 6H), 3.75-3.66 (m, 2H), 3.52-3.45 (m, 1H), 2.31 (t, J=7.6 Hz, 4H), 1.74-1.48 (m, 20H), 1.41-1.26 (m, 41H), 1.18-1.08 (m, 8H), 0.90-0.85 (m, 18H).

To a solution of compound 3 (1 g, 1.19 mmol, 1 eq) in DCM (10 mL) was added TEA (604.23 mg, 5.97 mmol, 831.13 μL, 5 eq) and MsCl (630 mg, 5.50 mmol, 425.68 μL, 4.61 eq). The mixture was stirred at 0° C. for 1 hr. The reaction mixture was quenched by addition ice-water (30 mL) at 0° C., and extracted with EtOAc (10 mL*3). The combined organic layers were washed with brine (10 mL*3), dried over Na₂SO₄, filtered and concentrated under reduced pressure to give a residue. The residue was purified by flash silica gel chromatography (ISCO®; 12 g SepaFlash® Silica Flash Column, Eluent of 0~15% Ethyl acetate/Petroleum ether gradient @40 mL/min) to give compound 4 (1 g, 1.09 mmol, 91.5% yield) as a yellow oil. ¹H NMR (400 MHz, CHLOROFORM-d, EW58311-335-P1A1) δ 4.36-4.24 (m, 2H), 4.12-4.03 (m, 6H), 3.54-3.44 (m, 1H), 3.03 (s, 3H), 2.31 (t, J=7.6 Hz, 4H), 1.99-1.79 (m, 2H), 1.69-1.52 (m, 20H), 1.40-1.27 (m, 38H), 1.19-1.06 (m, 8H), 0.94-0.83 (m, 18H).

A solution of compound 4 (900 mg, 983.14 mol, 1 eq) in Me₂NH/THF (2 M, 20 mL, 40.69 eq) was stirred at 55° C. for 12 hr. The mixture was concentrated in vacuo to give a residue. The residue was purified by flash silica gel chromatography (ISCO®; 20 g SepaFlash® Silica Flash Column, Eluent of 0~8% Ethyl acetate/(Petroleum ether/Dichloromethane=3/1, 0.1% TEA as eluant) @40 mL/min) and followed by reversed-phase HPLC(column: PHS XP Phenyl-Hexyl 150*25 mm*7 um; mobile phase: [H₂O (0.225% FA)-EtOH]; gradient: 65%-95% B over 15.0 min). KC3-X1-PH12 (0.094 eq. FA salt, 435.5 mg, 495.75 μmol, 53.6% yield, 98.4% purity) was obtained as a colorless oil. LCMS (EW58311-340-P1C1): CAD, m/z, M+H=865.0. HRMS (ESI) m/z: [M+H]⁺ Calcd for Cs₄H₁₀₆NO₆⁺864.8015, found 864.8044. ¹H NMR (400 MHz, CHLOROFORM-d, EW58311-340-P1A1) δ 8.55 (s, 0.094H, FA salt), 4.15-3.98 (m, 6H), 3.52-3.40 (m, 1H), 2.36 (br t, J=7.2 Hz, 2H), 2.32-2.26 (m, 10H), 1.67-1.47 (m, 18H), 1.41-1.19 (m, 39H), 1.16-1.00 (m, 8H), 0.93-0.76 (m, 18H).

FIG. 1C is a table of additional examples of KCn-X1 compounds that were prepared.

### Synthesis of KC3-X2 Ionizable Cationic Lipids

FIG. 2A is a chemical synthesis (Scheme 3 and Scheme 4) for preparing ionizable cationic lipids of Formula (I) wherein -A₁- and -A₂- are each *—C(O)—O—** wherein * is a covalent bond to —Y₁ and —Y₂, respectively, and ** is a covalent bond to —X₁— and —X₂—, respectively, wherein (n) is 2, 3 or 4. (designated “KCn-X2 ICLs”). Scheme 3 is a general synthesis for compounds of Formula (I) wherein W₁ and W₂ are each *—C(O)—**, * is a covalent bond to Y₁ or Y₂ and ** is a covalent bond to X₁ or X₂ (respectively), X₁ or X₂ are each —(CH₂)ₓ—, and Y₁ or Y₂ are each identical linear or branched hydrocarbon chains with (y) total carbons (i.e., “KCn-X2” ionizable cationic lipids). A general synthesis for compounds of formula (I) wherein W is —C(O)O— shown in Scheme 3 involves i) esterification of the dihydroxy ketone with a suitable carboxylic acid, ii) acid catalyzed trans ketal formation, iii) Mesylation of the primary alcohol, and finally iv) displacement of the mesyl group using dialkylamine resulting in a tertiary amine. A general synthesis of dihydroxy ketone starting material in Scheme 3 is presented below in Scheme 4. A general synthesis of dihydroxy ketone shown in Scheme 4 involves i) a suitable hydroxy alkyl bromide is treated with toluenesulfonylmethyl isocyanide in the presence of sodium hydride in DMSO, and ii) TosMIC adduct is then converted to a ketone in the presence of HCl.

FIG. 2B is a chemical synthesis for preparing an ionizable cationic lipid of Formula (I) designated KC3-X2-PH12, as described below.

A solution of NaH (5.90 g, 147.51 mmol, 60% purity, 2.4 eq) in DMSO (300 mL) was stirred at 25° C. for 1 hr. Then TBAI (2.27 g, 6.15 mmol, 0.1 eq) and Tosmic (12 g, 61.46 mmol, 1 eq) was added. The mixture was stirred at 25° C. for 15 min. After the addition of compound 1 (30.61 g, 129.07 mmol, 2.1 eq), the mixture was stirred at 25° C. for 1 hr. The reaction mixture was quenched by addition sat-NH₄Cl (aq.200 mL) at 25° C., and then diluted with ice-water (100 mL) and extracted with EtOAc (200 mL*3). The combined organic layers were washed with brine (200 mL*3), dried with anhydrous Na₂SO₄, filtered and concentrated in vacuum. The crude product was used directly for the next step without further purification. Compound 2 (32 g, crude) was obtained as a brown oil.

To a solution of compound 2 (32 g, 63.02 mmol, 1 eq) in DCM (210 mL) was added con.HCl (70 mL). The mixture was stirred at 25° C. for 2 hr. The reaction mixture was concentrated under reduced pressure to remove the solvent and then lyophilized. The crude product was triturated with (PE:EtOAc=1:1) at 0° C. for 30 min to give compound 3 (18.9 g, crude) as a yellow solid. 1H NMR (400 MHz, CHLOROFORM-d, EW58397-468-P1A1) δ 3.63 (t, J=6.8 Hz, 4H), 3.38 (t, J=6.8 Hz, 2H), 2.38 (t, J=7.2 Hz, 4H), 1.58-1.52 (m, 8H), 1.36-1.25 (m, 24H).

To a solution of compound 3 (1.6 g, 4.67 mmol, 1 eq) in DCM (20 mL) was added EDCI (3.58 g, 18.68 mmol, 4 eq), DIEA (6.04 g, 46.71 mmol, 8.14 mL, 10 eq), KC3-X2-PH11_4 (2.00 g, 9.34 mmol, 2 eq) and DMAP (1.43 g, 11.68 mmol, 2.5 eq). The mixture was stirred at 40° C. for 16 hr. The reaction mixture was concentrated in vacuum. The residue was purified by column chromatography (SiO₂, Petroleum ether/Ethyl acetate=I/O to 94/6) to give compound 4 (1.8 g, 2.41 mmol, 51% yield) as a yellow oil. ¹H NMR (400 MHz, CHLOROFORM-d, EW58397-474-P1A1) δ 4.05 (t, J=6.8 Hz, 4H), 2.38 (t, J=7.2 Hz, 4H), 2.30 (t, J=7.4 Hz, 4H), 1.65-1.50 (m, 14H), 1.41-1.21 (m, 38H), 1.17-1.02 (m, 8H), 0.90-0.80 (m, 18H).

A solution of compound 4 (2 g, 2.72 mmol, 1 eq), PPTS (205.08 mg, 816.09 mol, 0.3 eq) and compound 5 (871.64 mg, 5.44 mmol, 2 eq) in Tol. (30 mL) was heated to reflux and the water was removed by Dean-Stark trap. The mixture was stirred at 135° C. for 16 hr. The reaction mixture was quenched by addition sat-NaHCO₃ (aq. 80 mL) at 25° C., and extracted with EtOAc (30 mL*3). The combined organic layers were washed with brine (20 mL*3), dried with anhydrous Na₂SO₄, filtered and concentrated in vacuum. The residue was purified by flash silica gel chromatography (ISCO®; 40 g SepaFlash® Silica Flash Column, Eluent of 0~10% Ethyl acetate/Petroleum ether gradient @60 mL/min) to give compound 6 (850 mg, 832.40 mol, 31% yield) as a yellow oil. ¹H NMR (400 MHz, CHLOROFORM-d, EW58311-476-P1A1) δ 4.12-4.03 (m, 6H), 3.72-3.64 (m, 2H), 3.52-3.43 (m, 1H), 2.32-2.28 (m, 4H), 1.64-1.56 (m, 18H), 1.35-1.24 (m, 42H), 1.16-1.05 (m, 8H), 0.88-0.83 (m, 18H).

To a solution of compound 6 (840 mg, 1.00 mmol, 1 eq) in DCM (10 mL) was added TEA (507.56 mg, 5.02 mmol, 698.15 μL, 5 eq) and MsCl (270.00 mg, 2.36 mmol, 182.43 μL, 2.35 eq) at 0° C. The mixture was stirred at 0° C. for 1 hr. The reaction mixture was quenched by addition ice-water (30 mL) at 0° C., and extracted with DCM (10 mL*3). The combined organic layers were washed with brine (10 mL*3), dried with anhydrous Na₂SO₄, filtered and concentrated in vacuum. The residue was purified by flash silica gel chromatography (ISCO®; 12 g SepaFlash® Silica Flash Column, Eluent of 0~10% Ethylacetate/Petroleum ethergradient @40 mL/min) to give compound 7 (760 mg, 788.70 mol, 79% yield,) as a yellow oil. ¹H NMR (400 MHz, CHLOROFORM-d, EW58311-477-P1A1) δ 4.33-4.22 (m, 2H), 4.09-4.01 (m, 6H), 3.53-3.42 (m, 1H), 3.02 (s, 3H), 2.30 (t, J=7.6 Hz, 4H), 1.64-1.55 (m, 20H), 1.36-1.23 (m, 40H), 1.16-1.04 (m, 8H), 0.89-0.83 (m, 18H).

A solution of compound 7 (760 mg, 830.21 mol, 1 eq) in Me₂NH/THF (2 M, 10 mL) was stirred at 50° C. for 10 hr. The reaction mixture was concentrated under reduced pressure to remove solvent. The residue was purified by flash silica gel chromatography (ISCO®; 12 g SepaFlash® Silica Flash Column, Eluent of 0~8% Ethyl acetate/(Petroleum ether/Dichloromethane=3/1, 0.1% TEA as eluant)@40 mL/min) and followed by reversed-phase HPLC (column: PHS XP Phenyl-Hexyl 150*25 mm*7 um; mobile phase: [H₂O (0.225% FA)-ACN]; gradient: 65%-95% B over 15.0 min) to give AKG-KC3-X2-PH12 (371.1 mg, 422.87 mol, 48% yield, 98.5% purity) as a yellow oil. LCMS (EW58311-481-P1C1): CAD, m/z, M+H=865.0. HRMS (ESI) m/z: [M+H]⁺ Calcd for C₅₄H₁₀₆NO₆⁺864.8015, found 864.8032. ¹H NMR (400 MHz, CHLOROFORM-d, EW58311-481-P1A1) δ 4.13-3.99 (m, 6H), 3.52-3.40 (m, 1H), 2.44-2.21 (m, 12H), 1.67-1.48 (m, 18H), 1.39-1.19 (m, 42H), 1.17-1.04 (m, 8H), 0.92-0.82 (m, 18H).

FIG. 2C is a table of additional examples of KCn-X₂ compounds that were prepared.

### Example 2. LNP Preparation

Lipid nanoparticle (LNP) compositions encapsulating mRNA can be prepared as described in Example 9 (Preparation of lipidic nanoparticles) of Patent Cooperation Treaty Patent Application PCT/US2021/060877 (WO2022/115645), and Example 9 (Preparation of lipidic nanoparticles) of Patent Cooperation Treaty Patent Application PCT/US2023/067517 (WO2023/230587), are incorporated herein by reference in their entirety, modified by using a microfluidics chip from ChipShop (187) to make LNPs at flow rates of 18 mL/min instead of the Nanoprecision Systems microfluidics cassette at 6 ml/min whilst keeping downstream processing and analytical procedures the same as described in the published patents referenced above.

Stock solutions of each lipid are prepared. Ionizable lipids are weighed out in 4 mL glass vials (Thermo B7999-2) and dissolved in ethanol (Sigma-Aldrich 200 proof, RNase free) to a final concentration of 10 mM. Other lipids such as DSPC, DPPC-NH₄, DPPS-NH₄ and PEG-DMG are weighed out and dissolved in ethanol to a concentration of 1 mM, and cholesterol to concentration of 10 mM.

Lipid mixtures for each individual LNP are prepared by adding the desired volume of each lipid stock solution to a new vial, adding ethanol if needed to achieve a final volume of 1.2 mL.

mRNA solutions are prepared by thawing frozen mRNA (e.g. FFluc-mRNA) vials and diluting mRNA in 6.25 mM sodium acetate (pH 5.0) to a final concentration of 0.033 mg/mL To prepare LNPs, a NanoAssemblr Benchtop microfluidic device (from Precision Nanosystems) is used. 3 mL of mRNA solution is loaded into a 3 mL disposable syringe (BD 309656) and 1 ml of lipid mixture in a 1 ml syringe (BD309659) and placed in the NanoAssemblr heating block for 4 min prior to mixing. LNP formation is achieved by pumping the liquid streams through a disposable microfluidics cassette at 3:1 aqueous:alcohol volume ratio at 6 mL/min mixing speed, or through a ChipShop microfluidics (Jena, Germany) chip (#187) at 18 ml/min. After mixing, 3.6 mL of LNP mixture is collected, while the initial mixed volume of 0.35 mL and last 0.05 mL of mix is discarded. Ethanol is removed by buffer exchange using SpectraPor dialysis tubing (12-14 k MWCO) in PBS (Cytiva, SH30256.01) or by sequential concentration and dilution using Amicon Ultra-4 centrifugal concentrators (10 k MWCO, at 500 g).

LNPs are typically exchanged into PBS, pH 7.4 and then 10 mM Tris, pH 7.4, 10% sucrose, concentrated to 20-50 ug/mL mRNA using an Amicon-Ultra 4 (100,000 MWCO) spin column, sterile filtered (Thermo Nalgene 0.2 μm #720-1320) prior to freezing by immersion in liquid nitrogen for 5 min and long-term storage at −80° C.

### Example 3: Characterization of Lipidic Nanoparticles (LNPs) Containing mRNA

### A. MRNA Encapsulation Efficiency

Materials: Quant-iT™ RiboGreen™ RNA reagent (1 mL solution in DMSO), 20×TE (25 mL in DEPC-treated water), DNase/RNase-Free Distilled Water (Invitrogen Cat #10977-023, or equivalent), 96-Well Clear-Bottom Microplate MED BINDING (Greiner Bio-One Cat #655096), Detergent (Triton™): Surfact-Amps™ X-100 Cat #28314, RNase/DNase-free 1.5 mL Microcentrifuge tubes (ThermoFisher, Invitrogen Cat #AM12400) or equivalent sterile, nuclease-free tubes. A portion of 1×TE buffer prepared for the experiment was allotted for the dilution of RiboGreen dye. This dye was diluted 200× in 1×TE buffer. As the dye is light sensitive, allow to come to RT protected from light. Ensure the dye has melted (become liquid) as it will remain crystalline in temperatures below RT. Before pipetting the dye vortex mix for one second, three times. Prepare the volume of 1×TE buffer needed in a 15 mL conical tube. Vortex or invert the diluted dye and store protected from light. Standards corresponding to 1500, 1200, 1000, 800, 500, 250, 50, 25 and 0 ng/mL mRNA were prepared in 1×TE buffer from the mRNA used in the preparation of LNPs. The blank contains no mRNA. LNPs are diluted in 1×TE buffer to approximately 2 μg/mL. 100 μL of blank, standards and samples were added to the wells of a 96-well plate in triplicate to which 100 μL of diluted Ribogreen is added and mixed well by pipetting up and down 4-5 times. The plate is covered with foil to protect from light and after 5 min incubation at room temperature. The standards and samples were read using a microplate fluorometer (e.g., SpectraMax M2) using excitation at 485 nm, emission at 530 nm, and instrument default acquisition parameters. A 2% Triton solution is prepared from the 10% stock solution of Surfact-Amps™ X-100 by performing a 1:5 dilution in 1×TE buffer. Invert 5× to mix. Once the pre-Triton readings have been taken and saved, empty prepared Triton (diluted to 2% in 1×TE buffer) into a clean reagent reservoir. A multichannel pipettor was used to add 20 μL of 2% Triton to each plate well and slowly pipette mix 5×. The plate can be read 5 minutes after mixing. The pre-Triton mRNA concentrations are determined by extrapolation to the pre-Triton standard curves and the post-Triton mRNA concentrations are determined by extrapolation to the Triton containing standard using a linear curve fit.

% Encapsulation Efficiency (% E.E) expresses the concentration of encapsulated mRNA as a percent of the total, post-Triton concentration. The formula can be expressed as:

% Encapsulation Efficiency=100−((Pre-Triton concentration/Post-Triton concentration)*100)

### B. LNP Particle Size

30 μL of LNP was mixed with 1.5 mL of 0.1 μm filtered PBS in a polystyrene cuvette (Sarstedt, #67.754) and analyzed for particle size using a ZetaSizer Pro (Malvern) The Z-average size and polydispersity index value were obtained by the method of cumulants using ZS Xplorer software, version number 1.4.0.105. Typically, size measurements of LNPs were taken post LNP mixing, post buffer exchange, post sterile filtering and after freezing for 2 min in liquid nitrogen and thawing at 37° C.

### C. LNP Zeta Potential

30 μL of LNP is mixed with 1.5 mL 10 mM 2-(N-morpholino)ethanesulfonic acid (MES), 15 mM sodium chloride, pH 5 or 10 mM 4-(2-hydroxyethyl)-1-piperazineethanesulfonic acid (HEPES), 15 mM sodium chloride, pH 7 and injected into a disposable folded capillary cell (Malvern Nanoseries DTS1070) and zeta potential measured on a ZetaSizer Pro at 25° C.

### D. LNP Apparent pKa Determination

1.0 L of buffer consisting of 10 mM sodium phosphate, 10 mM ammonium acetate, 10 mM sodium citrate, and 150 mM sodium chloride solution in WFI was prepared. The solution is divided into 50 mL aliquots and the pH of each adjusted with 5 M HCl or 10N NaOH to span the pH range of 2-12 in approximately 0.5-unit increments. The actual pH of each aliquot was measured with a pH meter (Thermo Orion 4 Star with a glass combination micro pH electrode (8220BNWP) calibrated with Thermo Orion pH 4.01, 7.00, 10.01 standards).

A 10 mM solution of 6-p-Toluidino-2-naphthalenesulfonic acid (TNS, Abcam ab275049) in 10 mL of DMSO is prepared from which a 1 mM solution in water is prepared. LNPs were diluted to 1 μg/mL mRNA in a well of a 96-well plate in a final volume of 200 μL for each pH prepared above and TNS added to a final concentration of 10 μM after which each well is mixed by pipetting up and down 10 times with a multichannel pipette. The plate was incubated at room temperature for 3 min, then read on a plate reader at Ex.331/Em.445 nm. A plot of pH versus TNS fluorescence signal was prepared in Graph Pad Prism. The pH value at the half maximum value of fluorescence represents the pKa of nanoparticles and is calculated by performing a non-linear regression analysis for a half maximal fluorescence. This value is the pH at which the numbers of ionized (protonated) and deionized groups are equal.

### Example 4. In Vitro Assessment of Biodegradability of Ionizable Lipids Using Pancreatin Enzymes

An in vitro assay was developed to evaluate the biodegradability of ionizable cationic lipids (ICLs) in the presence of pancreatin enzymes (including trypsin, amylase and lipase). Individual ICL stock solutions were prepared in ethanol at a concentration of 50 mg/mL and stored at −80° C. until use. Bile salts (Millipore, Cat. No. 48305) were dissolved in Dulbecco's phosphate-buffered saline (DPBS; Sigma, Cat. No. 59300C) at 2 mg/mL, and pancreatin (Sigma, Cat. No. P7545) was prepared at 10 mg/mL in the same buffer. The pH of the DPBS was adjusted to 8.0 using 1 M NaOH. All solutions were maintained at 37° C. under continuous mixing. For each reaction, 50 nmol of ICL (from the 50 mg/mL ethanol stock) was added to 2 mL of pancreatin solution. Reactions were performed in duplicate across six time points. The mixture was vortexed for 30 seconds, after which 100 μL aliquots were transferred into separate 2 mL tubes (two replicates per time point). Samples were incubated at 37° C. with shaking. To terminate the reaction, 10 volumes of isopropanol/acetonitrile (IPA/MeCN, 70:30 v/v) were added. The resulting mixture was sonicated for 5 minutes to facilitate phase separation of lipids and enzymes, followed by centrifugation at 14,000×g for 15 minutes. The supernatant was collected and transferred to HPLC vials for analysis. Lipid degradation was quantified using a Thermo Scientific Vanquish Flex UHPLC system equipped with a Charged Aerosol Detector (CAD) and a C18 column (Phenomenex Kinetex 1.7 μm C18 100 Å 150×2.1 mm). Operating conditions for UHPLC are detailed in Table 6. The extent of lipid degradation was expressed as the percentage of the main ICL peak remaining at each time point relative to the peak area at time zero. Quantification was performed using ICL standards prepared in IPA at concentrations ranging from 5 to 40 g/mL.

TABLE 6
Chromatographic Conditions

| HPLC Instrument | Thermo Scientific Vanquish Flex UHPLC | | |
|---|---|---|---|
| HPLC Column | C18 column (Phenomenex Kinetex 1.7 μm C18 | | |
| | 100 Å 150 × 2.1 mm) | | |
| HPLC guard column | None | | |
| Column Temperature | 50° C. | | |
| Flow Rate, ml/min | 0.35 | | |
| Injection Volume | 5 μL | | |
| CAD | 10H | | |
| Run Time | 12 min | | |
| Sample Solvent | 70% IPA/30% MeCN | | |
| Mobile Phase A | 0.1% TFA in water | | |
| Mobile Phase B | 0.05% TFA in MeCN | | |
| | | Mobile | Mobile |
| Gradient | Time, min | Phase A % | Phase B % |
| | −0.5 | 15 | 85 |
| | 0 | 15 | 85 |
| | 0.5 | 15 | 85 |
| | 5 | 5 | 95 |
| | 11 | 5 | 95 |
| | 11.5 | 15 | 85 |
| | 12 | 15 | 85 |

Results are shown in the graphs in FIG. 4A and FIG. 4B showing biodegradability of ionizable cationic lipids (ICLs), measured after 15 minutes (FIG. 4A) and 3 hours (FIG. 41B) in the presence of pancreatin enzymes (including trypsin, amylase and lipase) measured according to the assay of Example 4. FIG. 4A and FIG. 4B are graphs of the biodegradability of ionizable cationic lipids (ICLs) over time in the presence of pancreatin enzymes (including trypsin, amylase and lipase) measured according to the assay of Example 4. The ionizable cationic lipids of Formula (I) that were tested (Table 7 with structures in Table 8) showed significantly greater degradation compared to the ALC-0315, SM-102 or KC3-OA ionizable lipid comparator compounds.

TABLE 7
Results Obtained from Ionizable Lipids Tested in Example 4.

| ICL | t = 15 min. | t = 3 hr |
|---|---|---|
| KC3-OA | 0.98 | 0.94 |
| ALC-0315 | 0.92 | 0.94 |
| SM-102 | 0.89 | 0.53 |
| KC3-X1-PH5 | 0.67 | 0.20 |
| KC3-X1-PH12 | 10.29 | 0.03 |
| KC3-X1-AA | 0.23 | 0.01 |
| KC3-X2-PH12 | 0.08 | 0.02 |

TABLE 8
Chemical structures of Ionizable Lipids Tested in Examples.

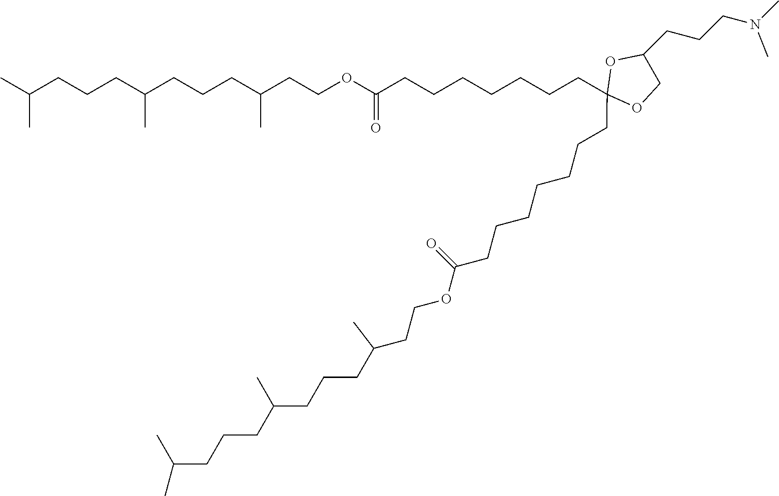 [SMILES: CC(C)CCCC(C)CCCC(C)CCOC(=O)CCCCCCCC1(CCCCCCCC(=O)OCCC(C)CCCC(C)CCCC(C)C)OCC(CCCN(C)C)O1]

KC3-X1-PH5

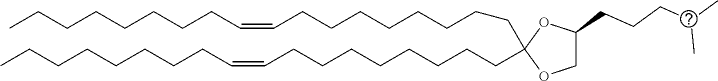 [SMILES: CCCCCCCC/C=C\CCCCCCCCC1(CCCCCCCC/C=C\CCCCCCCC)OC[C@H](CCCC(C)C)O1]

KC3-OA

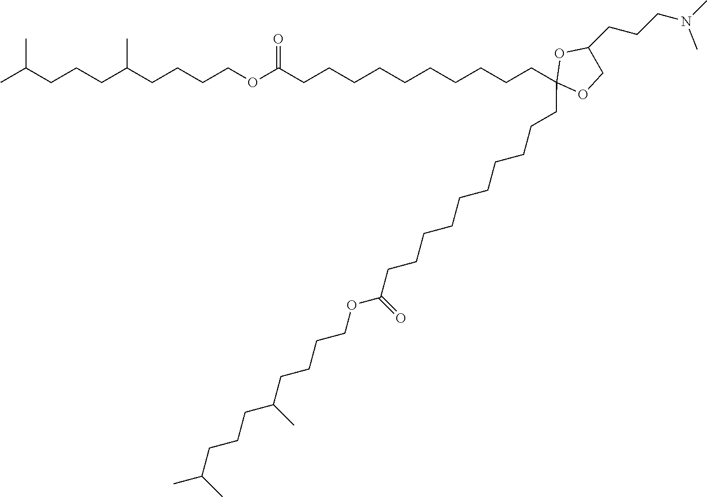 [SMILES: CC(C)CCCC(C)CCCCOC(=O)CCCCCCCCCCC1(CCCCCCCCCCC(=O)OCCCCC(C)CCCC(C)C)OCC(CCCN(C)C)O1]

KC3-X1-PH12

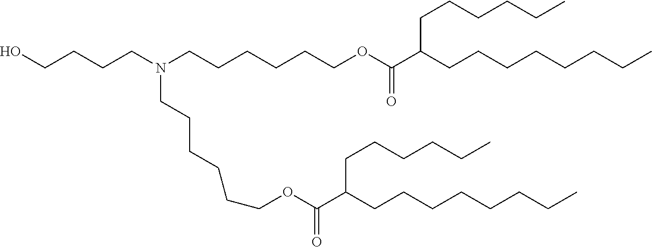 [SMILES: CCCCCCCCC(CCCCCC)C(=O)OCCCCCCN(CCCCO)CCCCCCOC(=O)C(CCCCCC)CCCCCCCC]

ALC-0315

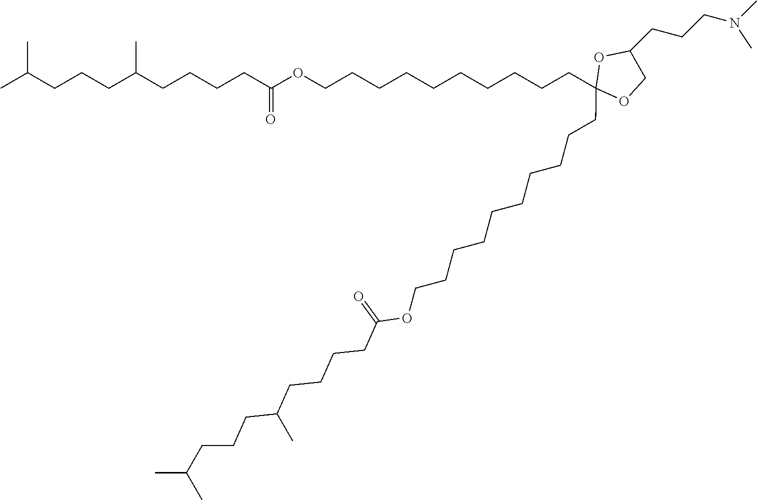 [SMILES: CC(C)CCCC(C)CCCCC(=O)OCCCCCCCCCCC1(CCCCCCCCCCOC(=O)CCCCC(C)CCCC(C)C)OCC(CCCN(C)C)O1]

KC3-X2-PH12

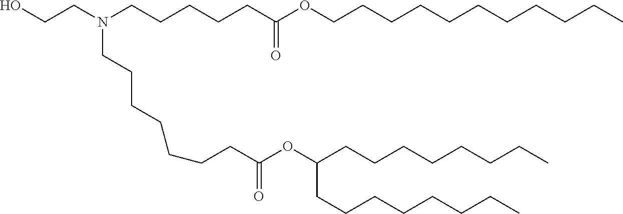 [SMILES: CCCCCCCCCCCOC(=O)CCCCCN(CCO)CCCCCCCC(=O)OC(CCCCCCCC)CCCCCCCC]

SM-102

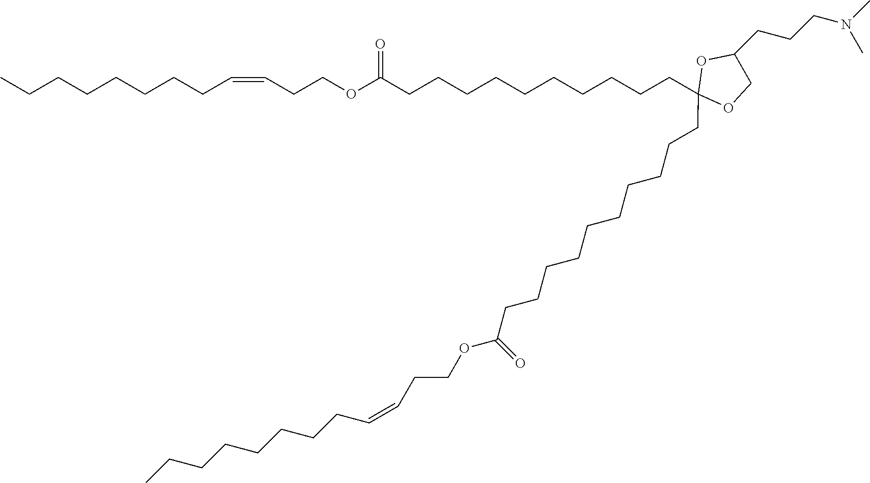 [SMILES: CCCCCCCC/C=C\CCOC(=O)CCCCCCCCCCC1(CCCCCCCCCCC(=O)OCC/C=C\CCCCCCCC)OCC(CCCN(C)C)O1]

KC3-X1-AA

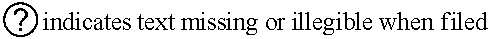

### Example 5. Determination of Transfection Efficiency in Murine Dendritic Cells of LNPs Using Firefly Luciferase mRNA

Immortalized mouse dendritic cells (MutuDC 1940, Fuertes Marraco et al., Front. Immunol. 2012, 3:331) are cultivated in Gibco™ IMDM medium with GlutaMax™ (Gibco cat. No 31980097), supplemented with 7.5 mg/ml NaHCO₃, 10 mM HEPES-Na pH 7, 50 μM beta-mercaptoethanol, 50 U/ml penicillin, 50 μg/mL streptomycin, and 10% fetal bovine serum, at 37° C., 5% CO₂. The cells are rinsed with phosphate-buffered saline (PBS, Cytiva, cat. No SH30526) harvested using 5 mM EDTA in PBS (PBS-EDTA), resuspended in the growth medium, inoculated in the white opaque 96-well cell culture plates (e.g., Costar cat. No 3917) in the wells in columns 3-10 of the plate at 15,000 cells/well in 0.1 ml of the medium and allowed to acclimate overnight. The media are aspirated, replaced with 0.1 ml of the media containing the desired concentration of mRNA LNP (0.1-1 μg/mL mRNA, typically 0.3 μg/mL mRNA—SEQ ID NO: 3 encoding SEQ ID NO: 2), and incubated at 37° C., 5% CO₂ for the desired transfection period, typically 24 hours. The mRNA can be an mRNA encoding a firefly luciferase protein sequence such as SEQ ID NO:2. Three to four hours prior to the end of the transfection period, the wells A2 and A11 of the plates are seeded with 0.1 ml of the cell suspension standard containing approximately 1,000,000 viable cells/mL (the viable cell concentration is accurately determined), and wells B2 to G2, and B11 to G11 are seeded with the serial dilutions of the standard at the dilution factor of 1.5 to generate eight cell quantity standards per plate in duplicate. In the wells A1-H1, A12-H12, 0.1 ml of the cell medium is added. At the end of the transfection period, 11 μL of 20 mM aqueous solution of Hoechst 33342 DNA fluorescent dye (Ultrapure grade, cat. No 17533, AAT Bio, USA) is added to each well, carefully mixed by gentle tapping and swirling of the plate by hand, and the plate is incubated at 37° C., 5% CO₂ for one hour to allow for the staining of the cellular DNA by added dye.

Luciferase standards are prepared using recombinant firefly luciferase (cat. No. SRE0045, Sigma-Aldrich, USA) reconstituted to 2 mg/ml protein in 1 M Tris-HCl buffer pH 8.5 and stored at −80° C. An interim standard is prepared by diluting the reconstituted luciferase 20-fold (to 0.1 mg/ml) in PBS-EDTA containing 1 mg/ml nuclease- and protease-free bovine serum albumin (PBS-EDTA-BSA). Eight working standards (DA to DH) are prepared by further diluting the interim standard in PBS-EDTA-BSA, first to 1000 ng/mL (DA), and then serially diluting DA down to DH with the dilution factor of three per step. The interim and working standards are prepared fresh.

At the end of one hour incubation, the media from the wells in columns 2-11 of the plate are aspirated, and 0.1 ml of PBS-EDTA is added. The plate is incubated at 37° C., 5% CO₂ for 10 min and on the microplate shaker at 700 rpm for 3 min to dislodge the cells. Then the media from the wells A1-H1, A12-H12 are aspirated and 0.1 ml aliquots of the luciferase working standards are added, from the highest (DA) in A1 and A12, to the lowest (DH) in H1 and H12, creating a series of eight luciferase standards per plate in duplicate (plate columns 1 and 12).

The Hoechst 33342 fluorescence of the cell suspensions in the wells is determined using SpectraMax M2 multimode plate reader (Molecular Devices, USA) using excitation at 360 nm, emission at 461 nm, and nine-point well scan option. The plate is auto-mixed for 5 sec prior to measurements. The average fluorescence signal per each well is determined, and the fluorescence readings of the cell standard wells A2-H2, A11-H11 are used to generate the linear standard curve of the average fluorescence vs. cell concentration in the well (0.1 ml). The cell concentration in the 0.1 ml of the cell suspension at the end of transfection is determined from this standard curve for each well in a conventional manner.

The luciferase concentration in the cell suspensions is determined using BrightGlo® Firefly Luciferase assay kit (Promega Corp., USA). To each of the luciferase standard wells, test sample wells, and blank (cells only) wells G2-H2, G1l-H11 in the same plate, 0.1 mL of the reconstituted complete Luciferase substrate buffer (prepared according to the manufacturer's recommendations) is added, the contents of the wells are thoroughly mixed, and the luminescence of the mixtures in the wells is determined at 10-15 min after the luciferase substrate addition using SpectraMax M2 multimode plate reader (Molecular Devices, USA) in the luminescence mode under the instrument default settings. The readings of the duplicate standards in the wells A1-H1 and A12-H12, after subtraction of the blank readings of the wells G2-H2 and G11-H11, are used to generate the Luciferase standard curve linearly approximated in double-log coordinates using the minimization of the mean squared relative error. The amount of luciferase formed in each well is calculated from the luminescence readings (with blank readings subtracted) using the luciferase standard curve, normalized to the number of cells per well determined using the Hoechst 33342 method above, and presented in ng of luciferase per 1 million (10⁶) cells. It was found that for the targeted LNP and the typical assay range of post-transfection cell concentrations of (99-1251)×10³ cell/ml in 0.1 ml of the cell harvesting solution ((9.9-125.1)×10³ cells/well), observed under the above assay conditions, the Luciferase expression normalized to the final number of the cells in the well was dependent on both initial (plating) cell density and final (post-transfection) cell density, and the dependence was in good agreement with the second-degree polynomial approximation against the logarithm of the post-transfection cell concentration (R²=0.9925). Therefore, for targeted LNPs the assayed luciferase activities in each individual well were corrected to the standard post-transfection cell concentration of 400×10³ cells/ml (cell density 40×10³ cells/well) using the formula: Luc=Luc(D)*100/(91.6×log²(D)−659.2×log(D)+1196.4), where D is the measured post-transfection cell concentration in 10³ cells/ml, Luc(D) is the measured luciferase expression at the post-transfection cell concentration D.

### Example 6. In Vitro Transfection of Ffluc-mRNA LNPs Composed of the Ionizable Lipids Formulated in the Presence and Absence of 5 Mol % DPPS-NH₄

A mRNA encoding Ffluc-mRNA was formulated separately into LNPs comprising an ICL/DSPC/Chol/PEG-DMG (52/10/36.5/1.5 mol %) or ICL/DSPC/DPPS-NH₄/Chol/PEG-DMG (52/5/5/36.5/1.5 mol %) formulated at an N/P ratio of 5.25. The LNPs were prepared and characterized as described in Example 2 and 3 respectively.

Particle size refers to the Z-Average particle size after freezing and thawing. Zeta refers to zeta potential measurements at either pH 5 or pH 7 in units of mV. mRNA encapsulation efficiency after freezing & thawing is referred to as (E.E,%).

TABLE 9A
Physicochemical properties and characterization of LNPs containing Ffluc
mRNA comparing different ICLs in a PS-targeted and non-targeted form.

| ICL | Targeted? | Z-Ave | PDI | % E.E | Zeta pH 5 | Zeta pH 7 |
|---|---|---|---|---|---|---|
| KC3-X1-PH12 | No PS | 84.54 | 0.006 | 95.71 ± 0.27 | 17.41 | 3.66 |
| KC3-X2-PH12 | No PS | 88.77 | 0.036 | 95.78 ± 0.19 | 17.94 | 2.73 |
| KC3-X1-PH5 | No PS | 85.84 | 0.06 | 96.64 ± 0.28 | 20.69 | 4.61 |
| KC3-OA | No PS | 81.68 | 0.077 | 97.20 ± 0.39 | 16.76 | 5.09 |
| KC3-X1-PH12 | 5 mol % PS | 87.15 | 0.065 | 96.78 ± 0.08 | 19.84 | −1.16 |
| KC3-X2-PH12 | 5 mol % PS | 87.65 | 0.017 | 96.69 ± 0.33 | 18.83 | 0.17 |
| KC3-X1-AA | 5 mol % PS | 86.16 | 0.052 | 96.47 ± 0.20 | 19.62 | −1.23 |
| KC3-X1-PH5 | 5 mol % PS | 83.27 | 0.048 | 98.22 ± 0.13 | 19.24 | −2.66 |
| KC3-OA | 5 mol % PS | 82.93 | 0.028 | 97.90 ± 0.09 | 15.77 | −0.89 |

The formulations ranged in size from approximately 82 to 93 nm in diameter The % entrapped mRNA ranged from 94-98% and LNPs exhibited positive surface charge at pH 5~+16-21 and a negative charge at pH 7-3 to +5 mV.

The activity of the LNPs was evaluated by transfection MutuDC1940 cells in vitro at 0.3 μg/mL mRNA concentration for 24 h as described in Example 5.

TABLE 9B
In vitro activity of LNPs containing Ffluc mRNA comparing
different ICLs in a PS-targeted or non-targeted format.

| ICL | Targeted? | Luc (ng/million cells) | T/NT |
|---|---|---|---|
| KC3-X1-PH12 | No PS | 5.03 ± 0.16 | |
| KC3-X2-PH12 | No PS | 8.12 ± 0.55 | |
| KC3-X1-PH5 | No PS | 12.40 ± 1.15 | |
| KC3-OA | No PS | 26.89 ± 4.67 | |
| KC3-X1-PH12 | 5 mol % PS | 334.27 ± 17.96 | 66.5 |
| KC3-X2-PH12 | 5 mol % PS | 447.74 ± 43.72 | 55.2 |
| KC3-X1-PH5 | 5 mol % PS | 199.92 ± 22.25 | 16.1 |
| KC3-OA | 5 mol % PS | 76.19 ± 6.52 | 2.8 |

It was found that the rank order of activity and the activity of the PS-targeted to non-targeted ratio (highest to lowest) was KC3-X2-PH12>KC3-X1-PH12>KC3-X1-PH5>KC3-OA at 0.3 μg/mL.

### Example 7. Preparation and Analysis of PS-Containing LNPs Containing KC3-X1-PH12 at Varying Mol % Encapsulating the mRNA FFluc which was Used to Evaluate the Effect of ICL Content on Activity in MutuDC1940 Murine Dendritic Cells In Vitro

LNPs were prepared comprising 48, 52, 54, 56, 60 or 64 mol % KC3-X1-PH12 ionizable lipid with the composition KC3-X1-PH12/DSPC/DPPS/Chol/PEG-DMG (x/5/5/(88.5-x)/1.5 mol %) and an N/P ratio of 4.5 encapsulating firefly luciferase mRNA (FFluc) were prepared as described in Example 2 (using a ChipShop 187 chip to mix and prepare the LNPs at a flow rate of 18 ml/min.) where x represents the mol % ionizable lipid. The LNPs were used to evaluate the effect of altering the mol % of KC3-X1-PH12 on in vitro luciferase activity in vitro. The LNPs were characterized using the method of Example 3. Particle size refers to the Z-Average particle size after freezing and thawing. Zeta refers to zeta potential measurements at either pH 5 or pH 7 in units of mV. mRNA encapsulation efficiency is referred to as (E.E,%).

TABLE 10A
Physicochemical properties and characterization of DPPS
containing KC3-X1-PH12 LNPs containing FFluc mRNA

| | Particle | E.E, %, | Zeta | Zeta |
|---|---|---|---|---|
| Mol % | Size (nm) | Stdev | pH 5 | pH 7 |
| 48 | 101.80 | 87.02 ± 0.76 | 18.68 | −1.84 |
| 52 | 104.20 | 87.42 ± 0.54 | 16.04 | −2.36 |
| 56 | 109.40 | 88.65 ± 0.45 | 15.34 | −3.38 |
| 60 | 111.70 | 88.76 ± 0.52 | 18.70 | −3.34 |
| 64 | 119.30 | 87.06 ± 0.67 | 13.73 | −4.40 |

The formulations ranged in size from approximately 104 to 112 nm in diameter. The % entrapped mRNA ranged from % and LNPs exhibited a surface charge range at pH 5 from approximately +14 to +19 mV and a range from −4 to −2 mV at pH 7.

TABLE 10B
Luciferase expression in MutuDC 1940 cells after exposure to LNPs
for 24 h at 0.3 μg/mL total (encapsulated + non-encapsulated)
mRNA concentration. A frozen KC3-OA LNP formulation described
in Example 11 was used in the luciferase assay as a control.

| | Luciferase activity | Luciferase Activity |
|---|---|---|
| Mol % | (ng/10\hat{ }6 cells) ± stdev | KC3-X1-PH12/KC30A |
| 48 | 421.7 ± 21.8 | 1.07 |
| 52 | 506.5 ± 24.8 | 1.29 |
| 56 | 662.4 ± 44.3 | 1.69 |
| 60 | 592.2 ± 27.8 | 1.51 |
| 64 | 417.8 ± 25.5 | 1.06 |

### Example 8. Preparation and Analysis of LNPs Containing KC3X1-PH5, KC3-X1-AA and KC3-X1-PH12 at Different N/P Ratios Encapsulating FFluc mRNA Used to Evaluate Activity In Vitro

LNPs were prepared as described in Example 2, comprising of ICL/DSPC/DPPS-NH₄/Chol/PEG-DMG (52/5/5/36.5/1.5 mol %) at varying N/P ratios containing the reporter mRNA FFluc and evaluated in vitro for luciferase activity. LNPs were characterized with the methods of Example 3. Particle size refers to the Z-Average particle size after freezing and thawing. Zeta refers to zeta potential measurements at either pH 5 or pH 7 in units of mV. mRNA encapsulation efficiency is referred to as (E.E,%).

TABLE 11A
Physicochemical properties and characterization
of KC3-X1-PH5 and KC3-X1-AA LNPs containing
FFluc mRNA at different N/P ratios.

| | | Particle | E.E, %, | Zeta | Zeta |
|---|---|---|---|---|---|
| ICL | N/P | Size (nm) | Stdev | pH 5 | pH 7 |
| KC3-X1-PH5 | 4.0 | 114.60 | 95.63 ± 7.04 | 16.12 | −7.35 |
| KC3-X1-PH5 | 4.25 | 114.60 | 94.76 ± 7.04 | 18.28 | −3.93 |
| KC3-X1-PH5 | 5.0 | 116.10 | 95.76 ± 7.04 | 21.06 | −3.50 |
| KC3-X1-PH5 | 5.5 | 114.40 | 95.42 ± 7.04 | 22.90 | −2.67 |
| KC3-X1-PH5 | 6.0 | 111.90 | 95.00 ± 7.04 | 22.09 | −2.91 |
| KC3-X1-PH5 | 6.5 | 114.50 | 95.61 ± 7.04 | 26.79 | −3.46 |

TABLE 11B
Luciferase expression (ng/million cells ± stdev) in MutuDC
1940 cells after exposure to LNPs for 24 h at 0.1 μg/mL
total (encapsulated + non-encapsulated) mRNA concentration.

| ICL | N/P | Luciferase Activity |
|---|---|---|
| KC3-X1-PH5 | 4.0 | 103.7 ± 9.9 |
| KC3-X1-PH5 | 4.25 | 118.9 ± 4.4 |
| KC3-X1-PH5 | 5.0 | 138.9 ± 6.0 |
| KC3-X1-PH5 | 5.5 | 142.8 ± 6.9 |
| KC3-X1-PH5 | 6.0 | 146.6 ± 4.8 |
| KC3-X1-PH5 | 6.5 | 157.8 ± 10.7 |

TABLE 11C
Luciferase expression (ng/million cells ± stdev) in MutuDC
1940 cells after exposure to LNPs for 24 h at 0.3 μg/mL
total (encapsulated + non-encapsulated) mRNA concentration.

| ICL | N/P | Luciferase Activity |
|---|---|---|
| KC3-X1-PH5 | 4.0 | 273.3 ± 19.8 |
| KC3-X1-PH5 | 4.25 | 284.4 ± 13.4 |
| KC3-X1-PH5 | 5.0 | 295.3 ± 9.3 |
| KC3-X1-PH5 | 5.5 | 287. ± 12.4 |
| KC3-X1-PH5 | 6.0 | 302.3 ± 13.1 |
| KC3-X1-PH5 | 6.5 | 293.5 ± 11.8 |

LNP formulations prepared with KC3-X1-PH5 and KC3-X1-AA demonstrated peak luciferase activity at an N/P of 6 and 4.25, respectively. All formulations demonstrated greater than 90% encapsulation efficiency and a particle size between 107 and 119 nm, independent of the N/P ratio.

TABLE 12A
Physicochemical properties and characterization of KC3-X1-
PH12 LNPs containing FFluc mRNA at different N/P ratios.
Some formulations were prepared on more than one occasion.

| | Particle | E.E, %, | Zeta | Zeta |
|---|---|---|---|---|
| N/P | Size (nm) | Stdev | pH 5 | pH 7 |
| 1.0 | 129.00 | 16.07 ± 0.48 | −0.08 | −27.50 |
| 2.0 | 151.20 | 56.87 ± 2.77 | 0.29 | −6.67 |
| 3.0 | 109.50 | 76.03 ± 2.39 | 6.80 | −1.54 |
| 3.25 | 110.20 | 82.23 ± 2.17 | 10.95 | −1.50 |
| 3.5 | 109.70 | 89.15 ± 3.14 | 12.50 | −2.95 |
| 3.75 | 111.30 | 93.00 ± 2.93 | 12.99 | −3.60 |
| 4.0 | 107.90 | 92.08 ± 1.99 | 15.52 | −4.01 |
| 4.25 | 107.40 | 92.27 ± 3.66 | 15.22 | −5.62 |
| 4.5 | 107.30 | 92.25 ± 2.68 | 14.96 | −5.37 |
| 4.75 | 110.40 | 92.06 ± 3.88 | 17.68 | −8.92 |
| 5.0 | 108.80 | 92.53 ± 2.02 | 18.66 | −6.93 |
| 4.0 | 108.30 | 89.58 ± 2.13 | 7.347 | −5.356 |
| 4.25 | 104.10 | 88.59 ± 3.19 | 7.788 | −6.262 |
| 4.5 | 105.30 | 89.21 ± 3.33 | 8.238 | −6.534 |
| 4.75 | 110.20 | 89.29 ± 2.45 | 11.34 | −4.663 |
| 5.0 | 105.50 | 88.37 ± 2.67 | 12.34 | −6.036 |
| 5.0 | 114.60 | 89.71 ± 3.51 | 23.56 | −0.36 |
| 5.25 | 113.40 | 91.80 ± 5.23 | 16.56 | −2.33 |
| 5.50 | 108.60 | 89.57 ± 2.81 | 17.31 | −2.29 |
| 5.75 | 109.30 | 90.33 ± 1.74 | 24.24 | −1.67 |
| 6.00 | 110.40 | 91.54 ± 4.31 | 19.47 | −3.63 |

TABLE 12B
Luciferase expression (ng/million cells ± stdev)
of KC3-X1-PH12 LNPs in MutuDC 1940 cells after
exposure to LNPs for 24 h at 0.3 μg/mL total
(encapsulated + non-encapsulated) mRNA concentration.

| N/P | Luc activity | Rel. Luc. Activity |
|---|---|---|
| 1.0 | 0.90 ± 0.13 | 1.00 |
| 2.0 | 1.89 ± 1.33 | 2.10 |
| 3.0 | 139.52 ± 5.09 | 155.02 |
| 3.25 | 252.30 ± 11.47 | 280.33 |
| 3.5 | 357.14 ± 11.79 | 396.82 |
| 3.75 | 439.23 ± 27.62 | 488.03 |
| 4.0 | 435.03 ± 38.70 | 483.37 |
| 4.25 | 441.10 ± 38.06 | 490.11 |
| 4.5 | 469.42 ± 37.90 | 521.58 |
| 4.75 | 400.85 ± 20.80 | 445.39 |
| 5.0 | 400.11 ± 36.32 | 444.57 |
| 4.0 | 536.19 ± 25.21 | 595.77 |
| 4.25 | 494.61 ± 42.91 | 549.57 |
| 4.5 | 471.19 ± 63.12 | 523.54 |
| 4.75 | 476.97 ± 60.50 | 529.97 |
| 5.0 | 498.15 ± 50.44 | 553.50 |
| 5.0 | 457.75 ± 28.64 | 508.61 |
| 5.25 | 429.82 ± 32.22 | 477.58 |
| 5.50 | 390.98 ± 19.98 | 434.42 |
| 5.75 | 379.55 ± 29.30 | 421.72 |
| 6.00 | 372.24 ± 26.92 | 413.60 |

LNP formulations prepared with KC3-X1-PH 12 demonstrated a peak luciferase activity at an N/P of 4.5-5.0 with activity dropping off rapidly below N/P 3.5, which also corresponded to increases in particle size and poly dispersity and a decrease in encapsulation efficiency.

### Example 10. Comparing the Immunogenicity of a Quadrivalent mRNA Vaccine in Mice Using KC3-X1-PH12 LNPs and KC3-X2-PH12 LNPs Composed with Different ICL Mol % and N/P Ratios

Four mRNAs encoding influenza A subtypes H1 (SEQ ID NO: 13 encoding SEQ ID NO: 12), H3 (SEQ ID NO: 914 encoding SEQ ID NO: 14), influenza B/Victoria (SEQ ID NO: 17 encoding SEQ ID NO: 915) and RSVpreF (SEQ ID NOs: 10 encoding SEQ ID NO: 9) mRNAs were combined at equal weight ratios and encapsulated into LNPs comprising of KC3-X1-PH12/DSPC/DPPS-NH4/Chol/PEG-DMG where the ICL mol % was 52 or 56 mol %, DSPC and DPPS-NH4 were present at 5 mol % each, PEG-DMG was 1.5 mol % and the remainder lipid was cholesterol. Similar LNPs were prepared with 52 or 56 mol % KC3-X2-PH12 and N/P ratios of 4.5 or 5.25. KC3-X1-PH12 was also used at 52 or 56 mol % and an N/P ratio of 5.25 to encapsulate a single influenza subtype H5 mRNA (SEQ ID NO: 15). The LNPs were prepared as in Example 2 and analyzed as in Example 3. An outline of the composition for formulation and a panel of physicochemical characteristics are shown below in Table 14A. Additionally, a portion of the quadrivalent mRNA-LNPs made with 52 mol % KC3-X1-PH12 and 56 mol % KC3-X1-PH12 were lyophilized. The physicochemical characteristics of these mRNA-LNPs after reconstitution are shown below in Table 14B. LNPs were characterized as described in Example 3. Particle size refers to the Z-Average particle size after freezing and at an N/P ratio. Zeta refers to zeta potential measurements at either pH 5 or pH 7 in units of mV. mRNA encapsulation efficiency after freezing & thawing is referred to as (E.E,%), PDI, polydispersity index.

TABLE 14A
The physicochemical details of the KC3-X1-PH12 or KC3-X2-PH12 based LNP formulations.

| Group # | ICL | N/P | mol % | Z-Ave | PDI | % EE ± stdev | Zeta pH 5 | Zeta pH 7 |
|---|---|---|---|---|---|---|---|---|
| 1-4 | KC3-X1-PH12 | 5.25 | 52 | 111.1 | 0.054 | 88.5 ± 0.25 | 16.76 | 0.47 |
| 5-8 | KC3-X1-PH12 | 5.25 | 56 | 116.1 | 0.047 | 88.4 ± 0.29 | 19.70 | 0.12 |
| 9-10 | KC3-X2-PH12 | 4.25 | 52 | 116.8 | 0.056 | 85.3 ± 1.01 | 9.13 | 0.30 |
| 11-12 | KC3-X2-PH12 | 5.25 | 56 | 106.1 | 0.048 | 93.2 ± 0.75 | 15.49 | 0.62 |
| 13-14 | KC3-X2-PH12 | 5.25 | 52 | 97.66 | 0.051 | 94.7 ± 0.29 | 15.20 | −0.61 |
| 15-16 | KC3-X1-PH12 | 5.25 | 52 | 114.1 | 0.044 | 88.2 ± 0.13 | 20.43 | 0.68 |
| 17-18 | KC3-X1-PH12 | 5.25 | 56 | 108.5 | 0.057 | 89.1 ± 0.43 | 13.16 | 0.72 |

The formulations ranged in size from approximately 97 to 117 nm in diameter. The % entrapped mRNA ranged from 85-94% and LNPs exhibited positive surface charge at pH 5 of approximately ~+9 to +20 mV and a charge at pH 7-1 to +1 mV.

TABLE 14B
Particle size (Z-ave), polydispersity (PDI) and mRNA encapsulation
efficiency (% EE) of LNPs that were lyophilized and reconstituted
(Post Lyo/R), made with either 52 or 56 mol % KC3-X1-PH12.
Compare post lyophilization and reconstitution characteristics
shown for Groups 3-4 to pre lyophilization characteristics
shown for Groups 1-4 in Table 14A. Likewise, compare post
lyophilization and reconstitution characteristics shown
for Groups 7-8 to pre lyophilization characteristics shown
for Groups 5-8 in Table 14A.

| | | | | Post Lyo/R | | |
|---|---|---|---|---|---|---|
| Group # | ICL | N/P | mol % | Z-ave | PDI | % EE |
| 3-4 | KC3-X1-PH12 | 5.25 | 52 | 136.40 | 0.08 | 78.49 ± 1.07 |
| 7-8 | KC3-X1-PH12 | 5.25 | 56 | 140.20 | 0.02 | 75.46 ± 1.60 |

After lyophilization, it was found that the particle size increased by about 25 nm for each, and the % EE was reduced by about 10-12% points.

### In Vivo Immunogenicity in Mice.

To measure the ability of these mRNA-LNPs to induce antibodies, BALB/c mice were immunized at a high and 3-fold lower dose in the caudal thigh in a volume of 50 μL. The experimental design and dosing schema is shown in FIG. 6A. Twenty-one days post vaccination, serum was collected and stored at −80° C. until analysis. Mouse IgG endpoint antibody titers against the matching H1, H3, B-Victora, RSVpreF or H5 antigens were determined by standard ELISA. For H1-specific Ab titers, we found for KC3-X1-PH12 that immunogenicity of freeze-dried material was not deleterious for biological activity (FIG. 6B and FIG. 6C). This was true for 52 and 56 mol % ICL with N/P=5.25, indicating that lyophilization may be an option for longer-term storage. Second, antibody titers were comparable between LNPs comprised of KC3-X1-PH12 and KC3-X2-PH12, showing that either orientation of the biodegradable ester bond can be used for PH12 with a KC3 headgroup. Third, we found for KC3-X1-PH12 that either 52 or 56 mol % with N/P=5.25 elicited similar immune responses; the same was true for X2-PH12. For H3-specific Ab titers (FIG. 6D and FIG. 6E), results were similar to H1 titers: lyophilization of the mRNA-LPN was not deleterious to biological activity (H3 titers elicited by lyophilized material trended higher than for non-lyophilized, X1-PH12 and X2-PH12 were comparably immunogenic, and these ICLs can be used with either 52 or 56 mol % without impacting antibody titers.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

All publications, patents and patent applications referenced in this specification are incorporated herein by reference in their entirety for all purposes to the same extent as if each individual publication, patent or patent application were specifically indicated to be so incorporated by reference.

1. (canceled)

2. A lipid nanoparticle (LNP) composition comprising a mRNA nucleic acid encapsulated in lipidic nanoparticles comprising:
a. a KC3-X1-PH12 or a KC3-X2-PH12 ionizable cationic lipid compound at a N/P ratio of 5.25 relative to the mRNA nucleic acid and in a total amount of 52-56 mol % of the total lipid content of the LNP composition: 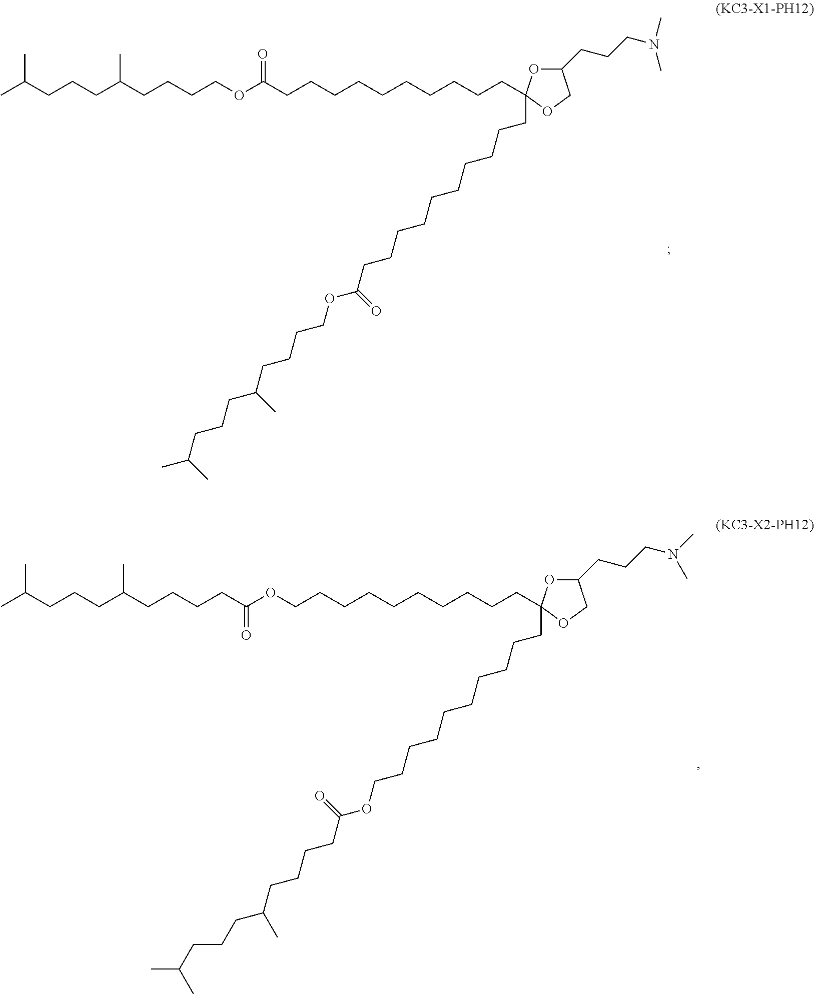
b. dipalmitoylphosphatidyl-L-serine (DPPS) in a total amount of 5 mol % of the total lipid content of the LNP composition;
c. 1,2-distearoyl-sn-glycero-3-phosphocholine (DSPC) in a total amount of 5 mol % of the total lipid content of the LNP composition; and
d. PEG(2000)-dimyristoylglycerol (PEG-DMG) in a total amount of 1.50 mol % of the total lipid content of the LNP composition; and
e. cholesterol in a total remaining amount of the total lipid content of the LNP composition.

3. The composition of claim 2, wherein the ionizable cationic lipid is KC3-X1-PH12.

4. The composition of claim 3, comprising the KC3-X1-PH12 ionizable cationic lipid compound in a total amount of 52 mol % of the total lipid content of the LNP composition.

5. The composition of claim 3, comprising the KC3-X1-PH12 ionizable cationic lipid compound in a total amount of 56 mol % of the total lipid content of the LNP composition.

6. The composition of claim 2, wherein the ionizable cationic lipid is KC3-X2-PH12.

7. The composition of claim 6, comprising the KC3-X2-PH12 ionizable cationic lipid compound in a total amount of 52 mol % of the total lipid content of the LNP composition.

8. The composition of claim 6, comprising the KC3-X2-PH12 ionizable cationic lipid compound in a total amount of 56 mol % of the total lipid content of the LNP composition.

9. The composition of claim 2, wherein the composition is prepared by a process comprising dissolving a DPPS-NH₄ salt in ethanol to obtain a lipid solution and mixing the lipid solution with a second solution comprising the mRNA to obtain the lipid nanoparticles encapsulating the mRNA.

10. The composition of claim 9, wherein the lipid solution further comprises a mixture of DSPC, PEG-DMG, and cholesterol, prior to mixing with the second solution.

11. The composition of claim 10, wherein 94-98% of the mRNA in the composition is entrapped within the lipidic nanoparticles.

12. The composition of claim 2, wherein the composition is frozen and thawed.

13. The composition of claim 2, wherein the lipidic nanoparticles have a Z-Average particle size of 84-140 nm.

14. The composition of claim 13, wherein the lipid nanoparticles have a polydispersity index of 0.006-0.08.

15. The composition of claim 2, wherein the composition is lyophilized.

16. The composition of claim 15, wherein the lyophilized composition is reconstituted.

17. The composition of claim 2, comprising the mRNA nucleic acid encapsulated in lipidic nanoparticles consisting of the ionizable cationic lipid, DPPS, DSPC, PEG-DMG and cholesterol.

18. The composition of claim 2, comprising the mRNA nucleic acid encapsulated in lipidic nanoparticles consisting of 52 mol % of the ionizable cationic lipid, 5 mol % DPPS, 5 mol % DSPC, 1.5 mol % PEG-DMG and 36.5 mol % cholesterol.

19. The composition of claim 18, wherein the ionizable cationic lipid is KC3-X1-PH12.

20. The composition of claim 18, wherein the ionizable cationic lipid is KC3-X2-PH12.

21. The composition of claim 17, wherein the lipidic nanoparticles have a Z-Average particle size of 84-140 nm, and a polydispersity index of 0.006-0.08; and at least 94% of the mRNA in the composition is entrapped within the lipidic nanoparticles.

22. The composition of claim 21, wherein the ionizable cationic lipid is KC3-X1-PH12.
